(19) United States
(12) Patent Application Publication
(10) Pub. No.: US 2025/0243474 A1
(43) Pub. Date: Jul. 31, 2025

(54) GENOME EDITING COMPOSITIONS AND METHODS FOR TREATMENT OF CHRONIC GRANULOMATOUS DISEASE

(71) Applicant: Prime Medicine, Inc., Cambridge, MA (US)

(72) Inventors: Jennifer L. Gori, Jamaica Plain, MA (US); David Waterman, Framingham, MA (US); Jack Heath, Winchester, MA (US); Andrew V. Anzalone, Cambridge, MA (US)

(21) Appl. No.: 18/903,447

(22) Filed: Oct. 1, 2024

Related U.S. Application Data
(63) Continuation of application No. 18/449,230, filed on Aug. 14, 2023, now abandoned.
Application No. 18/449,230 is a continuation of application No. PCT/US2022/074088, filed on Jul. 23, 2022.
(60) Provisional application No. 63/225,100, filed on Jul. 23, 2021.

(51) Int. Cl. C12N 9/22; A61P 37/02; C12N 15/10

(52) U.S. Cl. CPC C12N 9/22; A61P 37/02; C12N 15/102; C12N 2310/20

(57) ABSTRACT

Provided herein are compositions and methods of using prime editing systems comprising prime editors and prime editing guide RNAs for treatment of genetic disorders.

### CROSS-REFERENCE

This application is a continuation of U.S. patent application Ser. No. 18/449,230, filed Aug. 14, 2023, which is a continuation of International Application No. PCT/US2022/074088, filed on Jul. 23, 2022, which claims benefit to U.S. Provisional Application No. 63/225,100, filed Jul. 23, 2021, each of which is incorporated herein by reference in its entirety.

### SEQUENCE LISTING

The instant application contains a Sequence Listing which has been submitted electronically in XML file format and is hereby incorporated by reference in its entirety. Said XML copy, created on Jul. 21, 2022, is named 59761741601_SL.xml and is 31,837,637 bytes in size.

### BACKGROUND

Chronic granulomatous disease (CGD) is an inherited hematologic disorder that leads to a failure of innate immune defense against extracellular pathogens. CGD blood phagocytes (e.g., neutrophils) lack functional activity of the NADPH oxidase rendering patients susceptible to prolonged and recurrent bacterial and fungal infections and inflammatory complications. Despite progress in diagnostic tests that have led to development of more effective prophylactic treatments, the only curative option for patients remains hematopoietic stem cell transplantation (HSCT) from an HLA-matched allogeneic donor. However, HLA-matched donors are not available for all patients and allogeneic HSCT is often complicated by the risks of graft versus host disease and some CGD patients may be ineligible due to ongoing or severe infection.

CGD is caused by mutations in genes encoding the five subunits (gp91phox, p22phox, p40phox, p47phox and p67phox) that comprise the NADPH oxidase enzyme which mediates pathogen clearance via production of reactive oxygen species, activation of granule proteases and formation of neutrophil extracellular traps. p47phox deficiency is the most common among the autosomal recessive forms of CGD accounting for ˜25% of all CGD cases in Europe and North America. P47phox-deficient CGD is caused by mutations in the NCF1 gene which encodes the p47phox protein. A 2-nucleotide deletion (NCF1 c.73_74 GT deletion, also referred to herein as the AGT mutation or delGT mutation) at the start of the exon 2 is the predominant mutation identified in p47phox-deficient patients. NCF1 delGT results in premature termination, undetectable protein expression, and defective production of antimicrobial superoxide in neutrophils.

The human NCF1 gene is located on chromosome 7, adjacent to two pseudogenes, NCF1B and NCF1C. Compared to the wild type NCF1 gene, both NCF1B and NCF1C have a GT dinucleotide deletion in exon 2, which results in a frameshift and premature stop codon. Proximity to pseudogenes also limits the application of ‘classic’ CRISPR gene editing as this may induce multiple double strand breaks at the AGT target sites in NCF1, NCF1B, and NCF1C leading to chromosomal rearrangements that may have safety implications.

This disclosure provides prime editing methods and compositions for correcting mutations associated with CGD.

### SUMMARY OF THE DISCLOSURE

Provided herein, in some embodiments, are methods and compositions for prime editing of alterations in a target sequence in a target gene, for example, an NCF1 gene. The target NCF1 gene may comprise double stranded DNA. As exemplified in FIG. 1, in an embodiment, the target gene is edited by prime editing.

The NCF1 gene has a unique genetic context as it is flanked by two non-functional pseudogenes (NCF1B and NCF1C) that share high sequence homology with NCF1 and contain the exon 2 AGT mutation that is present in NCF1 in >80% p47phox CGD patients. Gene conversion resulting from proximity of NCF1, NCF1B, and NCF1C at chromosome 7q11.23 likely accounts for the predominant AGT mutation characteristic of p47phox-deficient CGD.

Without wishing to be bound by any particular theory, the prime editing process may search specific targets and edit endogenous sequences in a target gene, e.g., the NCF1 gene. As exemplified in FIG. 1, the spacer sequence of a PEgRNA recognizes and anneals with a search target sequence in a target strand of the target gene. A prime editing complex may generate a nick in the target gene on the edit strand which is the complementary strand of the target strand. The prime editing complex may then use a free 3′ end formed at the nick site of the edit strand to initiate DNA synthesis, where a primer binding site (PBS) of the PEgRNA complexes with the free 3′ end, and a single stranded DNA is synthesized using an editing template of the PEgRNA as a template. The editing template may comprise one or more nucleotide edits compared to the endogenous target NCF1 gene sequence. Accordingly, the newly-synthesized single stranded DNA also comprises the nucleotide edit(s) encoded by the editing template. Through removal of an editing target sequence on the edit strand of the target gene and DNA repair, the intended nucleotide edit(s) included in the newly synthesized single stranded DNA are incorporated into the target NCF1 gene.

In one aspect, provided herein is a prime editing guide RNA (PEgRNA) comprising: (a) a spacer that is complementary to a search target sequence on a first strand of a NCF1 gene, wherein the spacer comprises at its 3′ end SEQ ID NO: 19077; (b) a gRNA core capable of binding to a Cas9 protein; and (c) an extension arm comprising: (i) an editing template that comprises a region of complementarity to an editing target sequence on a second strand of the NCF1 gene, and (ii) a primer binding site (PBS) that comprises at its 5′ end a sequence that is a reverse complement of nucleotides 11-13 of SEQ ID NO: 19077; wherein the first strand and second strand are complementary to each other and wherein the editing target sequence comprises a c.73_74 AGT deletion compared to a wildtype NCF gene, and wherein the editing template comprises an AC, CC, GC, or UC dinucleotide insertion compared to the first strand of the NCF gene.

In one aspect, provided herein a prime editing guide RNA (PEgRNA) comprising: (a) a spacer comprising at its 3′ end nucleotides SEQ ID NO: 19077; (b) a gRNA core capable of binding to a Cas9 protein, and (c) an extension arm comprising: (i) an editing template comprising at its 3′ end any one of SEQ ID NOs: 19100-19103, and (ii) a primer binding site (PBS) comprising at its 5′ end a sequence that is a reverse complement of nucleotides 11-13 of SEQ ID NO: 19077.

In some embodiments, the spacer is from 16 to 22 nucleotides in length. In some embodiments, the spacer comprises at its 3 end any one of SEQ ID Nos: 19077-19083. In some embodiments, the spacer comprises at is 3′ end SEQ ID No: 19081. In some embodiments, the spacer is 20 nucleotides in length. In some embodiments, the PEgRNA of any one of aspects above, comprises from 5′ to 3′, the spacer, the gRNA core, the editing template, and the PBS. In some embodiments, the spacer, the gRNA core, and PBS form a contiguous sequence in a single molecule. In some embodiments, the editing template comprises at its 3′ end SEQ ID No: 19103. In some embodiments, the editing template comprises at its 3′ end any one of SEQ ID No:s 19100-19102. In some embodiments, the editing template is at least 13 nucleotides in length and comprises at its 3′ end the sequence corresponding to SEQ ID NO: 19112, 19113, 19114, or 19115. In some embodiments, the editing template comprises at its 3′ end SEQ ID NO: 19112. In some embodiments, the editing template is at least 15 nucleotides in length and comprises at its 3′ end the sequence corresponding to SEQ ID NO: 19120, 19121, 19122, or 19123.

In some embodiments, the editing template is at least 15 nucleotides in length and comprises at its 3′ end the sequence corresponding to SEQ ID NO: 19123. In some embodiments, the editing template is at least 16 nucleotides in length and comprises at its 3′ end the sequence corresponding to SEQ ID NO: 19124, 19125, 19126, or 19127. In some embodiments, the editing template is at least 16 nucleotides in length and comprises at its 3′ end the sequence corresponding to SEQ ID NO: 19125. In some embodiments, the editing template is at least 17 nucleotides in length and comprises at its 3′ end the sequence corresponding to SEQ ID NO: 19128, 19129, 19230, or 19131. In some embodiments, the editing template is at least 17 nucleotides in length and comprises at its 3′ end the sequence corresponding to SEQ ID NO: 19129. In some embodiments, the editing template has a length of 50 nucleotides or less, 40 nucleotides or less, 30 nucleotides or less, or 20 nucleotides or less. In some embodiments, the editing template is 13-17 nucleotide in length and comprises SEQ ID NO:19112, 19116, 19123, 19126, 19129, 19115, 19114, 19118, 19119, 19117, 19122, 19121, 19120, 19127, 19124, 19125, 19131, 19130, or 19128. In some embodiments, the editing template comprises SEQ ID NO: 19112, 19116, 19123, 19126, or 19129. In some embodiments, the editing template comprises SEQ ID NO: 19126 or 19129.

In some embodiments, the PBS is 3 to 19 nucleotides in length. In some embodiments, the PBS comprises at its 5′ a sequence that is a reverse complement of nucleotides 10-13, 9-13, 8-13, 7-13, 6-13, 5-13, 4-13, 3-13, 2-13, or 1-13 of SEQ ID NO. 19077. In some embodiments, the PBS is at least 12 nucleotides in length, at least 13 nucleotides in length, or at least 14 nucleotides in length.

In some embodiments, the PBS is at least 13 nucleotides in length. In some embodiments, the PBS is 12 to 14 nucleotides in length. In some embodiments, the PBS comprises SEQ ID NO: 19093. In some embodiments, the 3′ end of the editing template is adjacent to the 5′ end of the PBS. In some embodiments, the PEgRNA of any one of aspects above, comprises the sequence of SEQ ID NO: 19481, 19482, 19483, 19484, 19486, 19485, 19488, 19490, 19489, 19487, 19493, 19491, 19492, 19495, 19499, 19498, 19502, 19500, 19496, 19501, 19494, 19497, 19503, 19509, 19506, 19507, 19505, 19510, 19504, 19508, 19514, 19519, 19517, 19518, 19511, 19515, 19513, 19516, 19512, 19523, 19527, 19522, 19526, 19525, 19520, 19521, 19524, 19534, 19532, 19530, 19536, 19531, 19529, 19528, 19537, 19533, 19535, 19538, 19541, 19543, 19544, 19542, 19540, 19545, 19539, 19549, 19552, 19551, 19550, 19547, 19548, 19546, 19553, 19554, 19556, 19557, 19555, 19558, 19560, 19559, 19562, 19561, or 19563.

In some embodiments, the PEgRNA of any one of aspects above, comprises the sequence of SEQ ID NO: 19534, 19559, 19550, 19563, 19497, 19531, 19508, 19542, 19516, 19547, 19546, 19512, 19526, 19557, 19524, 19554, 19537, 19560, 19545, 19561, 19535, 19558, 19543, or 19562. In some embodiments, the PEgRNA comprises the sequence of SEQ ID NO. 19560 or 19537. In some embodiments, the PEgRNA comprises the sequence of SEQ ID NO: 19562 or 19543. In some embodiments, the PEgRNA of any one of aspects above, further comprises 3′ mN*mN*mN*N and 5′mN*mN*mN* modifications, where m indicates that the nucleotide contains a 2′-O-Me modification and a * indicates the presence of a phosphorothioate bond.

In one aspect, provided herein is a Prime Editing system comprising: (a) the prime editing guide RNA (PEgRNA) of any one of aspects above, or a polynucleotide encoding the PEgRNA; and (b) a nick guide RNA (ngRNA) comprising (i) a spacer that comprises at its 3′ end nucleotides 5-20 of any one of SEQ ID NOs: 840, 830, 809, 829, 431, 460, 838, 839, 2133, 848, 806, 461, 794, 803, 19478, 2131, 2130, 796, 842, 2139, 856, 849, 833, 828, 462, 467, 810, 464, 843, 832, 801, 2134, 804, 807, 802, 19479, 2138, 800, 857, 792, 2132, 808, 2137, 2135, 19480, 835, 841, 455, 19477, or 2136, and (ii) a gRNA core capable of binding to a Cas9 protein, or a polynucleotide encoding the ngRNA.

In some embodiments, the spacer of the ngRNA comprises at its 3′ end SEQ ID NO 766, 767, 768, 769, 770, 404, 2129, 409, 1820, 772, 774, 407, 406, 405, 777, 790, 408, 829, 431, 460, 838, 839, 2133, 848, 806, 461, 794, 803, 19478, 2131, 2130, 796, 842, 2139, 856, 849, 833, 462, 467, 464, 843, 2134, 19479, 2138, 2132, 2137, 2135, 19480, 841, 455, 19477, 2136, 473, 472, or 479, and the editing template of the PEgRNA comprises SEQ ID NO: 19103 at its 3′ end. In some embodiments, the spacer of the ngRNA comprises SEQ ID NO 766, 767, 768, 769, 770, 829, 839, 803, 849, or 833 at its 3′ end. In some embodiments, the spacer of ngRNA comprises at its 3′ end SEQ ID NO 832, 801, 807, 800, or 792, and the editing template of the PEgRNA comprises SEQ ID NO 19101 at its 3′ end.

In some embodiments, the spacer of ngRNA comprises at its 3′ end SEQ ID NO 809, 828, 802, 857, 808, and the editing template of the PEgRNA comprises SEQ ID NO 19102 at its 3′ end. In some embodiments, the spacer of ngRNA comprises at its 3′ end SEQ ID NO 840, 830, 810, 804, or 835, and the editing template of the PEgRNA comprises SEQ ID NO 19100 at its 3′ end. In some embodiments, the nick-to-nick distance between the nick generated by the ngRNA and the nick generated by the PEgRNA is 4-88 nucleotides.

In some embodiments, the nick-to-nick distance between the nick generated by the ngRNA and the nick generated by the PEgRNA is 4, 5, 6, 7, 61, 72, or 88 nucleotides. In some embodiments, the nick-to-nick distance between the nick generated by the ngRNA and the nick generated by the PEgRNA is 4-7 nucleotides. In some embodiments, the spacer of the ngRNA comprises at its 3′ end SEQ ID NO: 838, 2139, 2130, 2133, 19478, 849, 803, 839, 833, 843, 841, 848, 770, 767, 768, 769, 766, or 842. In some embodiments, the spacer of the ngRNA comprises at its 3′ end SEQ ID No 838, 2139, 2130, 2133, 19478, 849, 803, 839, 833, 843, 841, 848, or 842. In some embodiments, the spacer of the ngRNA comprises at its 3′ end 766, 767, 768, 769, 770, 849, 803, 839, 833, or 842. In some embodiments, the spacer of the ngRNA is 16-22 nucleotides in length, optionally wherein 17 nucleotides in length.

In some embodiments, the spacer of the ngRNA is 20 nucleotides in length. In some embodiments, the spacer of the ngRNA comprises at its 3′ end nucleotides 5-20 of SEQ ID NO 849, nucleotides 5-20 of SEQ ID NO 833, or nucleotides 2-17 of SEQ ID NO 770.

In some embodiments, the spacer of the ngRNA comprises at its 3′ end SEQ ID NO 849, 833, or 770.

In some embodiments, the ngRNA comprises the sequence of SEQ ID NO. 2140, 19564, 877, 878, 881, 879, 880, 19565, 2141, 892, 891, 884, 883, 882, 888, 887, 889, 885, 886, 2142, 19566, 890, 2143, 895, 893, 896, 894, 899, 906, 900, 904, 2144, 903, 905, 2145, 19567, 902, 897, 901, or 898. In some embodiments, the ngRNA comprises the sequence of SEQ ID No 896, 904, 890, 895, 903, 894, 906, 893, 901, 2145, 2144, 19567, 2141, 19565, 769, 803, 766, 768, 839, 767, 833, 770, 849, 2130, 2133, or 19478. In some embodiments, the ngRNA comprises the sequence of SEQ ID No 893, 894, 895, 896, 890, 901, 878, 880, 881, 879, 877, or 888. In some embodiments, the ngRNA comprises the sequence of SEQ ID No 893, 878, 901, 888, 906, or 883.

In one aspect, provided herein is a prime editing guide RNA (PEgRNA) comprising: (a) a spacer that is complementary to a search target sequence on a first strand of an NCF1 gene, wherein the spacer comprises at its 3′ end SEQ ID NO: 3995; (b) a gRNA core capable of binding to a Cas9 protein; and (c) an extension arm comprising: (i) an editing template that comprises a region of complementarity to an editing target sequence on a second strand of the NCF1 gene, and (ii) a primer binding site that comprises at its 5′ end a sequence that is a reverse complement of nucleotides 11-13 of SEQ ID NO: 3995; wherein the first strand and second strand are complementary to each other and wherein the editing target sequence on the second strand is complementary to a portion of the NCF1 gene comprising a c.73_74 ΔGT deletion compared to a wild type NCF1 gene, and wherein the editing template comprises a GU, GA, GG, or GC dinucleotide insertion compared to the first strand of the NCF1 gene.

In one aspect, provided herein is a prime editing guide RNA (PEgRNA) comprising: (a) a spacer comprising at its 3′ end nucleotides SEQ ID NO: 3995; (b) a gRNA core capable of binding to a Cas9 protein, and (c) an extension arm comprising: (i) an editing template comprising at its 3′ end SEQ ID NO: 4019, and (ii) a primer binding site (PBS) comprising at its 5′ end a sequence that is a reverse complement of nucleotides 11-13 of SEQ ID NO: 3995.

In some embodiments, the spacer is from 16 to 22 nucleotides in length. In some embodiments, the spacer comprises at its 3′ end any one of SEQ ID Nos: 3995-4001.

In some embodiments, the spacer comprises at its 3′ end SEQ ID NO: 3999. In some embodiments, the spacer is 20 nucleotides in length. In some embodiments, the PEgRNA of any one of aspects above, comprises from 5′ to 3′, the spacer, the gRNA core, the editing template, and the PBS. In some embodiments, the gRNA core, and PBS form a contiguous sequence in a single molecule. In some embodiments, the editing template comprises at its 3′ end SEQ ID NO 4019, 4020, 4021, 4037, 4054, 4072, 4098, 4102, 4119, 4136, 4149, 4171, 4193, 4209, 4213, 4230, 4245, 4263, 4277, 4305, 4313, 4330, 4348, 4358, 4375, 4402, 4409, 4427, 4439, 4460, 4472, 4490, 4504, 4517, 4533, 4557, 4568, 4584, 4601, 4618, 4630, 4650, 4671, 4681, 4702, 4715, 4732, 4744, 4764, 4785, 4791, 4809, 4826, 4844, 4859, 4870, 4893, 4910, 4920, 4945, 4949, 4971, 4984, 5007, 5020, 5030, 5047, 5061, 5087, 5099, 5110, 5131, 5152, 5157, 5175, 5203, 5217, 5234, 5243, 5258, 5275, 5286, 5309, 5319, 5339, 5349, 5369, 5385, 5399, 5414, 5433, 5458, 5464, or 5478.

In some embodiments, the editing template further encodes a PAM silencing edit. In some embodiments, the editing template encodes a GGG-to-GGC, GGG-to-GAA, GGG-to-GAT, GGG-to-GGT, GGT-to-GTA, GGG-to-GCC, GGG-to-GTG, GGG-to-GTC, GGG-to-GGA, GGG-to-GAC, GGG-to-GCA, GGG-to-GAG, GGG-to-GCT, GGG-to-GTT, or GGG-to-GCG PAM silencing edit on the edit strand, wherein the edit corresponds to positions 74777262-74777264 of human chromosome 7. In some embodiments, the editing template is at least 11 nucleotides in length and comprises at its 3′ end SEQ ID NO 4020. In some embodiments, the editing template is at least 12 nucleotides in length and comprises at its 3′ end SEQ ID NO 4023, 4021, 4034, 4031, 4029, 4025, 4028, 4033, 4026, 4030, 4036, 4035, 4032, 4027, 4024, or 4022. In some embodiments, the editing template is at least 14 nucleotides in length and comprises at its 3′ end SEQ ID NO. 4061, 4056, 4057, 4062, 4067, 4060, 4055, 4058, 4053, 4066, 4068, 4065, 4054, 4063, 4064, or 4059.

In some embodiments, the editing template is at least 16 nucleotides in length and comprises at its 3′ end SEQ ID NO. 4099, 4095, 4093, 4085, 4089, 4100, 4091, 4096, 4097, 4094, 4086, 4088, 4090, 4087, 4092, or 4098. In some embodiments, the editing template is 11-16 nucleotides in length. In some embodiments, the editing template comprises SEQ ID NO. 4020, 4021, 4037, 4054, 4072, or 4098. In some embodiments, the editing template is 12 nucleotides in length and comprises SEQ ID NO 4021. In some embodiments, the PBS is 3 to 19 nucleotides in length. In some embodiments, the PBS comprises at its 5′ a sequence that is a reverse complement of nucleotides 10-13, 9-13, 8-13, 7-13, 6-13, 5-13, 4-13, 3-13, 2-13, or 1-13 of SEQ ID NO. 3995. In some embodiments, the PBS is at least 8 nucleotides in length.

In some embodiments, the PBS is at least 11, 12, 13, or 14 nucleotides in length. In some embodiments, the PBS is 11 to 14 nucleotides in length and comprises SEQ ID NO 4010, 4011, 4012, or 4013.

In some embodiments, the PEgRNA of any one of aspects above, comprises the sequence of SEQ ID NO 5637, 5637, 5682, 5689, 5683, 5690, 5692, 5563, 5606, 5647, 5605, 5644, 5672, 5613, 5537, 5618, 5542, 5661, 5571, 5614, 5543, 5665, 5570, 5638, 5561, 5688, or 5634. In some embodiments, the PEgRNA of any one of aspects above, comprises the sequence of SEQ ID NO 5637, 5563, 5683, 5605, 5659, 5569, 5618, or 5542. In some embodiments, the PEgRNA of any one of aspects above, further comprises 3′ mN*mN*mN*N and 5′mN*mN*mN* modifications, where m indicates that the nucleotide contains a 2′-O-Me modification and a * indicates the presence of a phosphorothioate bond.

In one aspect, provided herein is a Prime Editing system comprising: (a) the prime editing guide RNA (PEgRNA) of any one of aspects above, or a polynucleotide encoding the PEgRNA; and (b) a nick guide RNA (ngRNA) comprising (i) a spacer at its 3′ end nucleotides 5-20 of any one of SEQ ID NOs: 840, 830, 809, 829, 431, 460, 838, 839, 2133, 848, 806, 461, 794, 803, 19478, 2131, 2130, 796, 842, 2139, 856, 849, 833, 828, 462, 467, 810, 464, 843, 832, 801, 2134, 804, 807, 802, 19479, 2138, 800, 857, 792, 2132, 808, 2137, 2135, 19480, 835, 841, 455, 19477, or 2136, and (ii) a gRNA core capable of binding to a Cas9 protein, or a polynucleotide encoding the ngRNA.

In some embodiments, the spacer of the ngRNA comprises at its 3′ end SEQ ID NO 461, 462, 467, 431, 464, 466, 454, 457, 459, 433, 417, 449, 425, 439, 460, 426, 458, 450, 429, 423, 434, 436, 420, 418, 455, 432, 443, 424, 421, 451, 445, 435, 430, 446, 427, 422, 463, 452, 453, 444, 419, 442, 441, 447, 440, 438, or 428. In some embodiments, the spacer of the ngRNA comprises at its 3′ end: SEQ ID NO 5495 wherein the editing template of the PEgRNA comprises at its 3′ end SEQ ID NO 4022, SEQ ID NO 5501 wherein the editing template of the PEgRNA comprises at its 3′ end SEQ ID NO 4025, SEQ ID NO 5510 wherein the editing template of the PEgRNA comprises at its 3′ end SEQ ID NO 4029, SEQ ID NO 5502 wherein the editing template of the PEgRNA comprises at its 3′ end SEQ ID NO 4028, SEQ ID NO 5509 wherein the editing template of the PEgRNA comprises at its 3′ end SEQ ID NO 4026, SEQ ID NO 5496 wherein the editing template of the PEgRNA comprises at its 3′ end SEQ ID NO 4035, SEQ ID NO 5497 wherein the editing template of the PEgRNA comprises at its 3′ end SEQ ID NO 4031, SEQ ID NO 5506 wherein the editing template of the PEgRNA comprises at its 3′ end SEQ ID NO 4024, SEQ ID NO 5504 wherein the editing template of the PEgRNA comprises at its 3′ end SEQ ID NO 4030, SEQ ID NO 5498 wherein the editing template of the PEgRNA comprises at its 3′ end SEQ ID NO 4032, SEQ ID NO 5499 wherein the editing template of the PEgRNA comprises at its 3′ end SEQ ID NO 4027, SEQ ID NO 5508 wherein the editing template of the PEgRNA comprises at its 3′ end SEQ ID NO 4023, SEQ ID NO 5505 wherein the editing template of the PEgRNA comprises at its 3′ end SEQ ID NO 4033, SEQ ID NO 5503 wherein the editing template of the PEgRNA comprises at its 3′ end SEQ ID NO 4034, or SEQ ID 5500 wherein the editing template of the PEgRNA comprises at its 3′ end SEQ ID NO 4036.

In some embodiments, the nick-to-nick distance between the nick generated by the ngRNA and the nick generated by the PEgRNA is 41 to 96 nucleotides. In some embodiments, the nick-to-nick distance between the nick generated by the ngRNA and the nick generated by the PEgRNA is 41, 44, 82, or 96 nucleotides. In some embodiments, the spacer of the ngRNA comprises at its 3′ end nucleotides 5-20 of SEQ ID NO 436, 2130, 442, 421, 445, 451, 424, 422, 429, 436, or 424. In some embodiments, the spacer of the ngRNA is 16-22 nucleotides in length. In some embodiments, the spacer of the ngRNA is 20 nucleotides in length. In some embodiments, the spacer of the ngRNA comprises SEQ ID NO 436, 2130, 442, 421, 445, 451, 424, 422, 429, 436, or 424. In some embodiments, the ngRNA comprises the sequence of 584, 606, 585, 2143, 595, 594, 583, 592, 591, 590, 599, 2145, 604, 597, 602, 596, 598, or 600.

In some embodiments, the ngRNA further comprises 3′ mN*mN*mN*N and 5′mN*mN*mN* modifications, where m indicates that the nucleotide contains a 2′-O-Me modification and a * indicates the presence of a phosphorothioate bond. In some embodiments, the Prime Editing system of any one of aspects above, further comprises: c. a Prime Editor comprising a Cas9 nickase having a nuclease inactivating mutation in the HNH domain, or a polynucleotide encoding the Cas9 nickase, and a reverse transcriptase, or a polynucleotide encoding the reverse transcriptase. In some embodiments, the Cas9 nickase comprises an amino acid sequence having at least 80%, 85%, 90%, 95%, 96%, 97%, 98%, 99%, or 100% identity to SEQ ID NO: 34365. In some embodiments, the reverse transcriptase comprises an amino acid sequence having at least 80%, 85%, 90%, 95%, 96%, 97%, 98%, 99%, or 100% identity to SEQ ID NO: 34363. In some embodiments, the sequence identities are determined by Needleman-Wunsch alignment of two protein sequences with Gap Costs set to Existence: 11 Extension: 1 where percent identity is calculated by dividing the number of identities by the length of the alignment.

In some embodiments, the prime editor is a fusion protein. In some embodiments, the polynucleotide encoding the Cas9 nickase and/or the polynucleotide encoding the reverse transcriptase are mRNA. In some embodiments, the polynucleotide encoding the Cas9 nickase and/or the polynucleotide encoding the reverse transcriptase are the same molecule.

In one aspect, the present disclosure provides a method for editing a NCF/gene, a NCF1B pseudogene, or a NCF1C pseudogene, the method comprising contacting the NCF1 gene, the NCF1B pseudogene, or the NCF1C pseudogene with (a) the PEgRNA of any one of aspects above, or (b) the Prime Editing system of any one of aspects above. In some embodiments, the NCF1 gene, the NCF1B pseudogene, or the NCF1C pseudogene is in a cell. In some embodiments, the cell is a mammalian cell. In some embodiments, the cell is a human cell. In some embodiments, the cell is a hematopoietic stem cell or a hematopoietic pluripotent stem cell.

In one aspect, provided herein is a cell generated by the method of any one of aspects above.

In one aspect, provided herein is a population of cells generated by the method of any one of aspects above.

In one aspect, the present disclosure provides a method of treating chronic granulomatous disease in a subject in need thereof, the method comprising administering to the subject the cell of any one of aspects above or the population of cells of any one of aspects above.

In some embodiments, the cell or the population of cells is obtained from the subject prior to the contacting. In some embodiments, the cell is an allogeneic cell. In one aspect, provides herein is a pharmaceutical composition comprising the cell of any one of aspects above or the population of any one of aspects above.

In one aspect, the present disclosure provides a prime editing guide RNA (PEgRNA) comprising: (a) a spacer comprising at its 3′ end nucleotides a PEgRNA Spacer sequence selected from any one of Tables 1-76; (b) a gRNA core capable of binding to a Cas9 protein, and (c) an extension arm comprising: i. an editing template comprising at its 3′ end an RTT sequence selected from the same Table as the PEgRNA Spacer sequence, and ii. a primer binding site (PBS) comprising at its 5′ end a PBS sequence selected from the same Table as the PEgRNA Spacer sequence.

In some embodiments, the spacer is 16 to 22 nucleotides in length. In some embodiments, the spacer is 20 nucleotides in length. In some embodiments, the PEgRNA of any one of aspects above, comprises from 5′ to 3′, the spacer, the gRNA core, the editing template, and the PBS. In some embodiments, the spacer, the gRNA core, the editing template, and the PBS form a contiguous sequence in a single molecule. In some embodiments, the PEgRNA of any one of aspects above, comprises a pegRNA sequence selected from the same Table as the PEgRNA Spacer sequence.

In one aspect, the present disclosure provides a Prime Editing system comprising (a) the prime editing guide RNA (PEgRNA) of any one of aspects above, or a polynucleotide encoding the PEgRNA; and (b) a nick guide RNA (ngRNA) comprising i. a spacer at its 3′ end nucleotides 5-20 of any ngRNA Spacer sequence selected from the same Table as the PEgRNA Spacer, wherein the ngRNA Spacer sequence in the Table is 20 nucleotides in length, and ii. a gRNA core capable of binding to a Cas9 protein, or a polynucleotide encoding the ngRNA.

In some embodiments, the spacer of the ngRNA is from 16 to 22 nucleotides in length. In some embodiments, the spacer of the ngRNA comprises at its 3′ end nucleotides 4-20, 3-20, 2-20, or 1-20 of the ngRNA Spacer sequence selected from the same Table as the PEgRNA Spacer sequence, wherein the ngRNA Spacer sequence in the Table is 20 nucleotides in length. In some embodiments, the spacer of the ngRNA is 20 nucleotides in length. In some embodiments, the ngRNA comprises a ngRNA sequence selected from the same Table as the PEgRNA Spacer sequence.

In some embodiments, the Prime Editing system of any one of aspects above, further comprises: c. a Prime Editor comprising a Cas9 nickase having a nuclease inactivating mutation in the HNH domain, or a polynucleotide encoding the Cas9 nickase, and a reverse transcriptase, or a polynucleotide encoding the reverse transcriptase. In some embodiments, the Cas9 nickase comprises an amino acid sequence having at least 80%, 85%, 90%, 95%, 96%, 97%, 98%, 99%, or 100% identity to SEQ ID NO: 34365. In some embodiments, the reverse transcriptase comprises an amino acid sequence having at least 80%, 85%, 90%, 95%, 96%, 97%, 98%, 99%, or 100% identity to SEQ ID NO: 34363. In some embodiments, the sequence identities are determined by Needleman-Wunsch alignment of two protein sequences with Gap Costs set to Existence: 11 Extension: 1 where percent identity is calculated by dividing the number of identities by the length of the alignment.

In some embodiments, the prime editor is a fusion protein. In some embodiments, the polynucleotide encoding the Cas9 nickase and/or the polynucleotide encoding the reverse transcriptase are mRNA. In some embodiments, the polynucleotide encoding the Cas9 nickase and/or the polynucleotide encoding the reverse transcriptase are the same molecule.

In one aspect, provides herein is a method for editing a NCF1 gene, a NCF1B pseudogene, or a NCF1C pseudogene, the method comprising contacting the NCF1 gene, the NCF1B pseudogene, or the NCF1C pseudogene with (a) the PEgRNA of any one of aspects above or (b) the Prime Editing system of any one of aspects above.

In some embodiments, the NCF1B pseudogene, or the NCF1C pseudogene is in a cell. In some embodiments, the cell is a mammalian cell. In some embodiments, the cell is a human cell. In some embodiments, the cell is a hematopoietic stem cell or a hematopoietic pluripotent stem cell.

In one aspect, provided herein a cell generated by the method of any one of aspects above.

In one aspect, provided herein is a population of cells generated by the method of any one of aspects above.

In one aspect, the present disclosure provides a method of treating chronic granulomatous disease in a subject in need thereof, the method comprising administering to the subject the cell of any one of aspects above or the population of cells of any one of aspects above.

In some embodiments, the cell or the population of cells is obtained from the subject prior to the contacting. In some embodiments, the cell is an allogeneic cell.

In one aspect, provided herein is a pharmaceutical composition comprising the cell of any one of aspects above, or the population of cells of any one of aspects above.

In one aspect, provided herein is a pharmaceutical composition comprising the PEgRNA of any one of aspects above, or the Prime Editing system of any one of aspects above.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 depicts a schematic of a prime editing guide RNA (PEgRNA) binding to a double stranded target DNA sequence.

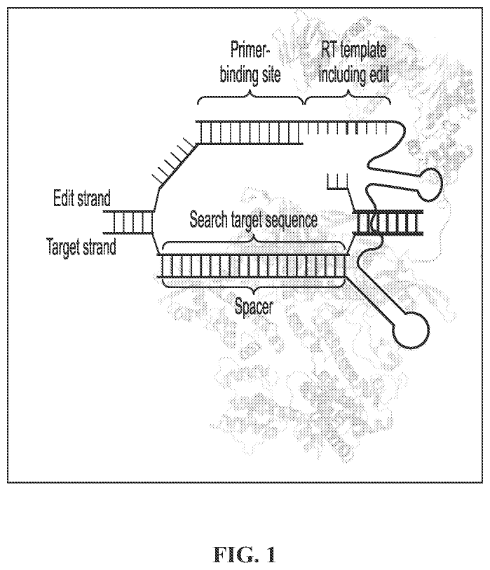

FIG. 2 depicts a PEgRNA architectural overview in an exemplary schematic of PEgRNA designed for a prime editor.

FIG. 3 is a schematic showing the spacer and gRNA core part of an exemplary guide RNA, in two separate molecules. The rest of the PEgRNA structure is not shown.

FIG. 4A is a schematic showing positions of NCF1B, NCF1, NCF1C, and the regions in between each two genes.

FIG. 4B is a schematic showing the probes targeting regions of chromosome 7 between NCF1B and NCF1, between NCF1 and NCF1C, and a reference probe.

### DETAILED DESCRIPTION OF THE DISCLOSURE

Provided herein, in some embodiments, are compositions and methods to edit the target gene NCF1 with prime editing. In some embodiments, provided herein are compositions and methods for editing NCF1B pseudogene or NCF1C pseudogene with prime editing. In certain embodiments, provided herein are compositions and methods for correction of mutations in genes encoding the NCF1 gene associated with chronic granulomatous disease (CGD). Compositions provided herein can comprise prime editors (PEs) that may use engineered guide polynucleotides, e.g., prime editing guide RNAs (PEgRNAs), that can direct PEs to specific DNA targets and can encode DNA edits on the target gene, e.g., NCF1, NCF1B, or NCF1C, that serve a variety of functions, including direct correction of disease-causing mutations.

The following description and examples illustrate embodiments of the present disclosure in detail. It is to be understood that this disclosure is not limited to the particular embodiments described herein and as such can vary. Those of skill in the art will recognize that there are numerous variations and modifications of this disclosure, which are encompassed within its scope. Although various features of the present disclosure can be described in the context of a single embodiment, the features can also be provided separately or in any suitable combination. Conversely, although the present disclosure can be described herein in the context of separate embodiments for clarity, the present disclosure can also be implemented in a single embodiment.

### Definitions

Unless defined otherwise, all technical and scientific terms used herein have the same meaning as is commonly understood by one of ordinary skill in the art.

The terminology used herein is for the purpose of describing particular embodiments only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof as used herein mean “comprising”.

Unless otherwise specified, the words “comprising”, “comprise”, “comprises”, “having”, “have”, “has”, “including”, “includes”, “include”, “containing”, “contains” and “contain” are inclusive or open-ended and do not exclude additional, unrecited elements or method steps.

Reference to “some embodiments”, “an embodiment”, “one embodiment”, or “other embodiments” means that a particular feature or characteristic described in connection with the embodiments is included in at least one or more embodiments, but not necessarily all embodiments, of the present disclosure.

The term “about” or “approximately” in relation to a numerical means, a range of values that fall within 10% greater than or less than the value. For example, about x means x±(10%*x).

In some embodiments, the cell is a human cell. A cell may be of or derived from different tissues, organs, and/or cell types. In some embodiments, the cell is a primary cell. As used herein, the term “primary cell” means a cell isolated from an organism, e.g., a mammal, which is grown in tissue culture (i.e., in vitro) for the first time before subdivision and transfer to a subculture. In some non-limiting examples, mammalian cells, including primary cells and stem cells, can be modified through introduction of one or more polynucleotides, polypeptide, and/or prime editing compositions (e.g., through transfection, transduction, electroporation, and the like) and further passaged. Such modified cells include hepatocytes, fibroblasts, keratinocytes, epithelial cells (e.g., mammary epithelial cells, intestinal epithelial cells), endothelial cells, glial cells, neural cells, formed elements of the blood (e.g., lymphocytes, bone marrow cells), muscle cells and precursors of these somatic cell types. In some embodiments, the cell is a stem cell. In some embodiments, the cell is a progenitor cell. In some embodiments, the cell is a human stem cell. In some embodiments, the cell is a human progenitor cell. In some embodiments, the cell is a pluripotent cell (e.g., a pluripotent stem cell). In some embodiments, the cell (e.g., a stem cell) is an embryonic stem cell, tissue-specific stem cell, mesenchymal stem cell, or an induced pluripotent stem cell. In some embodiments, the cell is an induced pluripotent stem cell (iPSC). In some embodiments, the cell is an embryonic stem cell (ESC). In some embodiments, the cell is a hematopoietic stem cell (HSC) or a hematopoietic stem and progenitor cell (HSPC). In some embodiments, the HSC is from bone marrow or mobilized peripheral blood. In some embodiments the human stem cell is an induced pluripotent stem cell (iPSC). In some embodiments, the cell is a human HSC. In some embodiments, the cell is a human CD34+ cell. In some embodiments, the cell is a hematopoietic stem and progenitor cell (HSPC). In some embodiments, the cell is a human hematopoietic stem and progenitor cell (HSPC). In some embodiments, the cell is a hematopoietic progenitor cell, multipotent progenitor cell, lymphoid progenitor cell, a myeloid progenitor cell, a megakaryocyte-erythroid progenitor cell, a granulocyte-megakaryocyte progenitor cell, a granulocyte, a promyelocyte, a neutrophil, an eosinophil, a basophil, an erythrocyte, a reticulocyte, a thrombocyte, a megakaryoblast, a platelet-producing megakaryocyte, a monocyte, a macrophage, a dendritic cell, a microglia, an osteoclast, a lymphocyte, a NK cell, a B-cell, or a T-cell. In some embodiments, the cell edited by prime editing can be differentiated into, or give rise to recovery of a population of cells, e.g., common lymphoid progenitor cells, common myeloid progenitor cells, megakaryocyte-erythroid progenitor cells, granulocyte-megakaryocyte progenitor cells, granulocytes, promyelocytes, neutrophils, eosinophils, basophils, erythrocytes, reticulocytes, thrombocytes, megakaryoblasts, platelet-producing megakaryocytes, platelets, monocytes, macrophages, dendritic cells, microglia, osteoclasts, lymphocytes, such as NK cells, B-cells or T-cells. In some embodiments, the cell edited by prime editing can be differentiated into, or give rise to recovery of a population of cells, e.g., neutrophils, platelets, red blood cells, monocytes, macrophages, antigen-presenting cells, microglia, osteoclasts, dendritic cells, and/or lymphocytes. In some embodiments, the cell is in a subject, e.g., a human subject. In some embodiments, the cell is obtained from a subject prior to editing. For example, in some embodiments, the cell is obtained from a CGD patient having a mutation in the NCF1 gene. NCF1 c.73_74 GT deletion, may also be referred to herein as the dGT mutation. A patient having a NCF1 c.73_74 GT deletion may be referred as a dGT patient. A patient not having a NCF1 c.73_74 GT deletion may be referred as a non-dGT patient.

In some embodiments, the cell comprises a prime editor, a PEgRNA, or a prime editing composition disclosed herein. In some embodiments, the cell further comprises an ngRNA. In some embodiments, the cell is from a human subject. In some embodiments, the human subject has a disease or condition, or is at a risk of developing a disease or a condition associated with a mutation to be corrected by prime editing, for example, chronic granulomatous disease (CGD). In some embodiments, the cell is from a human subject, and comprises a prime editor, a PEgRNA, or a prime editing composition for correction of the mutation. In some embodiments, the cell is from the human subject and the mutation has been edited or corrected by prime editing.

The term “substantially” as used herein may refer to a value approaching 100% of a given value. In some embodiments, the term may refer to an amount that may be at least about 70%, 80%, 90%, 91%, 92%, 93%, 94%, 95%, 96%, 97%, 98%, 99%, 99.9%, or 99.99% of a total amount. In some embodiments, the term may refer to an amount that may be about 100% of a total amount.

The terms “protein” and “polypeptide” can be used interchangeably to refer to a polymer of two or more amino acids joined by covalent bonds (e.g., an amide bond) that can adopt a three-dimensional conformation. In some embodiments, a protein or polypeptide comprises at least 10 amino acids, 15 amino acids, 20 amino acids, 30 amino acids or 50 amino acids joined by covalent bonds (e.g., amide bonds). In some embodiments, a protein comprises at least two amide bonds. In some embodiments, a protein comprises multiple amide bonds. In some embodiments, a protein comprises an enzyme, enzyme precursor proteins, regulatory protein, structural protein, receptor, nucleic acid binding protein, a biomarker, a member of a specific binding pair (e.g., a ligand or aptamer), or an antibody. In some embodiments, a protein may be a full-length protein (e.g., a fully processed protein having certain biological function). In some embodiments, a protein may be a variant or a fragment of a full-length protein. For example, in some embodiments, a Cas9 protein domain comprises an H840A amino acid substitution compared to a naturally occurring S. pyogenes Cas9 protein. A variant of a protein or enzyme, for example a variant reverse transcriptase, comprises a polypeptide having an amino acid sequence that is about 60% identical, about 70% identical, about 80% identical, about 90% identical, about 95% identical, about 96% identical, about 97% identical, about 98% identical, about 99% identical, about 99.5% identical, or about 99.9% identical to the amino acid sequence of a reference protein.

In some embodiments, a protein comprises one or more protein domains or subdomains. As used herein, the term “polypeptide domain”, “protein domain”, or “domain” when used in the context of a protein or polypeptide, refers to a polypeptide chain that has one or more biological functions, e.g., a catalytic function, a protein-protein binding function, or a protein-DNA function. In some embodiments, a protein comprises multiple protein domains. In some embodiments, a protein comprises multiple protein domains that are naturally occurring. In some embodiments, a protein comprises multiple protein domains from different naturally occurring proteins. For example, in some embodiments, a prime editor may be a fusion protein comprising a Cas9 protein domain of S. pyogenes and a reverse transcriptase protein domain of a retrovirus (e.g., a Moloney murine leukemia virus) or a variant of the retrovirus. A protein that comprises amino acid sequences from different origins or naturally occurring proteins may be referred to as a fusion, or chimeric protein.

In some embodiments, a protein comprises a functional variant or functional fragment of a full-length wild type protein. A “functional fragment” or “functional portion”, as used herein, refers to any portion of a reference protein (e.g., a wild type protein) that encompasses less than the entire amino acid sequence of the reference protein while retaining one or more of the functions, e.g., catalytic or binding functions. For example, a functional fragment of a reverse transcriptase may encompass less than the entire amino acid sequence of a wild type reverse transcriptase but retains the ability under at least one set of conditions to catalyze the polymerization of a polynucleotide. When the reference protein is a fusion of multiple functional domains, a functional fragment thereof may retain one or more of the functions of at least one of the functional domains. For example, a functional fragment of a Cas9 may encompass less than the entire amino acid sequence of a wild type Cas9 but retains its DNA binding ability and lacks its nuclease activity partially or completely.

A “functional variant” or “functional mutant”, as used herein, refers to any variant or mutant of a reference protein (e.g., a wild type protein) that encompasses one or more alterations to the amino acid sequence of the reference protein while retaining one or more of the functions, e.g., catalytic or binding functions. In some embodiments, the one or more alterations to the amino acid sequence comprises amino acid substitutions, insertions or deletions, or any combination thereof. In some embodiments, the one or more alterations to the amino acid sequence comprises amino acid substitutions. For example, a functional variant of a reverse transcriptase may comprise one or more amino acid substitutions compared to the amino acid sequence of a wild type reverse transcriptase but retains the ability under at least one set of conditions to catalyze the polymerization of a polynucleotide. When the reference protein is a fusion of multiple functional domains, a functional variant thereof may retain one or more of the functions of at least one of the functional domains. For example, in some embodiments, a functional fragment of a Cas9 may comprise one or more amino acid substitutions in a nuclease domain, e.g., an H840A amino acid substitution, compared to the amino acid sequence of a wild type Cas9, but retains the DNA binding ability and lacks the nuclease activity partially or completely.

The term “function” and its grammatical equivalents as used herein may refer to a capability of operating, having, or serving an intended purpose. Functional may comprise any percent from baseline to 100% of an intended purpose. For example, functional may comprise or comprise about 5%, 10%, 15%, 20%, 25%, 30%, 35%, 40%, 45%, 50%, 55%, 60%, 65%, 70%, 75%, 80%, 85%, 90%, 95%, or up to about 100% of an intended purpose. In some embodiments, the term functional may mean over or over about 100% of normal function, for example, 125%, 150%, 175%, 200%, 250%, 300%, 400%, 500%, 600%, 700% or up to about 1000% of an intended purpose.

In some embodiments, a protein or polypeptides includes naturally occurring amino acids (e.g., one of the twenty amino acids commonly found in peptides synthesized in nature, and known by the one letter abbreviations A, R, N, C, D, Q, E, G, H, I, L, K, M, F, P, S, T, W, Y and V). In some embodiments, a protein or polypeptides includes non-naturally occurring amino acids (e.g., amino acids which is not one of the twenty amino acids commonly found in peptides synthesized in nature, including synthetic amino acids, amino acid analogs, and amino acid mimetics). In some embodiments, a protein or polypeptide is modified.

In some embodiments, a protein comprises an isolated polypeptide. The term “isolated” means free or removed to varying degrees from components which normally accompany it as found in the natural state or environment. For example, a polypeptide naturally present in a living animal is not isolated, and the same polypeptide partially or completely separated from the coexisting materials of its natural state is isolated.

In some embodiments, a protein is present within a cell, a tissue, an organ, or a virus particle. In some embodiments, a protein is present within a cell or a part of a cell (e.g., a bacteria cell, a plant cell, or an animal cell). In some embodiments, the cell is in a tissue, in a subject, or in a cell culture. In some embodiments, the cell is a microorganism (e.g., a bacterium, fungus, protozoan, or virus). In some embodiments, a protein is present in a mixture of analytes (e.g., a lysate). In some embodiments, the protein is present in a lysate from a plurality of cells or from a lysate of a single cell.

The terms “homologous,” “homology,” or “percent homology” as used herein refer to the degree of sequence identity between an amino acid and a corresponding reference amino acid sequence or a polynucleotide sequence and a corresponding reference polynucleotide sequence. “Homology” can refer to polymeric sequences, e.g., polypeptide or DNA sequences that are similar. Homology can mean, for example, nucleic acid sequences with at least about: 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity. In other embodiments, a “homologous sequence” of nucleic acid sequences may exhibit 93%, 95% or 98% sequence identity to the reference nucleic acid sequence. For example, a “region of homology to a genomic region” can be a region of DNA that has a similar sequence to a given genomic region in the genome. A region of homology can be of any length that is sufficient to promote binding of a spacer, a primer binding site, or protospacer sequence to the genomic region. For example, the region of homology can comprise at least 5, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100 or more bases in length such that the region of homology has sufficient homology to undergo binding with the corresponding genomic region.

When a percentage of sequence homology or identity is specified, in the context of two nucleic acid sequences or two polypeptide sequences, the percentage of homology or identity generally refers to the alignment of two or more sequences across a portion of their length when compared and aligned for maximum correspondence. When a position in the compared sequence can be occupied by the same base or amino acid, then the molecules can be homologous at that position. Unless stated otherwise, sequence homology or identity is assessed over the specified length of the nucleic acid, polypeptide or portion thereof. In some embodiments, the homology or identity is assessed over a functional portion or specified portion of the length.

Alignment of sequences for assessment of sequence homology can be conducted by algorithms known in the art, such as the Basic Local Alignment Search Tool (BLAST) algorithm, which is described in Altschul et al, J. Mol. Biol. 215:403-410, 1990. A publicly available, internet interface, for performing BLAST analyses is accessible through the National Center for Biotechnology Information. Additional known algorithms include those published in: Smith & Waterman, “Comparison of Biosequences”, Adv. Appl. Math. 2:482, 1981; Needleman & Wunsch, “A general method applicable to the search for similarities in the amino acid sequence of two proteins” J. Mol. Biol. 48:443, 1970; Pearson & Lipman “Improved tools for biological sequence comparison”, Proc. Natl. Acad. Sci. USA 85:2444, 1988; or by automated implementation of these or similar algorithms. Global alignment programs may also be used to align similar sequences of roughly equal size. Examples of global alignment programs include NEEDLE (available at www.ebi.ac.uk/Tools/psa/emboss needle/) which is part of the EMBOSS package (Rice P et al., Trends Genet., 2000; 16: 276-277), and the GGSEARCH program https://fasta.bioch.virginia.edu/fasta_www2/, which is part of the FASTA package (Pearson W and Lipman D, 1988, Proc. Natl. Acad. Sci. USA, 85: 2444-2448). Both of these programs are based on the Needleman-Wunsch algorithm which is used to find the optimum alignment (including gaps) of two sequences along their entire length. A detailed discussion of sequence analysis can also be found in Unit 19.3 of Ausubel et al (“Current Protocols in Molecular Biology” John Wiley & Sons Inc, 1994-1998, Chapter 15, 1998). In some embodiments, alignment between a query sequence and a reference sequence is performed with Needleman-Wunsch alignment with Gap Costs set to Existence: 11 Extension: 1 where percent identity is calculated by dividing the number of identities by the length of the alignment, as further described in Altschul et al. (“Gapped BLAST and PSI-BLAST: a new generation of protein database search programs”, Nucleic Acids Res. 25:3389-3402, 1997) and Altschul et al, (“Protein database searches using compositionally adjusted substitution matrices”, FEBS J. 272:5101-5109, 2005).

A skilled person understands that amino acid (or nucleotide) positions may be determined in homologous sequences based on alignment, for example, “H840” in a reference Cas9 sequence may correspond to H839, or another position in a Cas9 homolog.

The term “polynucleotide” or “nucleic acid molecule” can be any polymeric form of nucleotides, including DNA, RNA, a hybridization thereof, or RNA-DNA chimeric molecules. In some embodiments, a polynucleotide comprises cDNA, genomic DNA, mRNA, tRNA, rRNA, or microRNA. In some embodiments, a polynucleotide is double stranded, e.g., a double-stranded DNA in a gene. In some embodiments, a polynucleotide is single-stranded or substantially single-stranded, e.g., single-stranded DNA or an mRNA. In some embodiments, a polynucleotide is a cell-free nucleic acid molecule. In some embodiments, a polynucleotide circulates in blood. In some embodiments, a polynucleotide is a cellular nucleic acid molecule. In some embodiments, a polynucleotide is a cellular nucleic acid molecule in a cell circulating in blood.

Polynucleotides can have any three-dimensional structure. The following are nonlimiting examples of polynucleotides: a gene or gene fragment (for example, a probe, primer, EST or SAGE tag), an exon, an intron, intergenic DNA (including, without limitation, heterochromatic DNA), messenger RNA (mRNA), transfer RNA (tRNA), ribosomal RNA (rRNA), a ribozyme, cDNA, a recombinant polynucleotide, a branched polynucleotide, a plasmid, a vector, isolated DNA, isolated RNA, sgRNA, guide RNA, a nucleic acid probe, a primer, an snRNA, a long non-coding RNA, a snoRNA, a siRNA, a miRNA, a tRNA-derived small RNA (tsRNA), an antisense RNA, an shRNA, or a small rDNA-derived RNA (srRNA).

In some embodiments, a polynucleotide comprises deoxyribonucleotides, ribonucleotides or analogs thereof. In some embodiments, a polynucleotide comprises modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component.

In some embodiments, a polynucleotide is composed of a specific sequence of four nucleotide bases: adenine (A); cytosine (C); guanine (G); thymine (T); and uracil (U) for thymine when the polynucleotide is RNA. In some embodiments, the polynucleotide may comprise one or more other nucleotide bases, such as inosine (I), which is read by the translation machinery as guanine (G).

In some embodiments, a polynucleotide may be modified. As used herein, the terms “modified” or “modification” refers to chemical modification with respect to the A, C, G, T and U nucleotides. In some embodiments, modifications may be on the nucleoside base and/or sugar portion of the nucleosides that comprise the polynucleotide. In some embodiments, the modification may be on the internucleoside linkage (e.g., phosphate backbone). In some embodiments, multiple modifications are included in the modified nucleic acid molecule. In some embodiments, a single modification is included in the modified nucleic acid molecule.

The term “complement”, “complementary”, or “complementarity” as used herein, refers to the ability of two polynucleotide molecules to base pair with each other. Complementary polynucleotides may base pair via hydrogen bonding, which may be Watson Crick, Hoogsteen or reversed Hoogsteen hydrogen bonding. For example, an adenine on one polynucleotide molecule will base pair to a thymine or an uracil on a second polynucleotide molecule and a cytosine on one polynucleotide molecule will base pair to a guanine on a second polynucleotide molecule. Two polynucleotide molecules are complementary to each other when a first polynucleotide molecule comprising a first nucleotide sequence can base pair with a second polynucleotide molecule comprising a second nucleotide sequence. For instance, the two DNA molecules 5′-ATGC-3′ and 5′-GCAT-3′ are complementary, and the complement of the DNA molecule 5′-ATGC-3′ is 5′-GCAT-3′. A percentage of complementarity indicates the percentage of nucleotides in a polynucleotide molecule which can base pair with a second polynucleotide molecule (e.g., 5, 6, 7, 8, 9, 10 out of 10 being 50%, 60%, 70%, 80%, 90%, and 100% complementary, respectively). “Perfectly complementary” means that all the contiguous nucleotides of a polynucleotide molecule will base pair with the same number of contiguous nucleotides in a second polynucleotide molecule. “Substantially complementary” as used herein refers to a degree of complementarity that can be 70%, 75%, 80%, 85%, 90%, 95%, 97%, 98%, or 99% over all or a portion of two polynucleotide molecules. In some embodiments, the portion of complementarity may be a region of 10, 15, 20, 25, 30, 35, 40, 45, 50, or more nucleotides. “Substantial complementary” can also refer to a 100% complementarity over a portion or a region of two polynucleotide molecules. In some embodiments, the portion or the region of complementarity between the two polynucleotide molecules is at least 60%, 65%, 70%, 75%, 80%, 85%, 90%, 95%, 97%, 98%, or 99% of the length of at least one of the two polynucleotide molecules or a functional or defined portion thereof.

As used herein, “expression” refers to the process by which polynucleotides are transcribed into mRNA and/or the process by which polynucleotides, e.g., the transcribed mRNA, translated into peptides, polypeptides, or proteins. If the polynucleotide is derived from genomic DNA, expression may include splicing of the mRNA in a eukaryotic cell. In some embodiments, expression of a polynucleotide, e.g., a gene or a DNA encoding a protein, is determined by the amount of the protein encoded by the gene after transcription and translation of the gene. In some embodiments, expression of a polynucleotide, e.g., a gene or a DNA encoding a protein, is determined by the amount of a functional form of the protein encoded by the gene after transcription and translation of the gene. In some embodiments, expression of a gene is determined by the amount of the mRNA, or transcript, that is encoded by the gene after transcription the gene. In some embodiments, expression of a polynucleotide, e.g., an mRNA, is determined by the amount of the protein encoded by the mRNA after translation of the mRNA. In some embodiments, expression of a polynucleotide, e.g., a mRNA or coding RNA, is determined by the amount of a functional form of the protein encoded by the polypeptide after translation of the polynucleotide.

The term “sequencing” as used herein, may comprise capillary sequencing, bisulfite-free sequencing, bisulfite sequencing, TET-assisted bisulfite (TAB) sequencing, ACE-sequencing, high-throughput sequencing, Maxam-Gilbert sequencing, massively parallel signature sequencing, Polony sequencing, 454 pyrosequencing, Sanger sequencing, Illumina sequencing, SOLiD sequencing, Ion Torrent semiconductor sequencing, DNA nanoball sequencing, Heliscope single molecule sequencing, single molecule real time (SMRT) sequencing, nanopore sequencing, shot gun sequencing, RNA sequencing, or any combination thereof.

The terms “equivalent” or “biological equivalent” are used interchangeably when referring to a particular molecule, or biological or cellular material, and means a molecule having minimal homology to another molecule while still maintaining a desired structure or functionality.

The term “encode” as it is applied to polynucleotides refers to a polynucleotide which is said to “encode” another polynucleotide, a polypeptide, or an amino acid if, in its native state or when manipulated by methods well known to those skilled in the art, it can be used as polynucleotide synthesis template, e.g., transcribed into an RNA, reverse transcribed into a DNA or cDNA, and/or translated to produce an amino acid, or a polypeptide or fragment thereof. In some embodiments, a polynucleotide comprising three contiguous nucleotides form a codon that encodes a specific amino acid. In some embodiments, a polynucleotide comprises one or more codons that encode a polypeptide. In some embodiments, a polynucleotide comprising one or more codons comprises a mutation in a codon compared to a wild-type reference polynucleotide. In some embodiments, the mutation in the codon encodes an amino acid substitution in a polypeptide encoded by the polynucleotide as compared to a wild-type reference polypeptide.

The term “mutation” as used herein refers to a change and/or alteration in an amino acid sequence of a protein or nucleic acid sequence of a polynucleotide. Such changes and/or alterations may comprise the substitution, insertion, deletion and/or truncation of one or more amino acids, in the case of an amino acid sequence, and/or nucleotides, in the case of nucleic acid sequence, compared to a reference amino acid or a reference nucleic acid sequence. In some embodiments, the reference sequence is a wild-type sequence. In some embodiments, a mutation in a nucleic acid sequence of a polynucleotide encodes a mutation in the amino acid sequence of a polypeptide. In some embodiments, the mutation in the amino acid sequence of the polypeptide or the mutation in the nucleic acid sequence of the polynucleotide is a mutation associated with a disease state.

The term “subject” and its grammatical equivalents as used herein may refer to a human or a non-human. A subject may be a mammal. A human subject may be male or female. A human subject may be of any age. A subject may be a human embryo. A human subject may be a newborn, an infant, a child, an adolescent, or an adult. A human subject may be in need of treatment for a genetic disease or disorder.

The terms “treatment” or “treating” and their grammatical equivalents may refer to the medical management of a subject with an intent to cure, ameliorate, or ameliorate a symptom of, a disease, condition, or disorder. Treatment may include active treatment, that is, treatment directed specifically toward the improvement of a disease, condition, or disorder. Treatment may include causal treatment, that is, treatment directed toward removal of the cause of the associated disease, condition, or disorder. In addition, this treatment may include palliative treatment, that is, treatment designed for the relief of symptoms rather than the curing of the disease, condition, or disorder. Treatment may include supportive treatment, that is, treatment employed to supplement another specific therapy directed toward the improvement of the disease, condition, or disorder. In some embodiments, a condition may be pathological. In some embodiments, a treatment may not completely cure or prevent a disease, condition, or disorder. In some embodiments, a treatment ameliorates, but does not completely cure or prevent a disease, condition, or disorder. In some embodiments, a subject may be treated for 12 hours, 24 hours, 2 days, 3 days, 4 days, 5 days, 6 days, 7 days, 2 weeks, 3 weeks, 4 weeks, 2 months, 3 months, 4 months, 5 months, 6 months, 1 year, 2 years, 3 years, 4 years, 5 years, 6 years, indefinitely, or life of the subject.

The term “ameliorate” and its grammatical equivalents means to decrease, suppress, attenuate, diminish, arrest, or stabilize the development or progression of a disease.

The terms “prevent” or “preventing” means delaying, forestalling, or avoiding the onset or development of a disease, condition, or disorder for a period of time. Prevent also means reducing risk of developing a disease, disorder, or condition. Prevention includes minimizing or partially or completely inhibiting the development of a disease, condition, or disorder. In some embodiments, a composition, e.g. a pharmaceutical composition, prevents a disorder by delaying the onset of the disorder for 12 hours, 24 hours, 2 days, 3 days, 4 days, 5 days, 6 days, 7 days, 2 weeks, 3 weeks, 4 weeks, 2 months, 3 months, 4 months, 5 months, 6 months, 1 year, 2 years, 3 years, 4 years, 5 years, 6 years, indefinitely, or life of a subject.

The term “effective amount” or “therapeutically effective amount” refers to a quantity of a composition, for example a prime editing composition comprising a construct, that can be sufficient to result in a desired activity upon introduction into a subject as disclosed herein. An effective amount of the prime editing compositions can be provided to the target gene or cell, whether the cell is ex vivo or in vivo. An effective amount can be the amount to induce, for example, at least about a 2-fold change (increase or decrease) or more in the amount of target nucleic acid modulation (e.g., expression of NCF1 gene, NCF1B pseudogene, or NCF1C pseudogene to produce functional p47phox or NCF1 protein) observed relative to a negative control. An effective amount or dose can induce, for example, about 2-fold increase, about 3-fold increase, about 4-fold increase, about 5-fold increase, about 6-fold increase, about 7-fold increase, about 8-fold increase, about 9-fold increase, about 10-fold increase, about 25-fold increase, about 50-fold increase, about 100-fold increase, about 200-fold increase, about 500-fold increase, about 700-fold increase, about 1000-fold increase, about 5000-fold increase, or about 10,000-fold increase in target gene modulation (e.g., expression of a target NCF1 gene to produce functional p47phox or NCF1 protein). The amount of target gene modulation may be measured by any suitable method known in the art. In some embodiments, the “effective amount” or “therapeutically effective amount” is the amount of a composition that is required to ameliorate the symptoms of a disease relative to an untreated patient. In some embodiments, an effective amount is the amount of a composition sufficient to introduce an alteration in a gene of interest in a cell (e.g., a cell in vitro or in vivo).

In some embodiments, an effective amount can be an amount to induce, when administered to a population of cells, a certain percentage of the population of cells to have a correction of a mutation. For example, in some embodiments, an effective amount can be the amount to induce, when administered to or introduced to a population of cells, installation of one or more intended nucleotide edits that correct a mutation in the target NCF1 gene, in at least about 1%, 2%, 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 99% of the population of cells.

As used herein, the terms “CGD,” “chronic granulomatous disease,” and “Chronic Granulomatous Disease” are used interchangeably. CGD is disease caused by defects in any 1 of 5 subunits of the phagocyte nicotinamide adenine dinucleotide phosphate (NADPH) oxidase (phox), characterized by a failure of phagocytes (neutrophils, monocytes, macrophages, and eosinophils) to generate superoxide anion radical and other related reactive oxygen species (ROS), leading to recurrent infections, inflammation, and increased mortality. In some embodiments, mutations in the NCF1 gene are associated with diseases including CGD. The NCF1 gene codes the p47phox protein. Alternate names for NCF1 include: 47 kDa autosomal chronic granulomatous disease protein, 47 kDa neutrophil oxidase factor, NCF-47K, Neutrophil NADPH oxidase factor 1, Nox organizer 2, Nox-organizing protein 2, SH3 and PX domain-containing protein 1A, or p47-phox. In the human genome the NCF1 gene is located on chromosome 7q11.23 and contains 10 introns and 11 exons, for a total genomic length of 1.459 kb.

### Prime Editing

The term “prime editing” refers to programmable editing of a target DNA using a prime editor complexed with a PEgRNA to incorporate an intended nucleotide edit (also referred to herein as a nucleotide change) into the target DNA through target-primed DNA synthesis. A target gene of prime editing may comprise a double stranded DNA molecule having two complementary strands: a first strand that may be referred to as a “target strand” or a “non-edit strand”, and a second strand that may be referred to as a “non-target strand,” or an “edit strand.” In some embodiments, in a prime editing guide RNA (PEgRNA), a spacer sequence is complementary or substantially complementary to a specific sequence on the target strand, which may be referred to as a “search target sequence”. In some embodiments, the spacer sequence anneals with the target strand at the search target sequence. The target strand may also be referred to as the “non-Protospacer Adjacent Motif (non-PAM strand).” In some embodiments, the non-target strand may also be referred to as the “PAM strand”. In some embodiments, the PAM strand comprises a protospacer sequence and optionally protospacer adjacent motif (PAM) sequence. In prime editing using a Cas-protein-based prime editor, a PAM sequence refers to a short DNA sequence immediately adjacent to the protospacer sequence on the PAM strand of the target gene. A PAM sequence may be specifically recognized by a programmable DNA binding protein, e.g., a Cas nickase or a Cas nuclease. In some embodiments, a specific PAM is characteristic of a specific programmable DNA binding protein, e.g., a Cas nickase or a Cas nuclease. A protospacer sequence refers to a specific sequence in the PAM strand of the target gene that is complementary to the search target sequence. In a PEgRNA, a spacer sequence may have a substantially identical sequence as the protospacer sequence on the edit strand of a target gene, except that the spacer sequence may comprise Uracil (U) and the protospacer sequence may comprise Thymine (T).

In some embodiments, the double stranded target DNA comprises a nick site on the PAM strand (or non-target strand). As used herein, a “nick site” refers to a specific position in between two nucleotides or two base pairs of the double stranded target DNA. In some embodiments, the position of a nick site is determined relative to the position of a specific PAM sequence. In some embodiments, the nick site is the particular position where a nick will occur when the double stranded target DNA is contacted with a nickase, for example, a Cas nickase, that recognizes a specific PAM sequence. In some embodiments, the nick site is upstream of a specific PAM sequence on the PAM strand of the double stranded target DNA. In some embodiments, the nick site is downstream of a specific PAM sequence on the PAM strand of the double stranded target DNA. In some embodiments, the nick site is upstream of a PAM sequence recognized by a Cas9 nickase, wherein the Cas9 nickase comprises a nuclease active RuvC domain and a nuclease inactive HNH domain. In some embodiments, the nick site is 3 nucleotides upstream of the PAM sequence, and the PAM sequence is recognized by a Streptococcus pyogenes Cas9 nickase, a P. lavamentivorans Cas9 nickase, a C. diphtheriae Cas9 nickase, a N. cinerea Cas9, a S. aureus Cas9, or a N. lari Cas9 nickase. In some embodiments, the nick site is 3 base pairs upstream of the PAM sequence, and the PAM sequence is recognized by a Cas9 nickase, wherein the Cas9 nickase that comprises a nuclease active RuvC domain and a nuclease inactive HNH domain. In some embodiments, the nick site is 2 nucleotides upstream of the PAM sequence, and the PAM sequence is recognized by a S. thermophilus Cas9 nickase that comprises a nuclease active RuvC domain and a nuclease inactive HNH domain.

A “primer binding site” (also referred to as PBS or primer binding site sequence) is a single-stranded portion of the PEgRNA that comprises a region of complementarity to the PAM strand (i.e., the non-target strand or the edit strand). The PBS is complementary or substantially complementary to a sequence on the PAM strand of the double stranded target DNA that is immediately upstream of the nick site. In some embodiments, in the process of prime editing, the PEgRNA complexes with and directs a prime editor to bind the search target sequence on the target strand of the double stranded target DNA, and generates a nick at the nick site on the non-target strand of the double stranded target DNA. In some embodiments, the PBS is complementary to or substantially complementary to, and can anneal to, a free 3′ end on the non-target strand of the double stranded target DNA at the nick site. In some embodiments, the PBS annealed to the free 3′ end on the non-target strand can initiate target-primed DNA synthesis.

An “editing template” of a PEgRNA is a single-stranded portion of the PEgRNA that is 5′ of the PBS and which encodes a single strand of DNA. The editing template may comprise a region of complementarity to the PAM strand (i.e., the non-target strand or the edit strand), and comprises one or more intended nucleotide edits compared to the endogenous sequence of the double stranded target DNA. In some embodiments, the editing template and the PBS are immediately adjacent to each other. Accordingly, in some embodiments, a PEgRNA in prime editing comprises a single-stranded portion that comprises the PBS and the editing template immediately adjacent to each other. In some embodiments, the single stranded portion of the PEgRNA comprising both the PBS and the editing template is complementary or substantially complementary to an endogenous sequence on the PAM strand (i.e., the non-target strand or the edit strand) of the double stranded target DNA except for one or more non-complementary nucleotides at the intended nucleotide edit position(s). As used herein, regardless of relative 5′-3′ positioning in other context, the relative positions as between the PBS and the editing template, and the relative positions as among elements of a PEgRNA, are determined by the 5′ to 3′ order of the PEgRNA regardless of the position of sequences in the double stranded target DNA that may have complementarity or identity to elements of the PEgRNA. In some embodiments, the editing template is complementary or substantially complementary to a sequence on the PAM strand that is immediately downstream of the nick site, except for one or more non-complementary nucleotides at the intended nucleotide edit positions. The endogenous, e.g., genomic, sequence that is complementary or substantially complementary to the editing template, except for the one or more non-complementary nucleotides at the position corresponding to the intended nucleotide edit, may be referred to as an “editing target sequence”. In some embodiments, the editing template has identity or substantial identity to a sequence on the target strand that is complementary to, or having the same position in the genome as, the editing target sequence, except for one or more insertions, deletions, or substitutions at the intended nucleotide edit positions. In some embodiments, the editing template encodes a single stranded DNA, wherein the single stranded DNA has identity or substantial identity to the editing target sequence except for one or more insertions, deletions, or substitutions at the positions of the one or more intended nucleotide edits. In some embodiments, the editing template may encode the wild-type or non-disease associated gene sequence (or its complement if the edit strand is the antisense strand of a gene). In some embodiments, the editing template may encode the wild-type or non-disease associated protein, but contain one or more synonymous mutations relative to the wild-type or non-disease associated protein coding region. Such synonymous mutations may include, for example, mutations that decrease the ability of a PEgRNA to rebind to the same target sequence once the desired edit is installed in the genome (e.g., synonymous mutations that silence the endogenous PAM sequence or that edit the endogenous protospacer).

In some embodiments, a PEgRNA complexes with and directs a prime editor to bind to the search target sequence of the target gene. In some embodiments, the bound prime editor generates a nick on the edit strand (PAM strand) of the target gene at the nick site. In some embodiments, a primer binding site (PBS) of the PEgRNA anneals with a free 3′ end formed at the nick site, and the prime editor initiates DNA synthesis from the nick site, using the free 3′ end as a primer. Subsequently, a single-stranded DNA encoded by the editing template of the PEgRNA is synthesized. In some embodiments, the newly synthesized single-stranded DNA comprises one or more intended nucleotide edits compared to an endogenous target gene sequence. Accordingly, in some embodiments, the editing template of a PEgRNA is complementary to a sequence in the edit strand except for one or more mismatches at the intended nucleotide edit positions in the editing template. The endogenous, e.g., genomic, sequence that is partially complementary to the editing template may be referred to as an “editing target sequence”. Accordingly, in some embodiments, the newly synthesized single stranded DNA has identity or substantial identity to a sequence in the editing target sequence, except for one or more insertions, deletions, or substitutions at the intended nucleotide edit positions. In some embodiments, the editing template comprises at least 4 contiguous nucleotides of complementarity with the edit strand wherein the at least 4 nucleotides contiguous are located upstream of the 5′ most edit in the editing template.

In some embodiments, the newly synthesized single-stranded DNA equilibrates with the editing target on the edit strand of the target gene for pairing with the target strand of the target gene. In some embodiments, the editing target sequence of the target gene is excised by a flap endonuclease (FEN), for example, FEN1. In some embodiments, the FEN is an endogenous FEN, for example, in a cell comprising the target gene. In some embodiments, the FEN is provided as part of the prime editor, either linked to other components of the prime editor or provided in trans. In some embodiments, the newly synthesized single stranded DNA, which comprises the intended nucleotide edit, replaces the endogenous single stranded editing target sequence on the edit strand of the target gene. In some embodiments, the newly synthesized single stranded DNA and the endogenous DNA on the target strand form a heteroduplex DNA structure at the region corresponding to the editing target sequence of the target gene. In some embodiments, the newly synthesized single-stranded DNA comprising the nucleotide edit is paired in the heteroduplex with the target strand of the target DNA that does not comprise the nucleotide edit, thereby creating a mismatch between the two otherwise complementary strands. In some embodiments, the mismatch is recognized by DNA repair machinery, e.g., an endogenous DNA repair machinery. In some embodiments, through DNA repair, the intended nucleotide edit is incorporated into the target gene.

### Prime Editor

The term “prime editor (PE)” refers to the polypeptide or polypeptide components involved in prime editing, or any polynucleotide(s) encoding the polypeptide or polypeptide components. In various embodiments, a prime editor includes a polypeptide domain having DNA binding activity and a polypeptide domain having DNA polymerase activity. In some embodiments, the prime editor further comprises a polypeptide domain having nuclease activity. In some embodiments, the polypeptide domain having DNA binding activity comprises a nuclease domain or nuclease activity. In some embodiments, the polypeptide domain having nuclease activity comprises a nickase, or a fully active nuclease. As used herein, the term “nickase” refers to a nuclease capable of cleaving only one strand of a double-stranded DNA target. In some embodiments, the prime editor comprises a polypeptide domain that is an inactive nuclease. In some embodiments, the polypeptide domain having programmable DNA binding activity comprises a nucleic acid guided DNA binding domain, for example, a CRISPR-Cas protein, for example, a Cas9 nickase, a Cpf1 nickase, or another CRISPR-Cas nuclease. In some embodiments, the polypeptide domain having DNA polymerase activity comprises a template-dependent DNA polymerase, for example, a DNA-dependent DNA polymerase or an RNA-dependent DNA polymerase. In some embodiments, the DNA polymerase is a reverse transcriptase. In some embodiments, the prime editor comprises additional polypeptides involved in prime editing, for example, a polypeptide domain having 5′ endonuclease activity, e.g., a 5′ endogenous DNA flap endonucleases (e.g., FEN1), for helping to drive the prime editing process towards the edited product formation. In some embodiments, the prime editor further comprises an RNA-protein recruitment polypeptide, for example, a MS2 coat protein.

A prime editor may be engineered. In some embodiments, the polypeptide components of a prime editor do not naturally occur in the same organism or cellular environment. In some embodiments, the polypeptide components of a prime editor may be of different origins or from different organisms. In some embodiments, a prime editor comprises a DNA binding domain and a DNA polymerase domain that are derived from different species. In some embodiments, a prime editor comprises a Cas polypeptide (DNA binding domain) and a reverse transcriptase polypeptide (DNA polymerase) that are derived from different species. For example, a prime editor may comprise a S. pyogenes Cas9 polypeptide and a Moloney murine leukemia virus (M-MLV) reverse transcriptase polypeptide.

In some embodiments, polypeptide domains of a prime editor may be fused or linked by a peptide linker to form a fusion protein. In other embodiments, a prime editor comprises one or more polypeptide domains provided in trans as separate proteins, which are capable of being associated to each other through non-peptide linkages or through aptamers or recruitment sequences. For example, a prime editor may comprise a DNA binding domain and a reverse transcriptase domain associated with each other by an RNA-protein recruitment aptamer, e.g., a MS2 aptamer, which may be linked to a PEgRNA. Prime editor polypeptide components may be encoded by one or more polynucleotides in whole or in part. In some embodiments, a single polynucleotide, construct, or vector encodes the prime editor fusion protein. In some embodiments, multiple polynucleotides, constructs, or vectors each encode a polypeptide domain or portion of a domain of a prime editor, or a portion of a prime editor fusion protein. For example, a prime editor fusion protein may comprise an N-terminal portion fused to an intein-N and a C-terminal portion fused to an intein-C, each of which is individually encoded by an AAV vector.

### Prime Editor Nucleotide Polymerase Domain

In some embodiments, a prime editor comprises a nucleotide polymerase domain, e.g., a DNA polymerase domain. The DNA polymerase domain may be a wild-type DNA polymerase domain, a full-length DNA polymerase protein domain, or may be a functional mutant, a functional variant, or a functional fragment thereof. In some embodiments, the polymerase domain is a template dependent polymerase domain. For example, the DNA polymerase may rely on a template polynucleotide strand, e.g., the editing template sequence, for new strand DNA synthesis. In some embodiments, the prime editor comprises a DNA-dependent DNA polymerase. For example, a prime editor having a DNA-dependent DNA polymerase can synthesize a new single stranded DNA using a PEgRNA editing template that comprises a DNA sequence as a template. In such cases, the PEgRNA is a chimeric or hybrid PEgRNA, and comprising an extension arm comprising a DNA strand. The chimeric or hybrid PEgRNA may comprise an RNA portion (including the spacer and the gRNA core) and a DNA portion (the extension arm comprising the editing template that includes a strand of DNA).

In some embodiments, the DNA polymerases can be wild type polymerases from eukaryotic, prokaryotic, archaeal, or viral organisms, and/or the polymerases may be modified by genetic engineering, mutagenesis, or directed evolution-based processes. The polymerases can be a T7 DNA polymerase, T5 DNA polymerase, T4 DNA polymerase, Klenow fragment DNA polymerase, DNA polymerase III and the like. The polymerases can be thermostable, and can include Taq, Tne, Tma, Pfu, Tfl, Tth, Stoffel fragment, VENT® and DEEPVENT® DNA polymerases, KOD, Tgo, JDF3, and mutants, variants and derivatives thereof.

In some embodiments, the DNA polymerase is a bacteriophage polymerase, for example, a T4, T7, or phi29 DNA polymerase. In some embodiments, the DNA polymerase is an archaeal polymerase, for example, pol I type archaeal polymerase or a pol II type archaeal polymerase. In some embodiments, the DNA polymerase comprises a thermostable archaeal DNA polymerase. In some embodiments, the DNA polymerase comprises a eubacterial DNA polymerase, for example, Pol I, Pol II, or Pol III polymerase. In some embodiments, the DNA polymerase is a Pol I family DNA polymerase. In some embodiments, the DNA polymerase is a E. coli Pol I DNA polymerase. In some embodiments, the DNA polymerase is a Pol II family DNA polymerase. In some embodiments, the DNA polymerase is a Pyrococcus furiosus (Pfu) Pol II DNA polymerase. In some embodiments, the DNA Polymerase is a Pol IV family DNA polymerase. In some embodiments, the DNA polymerase is a E. coli Pol IV DNA polymerase. In some embodiments, the DNA polymerase comprises a eukaryotic DNA polymerase. In some embodiments, the DNA polymerase is a Pol-beta DNA polymerase, a Pol-lambda DNA polymerase, a Pol-sigma DNA polymerase, or a Pol-mu DNA polymerase. In some embodiments, the DNA polymerase is a Pol-alpha DNA polymerase. In some embodiments, the DNA polymerase is a POLA1 DNA polymerase. In some embodiments, the DNA polymerase is a POLA2 DNA polymerase. In some embodiments, the DNA polymerase is a Pol-delta DNA polymerase. In some embodiments, the DNA polymerase is a POLD1 DNA polymerase. In some embodiments, the DNA polymerase is a POLD2 DNA polymerase. In some embodiments, the DNA polymerase is a human POLD1 DNA polymerase. In some embodiments, the DNA polymerase is a human POLD2 DNA polymerase. In some embodiments, the DNA polymerase is a POLD3 DNA polymerase. In some embodiments, the DNA polymerase is a POLD4 DNA polymerase. In some embodiments, the DNA polymerase is a Pol-epsilon DNA polymerase. In some embodiments, the DNA polymerase is a POLE1 DNA polymerase. In some embodiments, the DNA polymerase is a POLE2 DNA polymerase. In some embodiments, the DNA polymerase is a POLE3 DNA polymerase. In some embodiments, the DNA polymerase is a Pol-eta (POLH) DNA polymerase. In some embodiments, the DNA polymerase is a Pol-iota (POLI) DNA polymerase. In some embodiments, the DNA polymerase is a Pol-kappa (POLK) DNA polymerase. In some embodiments, the DNA polymerase is a Rev1 DNA polymerase. In some embodiments, the DNA polymerase is a human Rev1 DNA polymerase. In some embodiments, the DNA polymerase is a viral DNA-dependent DNA polymerase. In some embodiments, the DNA polymerase is a B family DNA polymerases. In some embodiments, the DNA polymerase is a herpes simplex virus (HSV) UL30 DNA polymerase. In some embodiments, the DNA polymerase is a cytomegalovirus (CMV) UL54 DNA polymerase.

In some embodiments, the DNA polymerase is an archaeal polymerase. In some embodiments, the DNA polymerase is a Family B/pol I type DNA polymerase. For example, in some embodiments, the DNA polymerase is a homolog of Pfu from Pyrococcus furiosus. In some embodiments, the DNA polymerase is a pol II type DNA polymerase. For example, in some embodiments, the DNA polymerase is a homolog of P. furiosus DP1/DP2 2-subunit polymerase. In some embodiments, the DNA polymerase lacks 5′ to 3′ nuclease activity. Suitable DNA polymerases (pol I or pol II) can be derived from archaea with optimal growth temperatures that are similar to the desired assay temperatures.

In some embodiments, the DNA polymerase comprises a thermostable archaeal DNA polymerase. In some embodiments, the thermostable DNA polymerase is isolated or derived from Pyrococcus species (furiosus, species GB-D, woesii, abysii, horikoshii), Thermococcus species (kodakaraensis KOD1, litoralis, species 9 degrees North-7, species JDF-3, gorgonarius), Pyrodictium occultum, and Archaeoglobus fulgidus.

Polymerases may also be from eubacterial species. In some embodiments, the DNA polymerase is a Pol I family DNA polymerase. In some embodiments, the DNA polymerase is an E. coli Pol I DNA polymerase. In some embodiments, the DNA polymerase is a Pol II family DNA polymerase. In some embodiments, the DNA polymerase is a Pyrococcus furiosus (Pfu) Pol II DNA polymerase. In some embodiments, the DNA Polymerase is a Pol III family DNA polymerase. In some embodiments, the DNA Polymerase is a Pol IV family DNA polymerase. In some embodiments, the DNA polymerase is an E. coli Pol IV DNA polymerase. In some embodiments, the Pol I DNA polymerase is a DNA polymerase functional variant that lacks or has reduced 5′ to 3′ exonuclease activity.

Suitable thermostable pol I DNA polymerases can be isolated from a variety of thermophilic eubacteria, including Thermus species and Thermotoga maritima such as Thermus aquaticus (Taq), Thermus thermophilus (Tth) and Thermotoga maritima (Tma UlTma).

In some embodiments, a prime editor comprises an RNA-dependent DNA polymerase domain, for example, a reverse transcriptase (RT). A RT or an RT domain may be a wild type RT domain, a full-length RT domain, or may be a functional mutant, a functional variant, or a functional fragment thereof. An RT or an RT domain of a prime editor may comprise a wild-type RT, or may be engineered or evolved to contain specific amino acid substitutions, truncations, or variants. An engineered RT may comprise sequences or amino acid changes different from a naturally occurring RT. In some embodiments, the engineered RT may have improved reverse transcription activity over a naturally occurring RT or RT domain. In some embodiments, the engineered RT may have improved features over a naturally occurring RT, for example, improved thermostability, reverse transcription efficiency, or target fidelity. In some embodiments, a prime editor comprising the engineered RT has improved prime editing efficiency over a prime editor having a reference naturally occurring RT.

In some embodiments, a prime editor comprises a virus RT, for example, a retrovirus RT. Non-limiting examples of virus RT include Moloney murine leukemia virus (M-MLV or MMLVRT or M-MLV RT); human T-cell leukemia virus type 1 (HTLV-1) RT; bovine leukemia virus (BLV) RT; Rous Sarcoma Virus (RSV) RT; human immunodeficiency virus (HIV) RT, M-MFV RT, Avian Sarcoma-Leukosis Virus (ASLV) RT, Rous Sarcoma Virus (RSV) RT, Avian Myeloblastosis Virus (AMV) RT, Avian Erythroblastosis Virus (AEV) Helper Virus MCAV RT, Avian Myelocytomatosis Virus MC29 Helper Virus MCAV RT, Avian Reticuloendotheliosis Virus (REV-T) Helper Virus REV-A RT, Avian Sarcoma Virus UR2 Helper Virus (UR2AV) RT, Avian Sarcoma Virus Y73 Helper Virus YAV RT, Rous Associated Virus (RAV) RT, and Myeloblastosis Associated Virus (MAV) RT, all of which may be suitably used in the methods and composition described herein.

In some embodiments, the prime editor comprises a wild-type M-MLV RT, a functional mutant, a functional variant, or a functional fragment thereof. In some embodiments, the prime editor comprises a reference M-MLV RT, a functional mutant, a functional variant, or a functional fragment thereof. In some embodiments, the RT domain or a RT is a M-MLV RT (e.g., wild-type M-MLV RT, a functional mutant, a functional variant, or a functional fragment thereof). In some embodiments, the RT domain or a RT is a M-MLV RT (e.g., a reference M-MLV RT, a functional mutant, a functional variant, or a functional fragment thereof). In some embodiments, a M-MLV RT, e.g., reference M-MLV RT, comprises an amino acid sequence as set forth in any one of SEQ ID NO: 34362.

In some embodiments, a reference M-MLV RT is a wild-type M-MLV RT. An exemplary amino acid sequence of a reference M-MLV RT is provided in SEQ ID NO: 34361.

In some embodiments, the prime editor comprises a wild type M-MLV RT. An exemplary amino acid sequence of a wild type M-MLV RT is provided in SEQ ID NO: 34361.

(SEQ ID NO: 34361)
TLNIEDEHRLHETSKEPDVSLGSTWLSDFPQAWAETGGMGLAVRQAPLII
PLKATSTPVSIKQYPMSQEARLGIKPHIQRLLDQGILVPCQSPWNTPLLP
VKKPGTNDYRPVQDLREVNKRVEDIHPTVPNPYNLLSGLPPSHQWYTVLD
LKDAFFCLRLHPTSQPLFAFEWRDPEMGISGQLTWTRLPQGFKNSPTLFD
EALHRDLADFRIQHPDLILLQYVDDLLLAATSELDCQQGTRALLQTLGNL
GYRASAKKAQICQKQVKYLGYLLKEGQRWLTEARKETVMGQPTPKTPRQL
REFLGTAGFCRLWIPGFAEMAAPLYPLTKTGTLFNWGPDQQKAYQEIKQA
LLTAPALGLPDLTKPFELFVDEKQGYAKGVLTQKLGPWRRPVAYLSKKLD
PVAAGWPPCLRMVAAIAVLTKDAGKLTMGQPLVILAPHAVEALVKQPPDR
WLSNARMTHYQALLLDTDRVQFGPVVALNPATLLPLPEEGLQHNCLDILA
EAHGTRPDLTDQPLPDADHTWYTDGSSLLQEGQRKAGAAVTTETEVIWAK
ALPAGTSAQRAELIALTQALKMAEGKKLNVYTDSRYAFATAHIHGEIYRR
RGLLTSEGKEIKNKDEILALLKALFLPKRLSIIHCPGHQKGHSAEARGNR
MADQAARKAAITETPDTSTLLIENSSP.

In some embodiments, the prime editor comprises a reference M-MLV RT. An exemplary amino acid sequence of a reference M-MLV RT is provided in SEQ ID NO: 34362.

(SEQ ID NO: 34362)
TLNIEDEYRLHETSKEPDVSLGSTWLSDFPQAWAETGGMGLAVRQAPLII
PLKATSTPVSIKQYPMSQEARLGIKPHIQRLLDQGILVPCQSPWNTPLLP
VKKPGTNDYRPVQDLREVNKRVEDIHPTVPNPYNLLSGLPPSHQWYTVLD
LKDAFFCLRLHPTSQPLFAFEWRDPEMGISGQLTWTRLPQGFKNSPTLFD
EALHRDLADFRIQHPDLILLQYVDDLLLAATSELDCQQGTRALLQTLGNL
GYRASAKKAQICQKQVKYLGYLLKEGQRWLTEARKETVMGQPTPKTPRQL
REFLGTAGFCRLWIPGFAEMAAPLYPLTKTGTLFNWGPDQQKAYQEIKQA
LLTAPALGLPDLTKPFELFVDEKQGYAKGVLTQKLGPWRRPVAYLSKKLD
PVAAGWPPCLRMVAAIAVLTKDAGKLTMGQPLVILAPHAVEALVKQPPDR
WLSNARMTHYQALLLDTDRVQFGPVVALNPATLLPLPEEGLQHNCLDILA
EAHGTRPDLTDQPLPDADHTWYTDGSSLLQEGQRKAGAAVTTETEVIWAK
ALPAGTSAQRAELIALTQALKMAEGKKLNVYTDSRYAFATAHIHGEIYRR
RGLLTSEGKEIKNKDEILALLKALFLPKRLSIIHCPGHQKGHSAEARGNR
MADQAARKAAITETPDTSTLLIENSSP.

In some embodiments, the prime editor comprises a M-MLV RT comprising one or more of amino acid substitutions P51X, S67X, E69X, L139X, T197X, D200X, H204X, F209X, E302X, T306X, F309X, W313X, T330X, L345X, L435X, N454X, D524X, E562X, D583X, H594X, L603X, E607X, or D653X as compared to the reference M-MLV RT as set forth in SEQ ID NO: 34362, where X is any amino acid other than the original amino acid in the reference M-MLV RT. In some embodiments, the prime editor comprises a M-MLV RT comprising one or more of amino acid substitutions P51L, S67K, E69K, L139P, T197A, D200N, H204R, F209N, E302K, E302R, T306K, F309N, W313F, T330P, L345G, L435G, N454K, D524G, E562Q, D583N, H594Q, L603W, E607K, and D653N as compared to the reference M-MLV RT as set forth in SEQ ID NO: 34362. In some embodiments, the prime editor comprises a M-MLV RT comprising one or more of amino acid substitutions D200N, T330P, L603W, T306K, and W313F as compared to the reference M-MLV RT as set forth in SEQ ID NO: 34362. In some embodiments, the prime editor comprises a M-MLV RT comprising amino acid substitutions D200N, T330P, L603W, T306K, and W313F as compared to a reference M-MLV RT as set forth in SEQ ID NO: 34362. In some embodiments, the prime editor comprises a M-MLV RT comprising one or more of amino acid substitutions D200N, T330P, L603W, T306K, and W313F as compared to a wild type M-MMLV RT as set forth in SEQ ID NO: 34361. In some embodiments, a prime editor may comprise amino acid substitutions D200N, T330P, L603W, T306K, and W313F as compared to a reference M-MLV RT as set forth in SEQ ID NO: 34362. In some embodiments, the prime editor comprises a M-MLV RT that comprises an amino acid sequence that is at least 70%, at least 75%, at least 80%, at least 85%, at least 90%, at least 95%, at least 96%, at least 97%, at least 98%, at least 99%, at least 99.5%, or at least 99.9% identical to an amino acid sequence set forth in any one of SEQ ID NOs: 34361, 34362, or 34363. In some embodiments, the prime editor comprises a M-MLV RT that comprises an amino acid sequence that is selected from the group consisting of SEQ ID NOs: 34361, 34362, and 34363 or a variant or fragment thereof. In some embodiments, the prime editor comprises a M-MLV RT that comprises an amino acid sequence set forth in SEQ ID NO: 34363.

(SEQ ID NO: 34363)
TLNIEDEYRLHETSKEPDVSLGSTWLSDFPQAWAETGGMGLAVRQAPLII
PLKATSTPVSIKQYPMSQEARLGIKPHIQRLLDQGILVPCQSPWNTPLLP
VKKPGTNDYRPVQDLREVNKRVEDIHPTVPNPYNLLSGLPPSHQWYTVLD
LKDAFFCLRLHPTSQPLFAFEWRDPEMGISGQLTWTRLPQGFKNSPTLFN
EALHRDLADFRIQHPDLILLQYVDDLLLAATSELDCQQGTRALLQTLGNL
GYRASAKKAQICQKQVKYLGYLLKEGQRWLTEARKETVMGQPTPKTPRQL
REFLGKAGFCRLFIPGFAEMAAPLYPLTKPGTLFNWGPDQQKAYQEIKQA
LLTAPALGLPDLTKPFELFVDEKQGYAKGVLTQKLGPWRRPVAYLSKKLD
PVAAGWPPCLRMVAAIAVLTKDAGKLTMGQPLVILAPHAVEALVKQPPDR
WLSNARMTHYQALLLDTDRVQFGPVVALNPATLLPLPEEGLQHNCLDILA
EAHGTRPDLTDQPLPDADHTWYTDGSSLLQEGQRKAGAAVTTETEVIWAK
ALPAGTSAQRAELIALTQALKMAEGKKLNVYTDSRYAFATAHIHGEIYRR
RGWLTSEGKEIKNKDEILALLKALFLPKRLSIIHCPGHQKGHSAEARGNR
MADQAARKAAITETPDTSTLLIENSSP.

In some embodiments, an RT variant may be a functional fragment of a reference RT that has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or up to 100, or up to 200, or up to 300, or up to 400, or up to 500 or more amino acid changes compared to a wild type RT (e.g., SEQ ID NO: 34361). In some embodiments, the RT variant comprises a fragment of a wild type RT (e.g., SEQ ID NO: 34361), such that the fragment is about 70% identical, about 80% identical, about 90% identical, about 95% identical, about 96% identical, about 97% identical, about 98% identical, about 99% identical, about 99.5% identical, or about 99.9% identical to the corresponding fragment of the wild type RT (e.g., SEQ ID NO: 34361). In some embodiments, the fragment is 30%, 35%, 40%, 45%, 50%, 55%, 60%, 65%, 70%, 75%, 80%, 85%, 90%, 95% identical, 96%, 97%, 98%, 99%, or 99.5% of the amino acid length of a corresponding wild type RT (M-MLV reverse transcriptase) (e.g., SEQ ID NO: 34361).

In some embodiments, an RT variant may be a functional fragment of a reference RT that has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or up to 100, or up to 200, or up to 300, or up to 400, or up to 500 or more amino acid changes compared to a reference RT, e.g., SEQ ID NO: 34362. In some embodiments, the RT variant comprises a fragment of a reference RT, such that the fragment is about 70% identical, about 80% identical, about 90% identical, about 95% identical, about 96% identical, about 97% identical, about 98% identical, about 99% identical, about 99.5% identical, or about 99.9% identical to the corresponding fragment of a reference RT (e.g., SEQ ID NO: 34362). In some embodiments, the fragment is 30%, 35%, 40%, 45%, 50%, 55%, 60%, 65%, 70%, 75%, 80%, 85%, 90%, 95% identical, 96%, 97%, 98%, 99%, or 99.5% of the amino acid length of a reference RT (e.g., a M-MLV RT) (e.g., SEQ ID NO: 34362).

In some embodiments, the RT functional fragment is at least 100 amino acids in length. In some embodiments, the fragment is at least 100, 150, 200, 250, 300, 350, 400, 450, 500, 550, or up to 600 or more amino acids in length.

In still other embodiments, the functional RT variant is truncated at the N-terminus or the C-terminus, or both, by a certain number of amino acids which results in a truncated variant which still retains sufficient DNA polymerase function. In some embodiments, the functional RT variant, e.g., a functional MMLV RT variant, is truncated at the C-terminus to abolish or reduce RNAaseH activity and still retain DNA polymerase activity.

In some embodiments, a prime editing composition or a prime editing system disclosed herein comprises a polynucleotide (e.g., a DNA, a RNA, e.g., a mRNA) that encodes a M-MLV RT. In some embodiments, the polynucleotide encodes a M-MLV RT that comprises an amino acid sequence that is at least 70%, at least 75%, at least 80%, at least 85%, at least 90%, at least 95%, at least 96%, at least 97%, at least 98%, at least 99%, at least 99.5%, or at least 99.9% identical to an amino acid sequence set forth in any one of SEQ ID NOs: 34361, 34362, or 34363. In some embodiments, the polynucleotide encodes a M-MLV RT that comprises an amino acid sequence that is selected from the group consisting of SEQ ID NOs: 34361, 34362, and 34363. In some embodiments, the polynucleotide encodes a M-MLV RT that comprises an amino acid sequence that is set forth in SEQ ID NO: 34363.

In some embodiments, a prime editor comprises a eukaryotic RT, for example, a yeast, drosophila, rodent, or primate RT. In some embodiments, the prime editor comprises a Group II intron RT, for example, a. Geobacillus stearothermophilus Group II Intron (GsI-IIC) RT or a Eubacterium rectale group II intron (Eu.re.I2) RT. In some embodiments, the prime editor comprises a retron RT. In some embodiments, a prime editor comprises a eukaryotic RT, for example, a yeast, drosophila, rodent, or primate RT. In some embodiments, the prime editor comprises a Group II intron RT, for example, a. Geobacillus stearothermophilus Group II Intron (GsI-IIC) RT or a Eubacterium rectale group II intron (Eu.re.I2) RT. In some embodiments, the prime editor comprises a retron RT.

### Programmable DNA Binding Domain

In some embodiments, the DNA-binding domain of a prime editor is a programmable DNA binding domain. In some embodiments, a prime editor comprises a DNA binding domain that comprises an amino acid sequence that is at least 50%, at least 60%, at least 70%, at least 75%, at least 80%, 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of the sequences set forth in SEQ ID NOs: 34364-34390. In some embodiments, the DNA binding domain comprises an amino acid sequence that has no more than 1, 2, 3, 4, 5, 6, 7, 8, 9, 10, 11, 12, 13, 14, 15, 16, 17, 18, 19, or 20 differences e.g., mutations e.g., deletions, substitutions and/or insertions compared to any one of the amino acid sequences set forth in SEQ ID NOs: 34364-34390. In some embodiments, the DNA binding domain of a prime editor is a programmable DNA binding domain. A programmable DNA binding domain refers to a protein domain that is designed to bind a specific nucleic acid sequence, e.g., a target DNA or a target RNA. In some embodiments, the DNA-binding domain is a polynucleotide programmable DNA-binding domain that can associate with a guide polynucleotide (e.g., a PEgRNA) that guides the DNA-binding domain to a specific DNA sequence, e.g., a search target sequence in a target gene. In some embodiments, the DNA-binding domain comprises a Clustered Regularly Interspaced Short Palindromic Repeats (CRISPR) Associated (Cas) protein. A Cas protein may comprise any Cas protein described herein or a functional fragment or functional variant thereof. In some embodiments, a DNA-binding domain may also comprise a zinc-finger protein domain. In other cases, a DNA-binding domain comprises a transcription activator-like effector domain (TALE). In some embodiments, the DNA-binding domain comprises a DNA nuclease. For example, the DNA-binding domain of a prime editor may comprise an RNA-guided DNA endonuclease, e.g., a Cas protein. In some embodiments, the DNA-binding domain comprises a zinc finger nuclease (ZFN) or a transcription activator like effector domain nuclease (TALEN), where one or more zinc finger motifs or TALE motifs are associated with one or more nucleases, e.g., a Fok I nuclease domain.

In some embodiments, the DNA-binding domain comprises a nuclease activity. In some embodiments, the DNA-binding domain of a prime editor comprises an endonuclease domain having single strand DNA cleavage activity. For example, the endonuclease domain may comprise a FokI nuclease domain. In some embodiments, the DNA-binding domain of a prime editor comprises a nuclease having full nuclease activity. In some embodiments, the DNA-binding domain of a prime editor comprises a nuclease having modified or reduced nuclease activity as compared to a wild type endonuclease domain. For example, the endonuclease domain may comprise one or more amino acid substitutions as compared to a wild type endonuclease domain. In some embodiments, the DNA-binding domain of a prime editor has a nickase activity. In some embodiments, the DNA-binding domain of a prime editor comprises a Cas protein domain that is a nickase. In some embodiments, compared to a wild type Cas protein, the Cas nickase comprises one or more amino acid substitutions in a nuclease domain that reduces or abolishes its double strand nuclease activity but retains DNA binding activity. In some embodiments, the Cas nickase comprises an amino acid substitution in a HNH domain. In some embodiments, the Cas nickase comprises an amino acid substitution in a RuvC domain.

In some embodiments, the DNA-binding domain comprises a CRISPR associated protein (Cas protein) domain. A Cas protein may be a Class 1 or a Class 2 Cas protein. A Cas protein can be a type I, type II, type III, type IV, type V Cas protein, or a type VI Cas protein. Non-limiting examples of Cas proteins include Cas1, Cas1B, Cas2, Cas3, Cas4, Cas5, Cas5d, Cas5t, Cas5h, Cas5a, Cas6, Cas7, Cas8, Cas8a, Cas8b, Cas8c, Cas9 (e.g., Csn1 or Csx12), Cas10, Cas10d, Cas12a/Cpf1, Cas12b/C2c1, Cas12c/C2c3, Cas12d/CasY, Cas12e/CasX, Cas12g, Cas12h, Cas12i, Csy1, Csy2, Csy3, Csy4, Cse1, Cse2, Cse3, Cse4, CseSe, Csc1, Csc2, Csa5, Csn1, Csn2, Csm1, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csx11, Csf1, Csf2, CsO, Csf4, Csd1, Csd2, Cst1, Cst2, Csh1, Csh2, Csa1, Csa2, Csa3, Csa4, Csa5, Type II Cas effector proteins, Type V Cas effector proteins, Type VI Cas effector proteins, CARF, DinG, Cpf1, Cas12b/C2c1, Cas12c/C2c3, Cas12b/C2c1, Cas12c/C2c3, SpCas9(K855A), eSpCas9(1.1), SpCas9-HF1, hyper accurate Cas9 variant (HypaCas9), Cas Φ, and homologues, modified or engineered variants, mutants, and/or functional fragments thereof. A Cas protein can be a chimeric Cas protein that is fused to other proteins or polypeptides. A Cas protein can be a chimera of various Cas proteins, for example, comprising domains of Cas proteins from different organisms. A Cas protein, e.g., Cas9, can be from any suitable organism. In some aspects, the organism is Streptococcus pyogenes (S. pyogenes). In some aspects, the organism is Staphylococcus aureus (S. aureus). In some aspects, the organism is Streptococcus thermophilus (S. thermophilus). In some embodiments, the organism is Staphylococcus lugdunensis.

Non-limiting examples of suitable organism include Streptococcus pyogenes, Streptococcus thermophilus, Streptococcus sp., Staphylococcus aureus, Nocardiopsis dassonvillei, Streptomyces pristinae spiralis, Streptomyces viridochromo genes, Streptomyces viridochromogenes, Streptosporangium roseum, Streptosporangium roseum, AlicyclobacHlus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Pseudomonas aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Acaryochloris marina, Leptotrichia shahii, and Francisella novicida. In some embodiments, the organism is Streptococcus pyogenes (S. pyogenes). In some embodiments, the organism is Staphylococcus aureus (S. aureus). In some embodiments, the organism is Streptococcus thermophilus (S. thermophilus). In some embodiments, the organism is Staphylococcus lugdunensis (S. lugdunensis).

In some embodiments, a Cas protein can be derived from a variety of bacterial species including, but not limited to, Veillonella atypical, Fusobacterium nucleatum, Filifactor alocis, Solobacterium moorei, Coprococcus catus, Treponema denticola, Peptoniphilus duerdenii, Catenibacterium mitsuokai, Streptococcus mutans, Listeria innocua, Staphylococcus pseudintermedius, Acidaminococcus intestine, Olsenella uli, Oenococcus kitaharae, Bifidobacterium bifidum, Lactobacillus rhamnosus, Lactobacillus gasseri, Finegoldia magna, Mycoplasma mobile, Mycoplasma gallisepticum, Mycoplasma ovipneumoniae, Mycoplasma canis, Mycoplasma synoviae, Eubacterium rectale, Streptococcus thermophilus, Eubacterium dolichum, Lactobacillus coryniformis subsp. Torquens, Ilyobacter polytropus, Ruminococcus albus, Akkermansia muciniphila, Acidothermus cellulolyticus, Bifidobacterium longum, Bifidobacterium dentium, Corynebacterium diphtheria, Elusimicrobium minutum, Nitratifractor salsuginis, Sphaerochaeta globus, Fibrobacter succinogenes subsp. Succinogenes, Bacteroides fragilis, Capnocytophaga ochracea, Rhodopseudomonas palustris, Prevotella micans, Prevotella ruminicola, Flavobacterium columnare, Aminomonas paucivorans, Rhodospirillum rubrum, Candidatus Puniceispirillum marinum, Verminephrobacter eiseniae, Ralstonia syzygii, Dinoroseobacter shibae, Azospirillum, Nitrobacter hamburgensis, Bradyrhizobium, Wolinella succinogenes, Campylobacter jejuni subsp. Jejuni, Helicobacter mustelae, Bacillus cereus, Acidovorax ebreus, Clostridium perfringens, Parvibaculum lavamentivorans, Roseburia intestinalis, Neisseria meningitidis, Pasteurella multocida subsp. Multocida, Sutterella wadsworthensis, proteobacterium, Legionella pneumophila, Parasutterella excrementihominis, Wolinella succinogenes, and Francisella novicida.

In some embodiments, a Cas protein, e.g., Cas9, can be a wild type or a modified form of a Cas protein. In some embodiments, a Cas protein, e.g., Cas9, can be a nuclease active variant, nuclease inactive variant, a nickase, or a functional variant or a functional fragment of a wild type Cas protein. In some embodiments, a Cas protein, e.g., Cas9, can be a wild type or a modified form of a Cas protein. A Cas protein, e.g., Cas9, can be a nuclease active variant, nuclease inactive variant, a nickase, or a functional variant or functional fragment of a wild type Cas protein. In some embodiments, a Cas protein, e.g., Cas9, can comprise an amino acid change such as a deletion, insertion, substitution, fusion, chimera, or any combination thereof relative to a corresponding wild-type version of the Cas protein. In some embodiments, a Cas protein can be a polypeptide with at least about 50%, 60%, 70%, 80%, 90%, 91%, 92%, 93%, 94%, 95%, 96%, 97%, 98%, 99%, or 100% sequence identity or sequence similarity to a wild type exemplary Cas protein.

A Cas protein, e.g., Cas9, may comprise one or more domains. Non-limiting examples of Cas domains include, guide nucleic acid recognition and/or binding domain, nuclease domains (e.g., DNase or RNase domains, RuvC, HNH), DNA binding domain, RNA binding domain, helicase domains, protein-protein interaction domains, and dimerization domains. In various embodiments, a Cas protein comprises a guide nucleic acid recognition and/or binding domain can interact with a guide nucleic acid, and one or more nuclease domains that comprise catalytic activity for nucleic acid cleavage.

In some embodiments, a Cas protein, e.g., Cas9, comprises one or more nuclease domains. A Cas protein can comprise an amino acid sequence having at least about 50%, 60%, 70%, 80%, 90%, 91%, 92%, 93%, 94%, 95%, 96%, 97%, 98%, 99%, or 100% sequence identity to a nuclease domain (e.g., RuvC domain, HNH domain) of a wild-type Cas protein. In some embodiments, a Cas protein comprises a single nuclease domain. For example, a Cpf1 may comprise a RuvC domain but lacks HNH domain. In some embodiments, a Cas protein comprises two nuclease domains, e.g., a Cas9 protein can comprise an HNH nuclease domain and a RuvC nuclease domain.

In some embodiments, a prime editor comprises a Cas protein, e.g., Cas9, wherein all nuclease domains of the Cas protein are active. In some embodiments, a prime editor comprises a Cas protein having one or more inactive nuclease domains. One or a plurality of the nuclease domains (e.g., RuvC, HNH) of a Cas protein can be deleted or mutated so that they are no longer functional or comprise reduced nuclease activity. In some embodiments, a Cas protein, e.g., Cas9, comprising mutations in a nuclease domain has reduced (e.g., nickase) or abolished nuclease activity while maintaining its ability to target a nucleic acid locus at a search target sequence when complexed with a guide nucleic acid, e.g., a PEgRNA.

In some embodiments, a prime editor comprises a Cas nickase that can bind to the target gene in a sequence-specific manner and generate a single-strand break at a protospacer within double-stranded DNA in the target gene, but not a double-strand break. For example, the Cas nickase can cleave the edit strand or the non-edit strand of the target gene, but may not cleave both. In some embodiments, a prime editor comprises a Cas nickase comprising two nuclease domains (e.g., Cas9), with one of the two nuclease domains modified to lack catalytic activity or deleted. In some embodiments, the Cas nickase of a prime editor comprises a nuclease inactive RuvC domain and a nuclease active HNH domain. In some embodiments, the Cas nickase of a prime editor comprises a nuclease inactive HNH domain and a nuclease active RuvC domain. In some embodiments, a prime editor comprises a Cas9 nickase having an amino acid substitution in the RuvC domain e.g., an amino acid substitution that reduces or abolishes nuclease activity of the RuvC domain. In some embodiments, the Cas9 nickase comprises a D10X amino acid substitution compared to a wild type S. pyogenes Cas9, wherein X is any amino acid other than D. In some embodiments, a prime editor comprises a Cas9 nickase having an amino acid substitution in the HNH domain e.g., an amino acid substitution that reduces or abolishes nuclease activity of the HNH domain. In some embodiments, the Cas9 nickase comprises a H840X amino acid substitution compared to a wild type S. pyogenes Cas9, wherein X is any amino acid other than H.

In some embodiments, a prime editor comprises a Cas protein that can bind to the target gene in a sequence-specific manner but lacks or has abolished nuclease activity and may not cleave either strand of a double stranded DNA in a target gene. Abolished activity or lacking activity can refer to an enzymatic activity less than 1%, less than 2%, less than 3%, less than 4%, less than 5%, less than 6%, less than 7%, less than 8%, less than 9%, or less than 10% activity compared to a wild-type exemplary activity (e.g., wild-type Cas9 nuclease activity). In some embodiments, a Cas protein of a prime editor completely lacks nuclease activity. A nuclease, e.g., Cas9, that lacks nuclease activity may be referred to as nuclease inactive or “nuclease dead” (abbreviated by “d”). A nuclease dead Cas protein (e.g., dCas, dCas9) can bind to a target polynucleotide but may not cleave the target polynucleotide. In some embodiments, a dead Cas protein is a dead Cas9 protein. In some embodiments, a prime editor comprises a nuclease dead Cas protein wherein all of the nuclease domains (e.g., both RuvC and HNH nuclease domains in a Cas9 protein; RuvC nuclease domain in a Cpf1 protein) are mutated to lack catalytic activity, or are deleted.

A Cas protein can be modified. A Cas protein, e.g., Cas9, can be modified to increase or decrease nucleic acid binding affinity, nucleic acid binding specificity, and/or enzymatic activity. Cas proteins can also be modified to change any other activity or property of the protein, such as stability. For example, one or more nuclease domains of the Cas protein can be modified, deleted, or inactivated, or a Cas protein can be truncated to remove domains that are not essential for the function of the protein or to optimize (e.g., enhance or reduce) the activity of the Cas protein.

A Cas protein can be a fusion protein. For example, a Cas protein can be fused to a cleavage domain, an epigenetic modification domain, a transcriptional regulation domain, or a polymerase domain. A Cas protein can also be fused to a heterologous polypeptide providing increased or decreased stability. The fused domain or heterologous polypeptide can be located at the N-terminus, the C-terminus, or internally within the Cas protein.

In some embodiments, the Cas protein of a prime editor is a Class 2 Cas protein. In some embodiments, the Cas protein is a type II Cas protein. In some embodiments, the Cas protein is a Cas9 protein, a modified version of a Cas9 protein, a Cas9 protein homolog, mutant, variant, or a functional fragment thereof. As used herein, a Cas9, Cas9 protein, Cas9 polypeptide or a Cas9 nuclease refers to an RNA guided nuclease comprising one or more Cas9 nuclease domains and a Cas9 gRNA binding domain having the ability to bind a guide polynucleotide, e.g., a PEgRNA. A Cas9 protein may refer to a wild type Cas9 protein from any organism or a homolog, ortholog, or paralog from any organisms; any functional mutants or functional variants thereof; or any functional fragments or domains thereof. In some embodiments, a prime editor comprises a full-length Cas9 protein. In some embodiments, the Cas9 protein can generally comprises at least about 50%, 60%, 70%, 80%, 90%, 100% sequence identity to a wild type reference Cas9 protein (e.g., Cas9 from S. pyogenes). In some embodiments, the Cas9 comprises an amino acid change such as a deletion, insertion, substitution, fusion, chimera, or any combination thereof as compared to a wild type reference Cas9 protein.

In some embodiments, a Cas9 protein may comprise a Cas9 protein from Streptococcus pyogenes (Sp), Staphylococcus aureus (Sa), Streptococcus canis (Sc), Streptococcus thermophilus (St), Staphylococcus lugdunensis (Slu), Neisseria meningitidis (Nm), Campylobacter jejuni (Cj), Francisella novicida (Fn), or Treponema denticola (Td), or any Cas9 homolog or ortholog from an organism known in the art. In some embodiments, a Cas9 polypeptide is a SpCas9 polypeptide, e.g., comprising an amino acid sequence as set forth in NCBI Accession No. WP_038431314 or a fragment or variant thereof. In some embodiments, a Cas9 polypeptide is a SaCas9 polypeptide, e.g., comprising an amino acid sequence as set forth in Uniprot Accession No. J7RUA5 or a fragment or variant thereof. In some embodiments, a Cas9 polypeptide is a ScCas9 polypeptide, e.g., comprising an amino acid sequence as set forth in Uniprot Accession No. A0A3P5YA78 or a fragment or variant thereof. In some embodiments, a Cas9 polypeptide is a StCas9 polypeptide, e.g., comprising an amino acid sequence as set forth in NCBI Accession No. WP_007896501.1 or a fragment or variant thereof. In some embodiments, a Cas9 polypeptide is a SluCas9 polypeptide, e.g., comprising an amino acid sequence as set forth in any of NCBI Accession No. WP_230580236.1 or WP_250638315.1 or WP_242234150.1, WP_241435384.1, WP_002460848.1, KAK58371.1, or a fragment or variant thereof. In some embodiments, a Cas9 polypeptide is a NmCas9 polypeptide, e.g., comprising an amino acid sequence as set forth in any of NCBI Accession No. WP_002238326.1 or WP_061704949.1 or a fragment or variant thereof. In some embodiments, a Cas9 polypeptide is a CjCas9 polypeptide, e.g., comprising an amino acid sequence as set forth in any of NCBI Accession No. WP_100612036.1, WP_116882154.1, WP_116560509.1, WP_116484194.1, WP_116479303.1, WP_115794652.1, WP_100624872.1, or a fragment or variant thereof. In some embodiments, a Cas9 polypeptide is a FnCas9 polypeptide, e.g., comprising the amino acid sequence as set forth in Uniprot Accession No. AOQ5Y3 or a fragment or variant thereof. In some embodiments, a Cas9 polypeptide is a TdCas9 polypeptide, e.g., comprising the amino acid sequence as set forth in NCBI Accession No. WP_147625065.1 or a fragment or variant thereof. In some embodiments, a Cas9 polypeptide is a chimera comprising domains from two or more of the organisms described herein or those known in the art. In some embodiments, a Cas9 polypeptide is a Cas9 polypeptide from Streptococcus macacae, e.g., comprising the amino acid sequence as set forth in NCBI Accession No. WP_003079701.1 or a fragment or variant thereof. In some embodiments, a Cas9 polypeptide is a Cas9 polypeptide generated by replacing a PAM interaction domain of a SpCas9 with that of a Streptococcus macacae Cas9 (Spy-mac Cas9). Exemplary Cas sequences are provided in Table 79 below.

In some embodiments, a Cas9 protein comprises an amino acid sequence that is at least 50%, at least 60%, at least 70%, at least 75%, at least 80%, 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of the sequences set forth in SEQ ID NOs: 34406, 34364-34390. In some embodiments, a Cas9 protein is a Cas9 nickase that comprises an amino acid sequence that is at least 50%, at least 60%, at least 70%, at least 75%, at least 80%, 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of the sequences set forth in SEQ ID NOs: 34406, 34365, 34366, 34368, 34369, 34371, 34372, 34374, 34375, 34377, 34378, 34380, 34381, 34383, 34384, 34386, 34387, 34389, or 34390. In some embodiments, a Cas9 protein comprises an amino acid sequence that is selected from the group consisting of SEQ ID NOs: 34406, 34364-34390. In some embodiments, a prime editor comprises a Cas9 protein that comprises an amino acid sequence that lacks a N-terminus methionine relative to an amino acid sequence set forth in any one of SEQ ID NOs: 34364, 34365, 34367, 34368, 34370, 34371, 34373, 34374, 34376, 34377, 34379, 34380, 34382, 34383, 34385, 34386, 34388, or 34389. In some embodiments, the prime editing compositions or prime editing systems disclosed herein comprises a polynucleotide (e.g., a DNA, or an RNA, e.g., an mRNA) that encodes a Cas9 protein that comprises an amino acid sequence that is at least 50%, at least 60%, at least 70%, at least 75%, at least 80%, 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of the sequences set forth in SEQ ID NOs: 34406, 34364-34390.

In some embodiments, a Cas9 protein comprises a Cas9 protein from Streptococcus pyogenes (Sp), e.g., as according to NC_002737.2:854751-858857 or the protein encoded by UniProt Q99ZW2, e.g., as according to SEQ ID NO: 34364. In some embodiments, a prime editor comprises a Cas9 protein (e.g., a SpCas9) as according to any one of the sequences set forth in SEQ ID NOs: 34406, 34364-34366 or a variant thereof. In some embodiments, the Cas9 protein is a SpCas9. In some embodiments, a SpCas9 can be a wild type SpCas9, a SpCas9 variant, or a nickase SpCas9. In some embodiments, the SpCas9 lacks the N-terminus methionine relative to a corresponding SpCas9 (e.g., a wild type SpCas9, a SpCas9 variant or a nickase SpCas9). In some embodiments, a prime editor comprises a Cas9 protein, having an amino acid sequence as according to SEQ ID NO: 34364, not including the N-terminus methionine. In some embodiments, a wild type SpCas9 comprises an amino acid sequence set forth in SEQ ID NO: 34364. In some embodiments, a prime editor comprises a Cas9 protein comprising one or more mutations (e.g., amino acid substitutions, insertions and/or deletions) relative to a corresponding wild type Cas9 protein (e.g., a wild type SpCas9). In some embodiments, the Cas9 protein comprising one or more mutations relative to a wild type Cas9 (e.g., a wild type SpCas9) protein comprises an amino acid sequence set forth in SEQ ID NO: 34365, SEQ ID NO: 34366 or SEQ ID NO: 34406. Exemplary Streptococcus pyogenes Cas9 (SpCas9) amino acid sequence useful in the prime editors disclosed herein are provided below in SEQ ID NOs: 34406, 34364-34366.

In some embodiments, a prime editor comprises a Cas9 protein (e.g., a SluCas9) as according to any one of the SEQ ID NOS: 34367-34369 or a variant thereof. In some embodiments, a prime editor comprises a Cas9 protein from Staphylococcus lugdunensis (SluCas9) e.g., as according to any one of the SEQ ID NOs: 34367-34369 or a variant thereof. In some embodiments, the Cas9 protein is a SluCas9. In some embodiments, a SluCas9 can be a wild type SluCas9, a SluCas9 variant, or a nickase SluCas9. In some embodiments, the SluCas9 lacks the N-terminus methionine relative to a corresponding SluCas9 (e.g., a wild type SluCas9, a SluCas9 variant or a nickase SluCas9). In some embodiments, a prime editor comprises a Cas9 protein, having an amino acid sequence as according to SEQ ID NO: 34367, not including the N-terminus methionine. In some embodiments, a wild type SluCas9 comprises an amino acid sequence set forth in SEQ ID NO: 34367. In some embodiments, a prime editor comprises a Cas9 protein comprising one or more mutations (e.g., amino acid substitutions, insertions and/or deletions) relative to a corresponding wild type Cas9 protein (e.g., a wild type SluCas9). In some embodiments, the Cas9 protein comprising one or mutations relative to a wild type Cas9 protein comprises an amino acid sequence set forth in SEQ ID NO: 34368 or SEQ ID NO: 34369. Exemplary Staphylococcus lugdunensis Cas9 (SluCas9) amino acid sequence useful in the prime editors disclosed herein are provided below in SEQ ID NOs: 34367-34369.

In some embodiments, a prime editor comprises a Cas9 protein from Staphylococcus aureus (SaCas9) e.g., as according to any of the SEQ ID NOS: 34370-34372, or a variant thereof. In some embodiments, a prime editor comprises a Cas9 protein from Staphylococcus aureus (SaCas9) e.g., as according to any one of the SEQ ID NOS: 34370-34372, or a variant thereof. In some embodiments, the Cas9 protein is a SaCas9. In some embodiments, a SaCas9 can be a wild type SaCas9, a SaCas9 variant, or a nickase SaCas9. In some embodiments, the SaCas9 lacks the N-terminus methionine relative to a corresponding SaCas9 (e.g., a wild type SaCas9, a SaCas9 variant or a nickase SaCas9). In some embodiments, a prime editor comprises a Cas9 protein, having an amino acid sequence as according to SEQ ID NO: 34370, not including the N-terminus methionine. In some embodiments, a wild type SaCas9 comprises an amino acid sequence set forth in SEQ ID NO: 34370. In some embodiments, a prime editor comprises a Cas9 protein comprising one or more mutations (e.g., amino acid substitutions, insertions and/or deletions relative to a corresponding wild type Cas9 protein (e.g., a wild type SaCas9). In some embodiments, the Cas9 protein comprising one or more mutations relative to a wild type Cas9 protein comprises an amino acid sequence set forth in SEQ ID NO: 34371 or SEQ ID NO: 34372. Exemplary Staphylococcus aureus Cas9 (SaCas9) amino acid sequence useful in the prime editors disclosed herein are provided below in SEQ ID NOs: 34370-34372.

In some embodiments, a prime editor comprises a Cas protein, e.g., a Cas9 variant, comprising modifications that allow altered PAM recognition. Exemplary Cas9 protein amino acid sequence (e.g., Cas9 variant with altered PAM recognition specificities) that are useful in the Prime editors of the disclosure are provided below in SEQ ID NOs 34373-34381, 34388-34390. In some embodiments, a prime editor comprises a Cas9 protein as according to any one of the sequences set forth in SEQ ID NOs: 34373-34381, 34388-34390 or a variant thereof. In some embodiments, the Cas9 protein is a Cas9 variant, for example, a SpCas9 variant (e.g., SpCas9-NG, SpCas9-NGA, SpRY, or SpG). In some embodiments, the Cas9 protein lacks the N-terminus methionine relative to a corresponding Cas9 protein (e.g., a Cas9 variant set forth in any one of SEQ ID NOs: 34373, 34376, 34379, or 34388). In some embodiments, a prime editor comprises a Cas9 protein (e.g., a Cas9 variant), having an amino acid sequence as according to any one of SEQ ID NOs: 34373, 34376, 34379, or 34388 not including the N-terminus methionine. In some embodiments, a prime editor comprises a Cas9 protein comprising one or more mutations (e.g., amino acid substitutions, insertions and/or deletions) relative to a corresponding Cas9 protein (e.g., a Cas9 protein set forth in any one of SEQ ID NOs: 34373, 34376, 34379, or 34388). In some embodiments, the Cas9 protein comprising one or mutations relative to a corresponding Cas9 protein comprises an amino acid sequence set forth in any one of SEQ ID NOs: 34374, 34375, 34377, 34378, 34380, 34381, 34389, or 34390.

In some embodiments, a Cas9 protein is a chimeric Cas9, e.g., modified Cas9, e.g., synthetic RNA-guided nucleases (sRGNs), e.g., modified by DNA family shuffling, e.g., sRGN3.1, sRGN3.3. In some embodiments, the DNA family shuffling comprises, fragmentation and reassembly of parental Cas9 genes, e.g., one or more of Cas9s from Staphylococcus hyicus (Shy), Staphylococcus lugdunensis (Slu), Staphylococcus microti (Smi), and Staphylococcus pasteuri (Spa). In some embodiments, a modified sluCas9 shows increased editing efficiency and/or specificity relative to a sluCas9 that is not modified. In some embodiments, a modified Cas9, e.g., a sRGN shows at least 10%, at least 20%, at least 30%, at least 40%, at least 50%, at least 60%, at least 70%, at least 80%, at least 90%, at least 100%, at least 150%, at least 200%, at least 300%, at least 400%, at least 500%, at least 600%, at least 700%, at least 800%, at least 900%, or at least 1000% increase in editing efficiency compared to a Cas9 that is not modified. In some embodiments, a Cas9, e.g., a sRGN shows at least 10%, at least 20%, at least 30%, at least 40%, at least 50%, at least 60%, at least 70%, at least 80%, at least 90%, at least 100%, at least 150%, at least 200%, at least 300%, at least 400%, at least 500%, at least 600%, at least 700%, at least 800%, at least 900%, or at least 1000% increase in specificity compared to a Cas9 that is not modified. In some embodiments, a Cas9, e.g., a sRGN shows at least 10%, at least 20%, at least 30%, at least 40%, at least 50%, at least 60%, at least 70%, at least 80%, at least 90%, at least 100%, at least 150%, at least 200%, at least 300%, at least 400%, at least 500%, at least 600%, at least 700%, at least 800%, at least 900%, or at least 1000% increase in cleavage activity compared to a Cas9 that is not modified. In some embodiments, a Cas9, e.g., a sRGN shows ability to cleave a 5′-NNGG-3′ PAM-containing target. In some embodiments, a prime editor comprises a Cas9 protein (e.g., a chimeric Cas9), e.g., as according any one of the sequences set forth in SEQ ID NOs: 34382-34387, or a variant thereof. Exemplary amino acid sequences of Cas9 protein (e.g., sRGN) useful in the prime editors disclosed herein are provided below in SEQ ID NOs: 34382-34387. In some embodiments, a prime editor comprises a Cas9 protein, that lacks a N-terminus methionine relative to SEQ ID NO: 34382 or SEQ ID NO: 34385. In some embodiments, a prime editor comprises a Cas9 protein comprising one or more mutations (e.g., amino acid substitutions, insertions and/or deletions) relative to a corresponding Cas9 protein (e.g., a Cas9 protein set forth in SEQ ID NO: 34382 or SEQ ID NO: 34385). In some embodiments, the Cas9 protein comprising one or mutations relative to a corresponding Cas9 protein comprises an amino acid sequence set forth in any one of SEQ ID NOs: 34383, 34384, 34386, or 34387.

TABLE 79
Exemplary Cas protein sequences

| SEQ | | |
|---|---|---|
| ID | Sequence | |
| NO: | description | Amino acid sequence |
| 34364 | wild type | MDKKYSIGLDIGTNSVGWAVITDEYKVPSKKFKVLGNTDRHSIKKNLIGALLFDSGE |
| | Streptococcus | TAEATRLKRTARRRYTRRKNRICYLQEIFSNEMAKVDDSFFHRLEESFLVEEDKKHE |
| | Pyogenes Cas9 | RHPIFGNIVDEVAYHEKYPTIYHLRKKLVDSTDKADLRLIYLALAHMIKFRGHFLIEG |
| | (SpCas9) | DLNPDNSDVDKLFIQLVQTYNQLFEENPINASGVDAKAILSARLSKSRRLENLIAQLP |
| | | GEKKNGLFGNLIALSLGLTPNFKSNFDLAEDAKLQLSKDTYDDDLDNLLAQIGDQY |
| | | ADLFLAAKNLSDAILLSDILRVNTEITKAPLSASMIKRYDEHHQDLTLLKALVRQQLP |
| | | EKYKEIFFDQSKNGYAGYIDGGASQEEFYKFIKPILEKMDGTEELLVKLNREDLLRK |
| | | QRTFDNGSIPHQIHLGELHAILRRQEDFYPFLKDNREKIEKILTFRIPYYVGPLARGNS |
| | | RFAWMTRKSEETITPWNFEEVVDKGASAQSFIERMTNFDKNLPNEKVLPKHSLLYE |
| | | YFTVYNELTKVKYVTEGMRKPAFLSGEQKKAIVDLLFKTNRKVTVKQLKEDYFKKI |
| | | ECFDSVEISGVEDRFNASLGTYHDLLKIIKDKDFLDNEENEDILEDIVLTLTLFEDREM |
| | | IEERLKTYAHLFDDKVMKQLKRRRYTGWGRLSRKLINGIRDKQSGKTILDFLKSDGF |
| | | ANRNFMQLIHDDSLTFKEDIQKAQVSGQGDSLHEHIANLAGSPAIKKGILQTVKVVD |
| | | ELVKVMGRHKPENIVIEMARENQTTQKGQKNSRERMKRIEEGIKELGSQILKEHPVE |
| | | NTQLQNEKLYLYYLQNGRDMYVDQELDINRLSDYDVDHIVPQSFLKDDSIDNKVLT |
| | | RSDKNRGKSDNVPSEEVVKKMKNYWRQLLNAKLITQRKFDNLTKAERGGLSELDK |
| | | AGFIKRQLVETRQITKHVAQILDSRMNTKYDENDKLIREVKVITLKSKLVSDFRKDF |
| | | QFYKVREINNYHHAHDAYLNAVVGTALIKKYPKLESEFVYGDYKVYDVRKMIAKS |
| | | EQEIGKATAKYFFYSNIMNFFKTEITLANGEIRKRPLIETNGETGEIVWDKGRDFATV |
| | | RKVLSMPQVNIVKKTEVQTGGFSKESILPKRNSDKLIARKKDWDPKKYGGFDSPTV |
| | | AYSVLVVAKVEKGKSKKLKSVKELLGITIMERSSFEKNPIDFLEAKGYKEVKKDLIIK |
| | | LPKYSLFELENGRKRMLASAGELQKGNELALPSKYVNFLYLASHYEKLKGSPEDNE |
| | | QKQLFVEQHKHYLDEIIEQISEFSKRVILADANLDKVLSAYNKHRDKPIREQAENIIH |
| | | LFTLTNLGAPAAFKYFDTTIDRKRYTSTKEVLDATLIHQSITGLYETRIDLSQLGGD |
| 34365 | SpCas9 H840A | MDKKYSIGLDIGTNSVGWAVITDEYKVPSKKFKVLGNTDRHSIKKNLIGALLFDSGE |
| | nickase | TAEATRLKRTARRRYTRRKNRICYLQEIFSNEMAKVDDSFFHRLEESFLVEEDKKHE |
| | | RHPIFGNIVDEVAYHEKYPTIYHLRKKLVDSTDKADLRLIYLALAHMIKFRGHFLIEG |
| | | DLNPDNSDVDKLFIQLVQTYNQLFEENPINASGVDAKAILSARLSKSRRLENLIAQLP |
| | | GEKKNGLFGNLIALSLGLTPNFKSNFDLAEDAKLQLSKDTYDDDLDNLLAQIGDQY |
| | | ADLFLAAKNLSDAILLSDILRVNTEITKAPLSASMIKRYDEHHQDLTLLKALVRQQLP |
| | | EKYKEIFFDQSKNGYAGYIDGGASQEEFYKFIKPILEKMDGTEELLVKLNREDLLRK |
| | | QRTFDNGSIPHQIHLGELHAILRRQEDFYPFLKDNREKIEKILTFRIPYYVGPLARGNS |
| | | RFAWMTRKSEETITPWNFEEVVDKGASAQSFIERMTNFDKNLPNEKVLPKHSLLYE |
| | | YFTVYNELTKVKYVTEGMRKPAFLSGEQKKAIVDLLFKTNRKVTVKQLKEDYFKKI |
| | | ECFDSVEISGVEDRFNASLGTYHDLLKIIKDKDFLDNEENEDILEDIVLTLTLFEDREM |
| | | IEERLKTYAHLFDDKVMKQLKRRRYTGWGRLSRKLINGIRDKQSGKTILDFLKSDGF |
| | | ANRNFMQLIHDDSLTFKEDIQKAQVSGQGDSLHEHIANLAGSPAIKKGILQTVKVVD |
| | | ELVKVMGRHKPENIVIEMARENQTTQKGQKNSRERMKRIEEGIKELGSQILKEHPVE |
| | | NTQLQNEKLYLYYLQNGRDMYVDQELDINRLSDYDVDAIVPQSFLKDDSIDNKVLT |
| | | RSDKNRGKSDNVPSEEVVKKMKNYWRQLLNAKLITQRKFDNLTKAERGGLSELDK |
| | | AGFIKRQLVETRQITKHVAQILDSRMNTKYDENDKLIREVKVITLKSKLVSDFRKDF |
| | | QFYKVREINNYHHAHDAYLNAVVGTALIKKYPKLESEFVYGDYKVYDVRKMIAKS |
| | | EQEIGKATAKYFFYSNIMNFFKTEITLANGEIRKRPLIETNGETGEIVWDKGRDFATV |
| | | RKVLSMPQVNIVKKTEVQTGGFSKESILPKRNSDKLIARKKDWDPKKYGGFDSPTV |
| | | AYSVLVVAKVEKGKSKKLKSVKELLGITIMERSSFEKNPIDFLEAKGYKEVKKDLIIK |
| | | LPKYSLFELENGRKRMLASAGELQKGNELALPSKYVNFLYLASHYEKLKGSPEDNE |
| | | QKQLFVEQHKHYLDEIIEQISEFSKRVILADANLDKVLSAYNKHRDKPIREQAENIIH |
| | | LFTLTNLGAPAAFKYFDTTIDRKRYTSTKEVLDATLIHQSITGLYETRIDLSQLGGD |
| 34366 | Met (-) SpCas9 | DKKYSIGLDIGTNSVGWAVITDEYKVPSKKFKVLGNTDRHSIKKNLIGALLFDSGET |
| | H840A nickase | AEATRLKRTARRRYTRRKNRICYLQEIFSNEMAKVDDSFFHRLEESFLVEEDKKHER |
| | | HPIFGNIVDEVAYHEKYPTIYHLRKKLVDSTDKADLRLIYLALAHMIKFRGHFLIEGD |
| | | LNPDNSDVDKLFIQLVQTYNQLFEENPINASGVDAKAILSARLSKSRRLENLIAQLPG |
| | | EKKNGLFGNLIALSLGLTPNFKSNFDLAEDAKLQLSKDTYDDDLDNLLAQIGDQYA |
| | | DLFLAAKNLSDAILLSDILRVNTEITKAPLSASMIKRYDEHHQDLTLLKALVRQQLPE |
| | | KYKEIFFDQSKNGYAGYIDGGASQEEFYKFIKPILEKMDGTEELLVKLNREDLLRKQ |
| | | RTFDNGSIPHQIHLGELHAILRRQEDFYPFLKDNREKIEKILTFRIPYYVGPLARGNSR |
| | | FAWMTRKSEETITPWNFEEVVDKGASAQSFIERMTNFDKNLPNEKVLPKHSLLYEY |
| | | FTVYNELTKVKYVTEGMRKPAFLSGEQKKAIVDLLFKTNRKVTVKQLKEDYFKKIE |
| | | CFDSVEISGVEDRFNASLGTYHDLLKIIKDKDFLDNEENEDILEDIVLTLTLFEDREMI |
| | | EERLKTYAHLFDDKVMKQLKRRRYTGWGRLSRKLINGIRDKQSGKTILDFLKSDGF |
| | | ANRNFMQLIHDDSLTFKEDIQKAQVSGQGDSLHEHIANLAGSPAIKKGILQTVKVVD |
| | | ELVKVMGRHKPENIVIEMARENQTTQKGQKNSRERMKRIEEGIKELGSQILKEHPVE |
| | | NTQLQNEKLYLYYLQNGRDMYVDQELDINRLSDYDVDAIVPQSFLKDDSIDNKVLT |
| | | RSDKNRGKSDNVPSEEVVKKMKNYWRQLLNAKLITQRKFDNLTKAERGGLSELDK |
| | | AGFIKRQLVETRQITKHVAQILDSRMNTKYDENDKLIREVKVITLKSKLVSDFRKDF |
| | | QFYKVREINNYHHAHDAYLNAVVGTALIKKYPKLESEFVYGDYKVYDVRKMIAKS |
| | | EQEIGKATAKYFFYSNIMNFFKTEITLANGEIRKRPLIETNGETGEIVWDKGRDFATV |
| | | RKVLSMPQVNIVKKTEVQTGGFSKESILPKRNSDKLIARKKDWDPKKYGGFDSPTV |
| | | AYSVLVVAKVEKGKSKKLKSVKELLGITIMERSSFEKNPIDFLEAKGYKEVKKDLIIK |
| | | LPKYSLFELENGRKRMLASAGELQKGNELALPSKYVNFLYLASHYEKLKGSPEDNE |
| | | QKQLFVEQHKHYLDEIIEQISEFSKRVILADANLDKVLSAYNKHRDKPIREQAENIIH |
| | | LFTLTNLGAPAAFKYFDTTIDRKRYTSTKEVLDATLIHQSITGLYETRIDLSQLGGD |
| 34367 | wild type | MNQKFILGLDIGITSVGYGLIDYETKNIIDAGVRLFPEANVENNEGRRSKRGSRRLKR |
| | Staphylococcus | RRIHRLERVKKLLEDYNLLDQSQIPQSTNPYAIRVKGLSEALSKDELVIALLHIAKRR |
| | lugdunensis | GIHKIDVIDSNDDVGNELSTKEQLNKNSKLLKDKFVCQIQLERMNEGQVRGEKNRF |
| | (Slu)Cas9 | KTADIIKEIIQLLNVQKNFHQLDENFINKYIELVEMRREYFEGPGKGSPYGWEGDPK |
| | | AWYETLMGHCTYFPDELRSVKYAYSADLFNALNDLNNLVIQRDGLSKLEYHEKYH |
| | | IIENVFKQKKKPTLKQIANEINVNPEDIKGYRITKSGKPQFTEFKLYHDLKSVLFDQSI |
| | | LENEDVLDQIAEILTIYQDKDSIKSKLTELDILLNEEDKENIAQLTGYTGTHRLSLKCI |
| | | RLVLEEQWYSSRNQMEIFTHLNIKPKKINLTAANKIPKAMIDEFILSPVVKRTFGQAI |
| | | NLINKIIEKYGVPEDIIIELARENNSKDKQKFINEMQKKNENTRKRINEIIGKYGNQNA |
| | | KRLVEKIRLHDEQEGKCLYSLESIPLEDLLNNPNHYEVDHIIPRSVSFDNSYHNKVLV |
| | | KQSENSKKSNLTPYQYFNSGKSKLSYNQFKQHILNLSKSQDRISKKKKEYLLEERDI |
| | | NKFEVQKEFINRNLVDTRYATRELTNYLKAYFSANNMNVKVKTINGSFTDYLRKV |
| | | WKFKKERNHGYKHHAEDALIIANADFLFKENKKLKAVNSVLEKPEIESKQLDIQVD |
| | | SEDNYSEMFIIPKQVQDIKDFRNFKYSHRVDKKPNRQLINDTLYSTRKKDNSTYIVQ |
| | | TIKDIYAKDNTTLKKQFDKSPEKFLMYQHDPRTFEKLEVIMKQYANEKNPLAKYHE |
| | | ETGEYLTKYSKKNNGPIVKSLKYIGNKLGSHLDVTHQFKSSTKKLVKLSIKPYRFDV |
| | | YLTDKGYKFITISYLDVLKKDNYYYIPEQKYDKLKLGKAIDKNAKFIASFYKNDLIK |
| | | LDGEIYKIIGVNSDTRNMIELDLPDIRYKEYCELNNIKGEPRIKKTIGKKVNSIEKLTT |
| | | DVLGNVFTNTQYTKPQLLFKRGN |
| 34368 | SluCas9 | MNQKFILGLDIGITSVGYGLIDYETKNIIDAGVRLFPEANVENNEGRRSKRGSRRLKR |
| | N582A | RRIHRLERVKKLLEDYNLLDQSQIPQSTNPYAIRVKGLSEALSKDELVIALLHIAKRR |
| | nickase | GIHKIDVIDSNDDVGNELSTKEQLNKNSKLLKDKFVCQIQLERMNEGQVRGEKNRF |
| | | KTADIIKEIIQLLNVQKNFHQLDENFINKYIELVEMRREYFEGPGKGSPYGWEGDPK |
| | | AWYETLMGHCTYFPDELRSVKYAYSADLFNALNDLNNLVIQRDGLSKLEYHEKYH |
| | | IIENVFKQKKKPTLKQIANEINVNPEDIKGYRITKSGKPQFTEFKLYHDLKSVLFDQSI |
| | | LENEDVLDQIAEILTIYQDKDSIKSKLTELDILLNEEDKENIAQLTGYTGTHRLSLKCI |
| | | RLVLEEQWYSSRNQMEIFTHLNIKPKKINLTAANKIPKAMIDEFILSPVVKRTFGQAI |
| | | NLINKIIEKYGVPEDIIIELARENNSKDKQKFINEMQKKNENTRKRINEIIGKYGNQNA |
| | | KRLVEKIRLHDEQEGKCLYSLESIPLEDLLNNPNHYEVDHIIPRSVSFDNSYHNKVLV |
| | | KQSEASKKSNLTPYQYFNSGKSKLSYNQFKQHILNLSKSQDRISKKKKEYLLEERDI |
| | | NKFEVQKEFINRNLVDTRYATRELTNYLKAYFSANNMNVKVKTINGSFTDYLRKV |
| | | WKFKKERNHGYKHHAEDALIIANADFLFKENKKLKAVNSVLEKPEIESKQLDIQVD |
| | | SEDNYSEMFIIPKQVQDIKDFRNFKYSHRVDKKPNRQLINDTLYSTRKKDNSTYIVQ |
| | | TIKDIYAKDNTTLKKQFDKSPEKFLMYQHDPRTFEKLEVIMKQYANEKNPLAKYHE |
| | | ETGEYLTKYSKKNNGPIVKSLKYIGNKLGSHLDVTHQFKSSTKKLVKLSIKPYRFDV |
| | | YLTDKGYKFITISYLDVLKKDNYYYIPEQKYDKLKLGKAIDKNAKFIASFYKNDLIK |
| | | LDGEIYKIIGVNSDTRNMIELDLPDIRYKEYCELNNIKGEPRIKKTIGKKVNSIEKLTT |
| | | DVLGNVFTNTQYTKPQLLFKRGN |
| 34369 | Met (-) | NQKFILGLDIGITSVGYGLIDYETKNIIDAGVRLFPEANVENNEGRRSKRGSRRLKRR |
| | SluCas9 | RIHRLERVKKLLEDYNLLDQSQIPQSTNPYAIRVKGLSEALSKDELVIALLHIAKRRGI |
| | nickase | HKIDVIDSNDDVGNELSTKEQLNKNSKLLKDKFVCQIQLERMNEGQVRGEKNRFKT |
| | | ADIIKEIIQLLNVQKNFHQLDENFINKYIELVEMRREYFEGPGKGSPYGWEGDPKAW |
| | | YETLMGHCTYFPDELRSVKYAYSADLFNALNDLNNLVIQRDGLSKLEYHEKYHIIE |
| | | NVFKQKKKPTLKQIANEINVNPEDIKGYRITKSGKPQFTEFKLYHDLKSVLFDQSILE |
| | | NEDVLDQIAEILTIYQDKDSIKSKLTELDILLNEEDKENIAQLTGYTGTHRLSLKCIRL |
| | | VLEEQWYSSRNQMEIFTHLNIKPKKINLTAANKIPKAMIDEFILSPVVKRTFGQAINLI |
| | | NKIIEKYGVPEDIIIELARENNSKDKQKFINEMQKKNENTRKRINEIIGKYGNQNAKR |
| | | LVEKIRLHDEQEGKCLYSLESIPLEDLLNNPNHYEVDHIIPRSVSFDNSYHNKVLVKQ |
| | | SEASKKSNLTPYQYFNSGKSKLSYNQFKQHILNLSKSQDRISKKKKEYLLEERDINKF |
| | | EVQKEFINRNLVDTRYATRELTNYLKAYFSANNMNVKVKTINGSFTDYLRKVWKF |
| | | KKERNHGYKHHAEDALIIANADFLFKENKKLKAVNSVLEKPEIESKQLDIQVDSEDN |
| | | YSEMFIIPKQVQDIKDFRNFKYSHRVDKKPNRQLINDTLYSTRKKDNSTYIVQTIKDI |
| | | YAKDNTTLKKQFDKSPEKFLMYQHDPRTFEKLEVIMKQYANEKNPLAKYHEETGE |
| | | YLTKYSKKNNGPIVKSLKYIGNKLGSHLDVTHQFKSSTKKLVKLSIKPYRFDVYLTD |
| | | KGYKFITISYLDVLKKDNYYYIPEQKYDKLKLGKAIDKNAKFIASFYKNDLIKLDGEI |
| | | YKIIGVNSDTRNMIELDLPDIRYKEYCELNNIKGEPRIKKTIGKKVNSIEKLTTDVLGN |
| | | VFTNTQYTKPQLLFKRGN |
| 34370 | Staphylococcus | MKRNYILGLDIGITSVGYGIIDYETRDVIDAGVRLFKEANVENNEGRRSKRGARRLK |
| | aureus Cas9 | RRRRHRIQRVKKLLFDYNLLTDHSELSGINPYEARVKGLSQKLSEEEFSAALLHLAK |
| | (SaCas9) | RRGVHNVNEVEEDTGNELSTKEQISRNSKALEEKYVAELQLERLKKDGEVRGSINR |
| | | FKTSDYVKEAKQLLKVQKAYHQLDQSFIDTYIDLLETRRTYYEGPGEGSPFGWKDI |
| | | KEWYEMLMGHCTYFPEELRSVKYAYNADLYNALNDLNNLVITRDENEKLEYYEKF |
| | | QIIENVFKQKKKPTLKQIAKEILVNEEDIKGYRVTSTGKPEFTNLKVYHDIKDITARK |
| | | EIIENAELLDQIAKILTIYQSSEDIQEELTNLNSELTQEEIEQISNLKGYTGTHNLSLKAI |
| | | NLILDELWHTNDNQIAIFNRLKLVPKKVDLSQQKEIPTTLVDDFILSPVVKRSFIQSIK |
| | | VINAIIKKYGLPNDIIIELAREKNSKDAQKMINEMQKRNRQTNERIEEIIRTTGKENAK |
| | | YLIEKIKLHDMQEGKCLYSLEAIPLEDLLNNPFNYEVDHIIPRSVSFDNSFNNKVLVK |
| | | QEENSKKGNRTPFQYLSSSDSKISYETFKKHILNLAKGKGRISKTKKEYLLEERDINR |
| | | FSVQKDFINRNLVDTRYATRGLMNLLRSYFRVNNLDVKVKSINGGFTSFLRRKWKF |
| | | KKERNKGYKHHAEDALIIANADFIFKEWKKLDKAKKVMENQMFEEKQAESMPEIE |
| | | TEQEYKEIFITPHQIKHIKDFKDYKYSHRVDKKPNRELINDTLYSTRKDDKGNTLIVN |
| | | NLNGLYDKDNDKLKKLINKSPEKLLMYHHDPQTYQKLKLIMEQYGDEKNPLYKYY |
| | | EETGNYLTKYSKKDNGPVIKKIKYYGNKLNAHLDITDDYPNSRNKVVKLSLKPYRF |
| | | DVYLDNGVYKFVTVKNLDVIKKENYYEVNSKCYEEAKKLKKISNQAEFIASFYNND |
| | | LIKINGELYRVIGVNNDLLNRIEVNMIDITYREYLENMNDKRPPRIIKTIASKTQSIKK |
| | | YSTDILGNLYEVKSKKHPQIIKKG |
| 34371 | SaCas9 N580A | MKRNYILGLDIGITSVGYGIIDYETRDVIDAGVRLFKEANVENNEGRRSKRGARRLK |
| | nickase | RRRRHRIQRVKKLLFDYNLLTDHSELSGINPYEARVKGLSQKLSEEEFSAALLHLAK |
| | | RRGVHNVNEVEEDTGNELSTKEQISRNSKALEEKYVAELQLERLKKDGEVRGSINR |
| | | FKTSDYVKEAKQLLKVQKAYHQLDQSFIDTYIDLLETRRTYYEGPGEGSPFGWKDI |
| | | KEWYEMLMGHCTYFPEELRSVKYAYNADLYNALNDLNNLVITRDENEKLEYYEKF |
| | | QIIENVFKQKKKPTLKQIAKEILVNEEDIKGYRVTSTGKPEFTNLKVYHDIKDITARK |
| | | EIIENAELLDQIAKILTIYQSSEDIQEELTNLNSELTQEEIEQISNLKGYTGTHNLSLKAI |
| | | NLILDELWHTNDNQIAIFNRLKLVPKKVDLSQQKEIPTTLVDDFILSPVVKRSFIQSIK |
| | | VINAIIKKYGLPNDIIIELAREKNSKDAQKMINEMQKRNRQTNERIEEIIRTTGKENAK |
| | | YLIEKIKLHDMQEGKCLYSLEAIPLEDLLNNPFNYEVDHIIPRSVSFDNSFNNKVLVK |
| | | QEEASKKGNRTPFQYLSSSDSKISYETFKKHILNLAKGKGRISKTKKEYLLEERDINR |
| | | FSVQKDFINRNLVDTRYATRGLMNLLRSYFRVNNLDVKVKSINGGFTSFLRRKWKF |
| | | KKERNKGYKHHAEDALIIANADFIFKEWKKLDKAKKVMENQMFEEKQAESMPEIE |
| | | TEQEYKEIFITPHQIKHIKDFKDYKYSHRVDKKPNRELINDTLYSTRKDDKGNTLIVN |
| | | NLNGLYDKDNDKLKKLINKSPEKLLMYHHDPQTYQKLKLIMEQYGDEKNPLYKYY |
| | | EETGNYLTKYSKKDNGPVIKKIKYYGNKLNAHLDITDDYPNSRNKVVKLSLKPYRF |
| | | DVYLDNGVYKFVTVKNLDVIKKENYYEVNSKCYEEAKKLKKISNQAEFIASFYNND |
| | | LIKINGELYRVIGVNNDLLNRIEVNMIDITYREYLENMNDKRPPRIIKTIASKTQSIKK |
| | | YSTDILGNLYEVKSKKHPQIIKKG |
| 34372 | Met (-) | KRNYILGLDIGITSVGYGIIDYETRDVIDAGVRLFKEANVENNEGRRSKRGARRLKR |
| | SaCas9 | RRRHRIQRVKKLLFDYNLLTDHSELSGINPYEARVKGLSQKLSEEEFSAALLHLAKR |
| | nickase | RGVHNVNEVEEDTGNELSTKEQISRNSKALEEKYVAELQLERLKKDGEVRGSINRF |
| | | KTSDYVKEAKQLLKVQKAYHQLDQSFIDTYIDLLETRRTYYEGPGEGSPFGWKDIK |
| | | EWYEMLMGHCTYFPEELRSVKYAYNADLYNALNDLNNLVITRDENEKLEYYEKFQ |
| | | IIENVFKQKKKPTLKQIAKEILVNEEDIKGYRVTSTGKPEFTNLKVYHDIKDITARKEI |
| | | IENAELLDQIAKILTIYQSSEDIQEELTNLNSELTQEEIEQISNLKGYTGTHNLSLKAIN |
| | | LILDELWHTNDNQIAIFNRLKLVPKKVDLSQQKEIPTTLVDDFILSPVVKRSFIQSIKVI |
| | | NAIIKKYGLPNDIIIELAREKNSKDAQKMINEMQKRNRQTNERIEEIIRTTGKENAKY |
| | | LIEKIKLHDMQEGKCLYSLEAIPLEDLLNNPFNYEVDHIIPRSVSFDNSFNNKVLVKQ |
| | | EEASKKGNRTPFQYLSSSDSKISYETFKKHILNLAKGKGRISKTKKEYLLEERDINRFS |
| | | VQKDFINRNLVDTRYATRGLMNLLRSYFRVNNLDVKVKSINGGFTSFLRRKWKFK |
| | | KERNKGYKHHAEDALIIANADFIFKEWKKLDKAKKVMENQMFEEKQAESMPEIETE |
| | | QEYKEIFITPHQIKHIKDFKDYKYSHRVDKKPNRELINDTLYSTRKDDKGNTLIVNNL |
| | | NGLYDKDNDKLKKLINKSPEKLLMYHHDPQTYQKLKLIMEQYGDEKNPLYKYYEE |
| | | TGNYLTKYSKKDNGPVIKKIKYYGNKLNAHLDITDDYPNSRNKVVKLSLKPYRFDV |
| | | YLDNGVYKFVTVKNLDVIKKENYYEVNSKCYEEAKKLKKISNQAEFIASFYNNDLI |
| | | KINGELYRVIGVNNDLLNRIEVNMIDITYREYLENMNDKRPPRIIKTIASKTQSIKKYS |
| | | TDILGNLYEVKSKKHPQIIKKG |
| 34373 | SpCas9-NG | MDKKYSIGLDIGTNSVGWAVITDEYKVPSKKFKVLGNTDRHSIKKNLIGALLFDSGE |
| | (VRVRFRR) | TAEATRLKRTARRRYTRRKNRICYLQEIFSNEMAKVDDSFFHRLEESFLVEEDKKHE |
| | | RHPIFGNIVDEVAYHEKYPTIYHLRKKLVDSTDKADLRLIYLALAHMIKFRGHFLIEG |
| | | DLNPDNSDVDKLFIQLVQTYNQLFEENPINASGVDAKAILSARLSKSRRLENLIAQLP |
| | | GEKKNGLFGNLIALSLGLTPNFKSNFDLAEDAKLQLSKDTYDDDLDNLLAQIGDQY |
| | | ADLFLAAKNLSDAILLSDILRVNTEITKAPLSASMIKRYDEHHQDLTLLKALVRQQLP |
| | | EKYKEIFFDQSKNGYAGYIDGGASQEEFYKFIKPILEKMDGTEELLVKLNREDLLRK |
| | | QRTFDNGSIPHQIHLGELHAILRRQEDFYPFLKDNREKIEKILTFRIPYYVGPLARGNS |
| | | RFAWMTRKSEETITPWNFEEVVDKGASAQSFIERMTNFDKNLPNEKVLPKHSLLYE |
| | | YFTVYNELTKVKYVTEGMRKPAFLSGEQKKAIVDLLFKTNRKVTVKQLKEDYFKKI |
| | | ECFDSVEISGVEDRFNASLGTYHDLLKIIKDKDFLDNEENEDILEDIVLTLTLFEDREM |
| | | IEERLKTYAHLFDDKVMKQLKRRRYTGWGRLSRKLINGIRDKQSGKTILDFLKSDGF |
| | | ANRNFMQLIHDDSLTFKEDIQKAQVSGQGDSLHEHIANLAGSPAIKKGILQTVKVVD |
| | | ELVKVMGRHKPENIVIEMARENQTTQKGQKNSRERMKRIEEGIKELGSQILKEHPVE |
| | | NTQLQNEKLYLYYLQNGRDMYVDQELDINRLSDYDVDHIVPQSFLKDDSIDNKVLT |
| | | RSDKNRGKSDNVPSEEVVKKMKNYWRQLLNAKLITQRKFDNLTKAERGGLSELDK |
| | | AGFIKRQLVETRQITKHVAQILDSRMNTKYDENDKLIREVKVITLKSKLVSDFRKDF |
| | | QFYKVREINNYHHAHDAYLNAVVGTALIKKYPKLESEFVYGDYKVYDVRKMIAKS |
| | | EQEIGKATAKYFFYSNIMNFFKTEITLANGEIRKRPLIETNGETGEIVWDKGRDFATV |
| | | RKVLSMPQVNIVKKTEVQTGGFSKESIRPKRNSDKLIARKKDWDPKKYGGFVSPTV |
| | | AYSVLVVAKVEKGKSKKLKSVKELLGITIMERSSFEKNPIDFLEAKGYKEVKKDLIIK |
| | | LPKYSLFELENGRKRMLASARFLQKGNELALPSKYVNFLYLASHYEKLKGSPEDNE |
| | | QKQLFVEQHKHYLDEIIEQISEFSKRVILADANLDKVLSAYNKHRDKPIREQAENIIH |
| | | LFTLTNLGAPRAFKYFDTTIDRKVYRSTKEVLDATLIHQSITGLYETRIDLSQLGGD |
| 34374 | spCas9-NG | MDKKYSIGLDIGTNSVGWAVITDEYKVPSKKFKVLGNTDRHSIKKNLIGALLFDSGE |
| | (H840A_ | TAEATRLKRTARRRYTRRKNRICYLQEIFSNEMAKVDDSFFHRLEESFLVEEDKKHE |
| | VRVRFRR) | RHPIFGNIVDEVAYHEKYPTIYHLRKKLVDSTDKADLRLIYLALAHMIKFRGHFLIEG |
| | Nickase | DLNPDNSDVDKLFIQLVQTYNQLFEENPINASGVDAKAILSARLSKSRRLENLIAQLP |
| | | GEKKNGLFGNLIALSLGLTPNFKSNFDLAEDAKLQLSKDTYDDDLDNLLAQIGDQY |
| | | ADLFLAAKNLSDAILLSDILRVNTEITKAPLSASMIKRYDEHHQDLTLLKALVRQQLP |
| | | EKYKEIFFDQSKNGYAGYIDGGASQEEFYKFIKPILEKMDGTEELLVKLNREDLLRK |
| | | QRTFDNGSIPHQIHLGELHAILRRQEDFYPFLKDNREKIEKILTFRIPYYVGPLARGNS |
| | | RFAWMTRKSEETITPWNFEEVVDKGASAQSFIERMTNFDKNLPNEKVLPKHSLLYE |
| | | YFTVYNELTKVKYVTEGMRKPAFLSGEQKKAIVDLLFKTNRKVTVKQLKEDYFKKI |
| | | ECFDSVEISGVEDRFNASLGTYHDLLKIIKDKDFLDNEENEDILEDIVLTLTLFEDREM |
| | | IEERLKTYAHLFDDKVMKQLKRRRYTGWGRLSRKLINGIRDKQSGKTILDFLKSDGF |
| | | ANRNFMQLIHDDSLTFKEDIQKAQVSGQGDSLHEHIANLAGSPAIKKGILQTVKVVD |
| | | ELVKVMGRHKPENIVIEMARENQTTQKGQKNSRERMKRIEEGIKELGSQILKEHPVE |
| | | NTQLQNEKLYLYYLQNGRDMYVDQELDINRLSDYDVDAIVPQSFLKDDSIDNKVLT |
| | | RSDKNRGKSDNVPSEEVVKKMKNYWRQLLNAKLITQRKFDNLTKAERGGLSELDK |
| | | AGFIKRQLVETRQITKHVAQILDSRMNTKYDENDKLIREVKVITLKSKLVSDFRKDF |
| | | QFYKVREINNYHHAHDAYLNAVVGTALIKKYPKLESEFVYGDYKVYDVRKMIAKS |
| | | EQEIGKATAKYFFYSNIMNFFKTEITLANGEIRKRPLIETNGETGEIVWDKGRDFATV |
| | | RKVLSMPQVNIVKKTEVQTGGFSKESIRPKRNSDKLIARKKDWDPKKYGGFVSPTV |
| | | AYSVLVVAKVEKGKSKKLKSVKELLGITIMERSSFEKNPIDFLEAKGYKEVKKDLIIK |
| | | LPKYSLFELENGRKRMLASARFLQKGNELALPSKYVNFLYLASHYEKLKGSPEDNE |
| | | QKQLFVEQHKHYLDEIIEQISEFSKRVILADANLDKVLSAYNKHRDKPIREQAENIIH |
| | | LFTLTNLGAPRAFKYFDTTIDRKVYRSTKEVLDATLIHQSITGLYETRIDLSQLGGD |
| 34375 | Met (-) | DKKYSIGLDIGTNSVGWAVITDEYKVPSKKFKVLGNTDRHSIKKNLIGALLFDSGET |
| | SpCas9-NG | AEATRLKRTARRRYTRRKNRICYLQEIFSNEMAKVDDSFFHRLEESFLVEEDKKHER |
| | Nikcase | HPIFGNIVDEVAYHEKYPTIYHLRKKLVDSTDKADLRLIYLALAHMIKFRGHFLIEGD |
| | | LNPDNSDVDKLFIQLVQTYNQLFEENPINASGVDAKAILSARLSKSRRLENLIAQLPG |
| | | EKKNGLFGNLIALSLGLTPNFKSNFDLAEDAKLQLSKDTYDDDLDNLLAQIGDQYA |
| | | DLFLAAKNLSDAILLSDILRVNTEITKAPLSASMIKRYDEHHQDLTLLKALVRQQLPE |
| | | KYKEIFFDQSKNGYAGYIDGGASQEEFYKFIKPILEKMDGTEELLVKLNREDLLRKQ |
| | | RTFDNGSIPHQIHLGELHAILRRQEDFYPFLKDNREKIEKILTFRIPYYVGPLARGNSR |
| | | FAWMTRKSEETITPWNFEEVVDKGASAQSFIERMTNFDKNLPNEKVLPKHSLLYEY |
| | | FTVYNELTKVKYVTEGMRKPAFLSGEQKKAIVDLLFKTNRKVTVKQLKEDYFKKIE |
| | | CFDSVEISGVEDRFNASLGTYHDLLKIIKDKDFLDNEENEDILEDIVLTLTLFEDREMI |
| | | EERLKTYAHLFDDKVMKQLKRRRYTGWGRLSRKLINGIRDKQSGKTILDFLKSDGF |
| | | ANRNFMQLIHDDSLTFKEDIQKAQVSGQGDSLHEHIANLAGSPAIKKGILQTVKVVD |
| | | ELVKVMGRHKPENIVIEMARENQTTQKGQKNSRERMKRIEEGIKELGSQILKEHPVE |
| | | NTQLQNEKLYLYYLQNGRDMYVDQELDINRLSDYDVDAIVPQSFLKDDSIDNKVLT |
| | | RSDKNRGKSDNVPSEEVVKKMKNYWRQLLNAKLITQRKFDNLTKAERGGLSELDK |
| | | AGFIKRQLVETRQITKHVAQILDSRMNTKYDENDKLIREVKVITLKSKLVSDFRKDF |
| | | QFYKVREINNYHHAHDAYLNAVVGTALIKKYPKLESEFVYGDYKVYDVRKMIAKS |
| | | EQEIGKATAKYFFYSNIMNFFKTEITLANGEIRKRPLIETNGETGEIVWDKGRDFATV |
| | | RKVLSMPQVNIVKKTEVQTGGFSKESIRPKRNSDKLIARKKDWDPKKYGGFVSPTV |
| | | AYSVLVVAKVEKGKSKKLKSVKELLGITIMERSSFEKNPIDFLEAKGYKEVKKDLIIK |
| | | LPKYSLFELENGRKRMLASARFLQKGNELALPSKYVNFLYLASHYEKLKGSPEDNE |
| | | QKQLFVEQHKHYLDEIIEQISEFSKRVILADANLDKVLSAYNKHRDKPIREQAENIIH |
| | | LFTLTNLGAPRAFKYFDTTIDRKVYRSTKEVLDATLIHQSITGLYETRIDLSQLGGD |
| 34376 | spCas9-NGA | MDKKYSIGLDIGTNSVGWAVITDEYKVPSKKFKVLGNTDRHSIKKNLIGALLFDSGE |
| | (VRQR) | TAEATRLKRTARRRYTRRKNRICYLQEIFSNEMAKVDDSFFHRLEESFLVEEDKKHE |
| | | RHPIFGNIVDEVAYHEKYPTIYHLRKKLVDSTDKADLRLIYLALAHMIKFRGHFLIEG |
| | | DLNPDNSDVDKLFIQLVQTYNQLFEENPINASGVDAKAILSARLSKSRRLENLIAQLP |
| | | GEKKNGLFGNLIALSLGLTPNFKSNFDLAEDAKLQLSKDTYDDDLDNLLAQIGDQY |
| | | ADLFLAAKNLSDAILLSDILRVNTEITKAPLSASMIKRYDEHHQDLTLLKALVRQQLP |
| | | EKYKEIFFDQSKNGYAGYIDGGASQEEFYKFIKPILEKMDGTEELLVKLNREDLLRK |
| | | QRTFDNGSIPHQIHLGELHAILRRQEDFYPFLKDNREKIEKILTFRIPYYVGPLARGNS |
| | | RFAWMTRKSEETITPWNFEEVVDKGASAQSFIERMTNFDKNLPNEKVLPKHSLLYE |
| | | YFTVYNELTKVKYVTEGMRKPAFLSGEQKKAIVDLLFKTNRKVTVKQLKEDYFKKI |
| | | ECFDSVEISGVEDRFNASLGTYHDLLKIIKDKDFLDNEENEDILEDIVLTLTLFEDREM |
| | | IEERLKTYAHLFDDKVMKQLKRRRYTGWGRLSRKLINGIRDKQSGKTILDFLKSDGF |
| | | ANRNFMQLIHDDSLTFKEDIQKAQVSGQGDSLHEHIANLAGSPAIKKGILQTVKVVD |
| | | ELVKVMGRHKPENIVIEMARENQTTQKGQKNSRERMKRIEEGIKELGSQILKEHPVE |
| | | NTQLQNEKLYLYYLQNGRDMYVDQELDINRLSDYDVDHIVPQSFLKDDSIDNKVLT |
| | | RSDKNRGKSDNVPSEEVVKKMKNYWRQLLNAKLITQRKFDNLTKAERGGLSELDK |
| | | AGFIKRQLVETRQITKHVAQILDSRMNTKYDENDKLIREVKVITLKSKLVSDFRKDF |
| | | QFYKVREINNYHHAHDAYLNAVVGTALIKKYPKLESEFVYGDYKVYDVRKMIAKS |
| | | EQEIGKATAKYFFYSNIMNFFKTEITLANGEIRKRPLIETNGETGEIVWDKGRDFATV |
| | | RKVLSMPQVNIVKKTEVQTGGFSKESILPKRNSDKLIARKKDWDPKKYGGFVSPTV |
| | | AYSVLVVAKVEKGKSKKLKSVKELLGITIMERSSFEKNPIDFLEAKGYKEVKKDLIIK |
| | | LPKYSLFELENGRKRMLASARELQKGNELALPSKYVNFLYLASHYEKLKGSPEDNE |
| | | QKQLFVEQHKHYLDEIIEQISEFSKRVILADANLDKVLSAYNKHRDKPIREQAENIIH |
| | | LFTLTNLGAPAAFKYFDTTIDRKQYRSTKEVLDATLIHQSITGLYETRIDLSQLGGD |
| 34377 | spCas9-NGA | MDKKYSIGLDIGTNSVGWAVITDEYKVPSKKFKVLGNTDRHSIKKNLIGALLFDSGE |
| | (H840A_VRQR) | TAEATRLKRTARRRYTRRKNRICYLQEIFSNEMAKVDDSFFHRLEESFLVEEDKKHE |
| | Nickase | RHPIFGNIVDEVAYHEKYPTIYHLRKKLVDSTDKADLRLIYLALAHMIKFRGHFLIEG |
| | | DLNPDNSDVDKLFIQLVQTYNQLFEENPINASGVDAKAILSARLSKSRRLENLIAQLP |
| | | GEKKNGLFGNLIALSLGLTPNFKSNFDLAEDAKLQLSKDTYDDDLDNLLAQIGDQY |
| | | ADLFLAAKNLSDAILLSDILRVNTEITKAPLSASMIKRYDEHHQDLTLLKALVRQQLP |
| | | EKYKEIFFDQSKNGYAGYIDGGASQEEFYKFIKPILEKMDGTEELLVKLNREDLLRK |
| | | QRTFDNGSIPHQIHLGELHAILRRQEDFYPFLKDNREKIEKILTFRIPYYVGPLARGNS |
| | | RFAWMTRKSEETITPWNFEEVVDKGASAQSFIERMTNFDKNLPNEKVLPKHSLLYE |
| | | YFTVYNELTKVKYVTEGMRKPAFLSGEQKKAIVDLLFKTNRKVTVKQLKEDYFKKI |
| | | ECFDSVEISGVEDRFNASLGTYHDLLKIIKDKDFLDNEENEDILEDIVLTLTLFEDREM |
| | | IEERLKTYAHLFDDKVMKQLKRRRYTGWGRLSRKLINGIRDKQSGKTILDFLKSDGF |
| | | ANRNFMQLIHDDSLTFKEDIQKAQVSGQGDSLHEHIANLAGSPAIKKGILQTVKVVD |
| | | ELVKVMGRHKPENIVIEMARENQTTQKGQKNSRERMKRIEEGIKELGSQILKEHPVE |
| | | NTQLQNEKLYLYYLQNGRDMYVDQELDINRLSDYDVDAIVPQSFLKDDSIDNKVLT |
| | | RSDKNRGKSDNVPSEEVVKKMKNYWRQLLNAKLITQRKFDNLTKAERGGLSELDK |
| | | AGFIKRQLVETRQITKHVAQILDSRMNTKYDENDKLIREVKVITLKSKLVSDFRKDF |
| | | QFYKVREINNYHHAHDAYLNAVVGTALIKKYPKLESEFVYGDYKVYDVRKMIAKS |
| | | EQEIGKATAKYFFYSNIMNFFKTEITLANGEIRKRPLIETNGETGEIVWDKGRDFATV |
| | | RKVLSMPQVNIVKKTEVQTGGFSKESILPKRNSDKLIARKKDWDPKKYGGFVSPTV |
| | | AYSVLVVAKVEKGKSKKLKSVKELLGITIMERSSFEKNPIDFLEAKGYKEVKKDLIIK |
| | | LPKYSLFELENGRKRMLASARELQKGNELALPSKYVNFLYLASHYEKLKGSPEDNE |
| | | QKQLFVEQHKHYLDEIIEQISEFSKRVILADANLDKVLSAYNKHRDKPIREQAENIIH |
| | | LFTLTNLGAPAAFKYFDTTIDRKQYRSTKEVLDATLIHQSITGLYETRIDLSQLGGD |
| 34378 | Met(-) | DKKYSIGLDIGTNSVGWAVITDEYKVPSKKFKVLGNTDRHSIKKNLIGALLFDSGET |
| | spCas9-NGA | AEATRLKRTARRRYTRRKNRICYLQEIFSNEMAKVDDSFFHRLEESFLVEEDKKHER |
| | Nickase | HPIFGNIVDEVAYHEKYPTIYHLRKKLVDSTDKADLRLIYLALAHMIKFRGHFLIEGD |
| | | LNPDNSDVDKLFIQLVQTYNQLFEENPINASGVDAKAILSARLSKSRRLENLIAQLPG |
| | | EKKNGLFGNLIALSLGLTPNFKSNFDLAEDAKLQLSKDTYDDDLDNLLAQIGDQYA |
| | | DLFLAAKNLSDAILLSDILRVNTEITKAPLSASMIKRYDEHHQDLTLLKALVRQQLPE |
| | | KYKEIFFDQSKNGYAGYIDGGASQEEFYKFIKPILEKMDGTEELLVKLNREDLLRKQ |
| | | RTFDNGSIPHQIHLGELHAILRRQEDFYPFLKDNREKIEKILTFRIPYYVGPLARGNSR |
| | | FAWMTRKSEETITPWNFEEVVDKGASAQSFIERMTNFDKNLPNEKVLPKHSLLYEY |
| | | FTVYNELTKVKYVTEGMRKPAFLSGEQKKAIVDLLFKTNRKVTVKQLKEDYFKKIE |
| | | CFDSVEISGVEDRFNASLGTYHDLLKIIKDKDFLDNEENEDILEDIVLTLTLFEDREMI |
| | | EERLKTYAHLFDDKVMKQLKRRRYTGWGRLSRKLINGIRDKQSGKTILDFLKSDGF |
| | | ANRNFMQLIHDDSLTFKEDIQKAQVSGQGDSLHEHIANLAGSPAIKKGILQTVKVVD |
| | | ELVKVMGRHKPENIVIEMARENQTTQKGQKNSRERMKRIEEGIKELGSQILKEHPVE |
| | | NTQLQNEKLYLYYLQNGRDMYVDQELDINRLSDYDVDAIVPQSFLKDDSIDNKVLT |
| | | RSDKNRGKSDNVPSEEVVKKMKNYWRQLLNAKLITQRKFDNLTKAERGGLSELDK |
| | | AGFIKRQLVETRQITKHVAQILDSRMNTKYDENDKLIREVKVITLKSKLVSDFRKDF |
| | | QFYKVREINNYHHAHDAYLNAVVGTALIKKYPKLESEFVYGDYKVYDVRKMIAKS |
| | | EQEIGKATAKYFFYSNIMNFFKTEITLANGEIRKRPLIETNGETGEIVWDKGRDFATV |
| | | RKVLSMPQVNIVKKTEVQTGGFSKESILPKRNSDKLIARKKDWDPKKYGGFVSPTV |
| | | AYSVLVVAKVEKGKSKKLKSVKELLGITIMERSSFEKNPIDFLEAKGYKEVKKDLIIK |
| | | LPKYSLFELENGRKRMLASARELQKGNELALPSKYVNFLYLASHYEKLKGSPEDNE |
| | | QKQLFVEQHKHYLDEIIEQISEFSKRVILADANLDKVLSAYNKHRDKPIREQAENIIH |
| | | LFTLTNLGAPAAFKYFDTTIDRKQYRSTKEVLDATLIHQSITGLYETRIDLSQLGGD |
| 34379 | SpRY Cas9 | MDKKYSIGLDIGTNSVGWAVITDEYKVPSKKFKVLGNTDRHSIKKNLIGALLFDSGE |
| | | TAERTRLKRTARRRYTRRKNRICYLQEIFSNEMAKVDDSFFHRLEESFLVEEDKKHE |
| | | RHPIFGNIVDEVAYHEKYPTIYHLRKKLVDSTDKADLRLIYLALAHMIKFRGHFLIEG |
| | | DLNPDNSDVDKLFIQLVQTYNQLFEENPINASGVDAKAILSARLSKSRRLENLIAQLP |
| | | GEKKNGLFGNLIALSLGLTPNFKSNFDLAEDAKLQLSKDTYDDDLDNLLAQIGDQY |
| | | ADLFLAAKNLSDAILLSDILRVNTEITKAPLSASMIKRYDEHHQDLTLLKALVRQQLP |
| | | EKYKEIFFDQSKNGYAGYIDGGASQEEFYKFIKPILEKMDGTEELLVKLNREDLLRK |
| | | QRTFDNGSIPHQIHLGELHAILRRQEDFYPFLKDNREKIEKILTFRIPYYVGPLARGNS |
| | | RFAWMTRKSEETITPWNFEEVVDKGASAQSFIERMTNFDKNLPNEKVLPKHSLLYE |
| | | YFTVYNELTKVKYVTEGMRKPAFLSGEQKKAIVDLLFKTNRKVTVKQLKEDYFKKI |
| | | ECFDSVEISGVEDRFNASLGTYHDLLKIIKDKDFLDNEENEDILEDIVLTLTLFEDREM |
| | | IEERLKTYAHLFDDKVMKQLKRRRYTGWGRLSRKLINGIRDKQSGKTILDFLKSDGF |
| | | ANRNFMQLIHDDSLTFKEDIQKAQVSGQGDSLHEHIANLAGSPAIKKGILQTVKVVD |
| | | ELVKVMGRHKPENIVIEMARENQTTQKGQKNSRERMKRIEEGIKELGSQILKEHPVE |
| | | NTQLQNEKLYLYYLQNGRDMYVDQELDINRLSDYDVDHIVPQSFLKDDSIDNKVLT |
| | | RSDKNRGKSDNVPSEEVVKKMKNYWRQLLNAKLITQRKFDNLTKAERGGLSELDK |
| | | AGFIKRQLVETRQITKHVAQILDSRMNTKYDENDKLIREVKVITLKSKLVSDFRKDF |
| | | QFYKVREINNYHHAHDAYLNAVVGTALIKKYPKLESEFVYGDYKVYDVRKMIAKS |
| | | EQEIGKATAKYFFYSNIMNFFKTEITLANGEIRKRPLIETNGETGEIVWDKGRDFATV |
| | | RKVLSMPQVNIVKKTEVQTGGFSKESIRPKRNSDKLIARKKDWDPKKYGGFLWPTV |
| | | AYSVLVVAKVEKGKSKKLKSVKELLGITIMERSSFEKNPIDFLEAKGYKEVKKDLIIK |
| | | LPKYSLFELENGRKRMLASAKQLQKGNELALPSKYVNFLYLASHYEKLKGSPEDNE |
| | | QKQLFVEQHKHYLDEIIEQISEFSKRVILADANLDKVLSAYNKHRDKPIREQAENIIH |
| | | LFTLTRLGAPRAFKYFDTTIDPKQYRSTKEVLDATLIHQSITGLYETRIDLSQLGGD |
| 34380 | SpRY Cas9 | MDKKYSIGLDIGTNSVGWAVITDEYKVPSKKFKVLGNTDRHSIKKNLIGALLFDSGE |
| | (H840A) | TAERTRLKRTARRRYTRRKNRICYLQEIFSNEMAKVDDSFFHRLEESFLVEEDKKHE |
| | Nickase | RHPIFGNIVDEVAYHEKYPTIYHLRKKLVDSTDKADLRLIYLALAHMIKFRGHFLIEG |
| | | DLNPDNSDVDKLFIQLVQTYNQLFEENPINASGVDAKAILSARLSKSRRLENLIAQLP |
| | | GEKKNGLFGNLIALSLGLTPNFKSNFDLAEDAKLQLSKDTYDDDLDNLLAQIGDQY |
| | | ADLFLAAKNLSDAILLSDILRVNTEITKAPLSASMIKRYDEHHQDLTLLKALVRQQLP |
| | | EKYKEIFFDQSKNGYAGYIDGGASQEEFYKFIKPILEKMDGTEELLVKLNREDLLRK |
| | | QRTFDNGSIPHQIHLGELHAILRRQEDFYPFLKDNREKIEKILTFRIPYYVGPLARGNS |
| | | RFAWMTRKSEETITPWNFEEVVDKGASAQSFIERMTNFDKNLPNEKVLPKHSLLYE |
| | | YFTVYNELTKVKYVTEGMRKPAFLSGEQKKAIVDLLFKTNRKVTVKQLKEDYFKKI |
| | | ECFDSVEISGVEDRFNASLGTYHDLLKIIKDKDFLDNEENEDILEDIVLTLTLFEDREM |
| | | IEERLKTYAHLFDDKVMKQLKRRRYTGWGRLSRKLINGIRDKQSGKTILDFLKSDGF |
| | | ANRNFMQLIHDDSLTFKEDIQKAQVSGQGDSLHEHIANLAGSPAIKKGILQTVKVVD |
| | | ELVKVMGRHKPENIVIEMARENQTTQKGQKNSRERMKRIEEGIKELGSQILKEHPVE |
| | | NTQLQNEKLYLYYLQNGRDMYVDQELDINRLSDYDVDAIVPQSFLKDDSIDNKVLT |
| | | RSDKNRGKSDNVPSEEVVKKMKNYWRQLLNAKLITQRKFDNLTKAERGGLSELDK |
| | | AGFIKRQLVETRQITKHVAQILDSRMNTKYDENDKLIREVKVITLKSKLVSDFRKDF |
| | | QFYKVREINNYHHAHDAYLNAVVGTALIKKYPKLESEFVYGDYKVYDVRKMIAKS |
| | | EQEIGKATAKYFFYSNIMNFFKTEITLANGEIRKRPLIETNGETGEIVWDKGRDFATV |
| | | RKVLSMPQVNIVKKTEVQTGGFSKESIRPKRNSDKLIARKKDWDPKKYGGFLWPTV |
| | | AYSVLVVAKVEKGKSKKLKSVKELLGITIMERSSFEKNPIDFLEAKGYKEVKKDLIIK |
| | | LPKYSLFELENGRKRMLASAKQLQKGNELALPSKYVNFLYLASHYEKLKGSPEDNE |
| | | QKQLFVEQHKHYLDEIIEQISEFSKRVILADANLDKVLSAYNKHRDKPIREQAENIIH |
| | | LFTLTRLGAPRAFKYFDTTIDPKQYRSTKEVLDATLIHQSITGLYETRIDLSQLGGD |
| 34381 | Met(-) SpRY | DKKYSIGLDIGTNSVGWAVITDEYKVPSKKFKVLGNTDRHSIKKNLIGALLFDSGET |
| | Cas9 Nickase | AERTRLKRTARRRYTRRKNRICYLQEIFSNEMAKVDDSFFHRLEESFLVEEDKKHER |
| | | HPIFGNIVDEVAYHEKYPTIYHLRKKLVDSTDKADLRLIYLALAHMIKFRGHFLIEGD |
| | | LNPDNSDVDKLFIQLVQTYNQLFEENPINASGVDAKAILSARLSKSRRLENLIAQLPG |
| | | EKKNGLFGNLIALSLGLTPNFKSNFDLAEDAKLQLSKDTYDDDLDNLLAQIGDQYA |
| | | DLFLAAKNLSDAILLSDILRVNTEITKAPLSASMIKRYDEHHQDLTLLKALVRQQLPE |
| | | KYKEIFFDQSKNGYAGYIDGGASQEEFYKFIKPILEKMDGTEELLVKLNREDLLRKQ |
| | | RTFDNGSIPHQIHLGELHAILRRQEDFYPFLKDNREKIEKILTFRIPYYVGPLARGNSR |
| | | FAWMTRKSEETITPWNFEEVVDKGASAQSFIERMTNFDKNLPNEKVLPKHSLLYEY |
| | | FTVYNELTKVKYVTEGMRKPAFLSGEQKKAIVDLLFKTNRKVTVKQLKEDYFKKIE |
| | | CFDSVEISGVEDRFNASLGTYHDLLKIIKDKDFLDNEENEDILEDIVLTLTLFEDREMI |
| | | EERLKTYAHLFDDKVMKQLKRRRYTGWGRLSRKLINGIRDKQSGKTILDFLKSDGF |
| | | ANRNFMQLIHDDSLTFKEDIQKAQVSGQGDSLHEHIANLAGSPAIKKGILQTVKVVD |
| | | ELVKVMGRHKPENIVIEMARENQTTQKGQKNSRERMKRIEEGIKELGSQILKEHPVE |
| | | NTQLQNEKLYLYYLQNGRDMYVDQELDINRLSDYDVDAIVPQSFLKDDSIDNKVLT |
| | | RSDKNRGKSDNVPSEEVVKKMKNYWRQLLNAKLITQRKFDNLTKAERGGLSELDK |
| | | AGFIKRQLVETRQITKHVAQILDSRMNTKYDENDKLIREVKVITLKSKLVSDFRKDF |
| | | QFYKVREINNYHHAHDAYLNAVVGTALIKKYPKLESEFVYGDYKVYDVRKMIAKS |
| | | EQEIGKATAKYFFYSNIMNFFKTEITLANGEIRKRPLIETNGETGEIVWDKGRDFATV |
| | | RKVLSMPQVNIVKKTEVQTGGFSKESIRPKRNSDKLIARKKDWDPKKYGGFLWPTV |
| | | AYSVLVVAKVEKGKSKKLKSVKELLGITIMERSSFEKNPIDFLEAKGYKEVKKDLIIK |
| | | LPKYSLFELENGRKRMLASAKQLQKGNELALPSKYVNFLYLASHYEKLKGSPEDNE |
| | | QKQLFVEQHKHYLDEIIEQISEFSKRVILADANLDKVLSAYNKHRDKPIREQAENIIH |
| | | LFTLTRLGAPRAFKYFDTTIDPKQYRSTKEVLDATLIHQSITGLYETRIDLSQLGGD |
| 34382 | sRGN3.1 | MNQKFILGLDIGITSVGYGLIDYETKNIIDAGVRLFPEANVENNEGRRSKRGSRRLKR |
| | | RRIHRLERVKLLLTEYDLINKEQIPTSNNPYQIRVKGLSEILSKDELAIALLHLAKRRG |
| | | IHNVDVAADKEETASDSLSTKDQINKNAKFLESRYVCELQKERLENEGHVRGVENR |
| | | FLTKDIVREAKKIIDTQMQYYPEIDETFKEKYISLVETRREYFEGPGQGSPFGWNGDL |
| | | KKWYEMLMGHCTYFPQELRSVKYAYSADLFNALNDLNNLIIQRDNSEKLEYHEKY |
| | | HIIENVFKQKKKPTLKQIAKEIGVNPEDIKGYRITKSGTPEFTSFKLFHDLKKVVKDH |
| | | AILDDIDLLNQIAEILTIYQDKDSIVAELGQLEYLMSEADKQSISELTGYTGTHSLSLK |
| | | CMNMIIDELWHSSMNQMEVFTYLNMRPKKYELKGYQRIPTDMIDDAILSPVVKRTF |
| | | IQSINVINKVIEKYGIPEDIIIELARENNSDDRKKFINNLQKKNEATRKRINEIIGQTGN |
| | | QNAKRIVEKIRLHDQQEGKCLYSLESIPLEDLLNNPNHYEVDHIIPRSVSFDNSYHNK |
| | | VLVKQSENSKKSNLTPYQYFNSGKSKLSYNQFKQHILNLSKSQDRISKKKKEYLLEE |
| | | RDINKFEVQKEFINRNLVDTRYATRELTNYLKAYFSANNMNVKVKTINGSFTDYLR |
| | | KVWKFKKERNHGYKHHAEDALIIANADFLFKENKKLKAVNSVLEKPEIETKQLDIQ |
| | | VDSEDNYSEMFIIPKQVQDIKDFRNFKYSHRVDKKPNRQLINDTLYSTRKKDNSTYI |
| | | VQTIKDIYAKDNTTLKKQFDKSPEKFLMYQHDPRTFEKLEVIMKQYANEKNPLAKY |
| | | HEETGEYLTKYSKKNNGPIVKSLKYIGNKLGSHLDVTHQFKSSTKKLVKLSIKNYRF |
| | | DVYLTEKGYKFVTIAYLNVFKKDNYYYIPKDKYQELKEKKKIKDTDQFIASFYKND |
| | | LIKLNGDLYKIIGVNSDDRNIIELDYYDIKYKDYCEINNIKGEPRIKKTIGKKTESIEKF |
| | | TTDVLGNLYLHSTEKAPQLIFKRGL |
| 34383 | sRGN3.1 | MNQKFILGLDIGITSVGYGLIDYETKNIIDAGVRLFPEANVENNEGRRSKRGSRRLKR |
| | (N585A) | RRIHRLERVKLLLTEYDLINKEQIPTSNNPYQIRVKGLSEILSKDELAIALLHLAKRRG |
| | Nickase | IHNVDVAADKEETASDSLSTKDQINKNAKFLESRYVCELQKERLENEGHVRGVENR |
| | | FLTKDIVREAKKIIDTQMQYYPEIDETFKEKYISLVETRREYFEGPGQGSPFGWNGDL |
| | | KKWYEMLMGHCTYFPQELRSVKYAYSADLFNALNDLNNLIIQRDNSEKLEYHEKY |
| | | HIIENVFKQKKKPTLKQIAKEIGVNPEDIKGYRITKSGTPEFTSFKLFHDLKKVVKDH |
| | | AILDDIDLLNQIAEILTIYQDKDSIVAELGQLEYLMSEADKQSISELTGYTGTHSLSLK |
| | | CMNMIIDELWHSSMNQMEVFTYLNMRPKKYELKGYQRIPTDMIDDAILSPVVKRTF |
| | | IQSINVINKVIEKYGIPEDIIIELARENNSDDRKKFINNLQKKNEATRKRINEIIGQTGN |
| | | QNAKRIVEKIRLHDQQEGKCLYSLESIPLEDLLNNPNHYEVDHIIPRSVSFDNSYHNK |
| | | VLVKQSEASKKSNLTPYQYFNSGKSKLSYNQFKQHILNLSKSQDRISKKKKEYLLEE |
| | | RDINKFEVQKEFINRNLVDTRYATRELTNYLKAYFSANNMNVKVKTINGSFTDYLR |
| | | KVWKFKKERNHGYKHHAEDALIIANADFLFKENKKLKAVNSVLEKPEIETKQLDIQ |
| | | VDSEDNYSEMFIIPKQVQDIKDFRNFKYSHRVDKKPNRQLINDTLYSTRKKDNSTYI |
| | | VQTIKDIYAKDNTTLKKQFDKSPEKFLMYQHDPRTFEKLEVIMKQYANEKNPLAKY |
| | | HEETGEYLTKYSKKNNGPIVKSLKYIGNKLGSHLDVTHQFKSSTKKLVKLSIKNYRF |
| | | DVYLTEKGYKFVTIAYLNVFKKDNYYYIPKDKYQELKEKKKIKDTDQFIASFYKND |
| | | LIKLNGDLYKIIGVNSDDRNIIELDYYDIKYKDYCEINNIKGEPRIKKTIGKKTESIEKF |
| | | TTDVLGNLYLHSTEKAPQLIFKRGL |
| 34384 | Met(-) | NQKFILGLDIGITSVGYGLIDYETKNIIDAGVRLFPEANVENNEGRRSKRGSRRLKRR |
| | sRGN3.1 | RIHRLERVKLLLTEYDLINKEQIPTSNNPYQIRVKGLSEILSKDELAIALLHLAKRRGI |
| | (N584A) | HNVDVAADKEETASDSLSTKDQINKNAKFLESRYVCELQKERLENEGHVRGVENRF |
| | Nickase | LTKDIVREAKKIIDTQMQYYPEIDETFKEKYISLVETRREYFEGPGQGSPFGWNGDLK |
| | | KWYEMLMGHCTYFPQELRSVKYAYSADLFNALNDLNNLIIQRDNSEKLEYHEKYHI |
| | | IENVFKQKKKPTLKQIAKEIGVNPEDIKGYRITKSGTPEFTSFKLFHDLKKVVKDHAI |
| | | LDDIDLLNQIAEILTIYQDKDSIVAELGQLEYLMSEADKQSISELTGYTGTHSLSLKC |
| | | MNMIIDELWHSSMNQMEVFTYLNMRPKKYELKGYQRIPTDMIDDAILSPVVKRTFI |
| | | QSINVINKVIEKYGIPEDIIIELARENNSDDRKKFINNLQKKNEATRKRINEIIGQTGNQ |
| | | NAKRIVEKIRLHDQQEGKCLYSLESIPLEDLLNNPNHYEVDHIIPRSVSFDNSYHNKV |
| | | LVKQSEASKKSNLTPYQYFNSGKSKLSYNQFKQHILNLSKSQDRISKKKKEYLLEER |
| | | DINKFEVQKEFINRNLVDTRYATRELTNYLKAYFSANNMNVKVKTINGSFTDYLRK |
| | | VWKFKKERNHGYKHHAEDALIIANADFLFKENKKLKAVNSVLEKPEIETKQLDIQV |
| | | DSEDNYSEMFIIPKQVQDIKDFRNFKYSHRVDKKPNRQLINDTLYSTRKKDNSTYIV |
| | | QTIKDIYAKDNTTLKKQFDKSPEKFLMYQHDPRTFEKLEVIMKQYANEKNPLAKYH |
| | | EETGEYLTKYSKKNNGPIVKSLKYIGNKLGSHLDVTHQFKSSTKKLVKLSIKNYRFD |
| | | VYLTEKGYKFVTIAYLNVFKKDNYYYIPKDKYQELKEKKKIKDTDQFIASFYKNDLI |
| | | KLNGDLYKIIGVNSDDRNIIELDYYDIKYKDYCEINNIKGEPRIKKTIGKKTESIEKFTT |
| | | DVLGNLYLHSTEKAPQLIFKRGL |
| 34385 | sRGN3.3 | MNQKFILGLDIGITSVGYGLIDYETKNIIDAGVRLFPEANVENNEGRRSKRGSRRLKR |
| | | RRIHRLERVKLLLTEYDLINKEQIPTSNNPYQIRVKGLSEILSKDELAIALLHLAKRRG |
| | | IHNVDVAADKEETASDSLSTKDQINKNAKFLESRYVCELQKERLENEGHVRGVENR |
| | | FLTKDIVREAKKIIDTQMQYYPEIDETFKEKYISLVETRREYFEGPGQGSPFGWNGDL |
| | | KKWYEMLMGHCTYFPQELRSVKYAYSADLFNALNDLNNLIIQRDNSEKLEYHEKY |
| | | HIIENVFKQKKKPTLKQIAKEIGVNPEDIKGYRITKSGTPEFTSFKLFHDLKKVVKDH |
| | | AILDDIDLLNQIAEILTIYQDKDSIVAELGQLEYLMSEADKQSISELTGYTGTHSLSLK |
| | | CMNMIIDELWHSSMNQMEVFTYLNMRPKKYELKGYQRIPTDMIDDAILSPVVKRTF |
| | | IQSINVINKVIEKYGIPEDIIIELARENNSDDRKKFINNLQKKNEATRKRINEIIGQTGN |
| | | QNAKRIVEKIRLHDQQEGKCLYSLESIPLEDLLNNPNHYEVDHIIPRSVSFDNSYHNK |
| | | VLVKQSENSKKSNLTPYQYFNSGKSKLSYNQFKQHILNLSKSQDRISKKKKEYLLEE |
| | | RDINKFEVQKEFINRNLVDTRYATRELTSYLKAYFSANNMDVKVKTINGSFTNHLR |
| | | KVWRFDKYRNHGYKHHAEDALIIANADFLFKENKKLQNTNKILEKPTIENNTKKVT |
| | | VEKEEDYNNVFETPKLVEDIKQYRDYKFSHRVDKKPNRQLINDTLYSTRMKDEHDY |
| | | IVQTITDIYGKDNTNLKKQFNKNPEKFLMYQNDPKTFEKLSIIMKQYSDEKNPLAKY |
| | | YEETGEYLTKYSKKNNGPIVKKIKLLGNKVGNHLDVTNKYENSTKKLVKLSIKNYR |
| | | FDVYLTEKGYKFVTIAYLNVFKKDNYYYIPKDKYQELKEKKKIKDTDQFIASFYKN |
| | | DLIKLNGDLYKIIGVNSDDRNIIELDYYDIKYKDYCEINNIKGEPRIKKTIGKKTESIEK |
| | | FTTDVLGNLYLHSTEKAPQLIFKRGL |
| 34386 | sRGN3.3 | MNQKFILGLDIGITSVGYGLIDYETKNIIDAGVRLFPEANVENNEGRRSKRGSRRLKR |
| | (N585A) | RRIHRLERVKLLLTEYDLINKEQIPTSNNPYQIRVKGLSEILSKDELAIALLHLAKRRG |
| | Nickase | IHNVDVAADKEETASDSLSTKDQINKNAKFLESRYVCELQKERLENEGHVRGVENR |
| | | FLTKDIVREAKKIIDTQMQYYPEIDETFKEKYISLVETRREYFEGPGQGSPFGWNGDL |
| | | KKWYEMLMGHCTYFPQELRSVKYAYSADLFNALNDLNNLIIQRDNSEKLEYHEKY |
| | | HIIENVFKQKKKPTLKQIAKEIGVNPEDIKGYRITKSGTPEFTSFKLFHDLKKVVKDH |
| | | AILDDIDLLNQIAEILTIYQDKDSIVAELGQLEYLMSEADKQSISELTGYTGTHSLSLK |
| | | CMNMIIDELWHSSMNQMEVFTYLNMRPKKYELKGYQRIPTDMIDDAILSPVVKRTF |
| | | IQSINVINKVIEKYGIPEDIIIELARENNSDDRKKFINNLQKKNEATRKRINEIIGQTGN |
| | | QNAKRIVEKIRLHDQQEGKCLYSLESIPLEDLLNNPNHYEVDHIIPRSVSFDNSYHNK |
| | | VLVKQSEASKKSNLTPYQYFNSGKSKLSYNQFKQHILNLSKSQDRISKKKKEYLLEE |
| | | RDINKFEVQKEFINRNLVDTRYATRELTSYLKAYFSANNMDVKVKTINGSFTNHLR |
| | | KVWRFDKYRNHGYKHHAEDALIIANADFLFKENKKLQNTNKILEKPTIENNTKKVT |
| | | VEKEEDYNNVFETPKLVEDIKQYRDYKFSHRVDKKPNRQLINDTLYSTRMKDEHDY |
| | | IVQTITDIYGKDNTNLKKQFNKNPEKFLMYQNDPKTFEKLSIIMKQYSDEKNPLAKY |
| | | YEETGEYLTKYSKKNNGPIVKKIKLLGNKVGNHLDVTNKYENSTKKLVKLSIKNYR |
| | | FDVYLTEKGYKFVTIAYLNVFKKDNYYYIPKDKYQELKEKKKIKDTDQFIASFYKN |
| | | DLIKLNGDLYKIIGVNSDDRNIIELDYYDIKYKDYCEINNIKGEPRIKKTIGKKTESIEK |
| | | FTTDVLGNLYLHSTEKAPQLIFKRGL |
| 34387 | Met(-) | NQKFILGLDIGITSVGYGLIDYETKNIIDAGVRLFPEANVENNEGRRSKRGSRRLKRR |
| | sRGN3.3 | RIHRLERVKLLLTEYDLINKEQIPTSNNPYQIRVKGLSEILSKDELAIALLHLAKRRGI |
| | (N584A) | HNVDVAADKEETASDSLSTKDQINKNAKFLESRYVCELQKERLENEGHVRGVENRF |
| | Nickase | LTKDIVREAKKIIDTQMQYYPEIDETFKEKYISLVETRREYFEGPGQGSPFGWNGDLK |
| | | KWYEMLMGHCTYFPQELRSVKYAYSADLFNALNDLNNLIIQRDNSEKLEYHEKYHI |
| | | IENVFKQKKKPTLKQIAKEIGVNPEDIKGYRITKSGTPEFTSFKLFHDLKKVVKDHAI |
| | | LDDIDLLNQIAEILTIYQDKDSIVAELGQLEYLMSEADKQSISELTGYTGTHSLSLKC |
| | | MNMIIDELWHSSMNQMEVFTYLNMRPKKYELKGYQRIPTDMIDDAILSPVVKRTFI |
| | | QSINVINKVIEKYGIPEDIIIELARENNSDDRKKFINNLQKKNEATRKRINEIIGQTGNQ |
| | | NAKRIVEKIRLHDQQEGKCLYSLESIPLEDLLNNPNHYEVDHIIPRSVSFDNSYHNKV |
| | | LVKQSEASKKSNLTPYQYFNSGKSKLSYNQFKQHILNLSKSQDRISKKKKEYLLEER |
| | | DINKFEVQKEFINRNLVDTRYATRELTSYLKAYFSANNMDVKVKTINGSFTNHLRK |
| | | VWRFDKYRNHGYKHHAEDALIIANADFLFKENKKLQNTNKILEKPTIENNTKKVTV |
| | | EKEEDYNNVFETPKLVEDIKQYRDYKFSHRVDKKPNRQLINDTLYSTRMKDEHDYI |
| | | VQTITDIYGKDNTNLKKQFNKNPEKFLMYQNDPKTFEKLSIIMKQYSDEKNPLAKY |
| | | YEETGEYLTKYSKKNNGPIVKKIKLLGNKVGNHLDVTNKYENSTKKLVKLSIKNYR |
| | | FDVYLTEKGYKFVTIAYLNVFKKDNYYYIPKDKYQELKEKKKIKDTDQFIASFYKN |
| | | DLIKLNGDLYKIIGVNSDDRNIIELDYYDIKYKDYCEINNIKGEPRIKKTIGKKTESIEK |
| | | FTTDVLGNLYLHSTEKAPQLIFKRGL |
| 34388 | SpG | MDKKYSIGLDIGTNSVGWAVITDEYKVPSKKFKVLGNTDRHSIKKNLIGALLFDSGE |
| | | TAEATRLKRTARRRYTRRKNRICYLQEIFSNEMAKVDDSFFHRLEESFLVEEDKKHE |
| | | RHPIFGNIVDEVAYHEKYPTIYHLRKKLVDSTDKADLRLIYLALAHMIKFRGHFLIEG |
| | | DLNPDNSDVDKLFIQLVQTYNQLFEENPINASGVDAKAILSARLSKSRRLENLIAQLP |
| | | GEKKNGLFGNLIALSLGLTPNFKSNFDLAEDAKLQLSKDTYDDDLDNLLAQIGDQY |
| | | ADLFLAAKNLSDAILLSDILRVNTEITKAPLSASMIKRYDEHHQDLTLLKALVRQQLP |
| | | EKYKEIFFDQSKNGYAGYIDGGASQEEFYKFIKPILEKMDGTEELLVKLNREDLLRK |
| | | QRTFDNGSIPHQIHLGELHAILRRQEDFYPFLKDNREKIEKILTFRIPYYVGPLARGNS |
| | | RFAWMTRKSEETITPWNFEEVVDKGASAQSFIERMTNFDKNLPNEKVLPKHSLLYE |
| | | YFTVYNELTKVKYVTEGMRKPAFLSGEQKKAIVDLLFKTNRKVTVKQLKEDYFKKI |
| | | ECFDSVEISGVEDRFNASLGTYHDLLKIIKDKDFLDNEENEDILEDIVLTLTLFEDREM |
| | | IEERLKTYAHLFDDKVMKQLKRRRYTGWGRLSRKLINGIRDKQSGKTILDFLKSDGF |
| | | ANRNFMQLIHDDSLTFKEDIQKAQVSGQGDSLHEHIANLAGSPAIKKGILQTVKVVD |
| | | ELVKVMGRHKPENIVIEMARENQTTQKGQKNSRERMKRIEEGIKELGSQILKEHPVE |
| | | NTQLQNEKLYLYYLQNGRDMYVDQELDINRLSDYDVDHIVPQSFLKDDSIDNKVLT |
| | | RSDKNRGKSDNVPSEEVVKKMKNYWRQLLNAKLITQRKFDNLTKAERGGLSELDK |
| | | AGFIKRQLVETRQITKHVAQILDSRMNTKYDENDKLIREVKVITLKSKLVSDFRKDF |
| | | QFYKVREINNYHHAHDAYLNAVVGTALIKKYPKLESEFVYGDYKVYDVRKMIAKS |
| | | EQEIGKATAKYFFYSNIMNFFKTEITLANGEIRKRPLIETNGETGEIVWDKGRDFATV |
| | | RKVLSMPQVNIVKKTEVQTGGFSKESILPKRNSDKLIARKKDWDPKKYGGFLWPTV |
| | | AYSVLVVAKVEKGKSKKLKSVKELLGITIMERSSFEKNPIDFLEAKGYKEVKKDLIIK |
| | | LPKYSLFELENGRKRMLASAKQLQKGNELALPSKYVNFLYLASHYEKLKGSPEDNE |
| | | QKQLFVEQHKHYLDEIIEQISEFSKRVILADANLDKVLSAYNKHRDKPIREQAENIIH |
| | | LFTLTNLGAPAAFKYFDTTIDRKQYRSTKEVLDATLIHQSITGLYETRIDLSQLGGD |
| 34389 | SpG(H840A) | MDKKYSIGLDIGTNSVGWAVITDEYKVPSKKFKVLGNTDRHSIKKNLIGALLFDSGE |
| | Nickase | TAEATRLKRTARRRYTRRKNRICYLQEIFSNEMAKVDDSFFHRLEESFLVEEDKKHE |
| | | RHPIFGNIVDEVAYHEKYPTIYHLRKKLVDSTDKADLRLIYLALAHMIKFRGHFLIEG |
| | | DLNPDNSDVDKLFIQLVQTYNQLFEENPINASGVDAKAILSARLSKSRRLENLIAQLP |
| | | GEKKNGLFGNLIALSLGLTPNFKSNFDLAEDAKLQLSKDTYDDDLDNLLAQIGDQY |
| | | ADLFLAAKNLSDAILLSDILRVNTEITKAPLSASMIKRYDEHHQDLTLLKALVRQQLP |
| | | EKYKEIFFDQSKNGYAGYIDGGASQEEFYKFIKPILEKMDGTEELLVKLNREDLLRK |
| | | QRTFDNGSIPHQIHLGELHAILRRQEDFYPFLKDNREKIEKILTFRIPYYVGPLARGNS |
| | | RFAWMTRKSEETITPWNFEEVVDKGASAQSFIERMTNFDKNLPNEKVLPKHSLLYE |
| | | YFTVYNELTKVKYVTEGMRKPAFLSGEQKKAIVDLLFKTNRKVTVKQLKEDYFKKI |
| | | ECFDSVEISGVEDRFNASLGTYHDLLKIIKDKDFLDNEENEDILEDIVLTLTLFEDREM |
| | | IEERLKTYAHLFDDKVMKQLKRRRYTGWGRLSRKLINGIRDKQSGKTILDFLKSDGF |
| | | ANRNFMQLIHDDSLTFKEDIQKAQVSGQGDSLHEHIANLAGSPAIKKGILQTVKVVD |
| | | ELVKVMGRHKPENIVIEMARENQTTQKGQKNSRERMKRIEEGIKELGSQILKEHPVE |
| | | NTQLQNEKLYLYYLQNGRDMYVDQELDINRLSDYDVDAIVPQSFLKDDSIDNKVLT |
| | | RSDKNRGKSDNVPSEEVVKKMKNYWRQLLNAKLITQRKFDNLTKAERGGLSELDK |
| | | AGFIKRQLVETRQITKHVAQILDSRMNTKYDENDKLIREVKVITLKSKLVSDFRKDF |
| | | QFYKVREINNYHHAHDAYLNAVVGTALIKKYPKLESEFVYGDYKVYDVRKMIAKS |
| | | EQEIGKATAKYFFYSNIMNFFKTEITLANGEIRKRPLIETNGETGEIVWDKGRDFATV |
| | | RKVLSMPQVNIVKKTEVQTGGFSKESILPKRNSDKLIARKKDWDPKKYGGFLWPTV |
| | | AYSVLVVAKVEKGKSKKLKSVKELLGITIMERSSFEKNPIDFLEAKGYKEVKKDLIIK |
| | | LPKYSLFELENGRKRMLASAKQLQKGNELALPSKYVNFLYLASHYEKLKGSPEDNE |
| | | QKQLFVEQHKHYLDEIIEQISEFSKRVILADANLDKVLSAYNKHRDKPIREQAENIIH |
| | | LFTLTNLGAPAAFKYFDTTIDRKQYRSTKEVLDATLIHQSITGLYETRIDLSQLGGD |
| 34390 | Met(-) | DKKYSIGLDIGTNSVGWAVITDEYKVPSKKFKVLGNTDRHSIKKNLIGALLFDSGET |
| | SpG(H839A) | AEATRLKRTARRRYTRRKNRICYLQEIFSNEMAKVDDSFFHRLEESFLVEEDKKHER |
| | Nickase | HPIFGNIVDEVAYHEKYPTIYHLRKKLVDSTDKADLRLIYLALAHMIKFRGHFLIEGD |
| | | LNPDNSDVDKLFIQLVQTYNQLFEENPINASGVDAKAILSARLSKSRRLENLIAQLPG |
| | | EKKNGLFGNLIALSLGLTPNFKSNFDLAEDAKLQLSKDTYDDDLDNLLAQIGDQYA |
| | | DLFLAAKNLSDAILLSDILRVNTEITKAPLSASMIKRYDEHHQDLTLLKALVRQQLPE |
| | | KYKEIFFDQSKNGYAGYIDGGASQEEFYKFIKPILEKMDGTEELLVKLNREDLLRKQ |
| | | RTFDNGSIPHQIHLGELHAILRRQEDFYPFLKDNREKIEKILTFRIPYYVGPLARGNSR |
| | | FAWMTRKSEETITPWNFEEVVDKGASAQSFIERMTNFDKNLPNEKVLPKHSLLYEY |
| | | FTVYNELTKVKYVTEGMRKPAFLSGEQKKAIVDLLFKTNRKVTVKQLKEDYFKKIE |
| | | CFDSVEISGVEDRFNASLGTYHDLLKIIKDKDFLDNEENEDILEDIVLTLTLFEDREMI |
| | | EERLKTYAHLFDDKVMKQLKRRRYTGWGRLSRKLINGIRDKQSGKTILDFLKSDGF |
| | | ANRNFMQLIHDDSLTFKEDIQKAQVSGQGDSLHEHIANLAGSPAIKKGILQTVKVVD |
| | | ELVKVMGRHKPENIVIEMARENQTTQKGQKNSRERMKRIEEGIKELGSQILKEHPVE |
| | | NTQLQNEKLYLYYLQNGRDMYVDQELDINRLSDYDVDAIVPQSFLKDDSIDNKVLT |
| | | RSDKNRGKSDNVPSEEVVKKMKNYWRQLLNAKLITQRKFDNLTKAERGGLSELDK |
| | | AGFIKRQLVETRQITKHVAQILDSRMNTKYDENDKLIREVKVITLKSKLVSDFRKDF |
| | | QFYKVREINNYHHAHDAYLNAVVGTALIKKYPKLESEFVYGDYKVYDVRKMIAKS |
| | | EQEIGKATAKYFFYSNIMNFFKTEITLANGEIRKRPLIETNGETGEIVWDKGRDFATV |
| | | RKVLSMPQVNIVKKTEVQTGGFSKESILPKRNSDKLIARKKDWDPKKYGGFLWPTV |
| | | AYSVLVVAKVEKGKSKKLKSVKELLGITIMERSSFEKNPIDFLEAKGYKEVKKDLIIK |
| | | LPKYSLFELENGRKRMLASAKQLQKGNELALPSKYVNFLYLASHYEKLKGSPEDNE |
| | | QKQLFVEQHKHYLDEIIEQISEFSKRVILADANLDKVLSAYNKHRDKPIREQAENIIH |
| | | LFTLTNLGAPAAFKYFDTTIDRKQYRSTKEVLDATLIHQSITGLYETRIDLSQLGGD |

In some embodiments, a Cas9 protein comprises a variant Cas9 protein containing one or more amino acid substitutions. In some embodiments, a wildtype Cas9 protein comprises a RuvC domain and an HNH domain. In some embodiments, a prime editor comprises a nuclease active Cas9 protein that may cleave both strands of a double stranded target DNA sequence. In some embodiments, the nuclease active Cas9 protein comprises a functional RuvC domain and a functional HNH domain. In some embodiments, a prime editor comprises a Cas9 nickase that can bind to a guide polynucleotide and recognize a target DNA, but can cleave only one strand of a double stranded target DNA. In some embodiments, the Cas9 nickase comprises only one functional RuvC domain or one functional HNH domain. In some embodiments, a prime editor comprises a Cas9 that has a non-functional HNH domain and a functional RuvC domain. In some embodiments, the prime editor can cleave the edit strand (i.e., the PAM strand), but not the non-edit strand of a double stranded target DNA sequence. In some embodiments, a prime editor comprises a Cas9 having a non-functional RuvC domain that can cleave the target strand (i.e., the non-PAM strand), but not the edit strand of a double stranded target DNA sequence. In some embodiments, a prime editor comprises a Cas9 that has neither a functional RuvC domain nor a functional HNH domain, which may not cleave any strand of a double stranded target DNA sequence.

In some embodiments, a prime editor comprises a Cas9 having a mutation in the RuvC domain that reduces or abolishes the nuclease activity of the RuvC domain. In some embodiments, the Cas9 comprises a mutation at amino acid D10 as compared to a wild type SpCas9 as set forth in SEQ ID NO: 34364, or a corresponding mutation thereof. In some embodiments, the Cas9 comprises a D10A mutation as compared to a wild type SpCas9 as set forth in SEQ ID NO: 34364, or a corresponding mutation thereof. In some embodiments, the Cas9 polypeptide comprises a mutation at amino acid D10, G12, and/or G17 as compared to a wild type SpCas9 as set forth in SEQ ID NO: 34364, or a corresponding mutation thereof. In some embodiments, the Cas9 polypeptide comprises a D10A mutation, a G12A mutation, and/or a G17A mutation as compared to a wild type SpCas9 as set forth in SEQ ID NO: 34364, or a corresponding mutation thereof.

In some embodiments, a prime editor comprises a Cas9 polypeptide having a mutation in the HNH domain that reduces or abolishes the nuclease activity of the HNH domain. In some embodiments, the Cas9 polypeptide comprises a mutation at amino acid H840 as compared to a wild type SpCas9 as set forth in SEQ ID NO: 34364, or a corresponding mutation thereof. In some embodiments, the Cas9 polypeptide comprises a H840A mutation as compared to a wild type SpCas9 as set forth in SEQ ID NO: 34364, or a corresponding mutation thereof. In some embodiments, the Cas9 polypeptide comprises a mutation at amino acid E762, D839, H840, N854, N856, N863, H982, H983, A984, D986, and/or a A987 as compared to a wild type SpCas9 as set forth in SEQ ID NO: 34364, or a corresponding mutation thereof. In some embodiments, the Cas9 polypeptide comprises a E762A, D839A, H840A, N854A, N856A, N863A, H982A, H983A, A984A, and/or a D986A mutation as compared to a wild type SpCas9 as set forth in SEQ ID NO: 34364, or a corresponding mutation thereof. In some embodiments, the Cas9 polypeptide comprises a mutation at amino acid residue R221, N394, and/or H840 as compared to a wild type SpCas9 (e.g., SEQ ID NO: 34364). In some embodiments, the Cas9 polypeptide comprises a R221K, N394L, and/or H840A mutation as compared to a wild type SpCas9 as set forth in SEQ ID NO: 34364, or a corresponding mutation thereof. In some embodiments, the Cas9 polypeptide comprises a mutation at amino acid residue R220, N393, and/or H839 as compared to a wild type SpCas9 (e.g., SEQ ID NO: 34364) lacking a N-terminal methionine, or a corresponding mutation thereof. In some embodiments, the Cas9 polypeptide comprises a R220K, N393K, and/or H839A mutation as compared to a wild type SpCas9 (as set forth in SEQ ID NO: 34364) lacking a N-terminal methionine, or a corresponding mutation thereof.

In some embodiments, a prime editor comprises a Cas9 having one or more amino acid substitutions in both the HNH domain and the RuvC domain that reduce or abolish the nuclease activity of both the HNH domain and the RuvC domain. In some embodiments, the prime editor comprises a nuclease inactive Cas9, or a nuclease dead Cas9 (dCas9). In some embodiments, the dCas9 comprises a H840X substitution and a D10X mutation compared to a wild type SpCas9 as set forth in SEQ ID NO: 34364 or corresponding mutations thereof, wherein X is any amino acid other than H for the H840X substitution and any amino acid other than D for the DIOX substitution. In some embodiments, the dead Cas9 comprises a H840A and a D10A mutation as compared to a wild type SpCas9 as set forth in SEQ ID NO: 34364, or corresponding mutations thereof.

In some embodiments, the N-terminal methionine is removed from the amino acid sequence of a Cas9 nickase, or from any Cas9 variant, ortholog, or equivalent disclosed or contemplated herein. For example, methionine-minus (Met (−)) Cas9 nickases include any one of the sequences set forth in SEQ ID NOs: 34366, 34369, 34372, 34375, 34378, 34381, 34384, 34387, 34390 or a variant thereof having an amino acid sequence that has at least 80%, at least 85%, at least 90%, at least 95%, or at least 99% sequence identity thereto.

Besides dead Cas9 and Cas9 nickase variants, the Cas9 proteins used herein may also include other Cas9 variants having at least about 70%, at least about 80%, at least about 90%, at least about 95%, at least about 96%, at least about 97%, at least about 98%, at least about 99%, at least about 99.5%, or at least about 99.9% sequence identity to any reference Cas9 protein, including any wild type Cas9, or mutant Cas9 (e.g., a dead Cas9 or Cas9 nickase), or fragment Cas9, or circular permutant Cas9, or other variant of Cas9 disclosed herein or known in the art. In some embodiments, a Cas9 variant may have 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50 or more amino acid changes compared to a reference Cas9, e.g., a wild type Cas9. In some embodiments, the Cas9 variant comprises a fragment of a reference Cas9 (e.g., a gRNA binding domain or a DNA-cleavage domain), such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the corresponding fragment of a reference Cas9, e.g., a wild type Cas9. In some embodiments, the fragment is at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or at least 99.5% of the amino acid length of a corresponding wild type Cas9.

In some embodiments, a Cas9 fragment is a functional fragment that retains one or more Cas9 activities. In some embodiments, the Cas9 fragment is at least 100 amino acids in length. In some embodiments, the fragment is at least 100, 150, 200, 250, 300, 350, 400, 450, 500, 550, 600, 650, 700, 750, 800, 850, 900, 950, 1000, 1050, 1100, 1150, 1200, 1250, or at least 1300 amino acids in length.

In some embodiments, a prime editor comprises a Cas protein, e.g., Cas9, containing modifications that allow altered PAM recognition. In prime editing using a Cas-protein-based prime editor, a “protospacer adjacent motif (PAM)”, PAM sequence, or PAM-like motif, may be used to refer to a short DNA sequence immediately following the protospacer sequence on the PAM strand of the target gene. In some embodiments, the PAM is recognized by the Cas nuclease in the prime editor during prime editing. In certain embodiments, the PAM is required for target binding of the Cas protein. The specific PAM sequence required for Cas protein recognition may depend on the specific type of the Cas protein. A PAM can be 1, 2, 3, 4, 5, 6, 7, 8, 9, 10 or more nucleotides in length. In some embodiments, a PAM is between 2-6 nucleotides in length. In some embodiments, the PAM can be a 5′ PAM (i.e., located upstream of the 5′ end of the protospacer). In other embodiments, the PAM can be a 3′ PAM (i.e., located downstream of the 5′ end of the protospacer). In some embodiments, the Cas protein of a prime editor recognizes a canonical PAM, for example, a SpCas9 recognizes 5′-NGG-3′ PAM. In some embodiments, the Cas protein of a prime editor has altered or non-canonical PAM specificities. Exemplary PAM sequences and corresponding Cas variants are described in Table 80 below. It should be appreciated that for each of the variants provided, the Cas protein comprises one or more of the amino acid substitutions as indicated compared to a wild type Cas protein sequence, for example, the Cas9 as set forth in SEQ ID NO: 34364. The PAM motifs as shown in Table 80 below are in the order of 5′ to 3′. In some embodiments, the Cas proteins of the disclosure can also be used to direct transcriptional control of target sequences, for example silencing transcription by sequence-specific binding to target sequences. In some embodiments, a Cas protein described herein may have one or mutations in a PAM recognition motif. In some embodiments, a Cas protein described herein may have altered PAM specificity.

As used in PAM sequences in Table 80, “N” refers to any one of nucleotides A, G, C, and T, “R” refers to nucleotide A or G, and “Y” refers to nucleotide C or T.

TABLE 80
Cas protein variants and
corresponding PAM sequences

| Variant | PAM |
|---|---|
| spCas9 (wild type) | NGG, NGA, |
| | NAG |
| spCas9- VRVRFRR | NG |
| R1335V/L1111R/D1135V/G1218R/ | |
| E1219F/A1322R/T1337R | |
| spCas9-VQR (D1135V/R1335Q/T1337R) | NGA |
| spCas9-EQR (D1135E/R1335Q/T1337R) | NGA |
| spCas9-VRER | NGCG |
| (D1135V/G1218R/R1335E/T1337R) | |
| spCas9-VRQR | NGA |
| (D1135V, G1218R, R1335Q, T1337R) | |
| Cas9-NG | NGN |
| (L1111R, D1135V, G1218R, E1219F, | |
| A1322R, T1337R, R1335V) | |
| SpG Cas9 | NGN |
| (D1135L, S1136W, G1218K, E1219Q, | |
| R1335Q, T1337R) | |
| SyRY Cas9 | NRN |
| (A61R, L1111R, N1317R, A1322R, | |
| and R1333P) | |
| xCas9 | NGN |
| (E480K, E543D, E1219V, K294R, | |
| Q1256K, A262T, S409I, M694I) | |
| SluCa9 | NNGG |
| sRGN1, sRGN2, sRGN4, sRGN3.1, | NNGG |
| sRGN3.3 | |
| saCas9 | NNGRRT, |
| | NNGRRN |
| saCas9-KKH (E782K, N968K, R1015H) | NNNRRT |
| spCas9-MQKSER | NGCG/NGCN |
| (D1135M, S1136Q, G1218K, E1219S, | |
| R1335E, T1337R) | |
| spCas9-LRKIQK | NGTN |
| (D1135L, S1136R, G1218K, E1219I, | |
| R1335Q, T1337K) | |
| spCas9-LRVSQK | NGTN |
| (D1135L, S1136R, G1218V, E1219S, | |
| R1335Q, T1337K) | |
| spCas9-LRVSQL | NGTN |
| (D1135L, S1136R, G1218V, E1219S, | |
| R1335Q, T1337L) | |
| Cpf1 | TTTV |
| Spy-Mac | NAA |
| NmCas9 | NNNNGATT |
| StCas9 | NNAGAAW |
| TdCas9 | NAAAAC |

In some embodiments, a prime editor comprises a Cas9 polypeptide comprising one or mutations selected from the group consisting of: A61R, L111R, D1135, R221K, A262T, R324L, N394K, S409I, S409I, E427G, E480K, M495V, N497A, Y515N, K526E, F539S, E5431D, R654L, R661A, R661L, R691A, N692A, M694A, M694I, Q695A, H698A, R753G, M763I, K848A, K890N, Q926A, K1003A, R1060A, L1111R, R1114G, D1135E, D11135L, D11135N, S11136W, V11139A, D11180G, G1218K, G1218R, G1218S, E1219Q, E1219V, E1219V, Q1221H, P1249S, E1253K, N1317R, A1320V, P1321S, A1322R, I1322V, D1332G, R1332N, A1332R, R1333K, R1333P, R1335L, R1335Q, R1335V, T1337N, T1337R, S1338T, H1349R, and any combinations thereof as compared to a wildtype SpCas9 polypeptide as set forth in SEQ ID NO: 34364.

In some embodiments, a prime editor comprises a SaCas9 polypeptide. In some embodiments, the SaCas9 polypeptide comprises one or more of mutations E782K, N968K, and R10115H as compared to a wild type SaCas9. In some embodiments, a prime editor comprises a FnCas9 polypeptide, for example, a wildtype FnCas9 polypeptide or a FnCas9 polypeptide comprising one or more of mutations E1369R, E1449H, or R1556A as compared to the wild type FnCas9. In some embodiments, a prime editor comprises a Sc Cas9, for example, a wild type ScCas9 or a ScCas9 polypeptide comprises one or more of mutations I367K, G3681D, I369K, H371L, T375S, T376G, and T1227K as compared to the wild type ScCas9. In some embodiments, a prime editor comprises a St1 Cas9 polypeptide, a St3 Cas9 polypeptide, or a SluCas9 polypeptide.

In some embodiments, a prime editor comprises a Cas polypeptide that comprises a circular permutant Cas variant. For example, a Cas9 polypeptide of a prime editor may be engineered such that the N-terminus and the C-terminus of a Cas9 protein (e.g., a wild type Cas9 protein, or a Cas9 nickase) are topically rearranged to retain the ability to bind DNA when complexed with a guide RNA (gRNA). An exemplary circular permutant configuration may be N-terminus-[original C-terminus]-[original N-terminus]-C-terminus. Any of the Cas9 proteins described herein, including any variant, ortholog, or naturally occurring Cas9 or equivalent thereof, may be reconfigured as a circular permutant variant.

In various embodiments, the circular permutants of a Cas protein, e.g., a Cas9, may have the following structure: N-terminus-[original C-terminus]-[optional linker]-[original N-terminus]-C-terminus. In some embodiments, a circular permutant Cas9 comprises any one of the following structures (amino acid positions as set forth in SEQ ID NO: 34364):
N-terminus-[1268-1368]-[optional linker]-[1-1267]-C-terminus;
N-terminus-[1168-1368]-[optional linker]-[1-1167]-C-terminus;
N-terminus-[1068-1368]-[optional linker]-[1-1067]-C-terminus;
N-terminus-[968-1368]-[optional linker]-[1-967]-C-terminus;
N-terminus-[868-1368]-[optional linker]-[1-867]-C-terminus;
N-terminus-[768-1368]-[optional linker]-[1-767]-C-terminus;
N-terminus-[668-1368]-[optional linker]-[1-667]-C-terminus;
N-terminus-[568-1368]-[optional linker]-[1-567]-C-terminus;
N-terminus-[468-1368]-[optional linker]-[1-467]-C-terminus;
N-terminus-[368-1368]-[optional linker]-[1-367]-C-terminus;
N-terminus-[268-1368]-[optional linker]-[1-267]-C-terminus;
N-terminus-[168-1368]-[optional linker]-[1-167]-C-terminus;
N-terminus-[68-1368]-[optional linker]-[1-67]-C-terminus;
N-terminus-[10-1368]-[optional linker]-[1-9]-C-terminus, or the corresponding circular permutants of other Cas9 proteins (including other Cas9 orthologs, variants, etc.).

In some embodiments, a circular permutant Cas9 comprises any one of the following structures (amino acid positions as set forth in SEQ ID NO: 34364-1368 amino acids of UniProtKB—Q99ZW2:
N-terminus-[102-1368]-[optional linker]-[1-101]-C-terminus;
N-terminus-[1028-1368]-[optional linker]-[1-1027]-C-terminus;
N-terminus-[1041-1368]-[optional linker]-[1-1043]-C-terminus;
N-terminus-[1249-1368]-[optional linker]-[1-1248]-C-terminus; or
N-terminus-[1300-1368]-[optional linker]-[1-1299]-C-terminus, or the corresponding circular permutants of other Cas9 proteins (including other Cas9 orthologs, variants, etc.).

In some embodiments, a circular permutant Cas9 comprises any one of the following structures (amino acid positions as set forth in SEQ ID NO: 34364-1368 amino acids of UniProtKB—Q99ZW2 N-terminus-[103-1368]-[optional linker]-[1-102]-C-terminus:
N-terminus-[1029-1368]-[optional linker]-[1-1028]-C-terminus;
N-terminus-[1042-1368]-[optional linker]-[1-1041]-C-terminus;
N-terminus-[1250-1368]-[optional linker]-[1-1249]-C-terminus; or
N-terminus-[1301-1368]-[optional linker]-[1-1300]-C-terminus, or the corresponding circular permutants of other Cas9 proteins (including other Cas9 orthologs, variants, etc.).

In some embodiments, the circular permutant can be formed by linking a C-terminal fragment of a Cas9 to an N-terminal fragment of a Cas9, either directly or by using a linker, such as an amino acid linker. In some embodiments, thee C-terminal fragment may correspond to the 95% or more of the C-terminal amino acids of a Cas9 (e.g., amino acids about 1300-1368 as set forth in SEQ ID No: 34364 or corresponding amino acid positions thereof), or the 90%, 85%, 80%, 75%, 70%, 65%, 60%, 55%, 50%, 45%, 40%, 35%, 30%, 25%, 20%, 15%, 10%, or 5% or more of the C-terminal amino acids of a Cas9 (e.g., SEQ ID NO: 34364 or a ortholog or a variant thereof). The N-terminal portion may correspond to 95% or more of the N-terminal amino acids of a Cas9 (e.g., amino acids about 1-1300 as set forth in SEQ ID No: 34364 or corresponding amino acid positions thereof), or 90%, 85%, 80%, 75%, 70%, 65%, 60%, 55%, 50%, 45%, 40%, 35%, 30%, 25%, 20%, 15%, 10%, or 5% or more of the N terminal amino acids of a Cas9 (e.g., as set forth in SEQ ID No: 34364 or corresponding amino acid positions thereof).

In some embodiments, the circular permutant can be formed by linking a C-terminal fragment of a Cas9 to an N-terminal fragment of a Cas9, either directly or by using a linker, such as an amino acid linker. In some embodiments, the C-terminal fragment that is rearranged to the N-terminus includes or corresponds to the C-terminal 30% or less of the amino acids of a Cas9 (e.g., amino acids 1012-1368 as set forth in SEQ ID No: 34364 or corresponding amino acid positions thereof). In some embodiments, the C-terminal fragment that is rearranged to the N-terminus, includes or corresponds to the C-terminal 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1% of the amino acids of a Cas9 (e.g., as set forth in SEQ ID No: 34364 or corresponding amino acid positions thereof). In some embodiments, the C-terminal fragment that is rearranged to the N-terminus, includes or corresponds to the C-terminal 410 residues or less of a Cas9 (e.g., as set forth in SEQ ID No: 34364 or corresponding amino acid positions thereof). In some embodiments, the C-terminal portion that is rearranged to the N-terminus, includes or corresponds to the C-terminal 410, 400, 390, 380, 370, 360, 350, 340, 330, 320, 310, 300, 290, 280, 270, 260, 250, 240, 230, 220, 210, 200, 190, 180, 170, 160, 150, 140, 130, 120, 110, 100, 90, 80, 70, 60, 50, 40, 30, 20, or 10 residues of a Cas9 (e.g. as set forth in SEQ ID No: 34364 or corresponding amino acid positions thereof). In some embodiments, the C-terminal portion that is rearranged to the N-terminus includes or corresponds to the C-terminal 357, 341, 328, 120, or 69 residues of a Cas9 (e.g., as set forth in SEQ ID No: 34364 or corresponding amino acid positions thereof).

In other embodiments, circular permutant Cas9 variants may be a topological rearrangement of a Cas9 primary structure based on the following method, which is based on S. pyogenes Cas9 of SEQ ID NO: 34364: (a) selecting a circular permutant (CP) site corresponding to an internal amino acid residue of the Cas9 primary structure, which dissects the original protein into two halves: an N-terminal region and a C-terminal region; (b) modifying the Cas9 protein sequence (e.g., by genetic engineering techniques) by moving the original C-terminal region (comprising the CP site amino acid) to precede the original N-terminal region, thereby forming a new N-terminus of the Cas9 protein that now begins with the CP site amino acid residue. The CP site can be located in any domain of the Cas9 protein, including, for example, the helical-II domain, the RuvCIII domain, or the CTD domain. For example, the CP site may be located (as set forth in SEQ ID No: 34364 or corresponding amino acid positions thereof) at original amino acid residue 181, 199, 230, 270, 310, 1010, 1016, 1023, 1029, 1041, 1247, 1249, or 1282. Thus, once relocated to the N-terminus, original amino acid 181, 199, 230, 270, 310, 1010, 1016, 1023, 1029, 1041, 1247, 1249, or 1282 would become the new N-terminal amino acid. Nomenclature of these CP-Cas9 proteins may be referred to as Cas9-CP¹⁸¹, Cas9-CP¹⁹⁹, Cas9-CP²³⁰, Cas9-CP²⁷⁰, Cas9-CP³¹⁰, Cas9-CP¹⁰¹⁰, Cas9-CP¹⁰¹⁶, Cas9-CP¹⁰²³, Cas9-CP¹⁰²⁹, Cas9-CP¹⁰⁴¹, Cas9-CP¹²⁴⁷, Cas9-CP¹²⁴⁹, and Cas9-CP¹²⁸², respectively. This description is not meant to be limited to making CP variants from SEQ ID NO: 34364 but may be implemented to make CP variants in any Cas9 sequence, either at CP sites that correspond to these positions, or at other CP sites entirely. This description is not meant to limit the specific CP sites in any way. Virtually any CP site may be used to form a CP-Cas9 variant.

In some embodiments, a prime editor comprises a Cas9 functional variant that is of smaller molecular weight than a wild type SpCas9 protein. In some embodiments, a smaller-sized Cas9 functional variant may facilitate delivery to cells, e.g., by an expression vector, nanoparticle, or other means of delivery. In certain embodiments, a smaller-sized Cas9 functional variant is a Class 2 Type II Cas protein. In certain embodiments, a smaller-sized Cas9 functional variant is a Class 2 Type V Cas protein. In certain embodiments, a smaller-sized Cas9 functional variant is a Class 2 Type VI Cas protein.

In some embodiments, a prime editor comprises a SpCas9 that is 1368 amino acids in length and has a predicted molecular weight of 158 kilodaltons. In some embodiments, a prime editor comprises a Cas9 functional variant or functional fragment that is less than 1300 amino acids, less than 1290 amino acids, than less than 1280 amino acids, less than 1270 amino acids, less than 1260 amino acid, less than 1250 amino acids, less than 1240 amino acids, less than 1230 amino acids, less than 1220 amino acids, less than 1210 amino acids, less than 1200 amino acids, less than 1190 amino acids, less than 1180 amino acids, less than 1170 amino acids, less than 1160 amino acids, less than 1150 amino acids, less than 1140 amino acids, less than 1130 amino acids, less than 1120 amino acids, less than 1110 amino acids, less than 1100 amino acids, less than 1050 amino acids, less than 1000 amino acids, less than 950 amino acids, less than 900 amino acids, less than 850 amino acids, less than 800 amino acids, less than 750 amino acids, less than 700 amino acids, less than 650 amino acids, less than 600 amino acids, less than 550 amino acids, or less than 500 amino acids, but at least larger than about 400 amino acids and retaining the one or more functions, e.g., DNA binding function, of the Cas9 protein.

In some embodiments, the Cas protein may include any CRISPR associated protein, including but not limited to, Cas12a, Cas12bl, Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof, and preferably comprising a nickase mutation (e.g., a mutation corresponding to the D10A mutation of the wild type Cas9 polypeptide of SEQ ID NO: 14829). In various other embodiments, the napDNAbp can be any of the following proteins: a Cas9, a Cas2a (Cpf1), a Cas2e (CaX), a Cas2d (CasY), a Cas12bl (C2c1), a Cas3a (C2c2), a Cas12c (C2c), a GeoCas9, a CjCas9, a Cas2g, a Cas12h, a Cas12i, a Cas13b, a Cas13c, a Cas13d, a Cas14, a Csn2, an xCas9, an SpCas9-NG, a circularly permuted Cas9, or an Argonaute (Ago) domain, or a functional variant or fragment thereof.

Exemplary Cas proteins and nomenclature are shown in Table 81 below:

TABLE 81
Exemplary Cas proteins and nomenclature

| | |
|---|---|
| Legacy nomenclature | Current nomenclature |

type II CRISPR-Cas enzymes

| | |
|---|---|
| Cas9 | same |

type V CRISPR-Cas enzymes

| | |
|---|---|
| Cpfl | Cas12a |
| CasX | Cas12e |
| C2c1 | Cas12b1 |
| Cas12b2 | same |
| C2c3 | Cas12c |
| CasY | Cas12d |
| C2c4 | same |
| C2c8 | same |
| C2c5 | same |
| C2c10 | same |
| C2c9 | same |

type VI CRISPR-Cas enzymes

| | |
|---|---|
| C2c2 | Cas13a |
| Cas13d | same |
| C2c7 | Cas13c |
| C2c6 | Cas13b |

In some embodiments, prime editors described herein may also comprise Cas proteins other than Cas9. For example, in some embodiments, a prime editor as described herein may comprise a Cas12a (Cpf1) polypeptide or functional variants thereof. In some embodiments, the Cas2a polypeptide comprises a mutation that reduces or abolishes the endonuclease domain of the Cas12a polypeptide. In some embodiments, the Cas12a polypeptide is a Cas12a nickase. In some embodiments, the Cas protein comprises an amino acid sequence that comprises at least about 50%, 60%, 70%, 80%, 90%, 91%, 92%, 93%, 94%, 95%, 96%, 97%, 98%, 99%, or 100% sequence identity to a naturally occurring Cas12a polypeptide.

In some embodiments, a prime editor comprises a Cas protein that is a Cas2b (C2c1) or a Cas12c (C2c3) polypeptide. In some embodiments, the Cas protein comprises an amino acid sequence that comprises at least about 50%, 60%, 70%, 80%, 90%, 91%, 92%, 93%, 94%, 95%, 96%, 97%, 98%, 99%, or 100% sequence identity to a naturally occurring Cas12b (C2c1) or Cas12c (C2c) protein. In some embodiments, the Cas protein is a Cas12b nickase or a Cas2c nickase. In some embodiments, the Cas protein is a Cas12e, a Cas12d, a Cas13, Cas14a, Cas14b, Cas14c, Cas14d, Cas14e, Cas14f, Cas14g, Cas14h, Cas14u, or a CasΦ polypeptide. In some embodiments, the Cas protein comprises an amino acid sequence that comprises at least about 50%, 60%, 70%, 80%, 90%, 91%, 92%, 93%, 94%, 95%, 96%, 97%, 98%, 99%, or 100% sequence identity to a naturally-occurring Cas12e, Cas12d, Cas13, Cas14a, Cas14b, Cas14c, Cas14d, Cas14e, Cas14f, Cas14g, Cas14h, Cas14u, or Cas Φ protein. In some embodiments, the Cas protein is a Cas12e, Cas12d, Cas13, or Cas Φ nickase.

### Nuclear Localization Sequences

In some embodiments, a prime editor further comprises one or more nuclear localization sequence (NLS). In some embodiments, the NLS helps promote translocation of a protein into the cell nucleus. In some embodiments, a prime editor comprises a fusion protein, e.g., a fusion protein comprising a DNA binding domain and a DNA polymerase, that comprises one or more NLSs. In some embodiments, one or more polypeptides of the prime editor are fused to or linked to one or more NLSs. In some embodiments, the prime editor comprises a DNA binding domain and a DNA polymerase domain that are provided in trans, wherein the DNA binding domain and/or the DNA polymerase domain is fused or linked to one or more NLSs.

In certain embodiments, a prime editor or prime editing complex comprises at least one NLS. In some embodiments, a prime editor or prime editing complex comprises at least two NLSs. In embodiments with at least two NLSs, the NLSs can be the same NLS, or they can be different NLSs.

In some instances, a prime editor may further comprise at least one nuclear localization sequence (NLS). In some cases, a prime editor may further comprise 1 NLS. In some cases, a prime editor may further comprise 2 NLSs. In other cases, a prime editor may further comprise 3 NLSs. In one case, a primer editor can further comprise more than 4, 5, 6, 7, 8, 9 or 10 NLSs.

In addition, the NLSs can be expressed as part of a prime editor complex. In some embodiments, a NLS can be positioned almost anywhere in a protein's amino acid sequence, and generally comprises a short sequence of three or more or four or more amino acids. The location of the NLS fusion can be at the N-terminus, the C-terminus, or positioned anywhere within a sequence of a prime editor or a component thereof (e.g., inserted between the DNA-binding domain and the DNA polymerase domain of a prime editor fusion protein, between the DNA binding domain and a linker sequence, between a DNA polymerase and a linker sequence, between two linker sequences of a prime editor fusion protein or a component thereof, in either N-terminus to C-terminus or C-terminus to N-terminus order). In some embodiments, a prime editor is fusion protein that comprises an NLS at the N terminus. In some embodiments, a prime editor is fusion protein that comprises an NLS at the C terminus. In some embodiments, a prime editor is fusion protein that comprises at least one NLS at both the N terminus and the C terminus. In some embodiments, the prime editor is a fusion protein that comprises two NLSs at the N terminus and/or the C terminus.

Any NLSs that are known in the art are also contemplated herein. The NLSs may be any naturally occurring NLS, or any non-naturally occurring NLS (e.g., an NLS with one or more mutations relative to a wild-type NLS). In some embodiments, the one or more NLSs of a prime editor comprise bipartite NLSs. In some embodiments, a nuclear localization signal (NLS) is predominantly basic. In some embodiments, the one or more NLSs of a prime editor are rich in lysine and arginine residues. In some embodiments, the one or more NLSs of a prime editor comprise proline residues.

In some embodiments, a nuclear localization signal (NLS) comprises the sequence

MDSLLMNRRKFLYQFKNVRWAKGRRETYLC (SEQ ID NO: 34393) or KRTADGSEFEPKKKRKV (SEQ ID NO: 34403).

In some embodiments, a NLS is a monopartite NLS. For example, in some embodiments, a NLS is a SV40 large T antigen NLS PKKKRKV (SEQ ID NO: 34391). In some embodiments, a NLS is a bipartite NLS. In some embodiments, a bipartite NLS comprises two basic domains separated by a spacer sequence comprising a variable number of amino acids. In some embodiments, a NLS is a bipartite NLS. In some embodiments, a bipartite NLS consists of two basic domains separated by a spacer sequence comprising a variable number of amino acids. In some embodiments, a NLS is a noncanonical sequences such as M9 of the hnRNP A1 protein, the influenza virus nucleoprotein NLS, and the yeast Gal4 protein NLS. In some embodiments, a NLS is a noncanonical sequences such as M9 of the hnRNP A1 protein, the influenza virus nucleoprotein NLS, and the yeast Gal4 protein NLS.

In some embodiments, a bipartite NLS consists of two basic domains separated by a spacer sequence comprising a variable number of amino acids. In some embodiments, a NLS comprises an amino acid sequence that is at least about 50%, 60%, 70%, 80%, 90%, 91%, 92%, 93%, 94%, 95%, 96%, 97%, 98%, 99%, or 100% identical to the amino acid sequence of any one of SEQ ID NOs: 34391-34403. In some embodiments, a NLS comprises an amino acid sequence selected from the group consisting of 34391-34403. In some embodiments, a prime editing composition comprises a polynucleotide that encodes a NLS that comprises an amino acid sequence that is at least about 50%, 60%, 70%, 80%, 90%, 91%, 92%, 93%, 94%, 95%, 96%, 97%, 98%, 99%, or 100% identical to the amino acid sequence of any one of SEQ ID NOs: 34391-34403. In some embodiments, a prime editing composition comprises a polynucleotide that encodes a NLS that comprises an amino acid sequence selected from the group consisting of 34391-34403.

Any NLSs that are known in the art are also contemplated herein. The NLSs may be any naturally occurring NLS, or any non-naturally occurring NLS (e.g., an NLS with one or more mutations relative to a wild-type NLS). In some embodiments, the one or more NLSs of a prime editor comprise bipartite NLSs. In some embodiments, the one or more NLSs of a prime editor are rich in lysine and arginine residues. In some embodiments, the one or more NLSs of a prime editor comprise proline residues. Non-limiting examples of NLS sequences are provided in Table 84 below.

TABLE 84
Exemplary nuclear localization sequences

| | | SEQ |
|---|---|---|
| Description | Sequence | ID NO: |
| NLS of SV40 Large | PKKKRKV | 34391 |
| T-AG | | |
| NLS | MKRTADGSEFESPKKKRKV | 34392 |
| NLS | MDSLLMNRRKFLYQFKNVRW | 34393 |
| | AKGRRETYLC | |
| NLS of Nucleoplasmin | AVKRPAATKKAGQAKKKKLD | 34394 |
| NLS of EGL-13 | MSRRRKANPTKLSENAKKLA | 34395 |
| | KEVEN | |
| NLS of C-Myc | PAAKRVKLD | 34396 |
| NLS of Tus-protein | KLKIKRPVK | 34397 |
| NLS of polyoma large | VSRKRPRP | 34398 |
| T-AG | | |
| NLS of Hepatitis D | EGAPPAKRAR | 34399 |
| virus antigen | | |
| NLS of Rev protein | RQARRNRRRRWRERNR | 34400 |
| NLS of murine p53 | PPQPKKKPLDGE | 34401 |
| C terminal linker | SGGSKRTADGSEFEPKKKRK | 34402 |
| and NLS of an | V | |
| exemplary prime | | |
| editor fusion | | |
| protein | | |
| NLS | KRTADGSEFEPKKKRKV | 34403 |

In some embodiments, a prime editing complex comprises a fusion protein comprising a DNA binding domain (e.g., Cas9(H840A)) and a reverse transcriptase (e.g., a variant MMLV RT) having the following structure: [NLS]-[Cas9(H840A)]-[linker]-[MMLV_RT(D200N)(T330P)(L603W)(T306K)(W313F)], and a desired PEgRNA. In some embodiments, the prime editing complex comprises a prime editor fusion protein that has the amino acid sequence of SEQ ID NO: 34404. Sequence of an exemplary prime editor fusion protein comprising a DNA binding domain (e.g., Cas9(H840A)) and a reverse transcriptase (e.g., a variant MMLV RT) having the following structure: [NLS]-[Cas9(H840A)]-[linker]-[MMLV_RT(D200N)(T330P)(L603W)(T306K)(W313F)] and its components are shown in Table 82.

In some embodiments, a prime editing complex comprises a fusion protein comprising a DNA binding domain (e.g., Cas9((R221K N394K H840A)) and a reverse transcriptase (e.g., a variant MMLV RT) having the following structure: [NLS]-[Cas9((R221K N394K H840A)]-[linker]-[MMLV_RT(D200N)(T330P)(L603W)(T306K)(W313F)], and a desired PEgRNA. In some embodiments, the prime editing complex comprises a prime editor fusion protein that has the amino acid sequence of SEQ ID NO: 34405. Sequence of an exemplary prime editor fusion protein comprising a DNA binding domain (e.g., Cas9(H840A)) and a reverse transcriptase (e.g., a variant MMLV RT) having the following structure: [NLS]-[Cas9 (R221K N394K H840A)]-[linker]-[MMLV_RT(D200N)(T330P)(L603W)(T306K)(W313F)] and its components are shown in Table 83.

Polypeptides comprising components of a prime editor may be fused via peptide linkers, or may be provided in trans relevant to each other. For example, a reverse transcriptase may be expressed, delivered, or otherwise provided as an individual component rather than as a part of a fusion protein with the DNA binding domain. In such cases, components of the prime editor may be associated through non-peptide linkages or co-localization functions. In some embodiments, a prime editor further comprises additional components capable of interacting with, associating with, or capable of recruiting other components of the prime editor or the prime editing system. For example, a prime editor may comprise an RNA-protein recruitment polypeptide that can associate with an RNA-protein recruitment RNA aptamer. In some embodiments, an RNA-protein recruitment polypeptide can recruit, or be recruited by, a specific RNA sequence. Non limiting examples of RNA-protein recruitment polypeptide and RNA aptamer pairs include a MS2 coat protein and a MS2 RNA hairpin, a PCP polypeptide and a PP7 RNA hairpin, a Com polypeptide and a Com RNA hairpin, a Ku protein and a telomerase Ku binding RNA motif, and a Sm7 protein and a telomerase Sm7 binding RNA motif. In some embodiments, the prime editor comprises a DNA binding domain fused or linked to an RNA-protein recruitment polypeptide. In some embodiments, the prime editor comprises a DNA polymerase domain fused or linked to an RNA-protein recruitment polypeptide. In some embodiments, the DNA binding domain and the DNA polymerase domain fused to the RNA-protein recruitment polypeptide, or the DNA binding domain fused to the RNA-protein recruitment polypeptide and the DNA polymerase domain are co-localized by the corresponding RNA-protein recruitment RNA aptamer of the RNA-protein recruitment polypeptide. In some embodiments, the corresponding RNA-protein recruitment RNA aptamer fused or linked to a portion of the PEgRNA or ngRNA. For example, an MS2 coat protein fused or linked to the DNA polymerase and a MS2 hairpin installed on the PEgRNA for co-localization of the DNA polymerase and the RNA-guided DNA binding domain (e.g., a Cas9 nickase). In certain embodiments, components of a prime editor are directly fused to each other. In certain embodiments, components of a prime editor are associated to each other via a linker.

As used herein, a linker can be any chemical group or a molecule linking two molecules or moieties, e.g., a DNA binding domain and a polymerase domain of a prime editor. In some embodiments, a linker is an organic molecule, group, polymer, or chemical moiety. In some embodiments, the linker comprises a non-peptide moiety. The linker may be as simple as a covalent bond, or it may be a polymeric linker many atoms in length, for example, a polynucleotide sequence. In certain embodiments, the linker is a covalent bond (e.g., a carbon-carbon bond, disulfide bond, carbon-heteroatom bond, etc.).

In certain embodiments, two or more components of a prime editor are linked to each other by a peptide linker. In some embodiments, a peptide linker is 5-100 amino acids in length, for example, 2, 3, 4, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. In some embodiments, the peptide linker is 16 amino acids in length, 24 amino acids in length, 64 amino acids in length, or 96 amino acids in length.

In some embodiments, the linker comprises the amino acid sequence (GGGGS)n (SEQ ID NO: 34410), (G)n (SEQ ID NO: 34411), (EAAAK)n (SEQ ID NO: 34412), (GGS)n (SEQ ID NO: 34413), (SGGS)n (SEQ ID NO: 34414), (XP)n (SEQ ID NO: 34415), or any combination thereof, wherein n is independently an integer between 1 and 30, and wherein X is any amino acid. In some embodiments, the linker comprises the amino acid sequence (GGS)n (SEQ ID NO: 34413), wherein n is 1, 3, or 7. In some embodiments, the linker comprises the amino acid sequence SGSETPGTSESATPES (SEQ ID NO: 34416). In some embodiments, the linker comprises the amino acid sequence SGGSSGGSSGS ETPGTSESATPESSGGSSGGS (SEQ ID NO: 34417). In some embodiments, the linker comprises the amino acid sequence SGGSGGSGGS (SEQ ID NO: 34419). In some embodiments, the linker comprises the amino acid sequence SGGS (SEQ ID NO: 34420). In other embodiments, the linker comprises the amino acid sequence

(SEQ ID NO: 34421)
SGGSSGGSSGSETPGTSESATPESAGSYPYDVPDYAGSAAPAAKKKKLD
GSGSGGSSGGS.

In certain embodiments, two or more components of a prime editor are linked to each other by a non-peptide linker. In some embodiments, the linker is a carbon-nitrogen bond of an amide linkage. In certain embodiments, the linker is a cyclic or acyclic, substituted or unsubstituted, branched or unbranched aliphatic or heteroaliphatic linker. In certain embodiments, the linker is polymeric (e.g., polyethylene, polyethylene glycol, polyamide, polyester, etc.). In certain embodiments, the linker comprises a monomer, dimer, or polymer of aminoalkanoic acid. In certain embodiments, the linker comprises an aminoalkanoic acid (e.g., glycine, ethanoic acid, alanine, beta-alanine, 3-aminopropanoic acid, 4-aminobutanoic acid, 5-pentanoic acid, etc.). In certain embodiments, the linker comprises a monomer, dimer, or polymer of aminohexanoic acid (Ahx). In certain embodiments, the linker is based on a carbocyclic moiety (e.g., cyclopentane, cyclohexane). In other embodiments, the linker comprises a polyethylene glycol moiety (PEG). In certain embodiments, the linker comprises an aryl or heteroaryl moiety. In certain embodiments, the linker is based on a phenyl ring. The linker may include functionalized moieties to facilitate attachment of a nucleophile (e.g., thiol, amino) from the peptide to the linker. Any electrophile may be used as part of the linker. Exemplary electrophiles include, but are not limited to, activated esters, activated amides, Michael acceptors, alkyl halides, aryl halides, acyl halides, and isothiocyanates.

Components of a prime editor may be connected to each other in any order. In some embodiments, the DNA binding domain and the DNA polymerase domain of a prime editor may be fused to form a fusion protein, or may be joined by a peptide or protein linker, in any order from the N terminus to the C terminus. In some embodiments, a prime editor comprises a DNA binding domain fused or linked to the C-terminal end of a DNA polymerase domain. In some embodiments, a prime editor comprises a DNA binding domain fused or linked to the N-terminal end of a DNA polymerase domain. In some embodiments, the prime editor comprises a fusion protein comprising the structure NH2-[DNA binding domain]-[polymerase]-COOH; or NH2-[polymerase]-[DNA binding domain]-COOH, wherein each instance of “]-[” indicates the presence of an optional linker sequence. In some embodiments, a prime editor comprises a fusion protein and a DNA polymerase domain provided in trans, wherein the fusion protein comprises the structure NH2-[DNA binding domain]-[RNA-protein recruitment polypeptide]-COOH. In some embodiments, a prime editor comprises a fusion protein and a DNA binding domain provided in trans, wherein the fusion protein comprises the structure NH2-[DNA polymerase domain]-[RNA-protein recruitment polypeptide]-COOH.

In some embodiments, a prime editor fusion protein, a polypeptide component of a prime editor, or a polynucleotide encoding the prime editor fusion protein or polypeptide component, may be split into an N-terminal half and a C-terminal half or polypeptides that encode the N-terminal half and the C terminal half, and provided to a target DNA in a cell separately. For example, in certain embodiments, a prime editor fusion protein may be split into a N-terminal and a C-terminal half for separate delivery in AAV vectors, and subsequently translated and colocalized in a target cell to reform the complete polypeptide or prime editor protein. In such cases, separate halves of a protein or a fusion protein may each comprise a split-intein to facilitate colocalization and reformation of the complete protein or fusion protein by the mechanism of intein facilitated trans splicing. In some embodiments, a prime editor comprises a N-terminal half fused to an intein-N, and a C-terminal half fused to an intein-C, or polynucleotides or vectors (e.g., AAV vectors) encoding each thereof. When delivered and/or expressed in a target cell, the intein-N and the intein-C can be excised via protein trans-splicing, resulting in a complete prime editor fusion protein in the target cell. In some embodiments, an exemplary protein described herein may lack a methionine residue at the N-terminus.

In some embodiments, a prime editor fusion protein comprises a Cas9(H840A) nickase and a wild type M-MLV RT. In some embodiments, a prime editor fusion protein comprises a Cas9(H840A) nickase and a M-MLV RT that comprises amino acid substitutions D200N, T330P, T306K, W313F, and L603W compared to a wild type M-MLV RT. In some embodiments, a prime editor fusion protein comprises a Cas9(H840A) nickase and a M-MLV RT that comprises amino acid substitutions D200N, T330P, T306K, W313F, and L603W compared to a wild type M-MLV RT. The amino acid sequence of an exemplary PE2 and its individual components in shown in Table 82. In some embodiments, a prime editor fusion protein comprises a Cas9 (R221K N394K H840A) nickase and a M-MLV RT that comprises amino acid substitutions D200N, T330P, T306K, W313F, and L603W compared to a wild type M-MLV RT. The amino acid sequence of an exemplary Prime editor fusion protein and its individual components in shown in Table 83. In some embodiments an exemplary prime editor protein may comprise an amino acid sequence as set forth in any of the SEQ ID NO: 34404 or SEQ ID NO: 34405.

In various embodiments, a prime editor fusion protein comprises an amino acid sequence that is at least about 70% identical, at least about 80% identical, at least about 90% identical, at least about 95% identical, at least about %% identical, at least about 97% identical, at least about 98% identical, at least about 99% identical, at least about 99.5% identical, or at least about 99.9% identical to any of the prime editor fusion sequences described herein or known in the art.

TABLE 82
lists exemplary prime editor and its components

| SEQ ID | | |
|---|---|---|
| NO. | DESCRIPTION | SEQUENCE |
| 34404 | Exemplary Prime Editor | MKRTADGSEFESPKKKRKVDKKYSIGLDIGTNSVGWAVITDEYKV |
| | [NLS]-[Cas9(H840A)]- | PSKKFKVLGNTDRHSIKKNLIGALLFDSGETAEATRLKRTARRR |
| | [linker]- | YTRRKNRICYLQEIFSNEMAKVDDSFFHRLEESFLVEEDKKHER |
| | [MMLV_RT(D200N)(T330P) | HPIFGNIVDEVAYHEKYPTIYHLRKKLVDSTDKADLRLIYLALAH |
| | (L603W)(T306K)(W313F)]- | MIKFRGHFLIEGDLNPDNSDVDKLFIQLVQTYNQLFEENPINASG |
| | [NLS] | VDAKAILSARLSKSRRLENLIAQLPGEKKNGLFGNLIALSLGLTP |
| | | NFKSNFDLAEDAKLQLSKDTYDDDLDNLLAQIGDQYADLFLAAK |
| | | NLSDAILLSDILRVNTEITKAPLSASMIKRYDEHHQDLTLLKALV |
| | | RQQLPEKYKEIFFDQSKNGYAGYIDGGASQEEFYKFIKPILEKMD |
| | | GTEELLVKLNREDLLRKQRTFDNGSIPHQIHLGELHAILRRQEDF |
| | | YPFLKDNREKIEKILTFRIPYYVGPLARGNSRFAWMTRKSEETIT |
| | | PWNFEEVVDKGASAQSFIERMTNFDKNLPNEKVLPKHSLLYEYF |
| | | TVYNELTKVKYVTEGMRKPAFLSGEQKKAIVDLLFKTNRKVTV |
| | | KQLKEDYFKKIECFDSVEISGVEDRFNASLGTYHDLLKIIKDKDF |
| | | LDNEENEDILEDIVLTLTLFEDREMIEERLKTYAHLFDDKVMKQ |
| | | LKRRRYTGWGRLSRKLINGIRDKQSGKTILDFLKSDGFANRNFM |
| | | QLIHDDSLTFKEDIQKAQVSGQGDSLHEHIANLAGSPAIKKGILQ |
| | | TVKVVDELVKVMGRHKPENIVIEMARENQTTQKGQKNSRERM |
| | | KRIEEGIKELGSQILKEHPVENTQLQNEKLYLYYLQNGRDMYVD |
| | | QELDINRLSDYDVDAIVPQSFLKDDSIDNKVLTRSDKNRGKSDNV |
| | | PSEEVVKKMKNYWRQLLNAKLITQRKFDNLTKAERGGLSELDK |
| | | AGFIKRQLVETRQITKHVAQILDSRMNTKYDENDKLIREVKVITL |
| | | KSKLVSDFRKDFQFYKVREINNYHHAHDAYLNAVVGTALIKKYP |
| | | KLESEFVYGDYKVYDVRKMIAKSEQEIGKATAKYFFYSNIMNFF |
| | | KTEITLANGEIRKRPLIETNGETGEIVWDKGRDFATVRKVLSMP |
| | | QVNIVKKTEVQTGGFSKESILPKRNSDKLIARKKDWDPKKYGGF |
| | | DSPTVAYSVLVVAKVEKGKSKKLKSVKELLGITIMERSSFEKNPI |
| | | DFLEAKGYKEVKKDLIIKLPKYSLFELENGRKRMLASAGELQKG |
| | | NELALPSKYVNFLYLASHYEKLKGSPEDNEQKQLFVEQHKHYL |
| | | DEIIEQISEFSKRVILADANLDKVLSAYNKHRDKPIREQAENIIHLF |
| | | TLTNLGAPAAFKYFDTTIDRKRYTSTKEVLDATLIHQSITGLYET |
| | | RIDLSQLGGDSGGSSGGSSGSETPGTSESATPESSGGSSGGSSTLNI |
| | | EDEYRLHETSKEPDVSLGSTWLSDFPQAWAETGGMGLAVRQAPLIIPLK |
| | | ATSTPVSIKQYPMSQEARLGIKPHIQRLLDQGILVPCQSPWNTPLLPVKK |
| | | PGTNDYRPVQDLREVNKRVEDIHPTVPNPYNLLSGLPPSHQWYTVLDLK |
| | | DAFFCLRLHPTSQPLFAFEWRDPEMGISGQLTWTRLPQGFKNSPTLFNE |
| | | ALHRDLADFRIQHPDLILLQYVDDLLLAATSELDCQQGTRALLQTLGNL |
| | | GYRASAKKAQICQKQVKYLGYLLKEGQRWLTEARKETVMGQPTPKTPR |
| | | QLREFLGKAGFCRLFIPGFAEMAAPLYPLTKPGTLFNWGPDQQKAYQEI |
| | | KQALLTAPALGLPDLTKPFELFVDEKQGYAKGVLTQKLGPWRRPVAYLS |
| | | KKLDPVAAGWPPCLRMVAAIAVLTKDAGKLTMGQPLVILAPHAVEALVK |
| | | QPPDRWLSNARMTHYQALLLDTDRVQFGPVVALNPATLLPLPEEGLQH |
| | | NCLDILAEAHGTRPDLTDQPLPDADHTWYTDGSSLLQEGQRKAGAAVTT |
| | | ETEVIWAKALPAGTSAQRAELIALTQALKMAEGKKLNVYTDSRYAFATAH |
| | | IHGEIYRRRGWLTSEGKEIKNKDEILALLKALFLPKRLSIIHCPGHQKGHS |
| | | AEARGNRMADQAARKAAITETPDTSTLLIENSSPSGGSKRTADGSEFEPK |
| | | KKRKV |
| | | KEY: |
| | | NUCLEAR LOCALIZATION SEQUENCE (NLS) |
| | | CAS9(H840A) |
| | | 33-AMINO ACID LINKER |
| | | M-MLV REVERSE TRANSCRIPTASE |
| 34392 | N-terminal NLS | MKRTADGSEFESPKKKRKV |
| 34366 | CAS9 (H840A) | DKKYSIGLDIGTNSVGWAVITDEYKVPSKKFKVLGNTDRHSIKKNLI |
| | | GALLFDSGETAEATRLKRTARRRYTRRKNRICYLQEIFSNEMAKVD |
| | | DSFFHRLEESFLVEEDKKHERHPIFGNIVDEVAYHEKYPTIYHLRKKL |
| | | VDSTDKADLRLIYLALAHMIKFRGHFLIEGDLNPDNSDVDKLFIQLV |
| | | QTYNQLFEENPINASGVDAKAILSARLSKSRRLENLIAQLPGEKKNG |
| | | LFGNLIALSLGLTPNFKSNFDLAEDAKLQLSKDTYDDDLDNLLAQIG |
| | | DQYADLFLAAKNLSDAILLSDILRVNTEITKAPLSASMIKRYDEHHQ |
| | | DLTLLKALVRQQLPEKYKEIFFDQSKNGYAGYIDGGASQEEFYKFIK |
| | | PILEKMDGTEELLVKLNREDLLRKQRTFDNGSIPHQIHLGELHAILRR |
| | | QEDFYPFLKDNREKIEKILTFRIPYYVGPLARGNSRFAWMTRKSEETI |
| | | TPWNFEEVVDKGASAQSFIERMTNFDKNLPNEKVLPKHSLLYEYFT |
| | | VYNELTKVKYVTEGMRKPAFLSGEQKKAIVDLLFKTNRKVTVKQL |
| | | KEDYFKKIECFDSVEISGVEDRFNASLGTYHDLLKIIKDKDFLDNEEN |
| | | EDILEDIVLTLTLFEDREMIEERLKTYAHLFDDKVMKQLKRRRYTG |
| | | WGRLSRKLINGIRDKQSGKTILDFLKSDGFANRNFMQLIHDDSLTFK |
| | | EDIQKAQVSGQGDSLHEHIANLAGSPAIKKGILQTVKVVDELVKVM |
| | | GRHKPENIVIEMARENQTTQKGQKNSRERMKRIEEGIKELGSQILKE |
| | | HPVENTQLQNEKLYLYYLQNGRDMYVDQELDINRLSDYDVDAIVP |
| | | QSFLKDDSIDNKVLTRSDKNRGKSDNVPSEEVVKKMKNYWRQLLN |
| | | AKLITQRKFDNLTKAERGGLSELDKAGFIKRQLVETRQITKHVAQIL |
| | | DSRMNTKYDENDKLIREVKVITLKSKLVSDFRKDFQFYKVREINNY |
| | | HHAHDAYLNAVVGTALIKKYPKLESEFVYGDYKVYDVRKMIAKSE |
| | | QEIGKATAKYFFYSNIMNFFKTEITLANGEIRKRPLIETNGETGEIVW |
| | | DKGRDFATVRKVLSMPQVNIVKKTEVQTGGFSKESILPKRNSDKLIA |
| | | RKKDWDPKKYGGFDSPTVAYSVLVVAKVEKGKSKKLKSVKELLGI |
| | | TIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGD |
| 34418 | linker between CAS9 | SGGSSGGSSGSETPGTSESATPESSGGSSGGSS |
| | domain and RT domain | |
| | (33 amino acids) | |
| 34363 | MMLV_RT D200N | TLNIEDEYRLHETSKEPDVSLGSTWLSDFPQAWAETGGMGLAVRQA |
| | T330P L603W T306K | PLIIPLKATSTPVSIKQYPMSQEARLGIKPHIQRLLDQGILVPCQSPWN |
| | W313F | TPLLPVKKPGTNDYRPVQDLREVNKRVEDIHPTVPNPYNLLSGLPPS |
| | | HQWYTVLDLKDAFFCLRLHPTSQPLFAFEWRDPEMGISGQLTWTRL |
| | | PQGFKNSPTLFNEALHRDLADFRIQHPDLILLQYVDDLLLAATSELD |
| | | CQQGTRALLQTLGNLGYRASAKKAQICQKQVKYLGYLLKEGQRWL |
| | | TEARKETVMGQPTPKTPRQLREFLGKAGFCRLFIPGFAEMAAPLYPL |
| | | TKPGTLFNWGPDQQKAYQEIKQALLTAPALGLPDLTKPFELFVDEK |
| | | QGYAKGVLTQKLGPWRRPVAYLSKKLDPVAAGWPPCLRMVAAIA |
| | | VLTKDAGKLTMGQPLVILAPHAVEALVKQPPDRWLSNARMTHYQA |
| | | LLLDTDRVQFGPVVALNPATLLPLPEEGLQHNCLDILAEAHGTRPDL |
| | | TDQPLPDADHTWYTDGSSLLQEGQRKAGAAVTTETEVIWAKALPA |
| | | GTSAQRAELIALTQALKMAEGKKLNVYTDSRYAFATAHIHGEIYRR |
| | | RGWLTSEGKEIKNKDEILALLKALFLPKRLSIIHCPGHQKGHSAEAR |
| | | GNRMADQAARKAAITETPDTSTLLIENSSP |
| 34402 | C-terminal NLS | SGGSKRTADGSEFEPKKKRKV |

TABLE 83
lists exemplary prime editor and its components

| SEQ ID | | |
|---|---|---|
| NO. | DESCRIPTION | SEQUENCE |
| 34405 | Exemplary prime editor | MKRTADGSEFESPKKKRKVDKKYSIGLDIGTNSVGWAVITDEYKV |
| | [NLS]-[Cas9((R220K) | PSKKFKVLGNTDRHSIKKNLIGALLFDSGETAEATRLKRTARRR |
| | (R393K)(H839A)]- | YTRRKNRICYLQEIFSNEMAKVDDSFFHRLEESFLVEEDKKHER |
| | [linker]- | HPIFGNIVDEVAYHEKYPTIYHLRKKLVDSTDKADLRLIYLALAH |
| | [MMLV_RT(D200N)(T330P) | MIKFRGHFLIEGDLNPDNSDVDKLFIQLVQTYNQLFEENPINASG |
| | (L603W)(T306K)(W313F)]- | VDAKAILSARLSKSRKLENLIAQLPGEKKNGLFGNLIALSLGLTP |
| | [NLS] | NFKSNFDLAEDAKLQLSKDTYDDDLDNLLAQIGDQYADLFLAAK |
| | | NLSDAILLSDILRVNTEITKAPLSASMIKRYDEHHQDLTLLKALV |
| | | RQQLPEKYKEIFFDQSKNGYAGYIDGGASQEEFYKFIKPILEKMD |
| | | GTEELLVKLKREDLLRKQRTFDNGSIPHQIHLGELHAILRRQED |
| | | FYPFLKDNREKIEKILTFRIPYYVGPLARGNSRFAWMTRKSEETI |
| | | TPWNFEEVVDKGASAQSFIERMTNFDKNLPNEKVLPKHSLLYEY |
| | | FTVYNELTKVKYVTEGMRKPAFLSGEQKKAIVDLLFKTNRKVT |
| | | VKQLKEDYFKKIECFDSVEISGVEDRFNASLGTYHDLLKIIKDKD |
| | | FLDNEENEDILEDIVLTLTLFEDREMIEERLKTYAHLFDDKVMK |
| | | QLKRRRYTGWGRLSRKLINGIRDKQSGKTILDFLKSDGFANRNF |
| | | MQLIHDDSLTFKEDIQKAQVSGQGDSLHEHIANLAGSPAIKKGIL |
| | | QTVKVVDELVKVMGRHKPENIVIEMARENQTTQKGQKNSRER |
| | | MKRIEEGIKELGSQILKEHPVENTQLQNEKLYLYYLQNGRDMY |
| | | VDQELDINRLSDYDVDAIVPQSFLKDDSIDNKVLTRSDKNRGKSD |
| | | NVPSEEVVKKMKNYWRQLLNAKLITQRKFDNLTKAERGGLSEL |
| | | DKAGFIKRQLVETRQITKHVAQILDSRMNTKYDENDKLIREVKV |
| | | ITLKSKLVSDFRKDFQFYKVREINNYHHAHDAYLNAVVGTALIK |
| | | KYPKLESEFVYGDYKVYDVRKMIAKSEQEIGKATAKYFFYSNIM |
| | | NFFKTEITLANGEIRKRPLIETNGETGEIVWDKGRDFATVRKVLS |
| | | MPQVNIVKKTEVQTGGFSKESILPKRNSDKLIARKKDWDPKKYG |
| | | GFDSPTVAYSVLVVAKVEKGKSKKLKSVKELLGITIMERSSFEKN |
| | | PIDFLEAKGYKEVKKDLIIKLPKYSLFELENGRKRMLASAGELQ |
| | | KGNELALPSKYVNFLYLASHYEKLKGSPEDNEQKQLFVEQHKH |
| | | YLDEIIEQISEFSKRVILADANLDKVLSAYNKHRDKPIREQAENII |
| | | HLFTLTNLGAPAAFKYFDTTIDRKRYTSTKEVLDATLIHQSITGL |
| | | YETRIDLSQLGGDSGGSSGGSKRTADGSEFESPKKKRKVSGGSSG |
| | | GSTLNIEDEYRLHETSKEPDVSLGSTWLSDFPQAWAETGGMGLAVRQAP |
| | | LIIPLKATSTPVSIKQYPMSQEARLGIKPHIQRLLDQGILVPCQSPWNTPLL |
| | | PVKKPGTNDYRPVQDLREVNKRVEDIHPTVPNPYNLLSGLPPSHQWYTV |
| | | LDLKDAFFCLRLHPTSQPLFAFEWRDPEMGISGQLTWTRLPQGFKNSPT |
| | | LFNEALHRDLADFRIQHPDLILLQYVDDLLLAATSELDCQQGTRALLQTL |
| | | GNLGYRASAKKAQICQKQVKYLGYLLKEGQRWLTEARKETVMGQPTPK |
| | | TPRQLREFLGKAGFCRLFIPGFAEMAAPLYPLTKPGTLFNWGPDQQKAY |
| | | QEIKQALLTAPALGLPDLTKPFELFVDEKQGYAKGVLTQKLGPWRRPVA |
| | | YLSKKLDPVAAGWPPCLRMVAAIAVLTKDAGKLTMGQPLVILAPHAVEA |
| | | LVKQPPDRWLSNARMTHYQALLLDTDRVQFGPVVALNPATLLPLPEEGL |
| | | QHNCLDILAEAHGTRPDLTDQPLPDADHTWYTDGSSLLQEGQRKAGAA |
| | | VTTETEVIWAKALPAGTSAQRAELIALTQALKMAEGKKLNVYTDSRYAFA |
| | | TAHIHGEIYRRRGWLTSEGKEIKNKDEILALLKALFLPKRLSIIHCPGHQK |
| | | GHSAEARGNRMADQAARKAAITETPDTSTLLIENSSPSGGSKRTADGSEF |
| | | ESPKKKRKVGSGPAAKRVKLD |
| | | KEY: |
| | | N-terminal bipartiteSV40NLS |
| | | CAS9(R221K N394K H840A) |
| | | SGGSx2-met-bpSV40NLS-SGGSx2 LINKER |
| | | M-MLV D200N T306K W313F T330P L603W REVERSE TRANSCRIPTASE |
| | | C-terminal linker-NLS1 |
| | | C-terminal linker-NLS2 |
| 34392 | N-terminal bpSV40NLS | MKRTADGSEFESPKKKRKV |
| 34406 | CAS9 (R221K N394K | DKKYSIGLDIGTNSVGWAVITDEYKVPSKKFKVLGNTDRHSIKKNLI |
| | H840A) | GALLFDSGETAEATRLKRTARRRYTRRKNRICYLQEIFSNEMAKVD |
| | | DSFFHRLEESFLVEEDKKHERHPIFGNIVDEVAYHEKYPTIYHLRKKL |
| | | VDSTDKADLRLIYLALAHMIKFRGHFLIEGDLNPDNSDVDKLFIQLV |
| | | QTYNQLFEENPINASGVDAKAILSARLSKSRKLENLIAQLPGEKKNG |
| | | LFGNLIALSLGLTPNFKSNFDLAEDAKLQLSKDTYDDDLDNLLAQIG |
| | | DQYADLFLAAKNLSDAILLSDILRVNTEITKAPLSASMIKRYDEHHQ |
| | | DLTLLKALVRQQLPEKYKEIFFDQSKNGYAGYIDGGASQEEFYKFIK |
| | | PILEKMDGTEELLVKLKREDLLRKQRTFDNGSIPHQIHLGELHAILRR |
| | | QEDFYPFLKDNREKIEKILTFRIPYYVGPLARGNSRFAWMTRKSEETI |
| | | TPWNFEEVVDKGASAQSFIERMTNFDKNLPNEKVLPKHSLLYEYFT |
| | | VYNELTKVKYVTEGMRKPAFLSGEQKKAIVDLLFKTNRKVTVKQL |
| | | KEDYFKKIECFDSVEISGVEDRFNASLGTYHDLLKIIKDKDFLDNEEN |
| | | EDILEDIVLTLTLFEDREMIEERLKTYAHLFDDKVMKQLKRRRYTG |
| | | WGRLSRKLINGIRDKQSGKTILDFLKSDGFANRNFMQLIHDDSLTFK |
| | | EDIQKAQVSGQGDSLHEHIANLAGSPAIKKGILQTVKVVDELVKVM |
| | | GRHKPENIVIEMARENQTTQKGQKNSRERMKRIEEGIKELGSQILKE |
| | | HPVENTQLQNEKLYLYYLQNGRDMYVDQELDINRLSDYDVDAIVP |
| | | QSFLKDDSIDNKVLTRSDKNRGKSDNVPSEEVVKKMKNYWRQLLN |
| | | AKLITQRKFDNLTKAERGGLSELDKAGFIKRQLVETRQITKHVAQIL |
| | | DSRMNTKYDENDKLIREVKVITLKSKLVSDFRKDFQFYKVREINNY |
| | | HHAHDAYLNAVVGTALIKKYPKLESEFVYGDYKVYDVRKMIAKSE |
| | | QEIGKATAKYFFYSNIMNFFKTEITLANGEIRKRPLIETNGETGEIVW |
| | | DKGRDFATVRKVLSMPQVNIVKKTEVQTGGFSKESILPKRNSDKLIA |
| | | RKKDWDPKKYGGFDSPTVAYSVLVVAKVEKGKSKKLKSVKELLGI |
| | | TIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGD |
| 34407 | SGGSx2-bpSV40NLS- | SGGSSGGSKRTADGSEFESPKKKRKVSGGSSGGS |
| | SGGSx2 linker | |
| 34363 | MMLV_RT D200N | TLNIEDEYRLHETSKEPDVSLGSTWLSDFPQAWAETGGMGLAVRQA |
| | T330P L603W T306K | PLIIPLKATSTPVSIKQYPMSQEARLGIKPHIQRLLDQGILVPCQSPWN |
| | W313F | TPLLPVKKPGTNDYRPVQDLREVNKRVEDIHPTVPNPYNLLSGLPPS |
| | | HQWYTVLDLKDAFFCLRLHPTSQPLFAFEWRDPEMGISGQLTWTRL |
| | | PQGFKNSPTLFNEALHRDLADFRIQHPDLILLQYVDDLLLAATSELD |
| | | CQQGTRALLQTLGNLGYRASAKKAQICQKQVKYLGYLLKEGQRWL |
| | | TEARKETVMGQPTPKTPRQLREFLGKAGFCRLFIPGFAEMAAPLYPL |
| | | TKPGTLFNWGPDQQKAYQEIKQALLTAPALGLPDLTKPFELFVDEK |
| | | QGYAKGVLTQKLGPWRRPVAYLSKKLDPVAAGWPPCLRMVAAIA |
| | | VLTKDAGKLTMGQPLVILAPHAVEALVKQPPDRWLSNARMTHYQA |
| | | LLLDTDRVQFGPVVALNPATLLPLPEEGLQHNCLDILAEAHGTRPDL |
| | | TDQPLPDADHTWYTDGSSLLQEGQRKAGAAVTTETEVIWAKALPA |
| | | GTSAQRAELIALTQALKMAEGKKLNVYTDSRYAFATAHIHGEIYRR |
| | | RGWLTSEGKEIKNKDEILALLKALFLPKRLSIIHCPGHQKGHSAEAR |
| | | GNRMADQAARKAAITETPDTSTLLIENSSP |
| 34408 | C-terminal linker-NLS | SGGSKRTADGSEFESPKKKRKV |
| 34409 | C-terminal linker-NLS2 | GSGPAAKRVKLD |

### PEgRNA for Editing of NCF1 Gene

The term “prime editing guide RNA”, or “PEgRNA”, refers to a guide polynucleotide that comprises one or more intended nucleotide edits for incorporation into the target DNA. In some embodiments, the PEgRNA associates with and directs a prime editor to incorporate the one or more intended nucleotide edits into the target gene via prime editing. “Nucleotide edit” or “intended nucleotide edit” refers to a specified deletion of one or more nucleotides at one specific position, insertion of one or more nucleotides at one specific position, substitution of a single nucleotide, or other alterations at one specific position to be incorporated into the sequence of the target gene. Intended nucleotide edit may refer to the edit on the editing template as compared to the sequence on the target strand of the target gene, or may refer to the edit encoded by the editing template on the newly synthesized single stranded DNA that replaces the editing target sequence, as compared to the editing target sequence. In some embodiments, a PEgRNA comprises a spacer sequence that is complementary or substantially complementary to a search target sequence on a target strand of the target gene. In some embodiments, the PEgRNA comprises a gRNA core that associates with a DNA binding domain, e.g., a CRISPR-Cas protein domain, of a prime editor. In some embodiments, the PEgRNA further comprises an extended nucleotide sequence comprising one or more intended nucleotide edits compared to the endogenous sequence of the target gene, wherein the extended nucleotide sequence may be referred to as an extension arm.

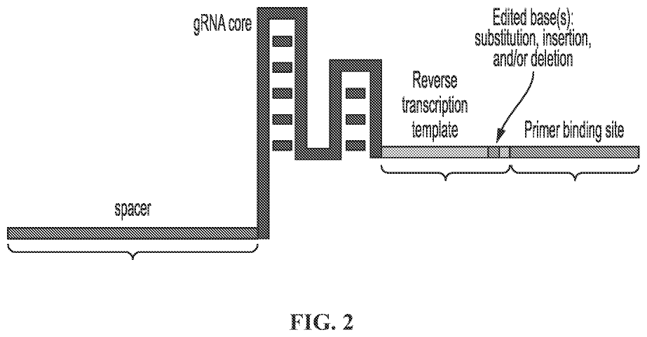

In certain embodiments, the extension arm comprises a primer binding site sequence (PBS) that can initiate target-primed DNA synthesis. In some embodiments, the PBS is complementary or substantially complementary to a free 3′ end on the edit strand of the target gene at a nick site generated by the prime editor. In some embodiments, the extension arm further comprises an editing template that comprises one or more intended nucleotide edits to be incorporated in the target gene by prime editing. In some embodiments, the editing template is a template for an RNA-dependent DNA polymerase domain or polypeptide of the prime editor, for example, a reverse transcriptase domain. The reverse transcriptase editing template may also be referred to herein as an RT template, or RTT. In some embodiments, the editing template comprises partial complementarity to an editing target sequence in the target gene, e.g., an NCF1 gene. In some embodiments, the editing template comprises substantial or partial complementarity to the editing target sequence except at the position of the intended nucleotide edits to be incorporated into the target gene. An exemplary architecture of a PEgRNA including its components is as demonstrated in FIG. 2.

In some embodiments, a PEgRNA includes only RNA nucleotides and forms an RNA polynucleotide. In some embodiments, a PEgRNA is a chimeric polynucleotide that includes both RNA and DNA nucleotides. For example, a PEgRNA can include DNA in the spacer sequence, the gRNA core, or the extension arm. In some embodiments, a PEgRNA comprises DNA in the spacer sequence. In some embodiments, the entire spacer sequence of a PEgRNA is a DNA sequence. In some embodiments, the PEgRNA comprises DNA in the gRNA core, for example, in a stem region of the gRNA core. In some embodiments, the PEgRNA comprises DNA in the extension arm, for example, in the editing template. An editing template that comprises a DNA sequence may serve as a DNA synthesis template for a DNA polymerase in a prime editor, for example, a DNA-dependent DNA polymerase. Accordingly, the PEgRNA may be a chimeric polynucleotide that comprises RNA in the spacer, gRNA core, and/or the PBS sequences and DNA in the editing template.

Components of a PEgRNA may be arranged in a modular fashion. In some embodiments, the spacer and the extension arm comprising a primer binding site sequence (PBS) and an editing template, e.g., a reverse transcriptase template (RTT), can be interchangeably located in the 5′ portion of the PEgRNA, the 3′ portion of the PEgRNA, or in the middle of the gRNA core. In some embodiments, a PEgRNA comprises a PBS and an editing template sequence in 5′ to 3′ order. In some embodiments, the gRNA core of a PEgRNA of this disclosure may be located in between a spacer and an extension arm of the PEgRNA. In some embodiments, the gRNA core of a PEgRNA may be located at the 3′ end of a spacer. In some embodiments, the gRNA core of a PEgRNA may be located at the 5′ end of a spacer. In some embodiments, the gRNA core of a PEgRNA may be located at the 3′ end of an extension arm. In some embodiments, the gRNA core of a PEgRNA may be located at the 5′ end of an extension arm. In some embodiments, the PEgRNA comprises, from 5′ to 3′: a spacer, a gRNA core, and an extension arm. In some embodiments, the PEgRNA comprises, from 5′ to 3′: a spacer, a gRNA core, an editing template, and a PBS. In some embodiments, the PEgRNA comprises, from 5′ to 3′: an extension arm, a spacer, and a gRNA core. In some embodiments, the PEgRNA comprises, from 5′ to 3′: an editing template, a PBS, a spacer, and a gRNA core.

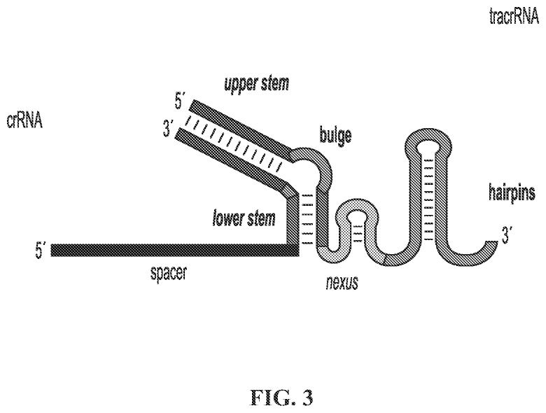

In some embodiments, a PEgRNA comprises a single polynucleotide molecule that comprises the spacer sequence, the gRNA core, and the extension arm. In some embodiments, a PEgRNA comprises multiple polynucleotide molecules, for example, two polynucleotide molecules. In some embodiments, a PEgRNA comprise a first polynucleotide molecule that comprises the spacer and a portion of the gRNA core, and a second polynucleotide molecule that comprises the rest of the gRNA core and the extension arm. In some embodiments, the gRNA core portion in the first polynucleotide molecule and the gRNA core portion in the second polynucleotide molecule are at least partly complementary to each other. In some embodiments, the PEgRNA may comprise a first polynucleotide comprising the spacer and a first portion of a gRNA core comprising, which may be also be referred to as a crRNA. In some embodiments, the PEgRNA comprise a second polynucleotide comprising a second portion of the gRNA core and the extension arm, wherein the second portion of the gRNA core may also be referred to as a trans-activating crRNA, or tracr RNA. In some embodiments, the crRNA portion and the tracr RNA portion of the gRNA core are at least partially complementary to each other. In some embodiments, the partially complementary portions of the crRNA and the tracr RNA form a lower stem, a bulge, and an upper stem, as exemplified in FIG. 3.

In some embodiments, a spacer sequence comprises a region that has substantial complementarity to a search target sequence on the target strand of a double stranded target DNA, e.g., an NCF1 gene, an NCF1B pseudogene, or an NCF1C pseudogene. In some embodiments, the spacer sequence of a PEgRNA is identical or substantially identical to a protospacer sequence on the edit strand of the target gene (except that the protospacer sequence comprises thymine and the spacer sequence may comprise uracil). In some embodiments, the spacer sequence is at least about 70%, 75%, 80%, 85%, 90%, 95%, or 100% complementary to a search target sequence in the target gene. In some embodiments, the spacer comprises is substantially complementary to the search target sequence.

In some embodiments, the length of the spacer varies from about 10 to about 100 nucleotides. In some embodiments, the spacer is 16 nucleotides, 17 nucleotides, 18 nucleotides, 19 nucleotides, 20 nucleotides, 21 nucleotides, 22 nucleotides, 23 nucleotides, 24 nucleotides, or 25 nucleotides in length. In some embodiments, the spacer is from 15 nucleotides to 30 nucleotides in length, 15 to 25 nucleotides in length, 18 to 22 nucleotides in length, 10 to 20 nucleotides in length, or 20 to 30 nucleotides in length. In some embodiments, the spacer is 16 to 22 nucleotides in length, e.g., about 16, 17, 18, 19, 20, 21, or 22 nucleotides in length. In some embodiments, the spacer is 20 nucleotides in length.

As used herein in a PEgRNA or a nick guide RNA sequence, or fragments thereof such as a spacer, PBS, or RT sequence, unless indicated otherwise, it should be appreciated that the letter “T” or “thymine” indicates a nucleobase in a DNA sequence that encodes the PEgRNA or guide RNA sequence, and is intended to refer to a uracil (U) nucleobase of the PEgRNA or guide RNA or any chemically modified uracil nucleobase known in the art, such as 5-methoxyuracil.

The extension arm of a PEgRNA may comprise a primer binding site (PBS) and an editing template (e.g., an RTT). The extension arm may be partially complementary to the spacer. In some embodiments, the editing template (e.g., RTT) is partially complementary to the spacer. In some embodiments, the editing template (e.g., RTT) and the primer binding site (PBS) are each partially complementary to the spacer.

An extension arm of a PEgRNA may comprise a primer binding site sequence (PBS, or PBS sequence) that comprises complementarity to and can hybridize with a free 3′ end of a single stranded DNA in the target gene (e.g., an NCF1 gene, an NCF1B pseudogene, or an NCF1C pseudogene) generated by nicking with a prime editor at the nick site on the PAM strand.

The length of the PBS sequence may vary depending on, e.g., the prime editor components, the search target sequence and other components of the PEgRNA. In some embodiments, the PBS is about 3 to 19 nucleotides in length. In some embodiments, the PBS is about 3 to 17 nucleotides in length. In some embodiments, the PBS is about 4 to 16 nucleotides, about 6 to 16 nucleotides, about 6 to 18 nucleotides, about 6 to 20 nucleotides, about 8 to 20 nucleotides, about 10 to 20 nucleotides, about 12 to 20 nucleotides, about 14 to 20 nucleotides, about 16 to 20 nucleotides, or about 18 to 20 nucleotides in length. In some embodiments, the PBS is 8 to 17 nucleotides in length. In some embodiments, the PBS is 8 to 16 nucleotides in length. In some embodiments, the PBS is 8 to 15 nucleotides in length. In some embodiments, the PBS is 8 to 14 nucleotides in length. In some embodiments, the PBS is 8 to 13 nucleotides in length. In some embodiments, the PBS is 8 to 12 nucleotides in length. In some embodiments, the PBS is 8 to 11 nucleotides in length. In some embodiments, the PBS is 8 to 10 nucleotides in length. In some embodiments, the PBS is 8 or 9 nucleotides in length. In some embodiments, the PBS is 16 or 17 nucleotides in length. In some embodiments, the PBS is 15 to 17 nucleotides in length. In some embodiments, the PBS is 14 to 17 nucleotides in length. In some embodiments, the PBS is 13 to 17 nucleotides in length. In some embodiments, the PBS is 12 to 17 nucleotides in length. In some embodiments, the PBS is 11 to 17 nucleotides in length. In some embodiments, the PBS is 10 to 17 nucleotides in length. In some embodiments, the PBS is 9 to 17 nucleotides in length. In some embodiments, the PBS is about 7 to 15 nucleotides in length. In some embodiments, the PBS is 6, 7, 8, 9, 10, 11, 12, 13, 14, 15, 16, 17, 18, or 19 nucleotides in length. In some embodiments, the PBS is 8 to 14 nucleotides in length. For example, the PBS can be 8, 9, 10, 11, 12, 13, or 14 nucleotides in length. In some embodiments, the PBS is 11 or 12 nucleotides in length. In some embodiments, the PBS is 11 to 13 nucleotides in length. In some embodiments, the PBS is 11 to 14 nucleotides in length.

The PBS may be complementary or substantially complementary to a DNA sequence in the edit strand of the target gene. By annealing with the edit strand at a free hydroxy group, e.g., a free 3′ end generated by prime editor nicking, the PBS may initiate synthesis of a new single stranded DNA encoded by the editing template at the nick site. In some embodiments, the PBS is at least about 70%, 75%, 80%, 85%, 90%, 95%, or 100% complementary to a region of the edit strand of the target gene (e.g., an NCF1 gene, an NCF1B pseudogene, or an NCF1C pseudogene). In some embodiments, the PBS is perfectly complementary, or 100% complementary, to a region of the edit strand of the target gene (e.g., an NCF1 gene, an NCF1B pseudogene, or an NCF1C pseudogene).

An extension arm of a PEgRNA may comprise an editing template that serves as a DNA synthesis template for the DNA polymerase in a prime editor during prime editing.

The length of an editing template may vary depending on, e.g., the prime editor components, the search target sequence and other components of the PEgRNA. In some embodiments, the editing template serves as a DNA synthesis template for a reverse transcriptase, and the editing template is referred to as a reverse transcription editing template (RTT).

The editing template (e.g., RTT), in some embodiments, is 2, 3, 4, 5, 6, 7, 8, 9, 10, 11, 12, 13, 14, 15, 16, 17, 18, 19, 20, 21, 22, 23, 24, or 25 nucleotides in length. In some embodiments, the RTT is 12, 13, 14, 15, 16, 17, 18, 19, 20, 21, 22, 23, 24, or 25 nucleotides in length. In some embodiments, the RTT is 16, 17, 18, 19, 20, 21, 22, 23, 24, 25, 26, 27, 28, 29, 30, 31, 32, 33, 34, 35, 36, 37, 38, 39, or 40 nucleotides in length. In some embodiments, the RTT is 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleotides in length. In some embodiments, the RTT is 10 to 110 nucleotides in length. In some embodiments, the RU is 10 to 109, 10 to 108, 10 to 107, 10 to 106, 10 to 105, 10 to 104, 10 to 103, 10 to 102, or 10 to 101 nucleotides in length. In some embodiments, the RTT is at least 8 and no more than 50 nucleotides in length. In some embodiments, the RU is at least 8 and no more than 25 nucleotides in length. In some embodiments, the RTT is about 10 to about 20 nucleotides in length. In some embodiments, the RU is about 11, 12, 13, 14, 15, 16, 17, 18, or 19 nucleotides in length. In some embodiments, the RTT is 11 to 17 nucleotides in length. In some embodiments, the RU is 12 to 17 nucleotides in length. In some embodiments, the RTT is 12 to 16 nucleotides in length. In some embodiments, the RTT is 13 to 17 nucleotides in length. In some embodiments, the RU is 11, 12, 13, 14, 15, 16, or 17 nucleotides in length. In some embodiments the RU is 12 nucleotides in length. In some embodiments the RTT is 16 nucleotides in length. In some embodiments the RU is 17 nucleotides in length.

In some embodiments, the editing template (e.g., RU) sequence is about 70%, 75%, 80%, 85%, 90%, 95%, or 99% complementary to the editing target sequence on the edit strand of the target gene (e.g., an NCF1 gene, an NCF1B pseudogene, or an NCF1C pseudogene). In some embodiments, the editing template sequence (e.g., RTT) is substantially complementary to the editing target sequence. In some embodiments, the editing template sequence (e.g., RTT) is complementary to the editing target sequence except at positions of the intended nucleotide edits to be incorporated int the target gene. In some embodiments, the editing template comprises a nucleotide sequence comprising about 85% to about 95% complementarity to an editing target sequence in the edit strand in the target gene (e.g., an NCF1 gene, an NCF1B pseudogene, or an NCF1C pseudogene). In some embodiments, the editing template comprises about 86%, about 87%, about 88%, about 89%, about 90%, about 91%, about 92%, about 93%, about 94%, about 95%, about 96%, about 97%, about 98%, or about 99% complementarity to an editing target sequence in the edit strand of the target gene (e.g., an NCF1 gene, an NCF1B pseudogene, or an NCF1C pseudogene).

An intended nucleotide edit in an editing template of a PEgRNA may comprise various types of alterations as compared to the target gene sequence. In some embodiments, the nucleotide edit is a single nucleotide substitution as compared to the target gene sequence. In some embodiments, the nucleotide edit is a deletion as compared to the target gene sequence. In some embodiments, the nucleotide edit is an insertion as compared to the target gene sequence. In some embodiments, the editing template comprises one to ten intended nucleotide edits as compared to the target gene sequence. In some embodiments, the editing template comprises one or more intended nucleotide edits as compared to the target gene sequence. In some embodiments, the editing template comprises two or more intended nucleotide edits as compared to the target gene sequence. In some embodiments, the editing template comprises three or more intended nucleotide edits as compared to the target gene sequence. In some embodiments, the editing template comprises four or more, five or more, or six or more intended nucleotide edits as compared to the target gene sequence. In some embodiments, the editing template comprises two single nucleotide substitutions, insertions, deletions, or any combination thereof, as compared to the target gene sequence. In some embodiments, the editing template comprises three single nucleotide substitutions, insertions, deletions, or any combination thereof, as compared to the target gene sequence. In some embodiments, the editing template comprises four, five, or six single nucleotide substitutions, insertions, deletions, or any combination thereof, as compared to the target gene sequence. In some embodiments, a nucleotide substitution comprises an adenine (A)-to-thymine (T) substitution. In some embodiments, a nucleotide substitution comprises an A-to-guanine (G) substitution. In some embodiments, a nucleotide substitution comprises an A-to-cytosine (C) substitution. In some embodiments, a nucleotide substitution comprises a T-A substitution. In some embodiments, a nucleotide substitution comprises a T-G substitution. In some embodiments, a nucleotide substitution comprises a T-C substitution. In some embodiments, a nucleotide substitution comprises a G-to-A substitution. In some embodiments, a nucleotide substitution comprises a G-to-T substitution. In some embodiments, a nucleotide substitution comprises a G-to-C substitution. In some embodiments, a nucleotide substitution comprises a C-to-A substitution. In some embodiments, a nucleotide substitution comprises a C-to-T substitution. In some embodiments, a nucleotide substitution comprises a C-to-G substitution.

In some embodiments, a nucleotide insertion is at least 1, at least 2, at least 3, at least 4, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, or at least 20 nucleotides in length. In some embodiments, a nucleotide insertion is from 1 to 2 nucleotides, from 1 to 3 nucleotides, from 1 to 4 nucleotides, from 1 to 5 nucleotides, form 2 to 5 nucleotides, from 3 to 5 nucleotides, from 3 to 6 nucleotides, from 3 to 8 nucleotides, from 4 to 9 nucleotides, from 5 to 10 nucleotides, from 6 to 11 nucleotides, from 7 to 12 nucleotides, from 8 to 13 nucleotides, from 9 to 14 nucleotides, from 10 to 15 nucleotides, from 11 to 16 nucleotides, from 12 to 17 nucleotides, from 13 to 18 nucleotides, from 14 to 19 nucleotides, from 15 to 20 nucleotides in length. In some embodiments, a nucleotide insertion is a single nucleotide insertion. In some embodiments, a nucleotide insertion comprises insertion of two nucleotides.

The editing template of a PEgRNA may comprise one or more intended nucleotide edits, compared to the target gene (e.g., an NCF1 gene, an NCF1B pseudogene, or an NCF1C pseudogene) to be edited. Position of the intended nucleotide edit(s) relevant to other components of the PEgRNA, or to particular nucleotides (e.g., mutations) in the target gene may vary. In some embodiments, the nucleotide edit is in a region of the PEgRNA corresponding to or homologous to the protospacer sequence. In some embodiments, the nucleotide edit is in a region of the PEgRNA corresponding to a region of the target gene outside of the protospacer sequence.

By “upstream” and “downstream” it is intended to define relevant positions at least two regions or sequences in a nucleic acid molecule orientated in a 5′-to-3′ direction. For example, a first sequence is upstream of a second sequence in a DNA molecule where the first sequence is positioned 5′ to the second sequence. Accordingly, the second sequence is downstream of the first sequence.

In some embodiments, the position of a nucleotide edit incorporation in the target gene can be determined based on position of the nick site. In some embodiments, position of an intended nucleotide edit is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, 100, 110, 120, 130, 140, or 150 nucleotides apart from the nick site. In some embodiments, position of an intended nucleotide edit is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, 100, 110, 120, 130, 140, or 150 nucleotides downstream of the nick site on the PAM strand (or the non-target strand, or the edit strand) of the double stranded target DNA. In some embodiments, position of the intended nucleotide edit in the editing template can be referred to by aligning the editing template with the partially complementary editing target sequence on the edit strand and referring to nucleotide positions on the editing strand where the intended nucleotide edit is incorporated. Accordingly, in some embodiments, a nucleotide edit in an editing template is at a position corresponding to a position about 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, 100, 110, 120, 130, 140, or 150 nucleotides apart from the nick site. In some embodiments, a nucleotide edit in an editing template is at a position corresponding to a position about 0 to 2 nucleotides, 0 to 4 nucleotides, 0 to 6 nucleotides, 0 to 8 nucleotides, 0 to 10 nucleotides, 2 to 4 nucleotides, 2 to 6 nucleotides, 2 to 8 nucleotides, 2 to 10 nucleotides, 2 to 12 nucleotides, 4 to 6 nucleotides, 4 to 8 nucleotides, 4 to 10 nucleotides, 4 to 12 nucleotides, 4 to 14 nucleotides, 6 to 8 nucleotides, 6 to 10 nucleotides, 6 to 12 nucleotides, 6 to 14 nucleotides, 6 to 16 nucleotides, 8 to 10 nucleotides, 8 to 12 nucleotides, 8 to 14 nucleotides, 8 to 16 nucleotides, 8 to 18 nucleotides, 10 to 12 nucleotides, 10 to 14 nucleotides, 10 to 16 nucleotides, 10 to 18 nucleotides, 10 to 20 nucleotides, 12 to 14 nucleotides, 12 to 16 nucleotides, 12 to 18 nucleotides, 12 to 20 nucleotides, 12 to 22 nucleotides, 14 to 16 nucleotides, 14 to 18 nucleotides, 14 to 20 nucleotides, 14 to 22 nucleotides, 14 to 24 nucleotides, 16 to 18 nucleotides, 16 to 20 nucleotides, 16 to 22 nucleotides, 16 to 24 nucleotides, 16 to 26 nucleotides, 18 to 20 nucleotides, 18 to 22 nucleotides, 18 to 24 nucleotides, 18 to 26 nucleotides, 18 to 28 nucleotides, 20 to 22 nucleotides, 20 to 24 nucleotides, 20 to 26 nucleotides, 20 to 28 nucleotides, 20 to 30 nucleotides, 30 to 40 nucleotides, 40 to 50 nucleotides, 50 to 60 nucleotides, 60 to 70 nucleotides, 70 to 80 nucleotides, 80 to 90 nucleotides, 90 to 100 nucleotides, 100 to 110 nucleotides, 110 to 120 nucleotides, 120 to 130 nucleotides, 130 to 140 nucleotides, or 140 to 150 nucleotides apart from the nick site. In some embodiments, when referred to in the context of the PAM strand (or the non-target strand, or the edit strand), a nucleotide edit in an editing template is at a position corresponding to a position about 0 to 2 nucleotides, 0 to 4 nucleotides, 0 to 6 nucleotides, 0 to 8 nucleotides, 0 to 10 nucleotides, 2 to 4 nucleotides, 2 to 6 nucleotides, 2 to 8 nucleotides, 2 to 10 nucleotides, 2 to 12 nucleotides, 4 to 6 nucleotides, 4 to 8 nucleotides, 4 to 10 nucleotides, 4 to 12 nucleotides, 4 to 14 nucleotides, 6 to 8 nucleotides, 6 to 10 nucleotides, 6 to 12 nucleotides, 6 to 14 nucleotides, 6 to 16 nucleotides, 8 to 10 nucleotides, 8 to 12 nucleotides, 8 to 14 nucleotides, 8 to 16 nucleotides, 8 to 18 nucleotides, 10 to 12 nucleotides, 10 to 14 nucleotides, 10 to 16 nucleotides, 10 to 18 nucleotides, 10 to 20 nucleotides, 12 to 14 nucleotides, 12 to 16 nucleotides, 12 to 18 nucleotides, 12 to 20 nucleotides, 12 to 22 nucleotides, 14 to 16 nucleotides, 14 to 18 nucleotides, 14 to 20 nucleotides, 14 to 22 nucleotides, 14 to 24 nucleotides, 16 to 18 nucleotides, 16 to 20 nucleotides, 16 to 22 nucleotides, 16 to 24 nucleotides, 16 to 26 nucleotides, 18 to 20 nucleotides, 18 to 22 nucleotides, 18 to 24 nucleotides, 18 to 26 nucleotides, 18 to 28 nucleotides, 20 to 22 nucleotides, 20 to 24 nucleotides, 20 to 26 nucleotides, 20 to 28 nucleotides, 20 to 30 nucleotides, 30 to 40 nucleotides, 40 to 50 nucleotides, 50 to 60 nucleotides, 60 to 70 nucleotides, 70 to 80 nucleotides, 80 to 90 nucleotides, 90 to 100 nucleotides, 100 to 110 nucleotides, 110 to 120 nucleotides, 120 to 130 nucleotides, 130 to 140 nucleotides, or 140 to 150 nucleotides downstream from the nick site. The relative positions of the intended nucleotide edit(s) and nick site may be referred to by numbers. For example, in some embodiments, the nucleotide immediately downstream of the nick site on a PAM strand (or the non-target strand, or the edit strand) may be referred to as at position 0. The nucleotide immediately upstream of the nick site on the PAM strand (or the non-target strand, or the edit strand) may be referred to as at position −1. The nucleotides downstream of position 0 on the PAM strand can be referred to as at positions +1, +2, +3, +4, . . . +n, and the nucleotides upstream of position −1 on the PAM strand may be referred to as at positions −2, −3, −4, . . . , −n. Accordingly, in some embodiments, the nucleotide in the editing template that corresponds to position 0 when the editing template is aligned with the partially complementary editing target sequence by complementarity can also be referred to as position 0 in the editing template, the nucleotides in the editing template corresponding to the nucleotides at positions +1, +2, +3, +4, . . . , +n on the PAM strand of the double stranded target DNA can also be referred to as at positions +1, +2, +3, +4, . . . , +n in the editing template, and the nucleotides in the editing template corresponding to the nucleotides at positions −1, −2, −3, −4, . . . , −n on the PAM strand on the double stranded target DNA may also be referred to as at positions −1, −2, −3, −4, . . . , −n on the editing template, even though when the PEgRNA is viewed as a standalone nucleic acid, positions +1, +2, +3, +4, . . . , +n are 5′ of position 0 and positions −1, −2, −3, −4, . . . −n are 3′ of position 0 in the editing template. In some embodiments, an intended nucleotide edit is at position +n of the editing template relative to position 0. Accordingly, the intended nucleotide edit may be incorporated at position +n of the PAM strand of the double stranded target DNA (and subsequently, the target strand of the double stranded target DNA) by prime editing. The corresponding positions of the intended nucleotide edit incorporated in the target gene may also be referred to based on the nicking position generated by a prime editor based on sequence homology and complementarity. For example, in embodiments, the distance between the nucleotide edit to be incorporated into the target gene (e.g., an NCF1 gene, an NCF1B pseudogene, or an NCF1C pseudogene) and the nick site (also referred to as the “nick to edit distance”) may be determined by the position of the nick site and the position of the nucleotide(s) corresponding to the intended nucleotide edit(s), for example, by identifying sequence complementarity between the spacer and the search target sequence and sequence complementarity between the editing template and the editing target sequence. In certain embodiments, the position of the nucleotide edit can be in any position downstream of the nick site on the edit strand (or the PAM strand). As used herein, the distance between the nick site and the nucleotide edit, for example, where the nucleotide edit comprises an insertion or deletion, refers to the 5′ most position of the nucleotide edit for a nick that creates a 3′ free end on the edit strand (i.e., the “near position” of the nucleotide edit to the nick site). For PEgRNAs designed to correct or edit the c.73_74delGT deletion in NCF1 (or corresponding edits in NCF1B/NCF1C), the nick-to-edit distance can be considered by the “edit” position being the position of the third and fourth nucleotides in exon 2 of NCF1 gene (because insertion of, e.g., one “GT” upstream or downstream of another “GT” results in the same “GTGT” sequence, which is the wildtype sequence of NCF1 at the first 4 nucleotides of exon 2). For example, a PEgRNA having a spacer sequence of SEQ ID NO: 3995 has a nick-to-edit distance of 2 nucleotides. In some embodiments, the nick-to-edit distance is 2 to 106 nucleotides. In some embodiments, the nick-to-edit distance is 2 to 105, 2 to 104, 2 to 103, 2 to 102, 2 to 101, 2 to 100, 2 to 99, 2 to 98, or 2 to 97 nucleotides. In some embodiments, the nick-to-edit distance is 2 to 90, 2 to 80, 2 to 70, 2 to 60, 2 to 50, 2 to 40, or 2 to 30 nucleotides. In some embodiments, the nick-to-edit distance is 2 to 25, 2 to 20, 2 to 15, or 2 to 10 nucleotides. In some embodiments, the nick-to-edit distance is 2, 3, 4, 5, 6, or 7 nucleotides in length.

The RT length and the nick-to-edit distance relate to the length of the portion of the RTT that is upstream of (i.e. 5′ to) the 5′-most edit in the RTT and is complementary to the edit strand. In some embodiments, the editing template comprises at least 4 contiguous nucleotides of complementarity with the edit strand wherein the at least 4 nucleotides contiguous are located upstream of the 5′ most edit in the editing template. In some embodiments, the editing template comprises at least 5, 6, 7, 8, 9, 10, 11, 12, 13, 14, 15, 16, 17, 18, 19, or more contiguous nucleotides of complementarity with the edit strand wherein the at least 5, 6, 7, 8, 9, 10, 11, 12, 13, 14, 15, 16, 17, 18, 19, or more contiguous nucleotides are located upstream of the 5′ most edit in the editing template. In some embodiments, the editing template comprises 20-25, 25-30, 30-35, 35-40, 45-45, or 45-50 contiguous nucleotides of complementarity with the edit strand wherein the 20-25, 25-30, 30-35, 35-40, 45-45, or 45-50 or more contiguous nucleotides are located upstream of the 5′ most edit in the editing template. In some embodiments, the editing template comprises 9-14 contiguous nucleotides of complementarity with the edit strand wherein the 9-14 contiguous nucleotides are located upstream of the 5′ most edit in the editing template. In some embodiments, the editing template comprises 6-10 contiguous nucleotides of complementarity with the edit strand wherein the 6-10 contiguous nucleotides are located upstream of the 5′ most edit in the editing template. In some embodiments, the editing template comprises 10 contiguous nucleotides of complementarity with the edit strand wherein the 10 contiguous nucleotides are located upstream of the 5′ most edit in the editing template. In some embodiments, the editing template comprises 9 contiguous nucleotides of complementarity with the edit strand wherein the 9 contiguous nucleotides are located upstream of the 5′ most edit in the editing template.

When referred to within the PEgRNA, positions of the one or more intended nucleotide edits may be referred to relevant to components of the PEgRNA. For example, an intended nucleotide edit may be 5′ or 3′ to the PBS. In some embodiments, a PEgRNA comprises the structure, from 5′ to 3′: a spacer, a gRNA core, an editing template, and a PBS. In some embodiments, the intended nucleotide edit is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides upstream to the 5′ most nucleotide of the PBS. In some embodiments, the intended nucleotide edit is 0 to 2 nucleotides, 0 to 4 nucleotides, 0 to 6 nucleotides, 0 to 8 nucleotides, 0 to 10 nucleotides, 2 to 4 nucleotides, 2 to 6 nucleotides, 2 to 8 nucleotides, 2 to 10 nucleotides, 2 to 12 nucleotides, 4 to 6 nucleotides, 4 to 8 nucleotides, 4 to 10 nucleotides, 4 to 12 nucleotides, 4 to 14 nucleotides, 6 to 8 nucleotides, 6 to 10 nucleotides, 6 to 12 nucleotides, 6 to 14 nucleotides, 6 to 16 nucleotides, 8 to 10 nucleotides, 8 to 12 nucleotides, 8 to 14 nucleotides, 8 to 16 nucleotides, 8 to 18 nucleotides, 10 to 12 nucleotides, 10 to 14 nucleotides, 10 to 16 nucleotides, 10 to 18 nucleotides, 10 to 20 nucleotides, 12 to 14 nucleotides, 12 to 16 nucleotides, 12 to 18 nucleotides, 12 to 20 nucleotides, 12 to 22 nucleotides, 14 to 16 nucleotides, 14 to 18 nucleotides, 14 to 20 nucleotides, 14 to 22 nucleotides, 14 to 24 nucleotides, 16 to 18 nucleotides, 16 to 20 nucleotides, 16 to 22 nucleotides, 16 to 24 nucleotides, 16 to 26 nucleotides, 18 to 20 nucleotides, 18 to 22 nucleotides, 18 to 24 nucleotides, 18 to 26 nucleotides, 18 to 28 nucleotides, 20 to 22 nucleotides, 20 to 24 nucleotides, 20 to 26 nucleotides, 20 to 28 nucleotides, or 20 to 30 nucleotides upstream to the 5′ most nucleotide of the PBS.

The corresponding positions of the intended nucleotide edit incorporated in the target gene may also be referred to based on the nicking position generated by a prime editor based on sequence homology and complementarity. For example, in embodiments, the distance between the nucleotide edit to be incorporated into the target gene (e.g., an NCF1 gene, an NCF1B pseudogene, or an NCF1C pseudogene) and the nick site (also referred to as the “nick to edit distance”) may be determined by the position of the nick site and the position of the nucleotide(s) corresponding to the intended nucleotide edit(s), for example, by identifying sequence complementarity between the spacer and the search target sequence and sequence complementarity between the editing template and the editing target sequence. In certain embodiments, the position of the nucleotide edit can be in any position downstream of the nick site on the edit strand (or the PAM strand) generated by the prime editor, such that the distance between the nick site and the intended nucleotide edit is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides in length. In some embodiments, the position of the nucleotide edit is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides upstream of the nick site on the edit strand. In some embodiments, the position of the nucleotide edit is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides downstream of the nick site on the edit strand. In some embodiments, the position of the nucleotide edit is 0 base pair from the nick site on the edit strand, that is, the editing position is at the same position as the nick site. As used herein, the distance between the nick site and the nucleotide edit, for example, where the nucleotide edit comprises an insertion or deletion, refers to the 5′ most position of the nucleotide edit for a nick that creates a 3′ free end on the edit strand (i.e., the “near position” of the nucleotide edit to the nick site). Similarly, as used herein, the distance between the nick site and a PAM position edit, for example, where the nucleotide edit comprises an insertion, deletion, or substitution of two or more contiguous nucleotides, refers to the 5′ most position of the nucleotide edit and the 5′ most position of the PAM sequence. In some embodiments, the editing template extends beyond a nucleotide edit to be incorporated to the target gene sequence (e.g., an NCF1 gene, an NCF1B pseudogene, or an NCF1C pseudogene sequence). For example, in some embodiments, the editing template comprises at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleotides.

In some embodiments, the editing template can comprise comprising a second edit relative to a target sequence. The second edit can be designed to mutate or otherwise silence a PAM sequence such that a corresponding nucleic acid guided nuclease or CRISPR nuclease is no longer able to cleave the target sequence (such edits referred to as “PAM silencing edits).

Without wishing to be bound by any particular theory, PAM silencing edits may prevent the Cas, e.g., Cas9, nickase, from re-nicking the edit strand before the edit is incorporated in the target strand, therefore improving prime editing efficiency. In some embodiments, a PAM silencing edit is a synonymous edit that does not alter the amino acid sequence encoded by the target gene after incorporation of the edit. In some embodiments, a PAM silencing edit is at a position corresponding to a non-coding region, e.g., an intron, of a target NCF1 (or NCF1B/NCF1C) gene. Preferrably, the edits in an intron of a target gene is not at a position that corresponds to intron-exon junction and the edit does not affect transcript splicing.

In some embodiments, the length of the editing template is at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleotides longer than the nick to edit distance. In some embodiments, for example, the nick to edit distance is 8 nucleotides, and the editing template is 10 to 15, 10 to 20, 10 to 25, 10 to 30, 10 to 35, 10 to 40, 10 to 45, 10 to 50, 10 to 55, 10 to 60, 10 to 65, 10 to 70, 10 to 75, or 10 to 80 nucleotides in length. In some embodiments, the nick to edit distance is 22 nucleotides, and the editing template is 24 to 28, 24 to 30, 24 to 32, 24 to 34, 24 to 36, 24 to 37, 24 to 38, 24 to 40, 24 to 45, 24 to 50, 24 to 55, 24 to 60, 24 to 65, 24 to 70, 24 to 75, 24 to 80, 24 to 85, 24 to 90, 24 to 95, 24 to 100, 24 to 105, 24 to 100, 24 to 105, or 24 to 110 nucleotides in length.

In some embodiments, the editing template comprises an adenine at the first nucleobase position (e.g., for a PEgRNA following 5′-spacer-gRNA core-RTT-PBS-3′ orientation, the 5′ most nucleobase is the “first base”). In some embodiments, the editing template comprises a guanine at the first nucleobase position (e.g., for a PEgRNA following 5′-spacer-gRNA core-RT-PBS-3′ orientation, the 5′ most nucleobase is the “first base”). In some embodiments, the editing template comprises an uracil at the first nucleobase position (e.g., for a PEgRNA following 5′-spacer-gRNA core-RT-PBS-3′ orientation, the 5′ most nucleobase is the “first base”). In some embodiments, the editing template comprises a cytosine at the first nucleobase position (e.g., for a PEgRNA following 5′-spacer-gRNA core-RT-PBS-3′ orientation, the 5′ most nucleobase is the “first base”). In some embodiments, the editing template does not comprise a cytosine at the first nucleobase position (e.g., for a PEgRNA following 5′-spacer-gRNA core-RT-PBS-3′ orientation, the 5′ most nucleobase is the “first base”).

The editing template of a PEgRNA may encode a new single stranded DNA (e.g. by reverse transcription) to replace an editing target sequence in the target gene. In some embodiments, the editing target sequence in the edit strand of the target gene is replaced by the newly synthesized strand, and the nucleotide edit(s) are incorporated in the region of the target gene. In some embodiments, the target gene is an NCF1 gene, an NCF1B pseudogene, or an NCF1C pseudogene. In some embodiments, the editing template of the PEgRNA encodes a newly synthesized single stranded DNA that comprises a wild type NCF1 gene sequence. In some embodiments, the newly synthesized DNA strand replaces the editing target sequence in the target gene, wherein the editing target sequence (or the endogenous sequence complementary to the editing target sequence on the target strand of the target gene,) comprises a mutation or a nucleotide alteration compared to a reference gene, e.g., a wild type gene.

In some embodiments, the newly synthesized DNA strand replaces the editing target sequence in a target NCF1 gene, wherein the editing target sequence (or the endogenous sequence complementary to the editing target sequence on the target strand of the target NCF1 gene) comprises a mutation compared to a reference gene, e.g., a wild type NCF1 gene. In some embodiments, the mutation is associated with CGD. In some embodiments, the newly synthesized single stranded DNA encoded by the editing target sequence replaces the editing target sequence, and corrects the mutation in the editing target sequence of the target NCF1 gene.

In some embodiments, the editing target sequence comprises a mutation in exon 1, exon 2, exon 3, exon 4, exon 5, exon 6, exon 7, exon 8, exon 9, exon 10, exon 11 of the NCF1 gene, as compared to a wild type NCF1 gene. In some embodiments, the editing target sequence comprises a mutation at an exon/intron junction of the NCF1 gene as compared to a wild type NCF1 gene.

Unless otherwise indicated, references to nucleotide positions in human chromosomes are as set forth in human genome assembly consortium Human build 38 (GRCh38), GenBank accession GCF_000001405.38.

In some embodiments, the editing target sequence is between positions 73220539-75172098 of human chromosome 7. In some embodiments, the editing target sequence is between positions 74777218-74777418 of human chromosome 7. In some embodiments, the editing target sequence is between positions 74778985-74779285 of human chromosome 7. In some embodiments, the editing target sequence is between positions 74779058-74779258 of human chromosome 7. In some embodiments, the editing target sequence is between positions 74779174-74779374 of human chromosome 7. In some embodiments, the editing target sequence is between positions 74779195-74779395 of human chromosome 7. In some embodiments, the editing target sequence is between positions 74779196-74779396 of human chromosome 7. In some embodiments, the editing target sequence is between positions 74779198-74779398 of human chromosome 7. In some embodiments, the editing target sequence is between positions 74779219-74779419 of human chromosome 7. In some embodiments, the editing target sequence is between positions 74779222-74779422 of human chromosome 7. In some embodiments, the editing target sequence is between positions 74779255-74779455 of human chromosome 7. In some embodiments, the editing target sequence is between positions 74779260-74779460 of human chromosome 7. In some embodiments, the editing target sequence is between positions 74779258-74779458 of human chromosome 7. In some embodiments, the editing target sequence is between positions 74779274-74779474 of human chromosome 7. In some embodiments, the editing target sequence is between positions 74779280-74779480 of human chromosome 7. In some embodiments, the editing target sequence is between positions 74780687-74780887 of human chromosome 7. In some embodiments, the editing target sequence is between positions 74780730-74780930 of human chromosome 7. In some embodiments, the editing target sequence is between positions 74782961-74783161 of human chromosome 7. In some embodiments, the editing target sequence is between positions 74782928-74783128 of human chromosome 7. In some embodiments, the editing target sequence is between positions 74783429-74783629 of human chromosome 7. In some embodiments, the editing target sequence is between positions 74783431-74783631 of human chromosome 7. In some embodiments, the editing target sequence is between positions 74783454-74783654 of human chromosome 7. In some embodiments, the editing target sequence is between positions 74783462-74783662 of human chromosome 7. In some embodiments, the editing target sequence is between positions 74783528-74783728 of human chromosome 7. In some embodiments, the editing target sequence is between positions 74785129-74785329 of human chromosome 7. In some embodiments, the editing target sequence is between positions 74785183-74785383 of human chromosome 7. In some embodiments, the editing target sequence is between positions 74785188-74785388 of human chromosome 7. In some embodiments, the editing target sequence is between positions 74785133-74785333 of human chromosome 7. In some embodiments, the editing target sequence is between positions 74787894-74788094 of human chromosome 7. In some embodiments, the editing target sequence is between positions 74787921-74788121 of human chromosome 7. In some embodiments, the editing target sequence is between positions 74788476-74788676 of human chromosome 7. In some embodiments, the editing target sequence is between positions 74774004-74774204 of human chromosome 7. In some embodiments, the editing target sequence is between positions 74774006-74774206 of human chromosome 7. In some embodiments, the editing target sequence is between positions 74777248-74777448 of human chromosome 7. In some embodiments, the editing target sequence is between positions 74777252-74777452 of human chromosome 7. In some embodiments, the editing target sequence is between positions 74782962-74783162 of human chromosome 7. In some embodiments, the editing target sequence is between positions 74783533-74783733 of human chromosome 7.

In some embodiments, the editing target sequence comprises a mutation that is located on exon 2 of the NCF1 gene as compared to a wild type NCF1 gene. In some embodiments, the editing target sequence comprises a mutation that is located between positions 74774011-74789315 of human chromosome 7. In some embodiments, the editing target sequence comprises a mutation that is located between positions 74777167-74777368 of human chromosome 7 as compared to a wild type NCF1 gene. In some embodiments, the editing target sequence is located between positions corresponding to 74777167-74777368 of human chromosome 7. In some embodiments, the editing target sequence comprises a mutation that is located at positions 74777267-74777268 of human chromosome 7 as compared to a wild type NCF1 gene. In some embodiments, the editing target sequence (or a complementary sequence in the target NCF1 gene) comprises a mutation that is located at positions 74777269-74777270 of human chromosome 7 as compared to a wild type NCF1 gene. In some embodiments, the mutation is a c. 73_74GT (ΔGT) mutation, resulting in the deletion of a 2-nucleotide sequence located at positions 74777267-74777268 as compared to a wild type NCF1 gene. In some embodiments, the editing target sequence (or a complementary sequence in the target NCF1 gene) comprises a mutation that encodes a R42Q amino acid substitution in a p47phox polypeptide as compared to a wild type p47phox polypeptide as set forth in SEQ ID No: 34422.

Wild Type p47phox Protein Sequence

(SEQ ID NO: 34422)
MGDTFIRHIALLGFEKRFVPSQHYVYMFLVKWQDLSEKVVYRRFTEIY
EFHKTLKEMFPIEAGAINPENRIIPHLPAPKWFDGQRAAENRQGTLTE
YCSTLMSLPTKISRCPHLLDFFKVRPDDLKLPTDNQTKKPETYLMPKD
GKSTATDITGPIILQTYRAIANYEKTSGSEMALSTGDVVEVVEKSESG
WWFCQMKAKRGWIPASFLEPLDSPDETEDPEPNYAGEPYVAIKAYTAV
EGDEVSLLEGEAVEVIHKLLDGWWVIRKDDVTGYFPSMYLQKSGQDVS
QAQRQIKRGAPPRRSSIRNAHSIHQRSRKRLSQDAYRRNSVRFLQQRR
RQARPGPQSPGSPLEEERQTQRSKPQPAVPPRPSADLILNRCSESTKR
KLASAV.

The p47phox protein may be encoded by a wildtype NCF1 genomic sequence: NG_009078.2.

In some embodiments, the editing target sequence (or a complementary sequence in the target NCF1 gene) comprises a G→A nucleotide substitution (c.125 G→A) at position 74777319 of human chromosome 7 as compared to a wild type NCF1 gene. In some embodiments, the editing target sequence (or a complementary sequence in the target NCF1 gene) comprises a mutation that encodes a R42W amino acid substitution in a p47phox polypeptide as compared to a wild type p47phox polypeptide as set forth in SEQ ID No: 34422. In some embodiments, the editing target sequence (or a complementary sequence in the target NCF1 gene) comprises a C→T nucleotide substitution (c.124 C→T) at position 74777318 of human chromosome 7 as compared to a wild type NCF1 gene.

Accordingly, in some embodiments, the editing template of a PEgRNA encodes the newly synthesized single stranded DNA that replaces the editing target sequence, thereby correcting the mutation in the target NCF1 gene. In some embodiments, one or more intended nucleotide edits in the newly synthesized single stranded DNA is incorporated in the target NCF1 gene, thereby correcting the mutation in the target NCF1 gene. In some embodiments, the editing template of the PEgRNA or the newly synthesized single stranded DNA comprises a two-nucleotide insertion as compared to the editing target sequence. The c.73_74 delGT mutation is a deletion of “GT” from a “GTGT” sequence, which are the first 4 nucleotides of exon 2 of wild type NCF1 (positions 74777267-74777270 of human chromosome 7, GRch 38). The mutation shifts the reading frame and thus abolishes expression of the p47 protein. Because inserting a GT either upstream or downstream of another GT results in the same “GTGT” sequence, the two-nucleotide insert can be considered at the position of the second “GT” in “GTGT”. Accordingly, the editing template maybe designed to contain a GT insertion (or a reverse complement thereof) that corresponds to the wildtype NCF1 sequence, or may contain s TT, AT, or CT insertion (or a reverse complement thereof) that restores the wildtype reading frame and does not alter the amino acid sequence encoded. In some embodiments, the editing template of the PEgRNA or the newly synthesized single stranded DNA comprises a two nucleotide GT insertion as compared to the editing target sequence (or a complementary sequence in the target NCF1 gene). In some embodiments, the editing template of the PEgRNA or the newly synthesized single stranded DNA comprises a two nucleotide GT insertion as compared to the editing target sequence (or a complementary sequence in the target NCF1 gene) corresponding to positions 74777267-74777268 (also referred to as insertion at position 74777267) of human chromosome 7. In some embodiments, the editing template of the PEgRNA or the newly synthesized single stranded DNA comprises a two nucleotide GT insertion as compared to the editing target sequence (or a complementary sequence in the target NCF1 gene) corresponding to positions 74777269-74777270 of human chromosome 7. In some embodiments, the editing template of the PEgRNA or the newly synthesized single stranded DNA comprises a two nucleotide TT insertion as compared to the editing target sequence corresponding to positions 74777269-74777270 of human chromosome 7. In some embodiments, the editing template of the PEgRNA or the newly synthesized single stranded DNA comprises a two nucleotide AT insertion as compared to the editing target sequence (or a complementary sequence in the target NCF1 gene) corresponding to positions 74777269-74777270 of human chromosome 7. In some embodiments, the editing template of the PEgRNA or the newly synthesized single stranded DNA comprises a two nucleotide CT insertion as compared to the editing target sequence (or a complementary sequence in the target NCF1 gene) corresponding to positions 74777269-74777270 of human chromosome 7. In some embodiments, the editing template of the PEgRNA or the newly synthesized single stranded DNA comprises a A→G nucleotide substitution as compared to the editing target sequence (or a complementary sequence in the target NCF1 gene) at a position corresponding to position 74777319 of human chromosome 7. In some embodiments, the editing template of the PEgRNA or the newly synthesized single stranded DNA comprises a T→C nucleotide substitution as compared to the editing target sequence (or a complementary sequence in the target NCF1 gene) at a position corresponding to position 74777318 of human chromosome 7. In some embodiments, the editing template of the PEgRNA or the newly synthesized single stranded DNA comprises a A→G nucleotide substitution at a position corresponding to position 74777319, a T→C nucleotide substitution at a position corresponding to position 74777318, a GT two-nucleotide insertion at positions corresponding to positions 74777267-74777268, and/or a GT, AT, CT, or TT two-nucleotide insertion at corresponding to positions 74777269-74777270 of human chromosome 7 as compared to the editing target sequence (or a complementary sequence in the target NCF1 gene).

In some embodiments, the editing target sequence comprises a mutation that is located in exon 3 of the NCF1 gene as compared to a wild type NCF1 gene. In some embodiments, the editing target sequence (or a complementary sequence in the target NCF1 gene) comprises a mutation that encodes a T53A amino acid substitution in a p47phox polypeptide as compared to a wild type p47phox polypeptide as set forth in SEQ ID No: 34422. In some embodiments, the editing target sequence (or a complementary sequence in the target NCF1 gene) comprises a A→G nucleotide substitution (c.157 A→G) at position 74779085 of human chromosome 7 as compared to a wild type NCF1 gene. In some embodiments, the editing target sequence comprises a deletion of a nucleotide G (c.229_IdelG) at a position corresponding to position 74779158 of human chromosome 7.

Accordingly, in some embodiments, one or more intended nucleotide edits in the newly synthesized single stranded DNA is incorporated in the target NCF1 gene, thereby correcting the mutation in the target NCF1 gene. In some embodiments, the editing template of the PEgRNA or the newly synthesized single stranded DNA comprises a G→A nucleotide substitution compared to the editing target sequence (or a complementary sequence in the target NCF1 gene) at a position corresponding to position 74779085 of human chromosome 7. In some embodiments, the editing template of the PEgRNA or the newly synthesized single stranded DNA comprises insertion of a guanine nucleotide compared to the editing target sequence (or a complementary sequence in the target NCF1 gene) at a position corresponding to position 74779158 of human chromosome 7.

In some embodiments, the editing target sequence comprises a mutation that is located in exon 4 of the NCF1 gene as compared to a wild type NCF1 gene. In some embodiments, the editing target sequence (or a complementary sequence in the target NCF1 gene) comprises a mutation that encodes a G83R amino acid substitution in a p47phox polypeptide as compared to a wild type p47phox polypeptide as set forth in SEQ ID No: 34422. In some embodiments, the editing target sequence (or a complementary sequence in the target NCF1 gene) comprises a GA nucleotide substitution (c.247 GA) at position 74779274 of human chromosome 7 as compared to a wild type NCF1 gene. In some embodiments, the editing target sequence (or a complementary sequence in the target NCF1 gene) comprises a mutation that encodes a R90C amino acid substitution in a p47phox polypeptide as compared to a wild type p47phox polypeptide as set forth in SEQ ID No: 34422. In some embodiments, the editing target sequence (or a complementary sequence in the target NCF1 gene) comprises a C→T nucleotide substitution (c.268 C→T) at position 74779295 of human chromosome 7 as compared to a wild type NCF1 gene. In some embodiments, the editing target sequence (or a complementary sequence in the target NCF1 gene) comprises a mutation that encodes a R90H amino acid substitution in a p47phox polypeptide as compared to a wild type p47phox polypeptide as set forth in SEQ ID No: 34422. In some embodiments, the editing target sequence (or a complementary sequence in the target NCF1 gene) comprises a G→A nucleotide substitution (c.269 G→A) at position 74779296 of human chromosome 7 as compared to a wild type NCF1 gene. In some embodiments, the editing target sequence (or a complementary sequence in the target NCF1 gene) comprises a nonsense mutation that results in a Q90* amino acid alteration in a p47phox polypeptide as compared to a wild type p47phox polypeptide as set forth in SEQ ID No: 34422, wherein * refers to a premature stop codon. In some embodiments, the editing target sequence (or a complementary sequence in the target NCF1 gene) comprises a C→T nucleotide substitution (c.271 C→T) at position 74779298 of human chromosome 7 as compared to a wild type NCF1 gene. In some embodiments, the editing target sequence (or a complementary sequence in the target NCF1 gene) comprises a mutation that encodes a C98G amino acid substitution in a p47phox polypeptide as compared to a wild type p47phox polypeptide as set forth in SEQ ID No: 34422. In some embodiments, the editing target sequence (or a complementary sequence in the target NCF1 gene) comprises a T→G nucleotide substitution (c.292 T→G) at position 74779319 of human chromosome 7 as compared to a wild type NCF1 gene. In some embodiments, the editing target sequence (or a complementary sequence in the target NCF1 gene) comprises a mutation that encodes a S99G amino acid substitution in a p47phox polypeptide as compared to a wild type p47phox polypeptide as set forth in SEQ ID No: 34422. In some embodiments, the editing target sequence (or a complementary sequence in the target NCF1 gene) comprises a A→G nucleotide substitution (c.295 A→G) at position 74779322 of human chromosome 7 as compared to a wild type NCF1 gene. In some embodiments, the editing target sequence (or a complementary sequence in the target NCF1 gene) comprises a mutation that encodes a R110C amino acid substitution in a p47phox polypeptide as compared to a wild type p47phox polypeptide as set forth in SEQ ID No: 34422. In some embodiments, the editing target sequence comprises (or a complementary sequence in the target NCF1 gene) a C→T nucleotide substitution (c.328 C→T) at position 74779355 of human chromosome 7 as compared to a wild type NCF1 gene. In some embodiments, the editing target sequence (or a complementary sequence in the target NCF1 gene) comprises a nonsense mutation that results in a C111* amino acid alteration in a p47phox polypeptide as compared to a wild type p47phox polypeptide as set forth in SEQ ID No: 34422, wherein * refers to a premature stop codon. In some embodiments, the editing target sequence (or a complementary sequence in the target NCF1 gene) comprises a T→A nucleotide substitution (c.333 T→A) at position 74779360 of human chromosome 7 as compared to a wild type NCF1 gene. In some embodiments, the editing target sequence (or a complementary sequence in the target NCF1 gene) comprises deletion of nucleotides TGTCCCCAC (c.331 339delTGTCCCCAC) at position 74779358 of human chromosome 7 as compared to a wild type NCF1 gene. In some embodiments, the editing target sequence (or a complementary sequence in the target NCF1 gene) comprises a deletion of nucleotide adenine (c.347delA) at position 74779374 of human chromosome 7 as compared to a wild type NCF1 gene. In some embodiments, the editing target sequence (or a complementary sequence in the target NCF1 gene) comprises a deletion of nucleotides TC and insertion of nucleotides AA (c.353_354delTCinsAA) at a position 74779380 of human chromosome 7 as compared to a wild type NCF1 gene.

Accordingly, in some embodiments, one or more intended nucleotide edits in the newly synthesized single stranded DNA is incorporated in the target NCF1 gene, thereby correcting the mutation in the target NCF1 gene. In some embodiments, the editing template of the PEgRNA or the newly synthesized single stranded DNA comprises a A→G nucleotide substitution compared to the editing target sequence (or a complementary sequence in the target NCF1 gene) at a position corresponding to position 74779274 of human chromosome 7. In some embodiments, the editing template of the PEgRNA or the newly synthesized single stranded DNA comprises a T→C nucleotide substitution compared to the editing target sequence (or a complementary sequence in the target NCF1 gene) at a position corresponding to position 74779295 of human chromosome 7. In some embodiments, the editing template of the PEgRNA or the newly synthesized single stranded DNA comprises a A→G nucleotide substitution compared to the editing target sequence (or a complementary sequence in the target NCF1 gene) at a position corresponding to position 74779296 of human chromosome 7. In some embodiments, the editing template of the PEgRNA or the newly synthesized single stranded DNA comprises a T→C nucleotide substitution compared to the editing target sequence (or a complementary sequence in the target NCF1 gene) at a position corresponding to position 74779298 of human chromosome 7. In some embodiments, the editing template of the PEgRNA or the newly synthesized single stranded DNA comprises a G→T nucleotide substitution compared to the editing target sequence (or a complementary sequence in the target NCF1 gene) at a position corresponding to position 74779319 of human chromosome 7. In some embodiments, the editing template of the PEgRNA or the newly synthesized single stranded DNA comprises a G→A nucleotide substitution compared to the editing target sequence (or a complementary sequence in the target NCF1 gene) at a position corresponding to position 74779322 of human chromosome 7. In some embodiments, the editing template of the PEgRNA or the newly synthesized single stranded DNA comprises a T→C nucleotide substitution compared to the editing target sequence (or a complementary sequence in the target NCF1 gene) at a position corresponding to position 74779355 of human chromosome 7. In some embodiments, the editing template of the PEgRNA or the newly synthesized single stranded DNA comprises a A4T nucleotide substitution compared to the editing target sequence (or a complementary sequence in the target NCF1 gene) at a position corresponding to position 74779360 of human chromosome 7. In some embodiments, the editing template of the PEgRNA or the newly synthesized single stranded DNA comprises an insertion of nucleotides TGTCCCCAC compared to the editing target sequence (or a complementary sequence in the target NCF1 gene) at a position corresponding to position 74779358 of human chromosome 7. In some embodiments, the editing template of the PEgRNA or the newly synthesized single stranded DNA comprises an insertion of an adenine nucleotide compared to the editing target sequence (or a complementary sequence in the target NCF1 gene) at a position corresponding to position 74779374 of human chromosome 7. In some embodiments, the editing template of the PEgRNA or the newly synthesized single stranded DNA comprises a substitution of nucleotides AA with nucleotides TC compared to the editing target sequence (or a complementary sequence in the target NCF1 gene) at a position 74779380 of human chromosome 7. In some embodiments, the editing template of the PEgRNA or the newly synthesized single stranded DNA comprises a A→G nucleotide substitution at a position corresponding to position 74779274, a T→C nucleotide substitution at a position corresponding to position 74779295, a A→G nucleotide substitution at a position corresponding to position 74779296, a T→C nucleotide substitution at a position corresponding to position 74779298, a G→T nucleotide substitution at a position corresponding to position 74779319, a G→A nucleotide substitution at a position corresponding to position 74779322, a T→C nucleotide substitution at a position corresponding to position 74779355, a A4T nucleotide substitution at a position corresponding to position 74779360, an insertion of nucleotides TGTCCCCAC at a position corresponding to position 74779358, an insertion of an adenine nucleotide at a position corresponding to position 74779374, and/or a substitution of nucleotides AA with nucleotides TC c at a position 74779380 human chromosome 7 compared to the editing target sequence (or a complementary sequence in the target NCF1 gene).

In some embodiments, the editing target sequence comprises a mutation that is located in exon 5 of the NCF1 gene as compared to a wild type NCF1 gene. In some embodiments, the editing target sequence (or a complementary sequence in the target NCF1 gene) comprises a mutation that encodes a K135E amino acid substitution in a p47phox polypeptide as compared to a wild type p47phox polypeptide as set forth in SEQ ID No: 34422. In some embodiments, the editing target sequence (or a complementary sequence in the target NCF1 gene) comprises a A→G nucleotide substitution (c.403 A→G) at position 74780787 of human chromosome 7 as compared to a wild type NCF1 gene. In some embodiments, the editing target sequence (or a complementary sequence in the target NCF1 gene) comprises a mutation that encodes a A149E amino acid substitution in a p47phox polypeptide as compared to a wild type p47phox polypeptide as set forth in SEQ ID No: 34422. In some embodiments, the editing target sequence (or a complementary sequence in the target NCF1 gene) comprises a C→A nucleotide substitution (c.446 C→A) at position 74780830 of human chromosome 77 as compared to a wild type NCF1 gene.

Accordingly, in some embodiments, one or more intended nucleotide edits in the newly synthesized single stranded DNA is incorporated in the target NCF1 gene, thereby correcting the mutation in the target NCF1 gene. In some embodiments, the editing template of the PEgRNA or the newly synthesized single stranded DNA comprises a G→A nucleotide substitution compared to the editing target sequence (or a complementary sequence in the target NCF1 gene) at a position corresponding to position 74780787 of human chromosome 7. In some embodiments, the editing template of the PEgRNA or the newly synthesized single stranded DNA comprises a A→C nucleotide substitution compared to the editing target sequence (or a complementary sequence in the target NCF1 gene) at a position corresponding to position 74780830 of human chromosome 7. In some embodiments, the editing template of the PEgRNA or the newly synthesized single stranded DNA comprises a A→C nucleotide substitution at a position corresponding to position 74780830 and/or a G→A nucleotide substitution at a position corresponding to position 74780787 of human chromosome 7 compared to the editing target sequence (or a complementary sequence in the target NCF1 gene).

In some embodiments, the editing target sequence comprises a mutation that is located in exon 6 of the NCF1 gene as compared to a wild type NCF1 gene. In some embodiments, the editing target sequence (or a complementary sequence in the target NCF1 gene) comprises a mutation that encodes a G192S amino acid substitution in a p47phox polypeptide as compared to a wild type p47phox polypeptide as set forth in SEQ ID No: 34422. In some embodiments, the editing target sequence (or a complementary sequence in the target NCF1 gene) comprises a G→A nucleotide substitution (c.574 G→A) at position 74783061 of human chromosome 7 as compared to a wild type NCF1 gene. In some embodiments, the editing target sequence (or a complementary sequence in the target NCF1 gene) comprises a deletion of a guanine nucleotide (c.541 delG) at position 74783028 of human chromosome 7 as compared to a wild type NCF1 gene. In some embodiments, the editing target sequence (or a complementary sequence in the target NCF1 gene) comprises a deletion of a guanine nucleotide (c.502 delG) at position 74782989 of human chromosome 7 as compared to a wild type NCF1 gene.

Accordingly, in some embodiments, one or more intended nucleotide edits in the newly synthesized single stranded DNA is incorporated in the target NCF1 gene, thereby correcting the mutation in the target NCF1 gene. In some embodiments, the editing template of the PEgRNA or the newly synthesized single stranded DNA comprises a A→G nucleotide substitution compared to the editing target sequence (or a complementary sequence in the target NCF1 gene) at a position corresponding to position 74783061 of human chromosome 7. In some embodiments, the editing template of the PEgRNA or the newly synthesized single stranded DNA comprises an insertion of a guanine nucleotide compared to the editing target sequence (or a complementary sequence in the target NCF1 gene) at a position corresponding to position 74783028 of human chromosome 7. In some embodiments, the editing template of the PEgRNA or the newly synthesized single stranded DNA comprises an insertion of a guanine nucleotide compared to the editing target sequence (or a complementary sequence in the target NCF1 gene) at a position corresponding to position 74782989 of human chromosome 7. In some embodiments, the editing template of the PEgRNA or the newly synthesized single stranded DNA comprises an insertion of a guanine nucleotide at a position corresponding to position 74783028, an insertion of a guanine nucleotide at a position corresponding to position 74782989, and/or an A→G nucleotide substitution at a position corresponding to position 74783061 of human chromosome 7 compared to the editing target sequence (or a complementary sequence in the target NCF1 gene).

In some embodiments, the editing target sequence comprises a mutation that is located in exon 7 of the NCF1 gene as compared to a wild type NCF1 gene. In some embodiments, the editing target sequence (or a complementary sequence in the target NCF1 gene) comprises a mutation that results in a W193* amino acid substitution in a p47phox polypeptide as compared to a wild type p47phox polypeptide as set forth in SEQ ID No: 34422, wherein * refers to a premature stop codon. In some embodiments, the editing target sequence (or a complementary sequence in the target NCF1 gene) comprises a G→A nucleotide substitution (c.579 G→A) at position 74783529 of human chromosome 7 as compared to a wild type NCF1 gene. In some embodiments, the editing target sequence (or a complementary sequence in the target NCF1 gene) comprises a mutation that results in a W194* amino acid substitution in a p47phox polypeptide as compared to a wild type p47phox polypeptide as set forth in SEQ ID No: 34422, wherein * refers to a premature stop codon. In some embodiments, the editing target sequence (or a complementary sequence in the target NCF1 gene) comprises a G→A nucleotide substitution (c.581 G→A) at position 74783531 of human chromosome 7 as compared to a wild type NCF1 gene. In some embodiments, the editing target sequence (or a complementary sequence in the target NCF1 gene) comprises a mutation that results in a R202* amino acid substitution in a p47phox polypeptide as compared to a wild type p47phox polypeptide as set forth in SEQ ID No: 34422, wherein * refers to a premature stop codon. In some embodiments, the editing target sequence (or a complementary sequence in the target NCF1 gene) comprises a C→T nucleotide substitution (c.604 C→T) at position 74783554 of human chromosome 7 as compared to a wild type NCF1 gene. In some embodiments, the editing target sequence (or a complementary sequence in the target NCF1 gene) comprises a mutation that results in a W204* amino acid substitution in a p47phox polypeptide as compared to a wild type p47phox polypeptide as set forth in SEQ ID No: 34422, wherein * refers to a premature stop codon. In some embodiments, the editing target sequence (or a complementary sequence in the target NCF1 gene) comprises a G→A nucleotide substitution (c.612 G→A) at position 74783562 of human chromosome 7 as compared to a wild type NCF1 gene. In some embodiments, the editing target sequence (or a complementary sequence in the target NCF1 gene) comprises a mutation that results in a Y226* amino acid substitution in a p47phox polypeptide as compared to a wild type p47phox polypeptide as set forth in SEQ ID No: 34422, wherein * refers to a premature stop codon. In some embodiments, the editing target sequence (or a complementary sequence in the target NCF1 gene) comprises a T→G nucleotide substitution (c.678 T→G) at position 74783628 of human chromosome 7 as compared to a wild type NCF1 gene.

Accordingly, in some embodiments, one or more intended nucleotide edits in the newly synthesized single stranded DNA is incorporated in the target NCF1 gene, thereby correcting the mutation in the target NCF1 gene. In some embodiments, the editing template of the PEgRNA or the newly synthesized single stranded DNA comprises a A→G nucleotide substitution compared to the editing target sequence (or a complementary sequence in the target NCF1 gene) at a position corresponding to position 74783529 of human chromosome 7. In some embodiments, the editing template of the PEgRNA or the newly synthesized single stranded DNA comprises a A→G nucleotide substitution compared to the editing target sequence (or a complementary sequence in the target NCF1 gene) at a position corresponding to position 74783531 of human chromosome 7. In some embodiments, the editing template of the PEgRNA or the newly synthesized single stranded DNA comprises a T→C nucleotide substitution compared to the editing target sequence (or a complementary sequence in the target NCF1 gene) at a position corresponding to position 74783554 of human chromosome 7. In some embodiments, the editing template of the PEgRNA or the newly synthesized single stranded DNA comprises a A→G nucleotide substitution compared to the editing target sequence (or a complementary sequence in the target NCF1 gene) at a position corresponding to position 74783562 of human chromosome 7. In some embodiments, the editing template of the PEgRNA or the newly synthesized single stranded DNA comprises a G→T nucleotide substitution compared to the editing target sequence (or a complementary sequence in the target NCF1 gene) at a position corresponding to position 74783628 of human chromosome 7. In some embodiments, the editing template of the PEgRNA or the newly synthesized single stranded DNA comprises a A→G nucleotide substitution at a position corresponding to position 74783529, a A→G nucleotide substitution at a position corresponding to position 74783531, a T→C nucleotide substitution at a position corresponding to position 74783554, a A→G nucleotide substitution at a position corresponding to position 74783562, and/or a G→T nucleotide substitution at a position corresponding to position 74783628 of human chromosome 7 compared to the editing target sequence (or a complementary sequence in the target NCF1 gene).

In some embodiments, the editing target sequence comprises a mutation that is located in exon 8 of the NCF1 gene as compared to a wild type NCF1 gene. In some embodiments, the editing target sequence (or a complementary sequence in the target NCF1 gene) comprises a mutation that encodes a E224K amino acid substitution in a p47phox polypeptide as compared to a wild type p47phox polypeptide as set forth in SEQ ID No: 34422. In some embodiments, the editing target sequence (or a complementary sequence in the target NCF1 gene) comprises a G→A nucleotide substitution (c.730 G→A) at position 74785229 of human chromosome 7 as compared to a wild type NCF1 gene. In some embodiments, the editing target sequence (or a complementary sequence in the target NCF1 gene) comprises a mutation that encodes a G262S amino acid substitution in a p47phox polypeptide as compared to a wild type p47phox polypeptide as set forth in SEQ ID No: 34422. In some embodiments, the editing target sequence (or a complementary sequence in the target NCF1 gene) comprises a G→A nucleotide substitution (c.784 G→A) at position 74785283 of human chromosome 7 as compared to a wild type NCF1 gene. In some embodiments, the editing target sequence (or a complementary sequence in the target NCF1 gene) comprises a mutation that encodes a W263C amino acid substitution in a p47phox polypeptide as compared to a wild type p47phox polypeptide as set forth in SEQ ID No: 34422. In some embodiments, the editing target sequence (or a complementary sequence in the target NCF1 gene) comprises a G→C nucleotide substitution (c.784 G→C) at position 74785288 of human chromosome 7 as compared to a wild type NCF1 gene. In some embodiments, the editing target sequence (or a complementary sequence in the target NCF1 gene) comprises a deletion of nucleotides TGTCCCTGCTCGAGG (c.734_748delTGTCCCTGCTCGAGG) at position 74785233 of human chromosome 7 as compared to a wild type NCF1 gene.

In some embodiments, the editing template of the PEgRNA or the newly synthesized single stranded DNA comprises a A→G nucleotide substitution compared to the editing target sequence (or a complementary sequence in the target NCF1 gene) at a position corresponding to position 74785229 of human chromosome 7. In some embodiments, the editing template of the PEgRNA or the newly synthesized single stranded DNA comprises a A→G nucleotide substitution compared to the editing target sequence (or a complementary sequence in the target NCF1 gene) at a position corresponding to position 74785283 of human chromosome 7. In some embodiments, the editing template of the PEgRNA or the newly synthesized single stranded DNA comprises a C→G nucleotide substitution compared to the editing target sequence (or a complementary sequence in the target NCF1 gene) at a position corresponding to position 74785288 of human chromosome 7. In some embodiments, the editing template of the PEgRNA or the newly synthesized single stranded DNA comprises an insertion of nucleotides TGTCCCTGCTCGAGG compared to the editing target sequence (or a complementary sequence in the target NCF1 gene) at position 74785233 of human chromosome 7. In some embodiments, the editing template of the PEgRNA or the newly synthesized single stranded DNA comprises an A→G nucleotide substitution at a position corresponding to position 74785229, an A→G nucleotide substitution at a position corresponding to position 74785283, a C→G nucleotide substitution at a position corresponding to position 74785288, and/or an insertion of nucleotides TGTCCCTGCTCGAGG at position 74785233 of human chromosome 7 compared to the editing target sequence (or a complementary sequence in the target NCF1 gene).

In some embodiments, the editing target sequence comprises a mutation that is located in exon 9 of the NCF1 gene as compared to a wild type NCF1 gene. In some embodiments, the editing target sequence (or a complementary sequence in the target NCF1 gene) comprises a deletion of nucleotide guanine (c.811delG) at position 74787994 of human chromosome 7 as compared to a wild type NCF1 gene. In some embodiments, the editing target sequence (or a complementary sequence in the target NCF1 gene) comprises a deletion of nucleotide cytidine (c.838delC) at position 74788021 of human chromosome 7 as compared to a wild type NCF1 gene.

Accordingly, in some embodiments, the editing template of the PEgRNA or the newly synthesized single stranded DNA comprises an insertion of a nucleotide guanine compared to the editing target sequence (or a complementary sequence in the target NCF1 gene) at a position corresponding to position 74787994 of human chromosome 7. In some embodiments, the editing template of the PEgRNA or the newly synthesized single stranded DNA comprises an insertion of a nucleotide cytidine compared to the editing target sequence (or a complementary sequence in the target NCF1 gene) at a position corresponding to position 74788021 of human chromosome 7. In some embodiments, the editing template of the PEgRNA or the newly synthesized single stranded DNA comprises an insertion of a nucleotide guanine at a position corresponding to position 74787994 and/or an insertion of a nucleotide cytidine at a position corresponding to position 74788021 of human chromosome 7 compared to the editing target sequence (or a complementary sequence in the target NCF1 gene).

In some embodiments, the editing target sequence comprises a mutation that is located in exon 10 of the NCF1 gene as compared to a wild type NCF1 gene. In some embodiments, the editing target sequence (or a complementary sequence in the target NCF1 gene) comprises a mutation that encodes a A308V amino acid substitution in a p47phox polypeptide as compared to a wild type p47phox polypeptide as set forth in SEQ ID No: 34422. In some embodiments, the editing target sequence (or a complementary sequence in the target NCF1 gene) comprises a C→T nucleotide substitution (c.923 C→T) at position 74788576 of human chromosome 7 as compared to a wild type NCF1 gene. Accordingly, in some embodiments, the editing template of the PEgRNA or the newly synthesized single stranded DNA comprises a T→C nucleotide substitution at position 74788576 of human chromosome 7 compared to the editing target sequence (or a complementary sequence in the target NCF1 gene).

In some embodiments, the editing target sequence comprises a mutation in a non-coding region of the target NCF1 gene compared to a wild type NCF1 gene. In some embodiments, the editing target sequence comprises a mutation in an intron of the target NCF1 gene compared to a wild type NCF1 gene.

In some embodiments, the editing target sequence comprises a mutation that is at a splice site of the target NCF1 gene compared to a wild type NCF1 gene. In some embodiments, the editing target sequence comprises a mutation that is at an intron/exon junction of the target NCF1 gene compared to a wild type NCF1 gene. In some embodiments, the editing target sequence (or a complementary sequence in the target NCF1 gene) comprises a mutation at the junction of the exon 1 and the intron 1 of the target NCF1 gene compared to a wild type NCF1 gene. In some embodiments, the editing target sequence (or a complementary sequence in the target NCF1 gene) comprises a mutation at the junction of intron 1 and the exon 2 of the target NCF1 gene compared to a wild type NCF1 gene. In some embodiments, the editing target sequence (or a complementary sequence in the target NCF1 gene) comprises a mutation at the junction of exon 2 and intron 2 of the target NCF1 gene compared to a wild type NCF1 gene. In some embodiments, the editing target sequence (or a complementary sequence in the target NCF1 gene) comprises a mutation at the junction of intron 2 and exon 3 of the target NCF1 gene compared to a wild type NCF1 gene. In some embodiments, the editing target sequence (or a complementary sequence in the target NCF1 gene) comprises a mutation at the junction of exon 3 and intron 3 of the target NCF1 gene compared to a wild type NCF1 gene. In some embodiments, the editing target sequence (or a complementary sequence in the target NCF1 gene) comprises a mutation at the junction of intron 3 and exon 4 of the target NCF1 gene compared to a wild type NCF1 gene. In some embodiments, the editing target sequence (or a complementary sequence in the target NCF1 gene) comprises a mutation at the junction of exon 4 and intron 4 of the target NCF1 gene compared to a wild type NCF1 gene. In some embodiments, the editing target sequence (or a complementary sequence in the target NCF1 gene) comprises a mutation at the junction of intron 4 and exon 5 of the target NCF1 gene compared to a wild type NCF1 gene. In some embodiments, the editing target sequence (or a complementary sequence in the target NCF1 gene) comprises a mutation at the junction of exon 5 and intron 5 of the target NCF1 gene compared to a wild type NCF1 gene. In some embodiments, the editing target sequence (or a complementary sequence in the target NCF1 gene) comprises a mutation at the junction of intron 5 and exon 6 of the target NCF1 gene compared to a wild type NCF1 gene. In some embodiments, the editing target sequence (or a complementary sequence in the target NCF1 gene) comprises a mutation at the junction of exon 6 and intron 6 of the target NCF1 gene compared to a wild type NCF1 gene. In some embodiments, the editing target sequence (or a complementary sequence in the target NCF1 gene) comprises a mutation at the junction of intron 6 and exon 7 of the target NCF1 gene compared to a wild type NCF1 gene. In some embodiments, the editing target sequence (or a complementary sequence in the target NCF1 gene) comprises a mutation at the junction of exon 7 and intron 7 of the target NCF1 gene compared to a wild type NCF1 gene. In some embodiments, the editing target sequence (or a complementary sequence in the target NCF1 gene) comprises a mutation at the junction of intron 7 and exon 8 of the target NCF1 gene compared to a wild type NCF1 gene. In some embodiments, the editing target sequence (or a complementary sequence in the target NCF1 gene) comprises a mutation at the junction of exon 8 and intron 8 of the target NCF1 gene compared to a wild type NCF1 gene. In some embodiments, the editing target sequence (or a complementary sequence in the target NCF1 gene) comprises a mutation at the junction of intron 8 and exon 9 of the target NCF1 gene compared to a wild type NCF1 gene. In some embodiments, the editing target sequence (or a complementary sequence in the target NCF1 gene) comprises a mutation at the junction of exon 9 and intron 9 of the target NCF1 gene compared to a wild type NCF1 gene. In some embodiments, the editing target sequence (or a complementary sequence in the target NCF1 gene) comprises a mutation at the junction of intron 9 and exon 10 of the target NCF1 gene compared to a wild type NCF1 gene. In some embodiments, the editing target sequence (or a complementary sequence in the target NCF1 gene) comprises a mutation at the junction of exon 10 and intron 10 of the target NCF1 gene compared to a wild type NCF1 gene. In some embodiments, the editing target sequence (or a complementary sequence in the target NCF1 gene) comprises a mutation at the junction of intron 10 and exon 11 of the target NCF1 gene compared to a wild type NCF1 gene. In some embodiments, the editing target sequence (or a complementary sequence in the target NCF1 gene) comprises a G→A nucleotide substitution at a position corresponding to position 74774104 (c.72+1 G→A) of human chromosome 7 as compared to a wild type NCF1 gene. In some embodiments, the editing target sequence (or a complementary sequence in the target NCF1 gene) comprises a G→T nucleotide substitution at a position corresponding to position 74774106 (c.72+3 G→T) of human chromosome 7 as compared to a wild type NCF1 gene. In some embodiments, the editing target sequence (or a complementary sequence in the target NCF1 gene) comprises a G→A nucleotide substitution at a position corresponding to position 74777348 (c.153+1 G→A) of human chromosome 7 as compared to a wild type NCF1 gene. In some embodiments, the editing target sequence (or a complementary sequence in the target NCF1 gene) comprises a G→C nucleotide substitution at a position corresponding to position 74777352 (c.153+5 G→C) of human chromosome 7 as compared to a wild type NCF1 gene. In some embodiments, the editing target sequence (or a complementary sequence in the target NCF1 gene) comprises a G→A nucleotide substitution at a position corresponding to position 74783061 (c.574 G→A) of human chromosome 7 as compared to a wild type NCF1 gene. In some embodiments, the editing target sequence (or a complementary sequence in the target NCF1 gene) comprises a G→A nucleotide substitution at a position corresponding to position 74783062 (c.574+1 G→A) of human chromosome 7 as compared to a wild type NCF1 gene. In some embodiments, the editing target sequence (or a complementary sequence in the target NCF1 gene) comprises a G→A nucleotide substitution at a position corresponding to position 74783633 (c.682+1 G→A) of human chromosome 7 as compared to a wild type NCF1 gene. In some embodiments, the editing target sequence (or a complementary sequence in the target NCF1 gene) comprises a G→C nucleotide substitution at a position corresponding to position 74783633 (c.682+1 G→C) of human chromosome 7 as compared to a wild type NCF1 gene.

Accordingly, in some embodiments, one or more intended nucleotide edits in the newly synthesized single stranded DNA is incorporated in the target NCF1 gene, thereby correcting the mutation in the target NCF1 gene. In some embodiments, the editing template of the PEgRNA or the newly synthesized single stranded DNA comprises a A→G nucleotide substitution compared to the editing target sequence (or a complementary sequence in the target NCF1 gene) at a position corresponding to position 74774104 of human chromosome 7. In some embodiments, the editing template of the PEgRNA or the newly synthesized single stranded DNA comprises a T→G nucleotide substitution at a position corresponding to position 74774106 of human chromosome 7 as compared editing target sequence (or a complementary sequence in the target NCF1 gene). In some embodiments, the editing template of the PEgRNA or the newly synthesized single stranded DNA comprises a A→G nucleotide substitution at a position corresponding to position 74777348 compared to the editing target sequence (or a complementary sequence in the target NCF1 gene). In some embodiments, t the editing template of the PEgRNA or the newly synthesized single stranded DNA comprises a C→G nucleotide substitution at a position corresponding to position 74777352 of human chromosome 7 as compared to the editing target sequence (or a complementary sequence in the target NCF1 gene). In some embodiments, the editing template of the PEgRNA or the newly synthesized single stranded DNA comprises a A→G nucleotide substitution at a position corresponding to position 74783061 of human chromosome 7 compared to the editing target sequence (or a complementary sequence in the target NCF1 gene). In some embodiments, the editing template of the PEgRNA or the newly synthesized single stranded DNA comprises a A→G nucleotide substitution at a position corresponding to position 74783062 of human chromosome 7 as compared to the editing target sequence (or a complementary sequence in the target NCF1 gene). In some embodiments, the editing template of the PEgRNA or the newly synthesized single stranded DNA comprises a A→G nucleotide substitution at a position corresponding to position 74783633 of human chromosome 7 compared to the editing target sequence (or a complementary sequence in the target NCF1 gene). In some embodiments, the editing template of the PEgRNA or the newly synthesized single stranded DNA comprises a C→G nucleotide substitution at a position corresponding to position 74783633 of human chromosome 7 as compared to a wild type NCF1 gene.

In some embodiments, the editing target sequence is in a target NCF1B pseudogene or a target NCF1C pseudogene. In some embodiments, the newly synthesized DNA strand replaces the editing target sequence in a target NCF1B pseudogene or a target NCF1C pseudogene, wherein the editing target sequence (or the endogenous sequence complementary to the editing target sequence on the target strand of the target NCF1B pseudogene or the NCF1C pseudogene) comprises a nucleotide alteration, e.g., a deletion, as compared to a reference gene, e.g., a wild type NCF1 gene.

In some embodiments, the editing target sequence comprises a sequence in a pseudogene of NCF1, wherein the pseudogene is NCF1B. In some embodiments, the editing target sequence comprises a sequence in an exon of NCF1B. In some embodiments, the editing target sequence comprises a sequence in exon 2 of NCF1B. In some embodiments, the editing target sequence is located between positions 73220639-73235945 of human chromosome 7. In some embodiments, the editing target sequence is between positions corresponding to positions 73223778-73223979 of human chromosome 7. In some embodiments, the editing target sequence comprises nucleotides corresponding to positions 73223878-73223979 of human chromosome 7. In some embodiments, the editing target sequence in the NCF1B pseudogene comprises a two nucleotide GT deletion compared to a wild type NCF1 gene. Accordingly, in some embodiments, the editing template of a PEgRNA encodes the newly synthesized single stranded DNA that replaces the editing target sequence, thereby editing the target NCF1B pseudogene. NCF1B differs from wildtype NCF1 in that it lacks a “GT” of a “GTGT” sequence, which are the first 4 nucleotides of exon 2 of wild type NCF1 (positions 74777267-74777270 of human chromosome 7, GRch 38). Because inserting a GT either upstream or downstream of another GT results in the same “GTGT” sequence, the two-nucleotide insert can be considered at the position of the second “GT” in “GTGT”. Accordingly, the editing template maybe designed to contain a GT insertion (or a reverse complement thereof) that corresponds to the wildtype NCF1 sequence, or may contain s TT, AT, or CT insertion (or a reverse complement thereof) that restores the wildtype NCF1 reading frame and does not alter the amino acid sequence encoded by wildtype NCF1. In some embodiments, one or more intended nucleotide edits in the newly synthesized single stranded DNA is incorporated in the target NCF1 gene, thereby editing the target NCF1B gene to comprise a sequence of a wild type NCF1 gene. In some embodiments, the editing template of the PEgRNA or the newly synthesized single stranded DNA comprises a two-nucleotide insertion as compared to the editing target sequence. In some embodiments, the intended nucleotide edit comprises a two-nucleotide insertion at a position corresponding to position 73223878 in human chromosome 7 as compared to the region corresponding to the editing target sequence in the NCF1B pseudogene. In some embodiments, the editing template of the PEgRNA or the newly synthesized single stranded DNA comprises a two nucleotide GT insertion as compared to the editing target sequence (or a complementary sequence in the target NCF1B pseudogene). In some embodiments, the intended nucleotide edit comprises a two-nucleotide insertion at a position corresponding to position 73223880 in human chromosome 7 as compared to the region corresponding to the editing target sequence in the NCF1B pseudogene. In some embodiments, the editing template of the PEgRNA or the newly synthesized single stranded DNA comprises a two nucleotide TT, AT, or CT insertion as compared to the editing target sequence (or a complementary sequence in the target NCF1B pseudogene).

In some embodiments, the editing target sequence comprises a sequence in a pseudogene of NCF1, wherein the pseudogene is NCF1C. In some embodiments, the editing target sequence comprises a sequence in an exon of NCF1C. In some embodiments, the editing target sequence comprises a sequence in exon 2 of NCF1C. In some embodiments, the editing target sequence is located between positions 75156578-75171998 of human chromosome 7 as set forth in human genome assembly consortium Human build 38 (GRCh38), GenBank accession GCF_000001405.39. In some embodiments, the editing target sequence is located between positions 75168609-75168810 of human chromosome 7. In some embodiments, the editing target sequence comprises nucleotides corresponding to positions 75168709-75168710 of human chromosome 7. In some embodiments, the editing target sequence in the NCF1 pseudogene comprises a two nucleotide GT deletion compared to a wild type NCF1 gene. Accordingly, in some embodiments, the editing template of a PEgRNA encodes the newly synthesized single stranded DNA that is replaces the editing target sequence, thereby editing the target NCF1C pseudogene. NCF1C differs from wildtype NCF1 in that it lacks a “GT” of a “GTGT” sequence, which are the first 4 nucleotides of exon 2 of wild type NCF1 (positions 74777267-74777270 of human chromosome 7, GRch 38). Because inserting a GT either upstream or downstream of another GT results in the same “GTGT” sequence, the two-nucleotide insert can be considered at the position of the second “GT” in “GTGT”. Accordingly, the editing template maybe designed to contain a GT insertion (or a reverse complement thereof) that corresponds to the wildtype NCF1 sequence, or may contain s TT, AT, or CT insertion (or a reverse complement thereof) that restores the wildtype NCF1 reading frame and does not alter the amino acid sequence encoded by wildtype NCF1. In some embodiments, one or more intended nucleotide edits in the newly synthesized single stranded DNA is incorporated in the target NCF1 gene, thereby editing the target NCF1C gene to comprise a sequence of a wild type NCF1 gene. In some embodiments, the editing template of the PEgRNA or the newly synthesized single stranded DNA comprises a two nucleotide insertion as compared to the editing target sequence. In some embodiments, the intended nucleotide edit comprises a two nucleotide insertion at a position corresponding to position 75168710 in human chromosome 7 as compared to the region corresponding to the editing target sequence in the NCF1C pseudogene. In some embodiments, the editing template of the PEgRNA or the newly synthesized single stranded DNA comprises a two nucleotide GT insertion as compared to the editing target sequence (or a complementary sequence in the target NCF1C pseudogene). In some embodiments, the intended nucleotide edit comprises a two nucleotide insertion at a position corresponding to position 75168708 in human chromosome 7 as compared to the region corresponding to the editing target sequence in the NCF1C pseudogene. In some embodiments, the editing template of the PEgRNA or the newly synthesized single stranded DNA comprises a two nucleotide TT, AT, or CT insertion as compared to the editing target sequence (or a complementary sequence in the target NCF1C pseudogene).

In some embodiments, the editing template comprises one or more intended nucleotide edits compared to the sequence on the target strand of the target gene (e.g., an NCF1 gene, an NCF1B pseudogene, or an NCF1C pseudogene). In some embodiments, the editing template encodes a single stranded DNA that comprises one or more intended nucleotide edits compared to the editing target sequence. In some embodiments, the single stranded DNA replaces the editing target sequence by prime editing, thereby incorporating the one or more intended nucleotide edits.

In some embodiments, incorporation of the one or more intended nucleotide edits corrects the mutation in the editing target sequence to wild type nucleotides at corresponding positions in the target gene: (e.g., an NCF1 gene, an NCF1B pseudogene, or an NCF1C pseudogene). As used herein, “correcting” a mutation means restoring a wild type sequence at the place of the mutation in the double stranded target DNA, e.g., target gene, by prime editing. In some embodiments, the editing template comprises and/or encodes a wild type target gene sequence (e.g., an NCF1 gene, an NCF1B pseudogene, or an NCF1C pseudogene).

In some embodiments, incorporation of the one or more intended nucleotide edits does not correct the mutation in the editing target sequence to wild type sequence, but allows for expression of a functional protein encoded by the target gene (e.g., an NCF1 gene, an NCF1B pseudogene, or an NCF1C pseudogene).). In the context of editing NCF1B or NCF1C pseudogenes, “correcting” the pseudogene may also refer to editing of the pseudogene to incorporate a GT (or AC) insertion such that the edited pseudogene has the same coding sequence as the wildtype NCF1 gene. In some embodiments, incorporation of the one or more intended nucleotide edits does not correct the mutation in the editing target sequence to wild type sequence, but allows for expression of a functional protein encoded by the target gene (e.g., an NCF1 gene, an NCF1B pseudogene, or an NCF1C pseudogene).

In some embodiments, the editing template comprises one or more intended nucleotide edits compared to the sequence on the target strand of the target gene (e.g., an NCF1 gene, an NCF1B pseudogene, or an NCF1C pseudogene) that is complementary to the editing target sequence, wherein the one or more intended nucleotide edits is a single nucleotide substitution, polynucleotide substitution, nucleotide insertion, or nucleotide deletion. In some embodiments, the intended nucleotide edit in the editing template comprises a single nucleotide substitution, polynucleotide substitution, nucleotide insertion, or nucleotide deletion compared to the sequence on the target strand of the target gene (e.g., an NCF1 gene, an NCF1B pseudogene, or an NCF1C pseudogene) that is complementary to the editing target at a position corresponding to a mutation in target gene, wherein the editing target sequence is on the sense strand of the target gene. In some embodiments, the intended nucleotide edit in the editing template comprises a single nucleotide substitution, polynucleotide substitution, nucleotide insertion, or nucleotide deletion compared to the sequence on the target strand of the target gene that is complementary to the editing target at a position corresponding to a mutation in target gene, wherein the editing target sequence is on the antisense strand of the target gene (e.g., an NCF1 gene, an NCF1B pseudogene, or an NCF1C pseudogene).

A guide RNA core (also referred to herein as the gRNA core, gRNA scaffold, or gRNA backbone sequence) of a PEgRNA may contain a polynucleotide sequence that binds to a DNA binding domain (e.g., Cas9) of a prime editor. The gRNA core may interact with a prime editor as described herein, for example, by association with a DNA binding domain, such as a DNA nickase of the prime editor.

One of skill in the art will recognize that different prime editors having different DNA binding domains from different DNA binding proteins may require different gRNA core sequences specific to the DNA binding protein. In some embodiments, the gRNA core is capable of binding to a Cas9-based prime editor. In some embodiments, the gRNA core is capable of binding to a Cpf1-based prime editor. In some embodiments, the gRNA core is capable of binding to a Cas12b-based prime editor.

In some embodiments, the gRNA core comprises regions and secondary structures involved in binding with specific CRISPR Cas proteins. For example, in a Cas9 based prime editing system, the gRNA core of a PEgRNA may comprise one or more regions of a base paired “lower stem” adjacent to the spacer sequence and a base paired “upper stem” following the lower stem, where the lower stem and upper stem may be connected by a “bulge” comprising unpaired RNAs. The gRNA core may further comprise a “nexus” distal from the spacer sequence, followed by a hairpin structure, e.g., at the 3′ end, as exemplified in FIG. 3. In some embodiments, the gRNA core comprises modified nucleotides as compared to a wild type gRNA core in the lower stem, upper stem, and/or the hairpin. For example, nucleotides in the lower stem, upper stem, an/or the hairpin regions may be modified, deleted, or replaced. In some embodiments, RNA nucleotides in the lower stem, upper stem, an/or the hairpin regions may be replaced with one or more DNA sequences. In some embodiments, the gRNA core comprises unmodified or wild type RNA sequences in the nexus and/or the bulge regions. In some embodiments, the gRNA core does not include long stretches of A-T pairs, for example, a GUUUU-AAAAC pairing element. In some embodiments, a prime editing system comprises a prime editor and a PEgRNA, wherein the prime editor comprises a SpCas9 nickase variant thereof, and the gRNA core of the PEgRNA comprises the sequence:

(SEQ ID NO: 34424)
GUUUUAGAGCUAGAAAUAGCAAGUUAAAAUAAGGCUAGUCCGUUAUCA
ACUUGAAAAAGUGGCACCGAGUCGGUGC;
(SEQ ID NO: 34425)
GUUUGAGAGCUAGAAAUAGCAAGUUUAAAUAAGGCUAGUCCGUUAUCA
ACUUGAAAAAGUGGGACCGAGUCGGUCC;
(SEQ ID NO: 34426)
GUUUAAGAGCUAUGCUGGAAACAGCAUAGCAAGUUUAAAUAAGGCUAG
UCCGUUAUCAACUUGAAAAAGUGGCACCGAGUCGGUGC;
(SEQ ID NO: 34427)
GUUUUAGUACUCUGGAAACAGAAUCUACUAAAACAAGGCAAAAUGCCG
UGUUUAUCUCGUCAACUUGUUGGCGAGA;
or
(SEQ ID NO: 34428)
GUUUUAGUACUCUGGAAACAGAAUCUACUGAAACAAGACAAUAUGUCG
UGUUUAUCCCAUCAAUUUAUUGGUGGGA.

In some embodiments, the gRNA core comprises the sequence GUUUUAGAGCUAGAAAUAGCAAGUUAAAAUAAGGCUAGUCCGUUAUCAACUUGAAAAAG UGGCACCGAGUCGGUGC (SEQ ID NO: 34424). Any gRNA core sequences known in the art are also contemplated in the prime editing compositions described herein.

A PEgRNA may also comprise optional modifiers, e.g., 3′ end modifier region and/or an 5′ end modifier region. In some embodiments, a PEgRNA comprises at least one nucleotide that is not part of a spacer, a gRNA core, or an extension arm. The optional sequence modifiers could be positioned within or between any of the other regions shown, and not limited to being located at the 3′ and 5′ ends. In certain embodiments, the PEgRNA comprises secondary RNA structure, such as, but not limited to, aptamers, hairpins, stem/loops, toeloops, and/or RNA-binding protein recruitment domains (e.g., the MS2 aptamer which recruits and binds to the MS2cp protein). In some embodiments, a PEgRNA comprises a short stretch of uracil at the 5′ end or the 3′ end. For example, in some embodiments, a PEgRNA comprising a 3′ extension arm comprises a “UUU” sequence at the 3′ end of the extension arm. In some embodiments, a PEgRNA comprises a toeloop sequence at the 3′ end. In some embodiments, the PEgRNA comprises a 3′ extension arm and a toeloop sequence at the 3′ end of the extension arm. In some embodiments, the PEgRNA comprises a 5′ extension arm and a toeloop sequence at the 5′ end of the extension arm. In some embodiments, the PEgRNA comprises a toeloop element having the sequence 5′-GAAANNNNN-3′, wherein N is any nucleobase. In some embodiments, the secondary RNA structure is positioned within the spacer. In some embodiments, the secondary structure is positioned within the extension arm. In some embodiments, the secondary structure is positioned within the gRNA core. In some embodiments, the secondary structure is positioned between the spacer and the gRNA core, between the gRNA core and the extension arm, or between the spacer and the extension arm. In some embodiments, the secondary structure is positioned between the PBS and the editing template. In some embodiments the secondary structure is positioned at the 3′ end or at the 5′ end of the PEgRNA. In some embodiments, the PEgRNA comprises a transcriptional termination signal at the 3′ end of the PEgRNA. In addition to secondary RNA structures, the PEgRNA may comprise a chemical linker or a poly(N) linker or tail, where “N” can be any nucleobase. In some embodiments, the chemical linker may function to prevent reverse transcription of the gRNA core.

In some embodiments, a prime editing system or composition further comprises a nick guide polynucleotide, such as a nick guide RNA (ngRNA). In some embodiments, a ngRNA comprises a spacer (referred to as a ngRNA spacer or ng spacer) and a gRNA core, wherein the spacer of the ngRNA comprises a region of complementarity to the edit strand, and wherein the gRNA core can interact with a Cas, e.g., Cas9, of a prime editor. Without wishing to be bound by any particular theory, an ngRNA may bind to the edit strand and direct the Cas nickase to generate a nick on the non-edit strand (or target strand). In some embodiments, the nick on the non-edit strand directs endogenous DNA repair machinery to use the edit strand as a template for repair of the non-edit strand, which may increase efficiency of prime editing. In some embodiments, the non-edit strand is nicked by a prime editor localized to the non-edit strand by the ngRNA. Accordingly, also provided herein are PEgRNA systems comprising at least one PEgRNA and at least one ngRNA.

A prime editing system comprising a PEgRNA (or one or more polynucleotide encoding the PEgRNA) and a prime editor protein (or one or more polynucleotides encoding the prime editor), may be referred to as a PE2 prime editing system and the corresponding editing approach referred to as PE2 approach or PE2 strategy. A PE2 system does not contain a ngRNA. A prime editing system comprising a PEgRNA (or one or more polynucleotide encoding the PEgRNA), a prime editor protein (or one or more polynucleotides encoding the prime editor), and a ngRNA (or one or more polynucleotides encoding the ngRNA) may be referred to as a “PE3” prime editing system. In some embodiments, an ngRNA spacer sequence is complementary to a portion of the edit strand that includes the intended nucleotide edit, and may hybridize with the edit strand only after the edit has been incorporated on the edit strand. Such ngRNA may be referred to a “PE3b” ngRNA, and the prime editing system a PE3b prime editing system.

In some embodiments, a PEgRNA or a nick guide RNA (ngRNA) can be chemically synthesized, or can be assembled or cloned and transcribed from a DNA sequence, e.g., a plasmid DNA sequence, or by any RNA oligonucleotide synthesis method known in the art. In some embodiments, a DNA sequence that encodes a PEgRNA (or ngRNA) can be designed to append one or more nucleotides at the 5′ end or the 3′ end of the PEgRNA (or nick guide RNA) encoding sequence to enhance PEgRNA transcription. For example, in some embodiments, a DNA sequence that encodes a PEgRNA (or nick guide RNA) (or an ngRNA) can be designed to append a nucleotide G at the 5′ end. Accordingly, in some embodiments, the PEgRNA (or nick guide RNA) can comprise an appended nucleotide G at the 5′ end. In some embodiments, a DNA sequence that encodes a PEgRNA (or nick guide RNA) can be designed to append a sequence that enhances transcription, e.g., a Kozak sequence, at the 5′ end. In some embodiments, a DNA sequence that encodes a PEgRNA (or nick guide RNA) can be designed to append the sequence CACC or CCACC at the 5′ end. Accordingly, in some embodiments, the PEgRNA (or nick guide RNA) can comprise an appended sequence CACC or CCACC at the 5′ end. In some embodiments, a DNA sequence that encodes a PEgRNA (or nick guide RNA) can be designed to append the sequence TTT. TTT, TITTT, TTTTT, TTTTTT at the 3′ end. Accordingly, in some embodiments, the PEgRNA (or nick guide RNA) can comprise an appended sequence UUU. UUUU, UUUUU, UUUUUU, or UUUUUUU at the 3′ end.

Lengthy table referenced here
US20250243474A1-20250731-T00001
Please refer to the end of the specification for access instructions.

Lengthy table referenced here
US20250243474A1-20250731-T00002
Please refer to the end of the specification for access instructions.

Lengthy table referenced here
US20250243474A1-20250731-T00003
Please refer to the end of the specification for access instructions.

Lengthy table referenced here
US20250243474A1-20250731-T00004
Please refer to the end of the specification for access instructions.

Lengthy table referenced here
US20250243474A1-20250731-T00005
Please refer to the end of the specification for access instructions.

Lengthy table referenced here
US20250243474A1-20250731-T00006
Please refer to the end of the specification for access instructions.

Lengthy table referenced here
US20250243474A1-20250731-T00007
Please refer to the end of the specification for access instructions.

Lengthy table referenced here
US20250243474A1-20250731-T00008
Please refer to the end of the specification for access instructions.

Lengthy table referenced here
US20250243474A1-20250731-T00009
Please refer to the end of the specification for access instructions.

Lengthy table referenced here
US20250243474A1-20250731-T00010
Please refer to the end of the specification for access instructions.

Lengthy table referenced here
US20250243474A1-20250731-T00011
Please refer to the end of the specification for access instructions.

Lengthy table referenced here
US20250243474A1-20250731-T00012
Please refer to the end of the specification for access instructions.

Lengthy table referenced here
US20250243474A1-20250731-T00013
Please refer to the end of the specification for access instructions.

Lengthy table referenced here
US20250243474A1-20250731-T00014
Please refer to the end of the specification for access instructions.

Lengthy table referenced here
US20250243474A1-20250731-T00015
Please refer to the end of the specification for access instructions.

Lengthy table referenced here
US20250243474A1-20250731-T00016
Please refer to the end of the specification for access instructions.

Lengthy table referenced here
US20250243474A1-20250731-T00017
Please refer to the end of the specification for access instructions.

Lengthy table referenced here
US20250243474A1-20250731-T00018
Please refer to the end of the specification for access instructions.

Lengthy table referenced here
US20250243474A1-20250731-T00019
Please refer to the end of the specification for access instructions.

Lengthy table referenced here
US20250243474A1-20250731-T00020
Please refer to the end of the specification for access instructions.

Lengthy table referenced here
US20250243474A1-20250731-T00021
Please refer to the end of the specification for access instructions.

Lengthy table referenced here
US20250243474A1-20250731-T00022
Please refer to the end of the specification for access instructions.

Lengthy table referenced here
US20250243474A1-20250731-T00023
Please refer to the end of the specification for access instructions.

Lengthy table referenced here
US20250243474A1-20250731-T00024
Please refer to the end of the specification for access instructions.

Lengthy table referenced here
US20250243474A1-20250731-T00025
Please refer to the end of the specification for access instructions.

Lengthy table referenced here
US20250243474A1-20250731-T00026
Please refer to the end of the specification for access instructions.

Lengthy table referenced here
US20250243474A1-20250731-T00027
Please refer to the end of the specification for access instructions.

Lengthy table referenced here
US20250243474A1-20250731-T00028
Please refer to the end of the specification for access instructions.

Lengthy table referenced here
US20250243474A1-20250731-T00029
Please refer to the end of the specification for access instructions.

Lengthy table referenced here
US20250243474A1-20250731-T00030
Please refer to the end of the specification for access instructions.

Lengthy table referenced here
US20250243474A1-20250731-T00031
Please refer to the end of the specification for access instructions.

Lengthy table referenced here
US20250243474A1-20250731-T00032
Please refer to the end of the specification for access instructions.

Lengthy table referenced here
US20250243474A1-20250731-T00033
Please refer to the end of the specification for access instructions.

Lengthy table referenced here
US20250243474A1-20250731-T00034
Please refer to the end of the specification for access instructions.

Lengthy table referenced here
US20250243474A1-20250731-T00035
Please refer to the end of the specification for access instructions.

Lengthy table referenced here
US20250243474A1-20250731-T00036
Please refer to the end of the specification for access instructions.

Lengthy table referenced here
US20250243474A1-20250731-T00037
Please refer to the end of the specification for access instructions.

Lengthy table referenced here
US20250243474A1-20250731-T00038
Please refer to the end of the specification for access instructions.

Lengthy table referenced here
US20250243474A1-20250731-T00039
Please refer to the end of the specification for access instructions.

Lengthy table referenced here
US20250243474A1-20250731-T00040
Please refer to the end of the specification for access instructions.

Lengthy table referenced here
US20250243474A1-20250731-T00041
Please refer to the end of the specification for access instructions.

Lengthy table referenced here
US20250243474A1-20250731-T00042
Please refer to the end of the specification for access instructions.

Lengthy table referenced here
US20250243474A1-20250731-T00043
Please refer to the end of the specification for access instructions.

Lengthy table referenced here
US20250243474A1-20250731-T00044
Please refer to the end of the specification for access instructions.

Lengthy table referenced here
US20250243474A1-20250731-T00045
Please refer to the end of the specification for access instructions.

Lengthy table referenced here
US20250243474A1-20250731-T00046
Please refer to the end of the specification for access instructions.

Lengthy table referenced here
US20250243474A1-20250731-T00047
Please refer to the end of the specification for access instructions.

Lengthy table referenced here
US20250243474A1-20250731-T00048
Please refer to the end of the specification for access instructions.

Lengthy table referenced here
US20250243474A1-20250731-T00049
Please refer to the end of the specification for access instructions.

Lengthy table referenced here
US20250243474A1-20250731-T00050
Please refer to the end of the specification for access instructions.

Lengthy table referenced here
US20250243474A1-20250731-T00051
Please refer to the end of the specification for access instructions.

Lengthy table referenced here
US20250243474A1-20250731-T00052
Please refer to the end of the specification for access instructions.

Lengthy table referenced here
US20250243474A1-20250731-T00053
Please refer to the end of the specification for access instructions.

Lengthy table referenced here
US20250243474A1-20250731-T00054
Please refer to the end of the specification for access instructions.

Lengthy table referenced here
US20250243474A1-20250731-T00055
Please refer to the end of the specification for access instructions.

Lengthy table referenced here
US20250243474A1-20250731-T00056
Please refer to the end of the specification for access instructions.

Lengthy table referenced here
US20250243474A1-20250731-T00057
Please refer to the end of the specification for access instructions.

Lengthy table referenced here
US20250243474A1-20250731-T00058
Please refer to the end of the specification for access instructions.

Lengthy table referenced here
US20250243474A1-20250731-T00059
Please refer to the end of the specification for access instructions.

Lengthy table referenced here
US20250243474A1-20250731-T00060
Please refer to the end of the specification for access instructions.

Lengthy table referenced here
US20250243474A1-20250731-T00061
Please refer to the end of the specification for access instructions.

Lengthy table referenced here
US20250243474A1-20250731-T00062
Please refer to the end of the specification for access instructions.

Lengthy table referenced here
US20250243474A1-20250731-T00063
Please refer to the end of the specification for access instructions.

Lengthy table referenced here
US20250243474A1-20250731-T00064
Please refer to the end of the specification for access instructions.

Lengthy table referenced here
US20250243474A1-20250731-T00065
Please refer to the end of the specification for access instructions.

Lengthy table referenced here
US20250243474A1-20250731-T00066
Please refer to the end of the specification for access instructions.

Lengthy table referenced here
US20250243474A1-20250731-T00067
Please refer to the end of the specification for access instructions.

Lengthy table referenced here
US20250243474A1-20250731-T00068
Please refer to the end of the specification for access instructions.

Lengthy table referenced here
US20250243474A1-20250731-T00069
Please refer to the end of the specification for access instructions.

Lengthy table referenced here
US20250243474A1-20250731-T00070
Please refer to the end of the specification for access instructions.

Lengthy table referenced here
US20250243474A1-20250731-T00071
Please refer to the end of the specification for access instructions.

Lengthy table referenced here
US20250243474A1-20250731-T00072
Please refer to the end of the specification for access instructions.

Lengthy table referenced here
US20250243474A1-20250731-T00073
Please refer to the end of the specification for access instructions.

Lengthy table referenced here
US20250243474A1-20250731-T00074
Please refer to the end of the specification for access instructions.

Lengthy table referenced here
US20250243474A1-20250731-T00075
Please refer to the end of the specification for access instructions.

In some embodiments, the ng search target sequence is located on the non-target strand, within 10 base pairs to 100 base pairs of an intended nucleotide edit incorporated by the PEgRNA on the edit strand. In some embodiments, the ng target search target sequence is within 10 bp, 20 bp, 30 bp, 40 bp, 50 bp, 60 bp, 70 bp, 80 bp, 90 bp, 91 bp, 92 bp, 93 bp, 94 bp, 95 bp, 96 bp, 97 bp, 98 bp, 99 bp, or 100 bp of an intended nucleotide edit incorporated by the PEgRNA on the edit strand. In some embodiments, the 5′ ends of the ng search target sequence and the PEgRNA search target sequence are within 1, 2, 3, 4, 5, 6, 7, 8, 9, or 10 bp apart from each other. In some embodiments, the 5′ ends of the ng search target sequence and the PEgRNA search target sequence are within 10 bp, 20 bp, 30 bp, 40 bp, 50 bp, 60 bp, 70 bp, 80 bp, 90 bp, 91 bp, 92 bp, 93 bp, 94 bp, 95 bp, % bp, 97 bp, 98 bp, 99 bp, or 100 bp apart from each other.

Exemplary combinations of PEgRNA components, e.g., spacer, PBS, and editing template/RTT, as well as combinations of each PEgRNA and corresponding ngRNA(s) are provided in Tables 1-76. Tables 1-76 each contain three columns. From left to right, the first column is the sequence number. The second column provides the sequence of the component as actual sequence or by reference to a SEQ ID NO. Although all the sequences provided in Tables 1-76 are RNA sequences, “T” is used instead of a “U” in the sequences for consistency with the ST.26 standard used in the accompanying sequence listing. The third column contains a description of the sequence, where each sequence is annotated with its specific information.

Each of Tables 1-76 provides Prime Editing guide RNAs (PEgRNAs) that can be used with any Prime Editor comprising a Cas9 protein capable of recognizing a specific PAM sequence, as provided in the Description column of the Tables for the PEgRNA spacer. The PEgRNAs of each of Tables 1-76 can also be used in Prime Editing systems further comprising a nick guide RNA (ngRNA). Such PEgRNAs and Prime Editing systems can be used, for example, to correct or edit an c.73_74 ΔGT mutation in NCF1, or to edit the corresponding site in NCF1B or NCF1C.

The PEgRNAs exemplified in Tables 1-76 comprise: (a) a spacer comprising at its 3′ end a sequence corresponding to a listed PEgRNA spacer sequence; (b) a gRNA core capable of complexing with a Cas9 protein, and (c) an extension arm comprising: (i) an editing template comprising at its 3′ end an RTT sequence from the same table as the PEgRNA spacer, and (ii) a prime binding site (PBS) comprising at its 5′ end a PBS sequence from the same table as the PEgRNA spacer.

The PEgRNA spacer can be, for example, 16-22 nucleotides in length. The PEgRNA spacers in Tables 1-76 are annotated with their PAM sequence(s), enabling the selection of an appropriate Cas9 protein. The indicated PAM sequences, unless otherwise noted, is in a 5′ to 3′ order. For example, a “NGG” PAM is a 5′-NGG-3′ PAM.

The editing template can be referred to as a reverse transcription template (RTT). The editing template can encode wildtype NCF1 gene sequence. For example, in some embodiments, the editing template encodes a “GT” or “AC” insertion to restore wildtype NCF1 sequence, the dinucleotide insertion that corrects the c.73_74 ΔGT mutation can be considered at positions corresponding to c.73_74, or at positions adjacent to c.73_74, as the mutation is a deletion of “GT” from a “GTGT” sequence. The GT (or AC on the opposite strand) insertion encoded by an editing template can thus also be considered at the position corresponding to the third and fourth nucleotides of exon 2 of NCF1 gene. In some embodiments, the editing template can encode one or more synonymous edits relative to the wildtype NCF1 gene. In some cases, a synonymous edit can be an edit that alters the third nucleotide of exon 2 of the wild type NCF1 gene sequence (or the reverse complement of the third nucleotide of exon 2 of the wild type NCF1 gene sequence). Because the first three nucleotides of NCF1 exon 2 encodes a Valine, such edits are referred to as Valine recode edits. In some embodiments, the editing template contains one or more intended edits that alters the PAM sequence of the PEgRNA (or the reverse complement of the PAM sequence) such that the PAM is no longer recognized by the Cas9. Such edits are referred to as PAM silencing edits. A PAM silencing edit can be a synonymous edit or can be at a position corresponding to a non-coding region of wildtype NCF1 gene. In some embodiments, the intended edit(s) in the editing template, for example, a dinucleotide insertion at position c.73_74 or c.75_76 in the NCF1 gene, a synonymous edit, or a PAM silencing edit, are in such position(s) in the editing template that incorporation of the edits alter the protospacer sequence that correspond to the PEgRNA spacer sequence. Such edits are referred to as protospacer edits. Without wishing to be bound by any particular theory, PAM silencing edits and/or protospacer edits may prevent the Cas9 nickase from re-nicking the edit strand, thereby improving Prime Editing efficiency or reduce indel formation. The edit type, including protospacer edit, Valine recode edit, and PAM silencing edit, as well as the particular edits made for PAM silencing and Valine recoding, are provided in the third column of each of Tables 1-76. When the third column in the Table does not include a specific description of the RT, e.g., a Valine recode edit, a protospacer edit, or a PAM silencing edit, the RU encodes a wildtype NCF1 sequence. Some of the RUs in Tables 1-76 have perfect complementarity to specific PE3b nick guide RNA (ngRNA) spacers. Such RUs are annotated in the third column with a * followed by a number code, to indicate the specific complementary PE3b ngRNA. Briefly, the RUs are annotated with *N wherein N is the same number annotated to the PE3b ngRNA spacer that has perfect complementarity to a specific edit encoded by the RU. In some embodiments, a RU contains edits e.g., PAM silencing edits, synonymous edits, and/or edits corresponding to a non-coding region of NCF1, in addition to a dinucleotide insertion corresponding to the nucleotides 3-4 of exon 2 of NCF1 gene. In some embodiments, such additional edits can allow for additional designs of PE3b ngRNA spacers that have perfect complementarity to a portion of the edit strand that includes one or more specific edits encoded by the RTT (i.e., having prefect identity to a portion of the RTT that includes the edit).

In some embodiments, the editing template comprises at least 4 nucleotides that is 5′ to the 5′-most edit, wherein the at least 4 nucleotides are complementary to the edit strand of the target gene. Accordingly, in cases where a listed RTT sequence contains less than 4 contiguous nucleotides 5′ to the 5′ most edit that are complementary to the edit strand, the editing template may further comprise 1, 2, 3, 4, or more contiguous nucleotides at the 5′ end that are complementary to the edit strand.

The PBS can be, for example, 3 to 19 nucleotides in length. In some cases, a PBS length of no more than 3 nucleotides less than the PEgRNA spacer length is chosen.

The PEgRNA provided in Tables 1-76 can comprise, from 5′ to 3′, the spacer, the gRNA core, the editing template, and the PBS. The 3′ end of the editing template can be contiguous with the 5′ end of the PBS. The PEgRNA can comprise multiple RNA molecules (e.g., a crRNA containing the PEgRNA spacer and a tracrRNA containing the extension arm) or can be a single gRNA molecule. Any PEgRNA exemplified in Tables 1-76 may comprise, or further comprise, a 3′ motif at the 3′ end of the extension arm, for example, a hairpin-forming motif or a series of 1, 2, 3, 4, 5, 6, 7 or more U nucleotides. In some embodiments, the PEgRNA comprises 4 U nucleotides at its 3′ end. Without being bound by theory, such 3′ motifs are believed to increase PEgRNA stability. The PEgRNA may alternatively or additionally comprise one or more chemical modifications, such as phosphorothioate (PS) bond(s), 2′-O-methylated (2′-Ome) nucleotides, or a combination thereof. In some embodiments, the PEgRNA comprise 3′ mN*mN*mN*N and 5′mN*mN*mN* modifications, where m indicates that the nucleotide contains a 2′-O-Me modification and a * indicates the presence of a phosphorothioate bond. PEgRNA sequences exemplified in Tables 1-76 may alternatively be adapted for expression from a DNA template, for example, by including a 5′ terminal G if the spacer of the PEgRNA begins with a nucleotide other than G, by including 6 or 7 U nucleotides at the 3′ end of the extension arm, or both.

Any of the PEgRNAs of Tables 1-76 can be used in a Prime Editing system further comprising a nick guide RNA (ngRNA). Such ngRNA can comprise a spacer comprising at its 3′ end a sequence corresponding to nucleotides 5-20 of any of the 20-nucleotide ngRNA spacers listed in the same table as the PEgRNA spacer and a gRNA core capable of complexing with a Cas9 protein. For example, the sequence in the spacer of the ngRNA can comprise nucleotides 5-20, 4-20, 3-20, 2-20, or 1-20 of a 20-nucleotide-long spacer listed in the table. In some embodiments, the spacer of the ngRNA is the complete sequence of an ngRNA spacer listed in the same table as the PEgRNA spacer. The ngRNA spacers in Tables 1-76 are annotated with their PAM sequences, enabling selection of an appropriate Cas9 protein. It can be advantageous to select an ngRNA spacer that has a PAM sequence compatible with the Cas9 protein used in the Prime Editor with the PEgRNA, thus avoiding the need to use two different Cas9 proteins. The ngRNA can comprise multiple RNA molecules (e.g., a crRNA containing the ngRNA spacer and a tracrRNA) or can be a single gRNA molecule. The ngRNA is capable of binding the edit strand of the NCF1 gene (or a NCF1B or NCF1C pseudogene) and directing a complexed Cas9 nickase protein to nick the non-edit strand. A PE3 ngRNA spacer has perfect complementarity to the edit strand both prior to and post incorporation of the intended edit(s) encoded by the PEgRNA; a PE3b ngRNA spacer has perfect complementarity to the edit strand only after the intended edit(s) are incorporated. Without wishing to be bound by any particular theory, a PE3b ngRNA spacer is believed to reduce indel formation resulted from PE3 nicking of the non-edit strand. In Tables 1-76, a PE3b*N ngRNA spacer, where N is the integer following the *, has perfect complementarity to the edit strand post-edit containing the specific edit encoded by the editing template in the same Table, wherein the editing template (RTT) is annotated with the same *N. For example, a PE3b*1 ngRNA spacer has 100% complementary with the portion of the edit strand containing the edit encoded by a RTT annotated with * 1.

Any ngRNA sequence provided in Tables 1-76 may comprise, or further comprise, a 3′ motif at their 3′ end, for example, a series of 1, 2, 3, 4, 5, 6, 7 or more U nucleotides. In some embodiments, the ngRNA comprises 4 U nucleotides at its 3′ end. Without being bound by theory, such 3′ motifs are believed to increase ngRNA stability. The ngRNA may alternatively or additionally comprise one or more chemical modifications, such as phosphorothioate (PS) bond(s), 2′-O-methylated (2′-Ome) nucleotides, or a combination thereof. In some embodiments, the ngRNA comprise 3′ mN*mN*mN*N and 5′mN*mN*mN* modifications, where m indicates that the nucleotide contains a 2′-O-Me modification and a * indicates the presence of a phosphorothioate bond. NgRNA sequences may alternatively be adapted for expression from a DNA template, for example, by including a 5′ terminal G if the spacer of the ngRNA begins with a nucleotide other than G, by including 6 or 7 U nucleotides at the 3′ end of the ngRNA, or both.

The gRNA core sequence of any of PEgRNA sequences in Tables 1-76 can be any gRNA scaffold sequence that can complex with a Cas9 that recognizes the PAM sequence annotated for each Table. In some embodiments, the gRNA core for the PEgRNA and/or the ngRNA comprises a sequence selected from SEQ ID Nos 34424, In some embodiments, the gRNA core for the PEgRNA and/or the ngRNA comprises SEQ ID No. 34428. In some embodiments, the gRNA core for the PEgRNA and/or the ngRNA comprises SEQ ID No. 34427.

### Table 1

Table 1 provides Prime Editing guide RNAs (PEgRNAs) that can be used with any Prime Editor containing a Cas9 protein capable of recognizing a TG, TGG, or TGGG PAM sequence. The PEgRNAs of Table 1 can also be used in Prime Editing systems further comprising a nick guide RNA (ngRNA). Such PEgRNAs and Prime Editing systems can be used, for example, to correct or edit an c.73_74 ΔGT mutation in NCF1, or to edit the corresponding site in NCF1B or NCF1C.

The PEgRNAs exemplified in Table 1 comprise: (a) a spacer comprising at its 3′ end a sequence corresponding to sequence number 1; (b) a gRNA core capable of complexing with a Cas9 protein, and (c) an extension arm comprising: (i) an editing template at least 10 nucleotides in length and comprising at its 3′ end a sequence corresponding to sequence number 27, 25, 26, or 28, and (ii) a prime binding site (PBS) comprising at its 5′ end a sequence corresponding to sequence number 8.

The PEgRNA spacer can be, for example, 16-22 nucleotides in length and can comprise the sequence corresponding to any one of sequence numbers 1-7. In some embodiments, the PEgRNA spacer comprises sequence number 5. The PEgRNA spacers in Table 1 are annotated with their PAM sequence(s), enabling the selection of an appropriate Cas9 protein.

The editing template can be referred to as a reverse transcription template (RTT). The editing template comprises one or more intended nucleotide edits for incorporation into the target NCF1 (or NCF1B/NCF1C) gene by Prime Editing. In some embodiments, the editing template comprises at least 4 nucleotides that is 5′ to the 5′-most edit, wherein the at least 4 nucleotides are complementary to the edit strand of the target gene. Accordingly, in cases where a listed RTT sequence contains less than 4 contiguous nucleotides 5′ to the 5′ most edit that are complementary to the edit strand, the editing template may further comprise 1, 2, 3, 4, or more contiguous nucleotides at the 5′ end that are complementary to the edit strand.

In some embodiments, the editing template encodes a wildtype NCF1 gene sequence. For example, the editing template can comprise at its 3′ end the sequence corresponding to sequence number 27, 31, 35, 39, 43, 47, 51, 56, 60, 64, 66, 71, 75, 78, 84, 85, 91, 94, 98, 103, 108, 110, 114, 118, 121, 125, 132, 135, 138, 144, 147, 151, 153, 159, 161, 165, 172, 175, 180, 182, 188, 191, 196, 200, 202, 207, 212, 215, 218, 221, 226, 230, 234, 238, 241, 246, 250, 256, 258, 262, 266, 272, 275, 280, 282, 286, 289, 294, 298, 303, 308, 311, 314, 317, 321, 326, 331, 335, 338, 341, 347, 350, 353, 359, 364, 367, 372, 374, 379, 384, 388, 391, 395, or 400. In some embodiments, the editing template can encode one or more synonymous edits relative to the wildtype NCF1 gene. In some cases, a synonymous edit is a Valine recode edit. For example, the editing template can comprise at its 3′ end the sequence corresponding to sequence number 290, 291, 292, 293, 296, 295, 299, 300, 297, 302, 304, 301, 307, 305, 306, 312, 310, 309, 313, 315, 316, 320, 318, 319, 322, 323, 324, 325, 328, 327, 332, 330, 329, 334, 336, 333, 339, 337, 340, 343, 344, 342, 345, 346, 348, 349, 351, 352, 355, 354, 356, 357, 358, 360, 362, 361, 363, 366, 365, 368, 370, 371, 369, 373, 375, 376, 380, 377, 378, 381, 383, 382, 386, 387, 385, 392, 389, 390, 394, 396, 393, 398, 399, or 397. In some embodiments, the editing template encodes a protospacer edit. For example, an editing template that encodes a protospacer edit can comprises at its 3′ end the sequence corresponding to sequence number 27, 31, 35, 39, 43, 47, 51, 56, 60, 64, 66, 71, 75, 78, 84, 85, 91, 94, 98, 103, 108, 110, 114, 118, 121, 125, 132, 135, 138, 144, 147, 151, 153, 159, 161, 165, 172, 175, 180, 182, 188, 191, 1%, 200, 202, 207, 212, 215, 218, 221, 226, 230, 234, 238, 241, 246, 250, 256, 258, 262, 266, 272, 275, 280, 282, 286, 289, 294, 298, 303, 308, 311, 314, 317, 321, 326, 331, 335, 338, 341, 347, 350, 353, 359, 364, 367, 372, 374, 379, 384, 388, 391, 395, 400, 290, 291, 292, 293, 296, 295, 299, 300, 297, 302, 304, 301, 307, 305, 306, 312, 310, 309, 313, 315, 316, 320, 318, 319, 322, 323, 324, 325, 328, 327, 332, 330, 329, 334, 336, 333, 339, 337, 340, 343, 344, 342, 345, 346, 348, 349, 351, 352, 355, 354, 356, 357, 358, 360, 362, 361, 363, 366, 365, 368, 370, 371, 369, 373, 375, 376, 380, 377, 378, 381, 383, 382, 386, 387, 385, 392, 389, 390, 394, 3%, 393, 398, 399, or 397.

The PBS can be, for example, 3 to 19 nucleotides in length and can comprise the sequence corresponding to any one of sequence numbers 8-24. In some cases, a PBS length of no more than 3 nucleotides less than the PEgRNA spacer length is chosen.

The PEgRNA can comprise, from 5′ to 3′, the spacer, the gRNA core, the editing template, and the PBS. The 3′ end of the editing template can be contiguous with the 5′ end of the PBS. The PEgRNA can comprise multiple RNA molecules or can be a single RNA molecule. Exemplary PEgRNAs provided in Table 1 can comprise a sequence corresponding to sequence number 482, 485, 483, 486, 484, 488, 487, 490, 491, 495, 494, 492, 493, 496, 489, 497, 501, 499, 498, 500, 502, 505, 503, 511, 512, 510, 506, 504, 508, 513, 507, 509, 518, 515, 519, 514, 517, 516, 526, 524, 520, 522, 530, 521, 528, 532, 531, 534, 527, 525, 533, 529, 523, 538, 541, 536, 539, 535, 540, 537, 546, 549, 545, 550, 543, 544, 548, 547, 542, 552, 551, 555, 557, 556, 554, 553, 560, 563, 561, 559, 562, 558, 566, 564, 569, 568, 567, 565, 570, 571, 574, 573, 572, 575, 576, or 577.

Any of the PEgRNAs of Table 1 can be used in a Prime Editing system further comprising a nick guide RNA (ngRNA). Such ngRNA can comprise (a) a spacer comprising at its 3′ end a sequence corresponding to nucleotides 5-20 of any ngRNA spacer corresponding to sequence number 449, 425, 417, 457, 444, 448, 440, 426, 424, 421, 422, 451, 447, 437, 445, 435, 446, 427, 436, 467, 454, 423, 441, 428, 465, 438, 434, 418, 464, 461, 452, 420, 460, 466, 453, 455, 459, 456, 439, 432, 433, 419, 462, 429, 431, 443, 458, 463, 450, 442, 430, 473, 472, 471, 468, 470, 469, 474, 476, 477, 480, 478, 475, or 479, and (b) a gRNA core capable of complexing with a Cas9 protein. For example, the sequence in the spacer of the ngRNA can comprise nucleotides 5-20, 4-20, 3-20, 2-20, or 1-20 of sequence number 449, 425, 417, 457, 444, 448, 440, 426, 424, 421, 422, 451, 447, 437, 445, 435, 446, 427, 436, 467, 454, 423, 441, 428, 465, 438, 434, 418, 464, 461, 452, 420, 460, 466, 453, 455, 459, 456, 439, 432, 433, 419, 462, 429, 431, 443, 458, 463, 450, 442, 430, 473, 472, 471, 468, 470, 469, 474, 476, 477, 480, 478, 475, or 479. In some embodiments, the spacer of the ngRNA is a ngRNA spacer listed in Table 1. The ngRNA spacers in Table 1 are annotated with their PAM sequences, enabling selection of an appropriate Cas9 protein. It can be advantageous to select a ngRNA spacer that has a PAM sequence compatible with the Cas9 protein used in the Prime Editor, thus avoiding the need to use two different Cas9 proteins. The ngRNA is capable of directing a complexed Cas9 protein to bind the edit strand of the NCF1 gene (or NCF1B/NCF1C pseudogene); thus, a complexed Cas9 nickase containing a nuclease inactivating mutation in the HNH domain will nick the non-edit strand. A PE3 ngRNA spacer has perfect complementarity to the edit strand both pre- and post-edit; a PE3b ngRNA spacer has perfect complementarity to the edit strand post-edit. A PE3b*N ngRNA spacer, where N is the integer following the * as indicated in the table, has perfect complementarity to the edit strand post-edit containing the specific edit encoded by the editing template annotated with the same *N. For example, a PE3b*1 ngRNA spacer has 100% complementary with the portion of the edit strand containing the edit encoded by a RTT annotated with *1.

Exemplary ngRNA provided in Table 1 can comprise a sequence corresponding to sequence number 579, 578, 581, 580, 592, 591, 589, 593, 582, 583, 586, 585, 595, 584, 594, 590, 587, 588, 602, 604, 607, 603, 596, 601, 598, 597, 600, 599, 608, 605, 606, or 481.

Any PEgRNA or ngRNA exemplified in Table 1 may comprise, or further comprise, a 3′ motif at the 3′ end of the extension arm, for example, a hairpin-forming motif or a series of 1, 2, 3, 4, 5, 6, 7 or more U nucleotides. In some embodiments, the PEgRNA or ngRNA comprises 4 U nucleotides at its 3′ end. Without being bound by theory, such 3′ motifs are believed to increase gRNA stability. The PEgRNA or ngRNA may alternatively or additionally comprise one or more chemical modifications, such as phosphorothioate (PS) bond(s), 2′-O-methylated (2′-Ome) nucleotides, or a combination thereof. In some embodiments, the PEgRNA or ngRNA comprise 3′ mN*mN*mN*N and 5′mN*mN*mN* modifications, where m indicates that the nucleotide contains a 2′-O-Me modification and a * indicates the presence of a phosphorothioate bond. PEgRNA and ngRNA sequences exemplified in Table 1 may alternatively be adapted for expression from a DNA template, for example, by including a 5′ terminal G if the spacer of the PEgRNA or ngRNA begins with a nucleotide other than G, by including 6 or 7 U nucleotides at the 3′ end of the extension arm, or both.

### Table 12

Table 12 provides Prime Editing guide RNAs (PEgRNAs) that can be used with any Prime Editor containing a Cas9 protein capable of recognizing a GG, GGG, or GGGG PAM sequence. The PEgRNAs of Table 12 can also be used in Prime Editing systems further comprising a nick guide RNA (ngRNA). Such PEgRNAs and Prime Editing systems can be used, for example, to correct or edit an c.73_74 ΔGT mutation in NCF1, or to edit the corresponding site in NCF1B or NCF1C.

The PEgRNAs exemplified in Table 12 comprise: (a) a spacer comprising at its 3′ end a sequence corresponding to sequence number 3995; (b) a gRNA core capable of complexing with a Cas9 protein, and (c) an extension arm comprising: (i) an editing template at least 10 nucleotides in length and comprising at its 3′ end a sequence corresponding to sequence number 4019, and (ii) a prime binding site (PBS) comprising at its 5′ end a sequence corresponding to sequence number 4002.

The PEgRNA spacer can be, for example, 16-22 nucleotides in length and can comprise the sequence corresponding to any one of sequence numbers 3995-4001. In some embodiments, the PEgRNA spacer comprises sequence number 3999. The PEgRNA spacers in Table 12 are annotated with their PAM sequence(s), enabling the selection of an appropriate Cas9 protein.

The editing template can be referred to as a reverse transcription template (RTT). The editing template comprises one or more intended nucleotide edits for incorporation into the target NCF1 (or NCF1B/NCF1C) gene by Prime Editing. In some embodiments, the editing template comprises at least 4 nucleotides that is 5′ to the 5′ most intended edit, wherein the at least 4 nucleotides are complementary to the edit strand of the target gene.

In some embodiments, the editing template encodes a wildtype NCF1 gene sequence. For example, the editing template can comprise at its 3′ end the sequence corresponding to sequence number 4019, 4020, 4021, 4037, 4054, 4072, 4098, 4102, 4119, 4136, 4149, 4171, 4193, 4209, 4213, 4230, 4245, 4263, 4277, 4305, 4313, 4330, 4348, 4358, 4375, 4402, 4409, 4427, 4439, 4460, 4472, 4490, 4504, 4517, 4533, 4557, 4568, 4584, 4601, 4618, 4630, 4650, 4671, 4681, 4702, 4715, 4732, 4744, 4764, 4785, 4791, 4809, 4826, 4844, 4859, 4870, 4893, 4910, 4920, 4945, 4949, 4971, 4984, 5007, 5020, 5030, 5047, 5061, 5087, 5099, 5110, 5131, 5152, 5157, 5175, 5203, 5217, 5234, 5243, 5258, 5275, 5286, 5309, 5319, 5339, 5349, 5369, 5385, 5399, 5414, 5433, 5458, 5464, or 5478. In some embodiments, the editing template encodes one or more PAM silencing edits relative to the wildtype NCF1 gene. The editing template that encodes one or more PAM silencing edits can comprise at its 3′ end the sequence corresponding to sequence number 4023, 4034, 4031, 4029, 4025, 4028, 4033, 4026, 4030, 4036, 4035, 4032, 4027, 4024, 4022, 4043, 4042, 4050, 4051, 4044, 4049, 4041, 4040, 4052, 4045, 4039, 4038, 4048, 4046, 4047, 4061, 4056, 4057, 4062, 4067, 4060, 4055, 4058, 4053, 4066, 4068, 4065, 4063, 4064, 4059, 4070, 4078, 4082, 4081, 4069, 4074, 4080, 4073, 4075, 4071, 4083, 4077, 4084, 4079, 4076, 4099, 4095, 4093, 4085, 4089, 4100, 4091, 4096, 4097, 4094, 4086, 4088, 4090, 4087, 4092, 4116, 4108, 4114, 4115, 4105, 4104, 4106, 4112, 4113, 4109, 4111, 4101, 4110, 4107, 4103, 4117, 4128, 4126, 4120, 4132, 4123, 4127, 4129, 4122, 4124, 4125, 4121, 4130, 4131, 4118, 4138, 4139, 4143, 4140, 4141, 4145, 4147, 4144, 4146, 4133, 4142, 4148, 4137, 4135, 4134, 4152, 4159, 4164, 4157, 4158, 4156, 4161, 4162, 4163, 4154, 4151, 4150, 4155, 4153, 4160, 4173, 4166, 4175, 4177, 4178, 4176, 4174, 4180, 4170, 4165, 4168, 4172, 4179, 4167, 4169, 4192, 4183, 4190, 4196, 4188, 4194, 4189, 4182, 4181, 4184, 4186, 4195, 4185, 4187, 4191, 4203, 4204, 4205, 4210, 4206, 4198, 4202, 4208, 4199, 4211, 4207, 4212, 4197, 4200, 4201, 4214, 4225, 4219, 4221, 4224, 4227, 4220, 4217, 4226, 4228, 4218, 4222, 4223, 4215, 4216, 4231, 4237, 4240, 4233, 4229, 4243, 4235, 4236, 4238, 4241, 4242, 4239, 4234, 4244, 4232, 4252, 4247, 4253, 4257, 4254, 4250, 4255, 4246, 4251, 4249, 4260, 4259, 4256, 4248, 4258, 4265, 4267, 4271, 4270, 4275, 4262, 4272, 4264, 4276, 4274, 4266, 4268, 4261, 4273, 4269, 4283, 4289, 4285, 4278, 4288, 4290, 4279, 4280, 4286, 4281, 4287, 4282, 4291, 4284, 4292, 4299, 4302, 4296, 4306, 4304, 4297, 4301, 4295, 4298, 4308, 4303, 4300, 4294, 4293, 4307, 4324, 4309, 4314, 4312, 4318, 4321, 4323, 4311, 4310, 4316, 4319, 4317, 4320, 4322, 4315, 4326, 4336, 4331, 4335, 4337, 4334, 4328, 4325, 4340, 4339, 4333, 4329, 4327, 4338, 4332, 4349, 4347, 4344, 4355, 4352, 4356, 4342, 4350, 4354, 4351, 4345, 4341, 4353, 4343, 4346, 4362, 4371, 4359, 4367, 4370, 4368, 4363, 4372, 4369, 4360, 4357, 4361, 4366, 4365, 4364, 4376, 4374, 4382, 4373, 4380, 4384, 4381, 4388, 4378, 4387, 4379, 4386, 4377, 4385, 4383, 4390, 4393, 4404, 4395, 4401, 4389, 4392, 4400, 4399, 4403, 4396, 4397, 4394, 4398, 4391, 4407, 4405, 4412, 4413, 4414, 4408, 4411, 4415, 4416, 4410, 4406, 4417, 4419, 4420, 4418, 4434, 4433, 4432, 4423, 4431, 4425, 4435, 4422, 4428, 4424, 4436, 4430, 4421, 4429, 4426, 4451, 4441, 4440, 4437, 4446, 4449, 4443, 4448, 4442, 4447, 4438, 4450, 4452, 4444, 4445, 4458, 4455, 4464, 4456, 4457, 4463, 4461, 4462, 4454, 4459, 4465, 4467, 4453, 4468, 4466, 4477, 4481, 4480, 4471, 4484, 4483, 4476, 4474, 4478, 4482, 4470, 4469, 4479, 4475, 4473, 4500, 4496, 4491, 4485, 4489, 4493, 4499, 4486, 4495, 4487, 4498, 4488, 4492, 4494, 4497, 4511, 4510, 4512, 4514, 4516, 4502, 4509, 4501, 4506, 4508, 4515, 4507, 4503, 4513, 4505, 4520, 4519, 4522, 4523, 4530, 4524, 4528, 4531, 4532, 4521, 4527, 4525, 4526, 4529, 4518, 4534, 4548, 4544, 4540, 4546, 4537, 4545, 4536, 4541, 4543, 4542, 4535, 4547, 4538, 4539, 4564, 4555, 4551, 4559, 4552, 4561, 4562, 4556, 4560, 4563, 4553, 4554, 4558, 4550, 4549, 4570, 4579, 4567, 4566, 4575, 4565, 4578, 4573, 4572, 4569, 4574, 4576, 4580, 4577, 4571, 4581, 4586, 4585, 4591, 4590, 4583, 4593, 4589, 4595, 4594, 4588, 4582, 4596, 4592, 4587, 4602, 4609, 4606, 4608, 4603, 4610, 4599, 4605, 4611, 4597, 4600, 4604, 4598, 4612, 4607, 4623, 4627, 4613, 4619, 4628, 4617, 4620, 4626, 4621, 4625, 4624, 4622, 4616, 4615, 4614, 4640, 4632, 4633, 4638, 4643, 4635, 4629, 4639, 4641, 4631, 4634, 4644, 4637, 4636, 4642, 4646, 4656, 4652, 4647, 4649, 4658, 4654, 4648, 4659, 4660, 4657, 4653, 4651, 4645, 4655, 4661, 4676, 4666, 4670, 4669, 4662, 4672, 4663, 4668, 4664, 4665, 4667, 4673, 4674, 4675, 4690, 4691, 4683, 4680, 4687, 4688, 4682, 4684, 4689, 4678, 4685, 4692, 4686, 4677, 4679, 4707, 4706, 4698, 4699, 4703, 4704, 4695, 4694, 4693, 4705, 4697, 4700, 4701, 4708, 4696, 4717, 4719, 4722, 4712, 4711, 4714, 4718, 4720, 4723, 4721, 4709, 4710, 4724, 4716, 4713, 4739, 4733, 4731, 4738, 4735, 4725, 4730, 4729, 4727, 4737, 4740, 4726, 4734, 4736, 4728, 4746, 4747, 4755, 4742, 4751, 4743, 4752, 4753, 4756, 4748, 4745, 4749, 4750, 4741, 4754, 4761, 4766, 4759, 4767, 4760, 4758, 4757, 4770, 4769, 4768, 4765, 4763, 4772, 4762, 4771, 4782, 4784, 4778, 4779, 4774, 4773, 4777, 4787, 4776, 4780, 4783, 4788, 4781, 4786, 4775, 4792, 4804, 4800, 4794, 4795, 4796, 4797, 4790, 4801, 4793, 4789, 4799, 4802, 4803, 4798, 4815, 4816, 4817, 4812, 4806, 4811, 4819, 4808, 4818, 4814, 4805, 4820, 4810, 4813, 4807, 4827, 4822, 4833, 4834, 4831, 4823, 4825, 4829, 4828, 4836, 4821, 4835, 4830, 4824, 4832, 4849, 4839, 4843, 4852, 4847, 4837, 4850, 4848, 4841, 4842, 4838, 4840, 4846, 4851, 4845, 4866, 4856, 4864, 4853, 4862, 4858, 4855, 4854, 4865, 4860, 4868, 4867, 4857, 4861, 4863, 4884, 4869, 4874, 4875, 4879, 4880, 4882, 4878, 4883, 4876, 4881, 4877, 4871, 4872, 4873, 4890, 4892, 4897, 4900, 4888, 4885, 4887, 4889, 4891, 4899, 4894, 4898, 4896, 4886, 4895, 4912, 4903, 4907, 4905, 4904, 4909, 4913, 4916, 4902, 4914, 4906, 4911, 4901, 4915, 4908, 4926, 4932, 4919, 4918, 4929, 4925, 4924, 4921, 4927, 4923, 4930, 4917, 4931, 4928, 4922, 4940, 4947, 4933, 4934, 4935, 4942, 4943, 4938, 4939, 4936, 4948, 4946, 4941, 4944, 4937, 4955, 4958, 4964, 4963, 4961, 4959, 4950, 4957, 4953, 4951, 4960, 4954, 4962, 4956, 4952, 4973, 4974, 4979, 4975, 4970, 4966, 4967, 4965, 4978, 4977, 4969, 4972, 4976, 4968, 4980, 4981, 4996, 4983, 4982, 4986, 4994, 4993, 4989, 4992, 4987, 4995, 4988, 4991, 4985, 4990, 5003, 4999, 5006, 5009, 4998, 5002, 5001, 5012, 5000, 4997, 5005, 5008, 5011, 5004, 5010, 5019, 5021, 5024, 5022, 5026, 5015, 5017, 5027, 5028, 5018, 5014, 5023, 5025, 5013, 5016, 5034, 5039, 5040, 5041, 5038, 5029, 5036, 5044, 5042, 5032, 5037, 5033, 5043, 5031, 5035, 5053, 5048, 5051, 5050, 5045, 5058, 5049, 5060, 5059, 5057, 5052, 5046, 5055, 5056, 5054, 5071, 5067, 5069, 5075, 5064, 5076, 5066, 5070, 5068, 5074, 5063, 5062, 5073, 5072, 5065, 5090, 5084, 5079, 5091, 5083, 5086, 5081, 5085, 5077, 5080, 5078, 5092, 5088, 5082, 5089, 5098, 5103, 5104, 5093, 5096, 5106, 5105, 5108, 5097, 5094, 5100, 5107, 5102, 5095, 5101, 5114, 5112, 5124, 5118, 5115, 5120, 5111, 5121, 5119, 5122, 5116, 5123, 5109, 5113, 5117, 5133, 5140, 5136, 5137, 5127, 5139, 5129, 5126, 5125, 5138, 5128, 5130, 5132, 5135, 5134, 5153, 5141, 5144, 5155, 5154, 5156, 5148, 5149, 5147, 5150, 5142, 5146, 5151, 5145, 5143, 5168, 5164, 5171, 5161, 5172, 5159, 5163, 5166, 5167, 5170, 5165, 5162, 5160, 5169, 5158, 5185, 5186, 5177, 5180, 5173, 5174, 5183, 5178, 5176, 5182, 5184, 5188, 5179, 5187, 5181, 5192, 5200, 5197, 5190, 5193, 5202, 5191, 5201, 5194, 5198, 5189, 5199, 5196, 5204, 5195, 5213, 5206, 5219, 5220, 5208, 5216, 5205, 5214, 5210, 5209, 5207, 5212, 5218, 5215, 5211, 5231, 5221, 5223, 5235, 5233, 5224, 5226, 5222, 5229, 5236, 5232, 5230, 5227, 5225, 5228, 5239, 5250, 5246, 5240, 5238, 5249, 5248, 5237, 5252, 5247, 5251, 5244, 5241, 5245, 5242, 5268, 5259, 5254, 5257, 5267, 5263, 5260, 5265, 5264, 5255, 5262, 5261, 5253, 5256, 5266, 5274, 5280, 5271, 5279, 5277, 5276, 5281, 5278, 5284, 5270, 5283, 5269, 5273, 5282, 5272, 5298, 5293, 5297, 5288, 5295, 5290, 5299, 5292, 5300, 5296, 5291, 5289, 5285, 5287, 5294, 5302, 5311, 5313, 5310, 5316, 5301, 5308, 5306, 5315, 5312, 5307, 5305, 5303, 5304, 5314, 5326, 5332, 5329, 5320, 5317, 5330, 5323, 5322, 5321, 5325, 5318, 5328, 5324, 5327, 5331, 5345, 5348, 5334, 5338, 5346, 5333, 5343, 5342, 5335, 5340, 5344, 5347, 5341, 5337, 5336, 5364, 5359, 5361, 5354, 5363, 5357, 5355, 5350, 5351, 5352, 5356, 5360, 5353, 5358, 5362, 5368, 5370, 5371, 5379, 5373, 5378, 5375, 5365, 5380, 5367, 5366, 5372, 5377, 5376, 5374, 5384, 5387, 5392, 5389, 5388, 5386, 5383, 5390, 5382, 5393, 5391, 5396, 5381, 5394, 5395, 5406, 5401, 5411, 5405, 5409, 5404, 5403, 5400, 5407, 5402, 5408, 5397, 5412, 5398, 5410, 5413, 5425, 5416, 5422, 5419, 5426, 5424, 5420, 5421, 5415, 5418, 5417, 5423, 5428, 5427, 5440, 5431, 5441, 5429, 5444, 5442, 5430, 5436, 5443, 5438, 5437, 5432, 5434, 5435, 5439, 5451, 5454, 5445, 5448, 5453, 5447, 5449, 5456, 5460, 5457, 5450, 5455, 5452, 5446, 5459, 5465, 5461, 5469, 5474, 5473, 5466, 5467, 5468, 5462, 5470, 5463, 5475, 5476, 5471, 5472, 5480, 5487, 5483, 5485, 5488, 5479, 5481, 5482, 5490, 5491, 5484, 5477, 5492, 5486, or 5489. In some embodiments, the editing template that encodes a protospacer edit comprises at its 3′ end any of the RTT sequences in Table 12.

In some embodiments, the editing template is at least 11 nucleotides in length and comprises at its 3′ end the sequence corresponding to sequence number 4020.

In some embodiments, the editing template is at least 12 nucleotides in length and comprises at its 3′ end the sequence corresponding to sequence number 4023, 4021, 4034, 4031, 4029, 4025, 4028, 4033, 4026, 4030, 4036, 4035, 4032, 4027, 4024, or 4022. In some embodiments, the editing template encodes a wild type NCF1 sequence and comprises at its 3′ end the sequence corresponding to sequence number 4021. In some embodiments, the editing template encodes a PAM silencing edit and comprises at its 3′ end the sequence corresponding to sequence number 4023, 4034, 4031, 4029, 4025, 4028, 4033, 4026, 4030, 4036, 4035, 4032, 4027, 4024, or 4022. In some embodiments, the editing template is 12 nucleotide in length.

In some embodiments, the editing template is at least 14 nucleotides in length and comprises at its 3′ end the sequence corresponding to sequence number 4061, 4056, 4057, 4062, 4067, 4060, 4055, 4058, 4053, 4066, 4068, 4065, 4054, 4063, 4064, or 4059. In some embodiments, the editing template encodes a wild type NCF1 sequence and comprises at its 3′ end the sequence corresponding to sequence number 4054. In some embodiments, the editing template encodes a PAM silencing edit and comprises at its 3′ end the sequence corresponding to sequence number 4061, 4056, 4057, 4062, 4067, 4060, 4055, 4058, 4053, 4066, 4068, 4065, 4063, 4064, or 4059. In some embodiments, the editing template is 14 nucleotides in length.

In some embodiments, the editing template is at least 16 nucleotides in length and comprises at its 3′ end the sequence corresponding to sequence number 4099, 4095, 4093, 4085, 4089, 4100, 4091, 4096, 4097, 4094, 4086, 4088, 4090, 4087, 4092, or 4098. In some embodiments, the editing template encodes a wild type NCF1 sequence and comprises at its 3′ end the sequence corresponding to sequence number 4098. In some embodiments, the editing template encodes a PAM silencing edit and comprises at its 3′ end the sequence corresponding to sequence number 4099, 4095, 4093, 4085, 4089, 4100, 4091, 4096, 4097, 4094, 4086, 4088, 4090, 4087, or 4092. In some embodiments, the editing template is 16 nucleotides in length.

In some embodiments, the editing template is 11 to 16 nucleotides in length, and comprises the sequence corresponding to sequence number 4020, 4023, 4021, 4034, 4031, 4029, 4025, 4028, 4033, 4026, 4030, 4036, 4035, 4032, 4027, 4024, 4022, 4043, 4042, 4037, 4050, 4051, 4044, 4049, 4041, 4040, 4052, 4045, 4039, 4038, 4048, 4046, 4047, 4061, 4056, 4057, 4062, 4067, 4060, 4055, 4058, 4053, 4066, 4068, 4065, 4054, 4063, 4064, 4059, 4070, 4078, 4072, 4082, 4081, 4069, 4074, 4080, 4073, 4075, 4071, 4083, 4077, 4084, 4079, 4076, 4099, 4095, 4093, 4085, 4089, 4100, 4091, 4096, 4097, 4094, 4086, 4088, 4090, 4087, 4092, or 4098. In some embodiments, the 11-16 nucleotide editing template encodes a wildtype NCF1 gene sequence and comprises the sequence corresponding to sequence number 4020, 4021, 4037, 4054, 4072, or 4098. In some embodiments, the 11-16 nucleotide editing template encodes one or more PAM silencing edits can comprise and comprises the sequence corresponding to sequence number 4023, 4034, 4031, 4029, 4025, 4028, 4033, 4026, 4030, 4036, 4035, 4032, 4027, 4024, 4022, 4043, 4042, 4050, 4051, 4044, 4049, 4041, 4040, 4052, 4045, 4039, 4038, 4048, 4046, 4047, 4061, 4056, 4057, 4062, 4067, 4060, 4055, 4058, 4053, 4066, 4068, 4065, 4063, 4064, 4059, 4070, 4078, 4082, 4081, 4069, 4074, 4080, 4073, 4075, 4071, 4083, 4077, 4084, 4079, 4076, 4099, 4095, 4093, 4085, 4089, 4100, 4091, 4096, 4097, 4094, 4086, 4088, 4090, 4087, or 4092.

In some embodiments, the editing template is 12 to 16 nucleotides in length and comprises the sequence corresponding to sequence number 4023, 4021, 4034, 4031, 4029, 4025, 4028, 4033, 4026, 4030, 4036, 4035, 4032, 4027, 4024, 4022, 4043, 4042, 4037, 4050, 4051, 4044, 4049, 4041, 4040, 4052, 4045, 4039, 4038, 4048, 4046, 4047, 4061, 4056, 4057, 4062, 4067, 4060, 4055, 4058, 4053, 4066, 4068, 4065, 4054, 4063, 4064, 4059, 4070, 4078, 4072, 4082, 4081, 4069, 4074, 4080, 4073, 4075, 4071, 4083, 4077, 4084, 4079, 4076, 4099, 4095, 4093, 4085, 4089, 4100, 4091, 4096, 4097, 4094, 4086, 4088, 4090, 4087, 4092, or 4098. In some embodiments, the 12-16 nucleotide editing template encodes one or more PAM silencing edits can comprise and comprises the sequence corresponding to sequence number 4023, 4034, 4031, 4029, 4025, 4028, 4033, 4026, 4030, 4036, 4035, 4032, 4027, 4024, 4022, 4043, 4042, 4050, 4051, 4044, 4049, 4041, 4040, 4052, 4045, 4039, 4038, 4048, 4046, 4047, 4061, 4056, 4057, 4062, 4067, 4060, 4055, 4058, 4053, 4066, 4068, 4065, 4063, 4064, 4059, 4070, 4078, 4082, 4081, 4069, 4074, 4080, 4073, 4075, 4071, 4083, 4077, 4084, 4079, 4076, 4099, 4095, 4093, 4085, 4089, 4100, 4091, 4096, 4097, 4094, 4086, 4088, 4090, 4087, or 4092. In some embodiments, the 12-16 nucleotide editing template encodes a wildtype NCF1 gene sequence and comprises the sequence corresponding to sequence number 4021, 4037, 4054, 4072, or 4098.

In some embodiments, the editing template is 12 nucleotides in length. In some embodiments, the 12-nucleotide editing template comprises the sequence corresponding to sequence number 4023, 4021, 4034, 4031, 4029, 4025, 4028, 4033, 4026, 4030, 4036, 4035, 4032, 4027, 4024, or 4022. In some embodiments, the 12-nucleotide editing template comprises the sequence corresponding to sequence number 4021.

The PBS can be, for example, 3 to 19 nucleotides in length and can comprise the sequence corresponding to any one of sequence numbers 4002-4018. In some cases, a PBS length of no more than 3 nucleotides less than the PEgRNA spacer length is chosen. In some embodiments, the PBS is at least 8 nucleotides in length and comprises at its 5′ end the sequence corresponding to any one of sequence numbers 4007-4018. In some embodiments, the PBS is at least 9, 10, or 11 nucleotides in length. In some embodiments, the PBS is at least 12 nucleotides in length and comprises at its 5′ end the sequence corresponding to any one of sequence numbers 4011-4018. In some embodiments, the PBS is 11 to 14 nucleotides in length, and comprises the sequence corresponding to anyone of sequence numbers 4010, 4011, 4012, and 4013. In some embodiments, the PBS is 12 to 14 nucleotide in length, and comprises the sequence corresponding to anyone of sequence numbers 4011, 4012, and 4013. In some embodiments, the PBS is 11 nucleotides in length and comprises the sequence corresponding to sequence number 4010. In some embodiments, the PBS is 12 nucleotides in length and comprises the sequence corresponding to sequence number 4011. In some embodiments, the PBS is 13 nucleotides in length and comprises the sequence corresponding to sequence number 4012. In some embodiments, the PBS is 13 nucleotides in length and comprises the sequence corresponding to sequence number 4013.

The PEgRNA can comprise, from 5′ to 3′, the spacer, the gRNA core, the editing template, and the PBS. The 3′ end of the editing template can be contiguous with the 5′ end of the PBS. The PEgRNA can comprise multiple RNA molecules or can be a single RNA molecule. Exemplary PEgRNAs provided in Table 12 can comprise a sequence corresponding to sequence number 5529, 5528, 5530, 5532, 5531, 5534, 5533, 5537, 5538, 5535, 5540, 5539, 5536, 5541, 5545, 5544, 5542, 5543, 5558, 5552, 5546, 5556, 5563, 5549, 5560, 5565, 5562, 5561, 5553, 5550, 5555, 5554, 5557, 5548, 5551, 5567, 5559, 5547, 5566, 5564, 5584, 5585, 5590, 5572, 5570, 5581, 5583, 5586, 5588, 5589, 5574, 5575, 5580, 5578, 5579, 5587, 5577, 5582, 5569, 5568, 5573, 5576, 5571, 5602, 5597, 5600, 5596, 5606, 5604, 5595, 5605, 5594, 5593, 5613, 5609, 5610, 5608, 5601, 5599, 5598, 5591, 5607, 5592, 5603, 5611, 5612, 5615, 5630, 5627, 5632, 5634, 5626, 5622, 5619, 5633, 5635, 5617, 5618, 5624, 5623, 5614, 5620, 5628, 5616, 5621, 5625, 5629, 5631, 5648, 5652, 5647, 5637, 5638, 5640, 5651, 5636, 5646, 5655, 5641, 5639, 5656, 5654, 5642, 5645, 5649, 5650, 5658, 5643, 5644, 5657, 5653, 5665, 5661, 5663, 5660, 5664, 5659, 5662, 5676, 5677, 5672, 5673, 5680, 5670, 5669, 5674, 5684, 5675, 5681, 5682, 5666, 5667, 5668, 5683, 5671, 5678, 5685, 5679, 5687, 5686, 5688, 5689, 5690, 5691, or 5692. In some embodiments, the PEgRNA comprises a sequence corresponding to sequence number 5637, 5682, 5689, 5683, 5690, 5692, 5563, 5606, 5647, 5605, 5644, 5672, 5538, 5613, 5614, 5638, 5665, 5618, 5659, 5661, 5688, 5529, 5537, 5543, 5561, 5570, 5542, 5569, 5571, 5634, 5667, 5668, 5666, 5592, 5604, or 5602. In some embodiments, the PEgRNA comprises a sequence corresponding to sequence number 5637, 5682, 5689, 5683, 5690, 5692, 5563, 5606, 5647, 5605, 5644, 5672, 5613, 5537, 5618, 5542, 5661, 5571, 5614, 5543, 5665, 5570, 5638, 5561, 5688, or 5634. In some embodiments, the PEgRNA comprises a sequence corresponding to sequence number 5637, 5563, 5683, 5605, 5659, 5569, 5618, or 5542.

Any PEgRNA exemplified in Table 12 may comprise, or further comprise, a 3′ motif at the 3′ end of the extension arm, for example, a hairpin-forming motif or a series of 1, 2, 3, 4, 5, 6, 7 or more U nucleotides. In some embodiments, the PEgRNA comprises 4 U nucleotides at its 3′ end. Without being bound by theory, such 3′ motifs are believed to increase PEgRNA stability. The PEgRNA may alternatively or additionally comprise one or more chemical modifications, such as phosphorothioate (PS) bond(s), 2′-O-methylated (2′-Ome) nucleotides, or a combination thereof. In some embodiments, the PEgRNA comprise 3′ mN*mN*mN*N and 5′mN*mN*mN* modifications, where m indicates that the nucleotide contains a 2′-O-Me modification and a * indicates the presence of a phosphorothioate bond. PEgRNA sequences exemplified in Table 12 may alternatively be adapted for expression from a DNA template, for example, by including a 5′ terminal G if the spacer of the PEgRNA begins with a nucleotide, other than G by including 6 or 7 U nucleotides at the 3′ end of the extension arm, or both.

Any of the PEgRNAs of Table 12 can be used in a Prime Editing system further comprising a nick guide RNA (ngRNA). Such ngRNA can comprise (a) a spacer comprising at its 3′ end a sequence corresponding to nucleotides 5-20 of any ngRNA spacer corresponding to sequence number 461, 462, 467, 431, 464, 466, 454, 457, 459, 433, 417, 449, 425, 439, 460, 426, 5495, 5501, 5510, 5502, 5509, 5496, 5497, 458, 5506, 5504, 5498, 450, 429, 423, 434, 436, 420, 418, 455, 5499, 432, 443, 424, 421, 451, 445, 435, 430, 446, 427, 422, 463, 452, 453, 444, 419, 442, 5508, 441, 5505, 447, 440, 438, 428, 5503, or 5500 and (b) a gRNA core capable of complexing with a Cas9 protein. For example, the sequence in the spacer of the ngRNA can comprise nucleotides 5-20, 4-20, 3-20, 2-20, or 1-20 of sequence number 461, 462, 467, 431, 464, 466, 454, 457, 459, 433, 417, 449, 425, 439, 460, 426, 5495, 5501, 5510, 5502, 5509, 5496, 5497, 458, 5506, 5504, 5498, 450, 429, 423, 434, 436, 420, 418, 455, 5499, 432, 443, 424, 421, 451, 445, 435, 430, 446, 427, 422, 463, 452, 453, 444, 419, 442, 5508, 441, 5505, 447, 440, 438, 428, 5503, or 5500. In some embodiments, the spacer of the ngRNA is a ngRNA spacer listed in Table 12. The ngRNA spacers in Table 12 are annotated with their PAM sequences, enabling selection of an appropriate Cas9 protein. It can be advantageous to select a ngRNA spacer that has a PAM sequence compatible with the Cas9 protein used in the Prime Editor, thus avoiding the need to use two different Cas9 proteins. The ngRNA is capable of directing a complexed Cas9 protein to bind the edit strand of the NCF1 gene (or NCF1B/NCF1C pseudogene); thus, a complexed Cas9 nickase containing a nuclease inactivating mutation in the HNH domain will nick the non-edit strand.

A PE3 ngRNA spacer has perfect complementarity to the edit strand both pre- and post-edit; a PE3b ngRNA spacer has perfect complementarity to the edit strand post-edit. A PE3b*N ngRNA spacer, where N is the integer following the * as indicated in the table, has perfect complementarity to the edit strand post-edit containing the specific edit encoded by the editing template annotated with the same *N. For example, a PE3b*1 ngRNA spacer has 100% complementary with the portion of the edit strand containing the edit encoded by a RTT annotated with * 1. In some embodiments, a PE3 ngRNA spacer comprises perfect complementarity to the edit strand pre- and post-edit, and comprises at its 3′ end a sequence corresponding to nucleotides 15-20 of sequence numbers 461, 462, 467, 431, 464, 466, 454, 457, 459, 433, 417, 449, 425, 439, 460, 426, 458, 450, 429, 423, 434, 436, 420, 418, 455, 432, 443, 424, 421, 451, 445, 435, 430, 446, 427, 422, 463, 452, 453, 444, 419, 442, 441, 447, 440, 438, or 428. In some embodiments, a PE3 ngRNA spacer comprises perfect complementarity to the edit strand pre- and post-edit, and comprises at its 3′ end a sequence corresponding to sequence numbers 405, 403, 411, 406, 407, 413, 414, 409, 415, 404, 412, 416, 410, 408, 461, 462, 467, 431, 464, 466, 454, 457, 459, 433, 417, 449, 425, 439, 460, 426, 458, 450, 429, 423, 434, 436, 420, 418, 455, 432, 443, 424, 421, 451, 445, 435, 430, 446, 427, 422, 463, 452, 453, 444, 419, 442, 441, 447, 440, 438, 428, 473, 472, 471, 474, 469, 470, 468, 476, 478, 479, 477, 480, or 475.

In some embodiments, a PE3b ngRNA spacer comprises at its 3′ end:
SEQ ID NO 5495 wherein the RT of the PEgRNA comprises at its 3′ end SEQ ID NO 4022,
SEQ ID NO 5501 wherein the RT of the PEgRNA comprises at its 3′ end SEQ ID NO 4025,
SEQ ID NO 5510 wherein the RT of the PEgRNA comprises at its 3′ end SEQ ID NO 4029,
SEQ ID NO 5502 wherein the RT of the PEgRNA comprises at its 3′ end SEQ ID NO 4028,
SEQ ID NO 5509 wherein the RTT of the PEgRNA comprises at its 3′ end SEQ ID NO 4026,
SEQ ID NO 5496 wherein the RTT of the PEgRNA comprises at its 3′ end SEQ ID NO 4035,
SEQ ID NO 5497 wherein the RT of the PEgRNA comprises at its 3′ end SEQ ID NO 4031,
SEQ ID NO 5506 wherein the RTT of the PEgRNA comprises at its 3′ end SEQ ID NO 4024,
SEQ ID NO 5504 wherein the RT of the PEgRNA comprises at its 3′ end SEQ ID NO 4030,
SEQ ID NO 5498 wherein the RT of the PEgRNA comprises at its 3′ end SEQ ID NO 4032,
SEQ ID NO 5499 wherein the RTT of the PEgRNA comprises at its 3′ end SEQ ID NO 4027,
SEQ ID NO 5508 wherein the RT of the PEgRNA comprises at its 3′ end SEQ ID NO 4023,
SEQ ID NO 5505 wherein the RIT of the PEgRNA comprises at its 3′ end SEQ ID NO 4033,
SEQ ID NO 5503 wherein the RIT of the PEgRNA comprises at its 3′ end SEQ ID NO 4034,
SEQ ID 5500 NO wherein the RTT of the PEgRNA comprises at its 3′ end SEQ ID NO 4036.

In some embodiments, the nick-to-nick distance between the ngRNA generated nick and the PEgRNA generated nick is 41 to 96 nucleotides. In some embodiments, the sequence in the spacer of the ngRNA can comprise nucleotides 5-20 of sequence number 436, 2130, 442, 421, 445, 451, 424, 422, 429, 436, or 424. In some embodiments, the sequence in the spacer of the ngRNA can comprise the sequence corresponding to sequence number 436, 2130, 442, 421, 445, 451, 424, 422, 429, 436, or 424.

Exemplary ngRNA provided in Table 12 can comprise a sequence corresponding to sequence number 579, 578, 581, 580, 5698, 595, 592, 594, 5700, 5699, 5702, 584, 582, 586, 593, 5694, 5707, 5701, 5697, 5704, 591, 589, 583, 587, 5703, 588, 5696, 590, 5693, 5705, 5695, 5706, 585, 5712, 5708, 5721, 5710, 5720, 5718, 5709, 5716, 5719, 5713, 5715, 5711, 5714, 5722, 5717, 607, 603, 602, 596, 5521, 481, 5515, 5523, 5526, 5520, 5522, 5516, 5525, 5527, 5517, 598, 605, 5518, 608, 600, 5514, 5524, 5513, 601, 597, 604, 599, 606, or 5519. In some embodiments, the ngRNA comprises a sequence corresponding to sequence number 584, 606, 585, 2143, 595, 594, 583, 592, 591, 590, 599, 2145, 604, 597, 602, 596, 598, or 600.

Any ngRNA sequence provided in Table 12 may comprise, or further comprise, a 3′ motif at their 3′ end, for example, a series of 1, 2, 3, 4, 5, 6, 7 or more U nucleotides. In some embodiments, the ngRNA comprises 4 U nucleotides at its 3′ end. Without being bound by theory, such 3′ motifs are believed to increase ngRNA stability. The ngRNA may alternatively or additionally comprise one or more chemical modifications, such as phosphorothioate (PS) bond(s), 2′-O-methylated (2′-Ome) nucleotides, or a combination thereof. In some embodiments, the ngRNA comprise 3′ mN*mN*mN*N and 5′mN*mN*mN* modifications, where m indicates that the nucleotide contains a 2′-O-Me modification and a * indicates the presence of a phosphorothioate bond. NgRNA sequences may alternatively be adapted for expression from a DNA template, for example, by including a 5′ terminal G if the spacer of the ngRNA begins with a nucleotide other than G, by including 6 or 7 U nucleotides at the 3′ end of the ngRNA, or both.

Exemplary PEgRNA and ngRNA from Table 12 are further excerpted in Table 77 below. All these sequences contained in Table 77 are RNA sequences; however, the Us are presented as Ts to be consistent with ST.26 convention.

TABLE 77
Exemplary PEgRNA and ngRNA from Table 12

| Description | Sequence |
|---|---|
| PEgRNA | TCACCAGGAACATGTACCTGGTTTTAGAGCTAGAAATAGCAAGTTA |
| | AAATAAGGCTAGTCCGTTATCAACTTGAAAAAGTGGCACCGAGTCG |
| | GTGCTTTCCCCCAGGTGTACATGTTCCTTTT |
| | (SEQ ID NO: 5618; Length: 123) |
| | spacer: TCACCAGGAACATGTACCTG(SEQ ID NO: 3999; Length: 20) |
| | RTT: TTTCCCCCAGGT(SEQ ID NO: 4021; Length: 12) |
| | PBS: GTACATGTTCC(SEQ ID NO: 4010; Length: 11) |
| PEgRNA | TCACCAGGAACATGTACCTGGTTTTAGAGCTAGAAATAGCAAGTTA |
| | AAATAAGGCTAGTCCGTTATCAACTTGAAAAAGTGGCACCGAGTCG |
| | GTGCTTTCCCCCAGGTGTACATGTTCC |
| | (SEQ ID NO: 5542; Length: 119) |
| | spacer: TCACCAGGAACATGTACCTG(SEQ ID NO: 3999; Length: 20) |
| | RTT: TTTCCCCCAGGT(SEQ ID NO: 4021; Length: 12) |
| | PBS: GTACATGTTCC(SEQ ID NO: 4010; Length: 11) |
| PEgRNA | TCACCAGGAACATGTACCTGGTTTTAGAGCTAGAAATAGCAAGTTA |
| | AAATAAGGCTAGTCCGTTATCAACTTGAAAAAGTGGCACCGAGTCG |
| | GTGCTTTCCCCCAGGTGTACATGTTCCTTTTT |
| | (SEQ ID NO: 5637; Length: 124) |
| | spacer: TCACCAGGAACATGTACCTG(SEQ ID NO: 3999; Length: 20) |
| | RTT: TTTCCCCCAGGT(SEQ ID NO: 4021; Length: 12) |
| | PBS: GTACATGTTCCT(SEQ ID NO: 4011; Length: 12) |
| PEgRNA | TCACCAGGAACATGTACCTGGTTTTAGAGCTAGAAATAGCAAGTTA |
| | AAATAAGGCTAGTCCGTTATCAACTTGAAAAAGTGGCACCGAGTCG |
| | GTGCTTTCCCCCAGGTGTACATGTTCCT |
| | (SEQ ID NO: 5563; Length: 120) |
| | spacer: TCACCAGGAACATGTACCTG(SEQ ID NO: 3999; Length: 20) |
| | RTT: TTTCCCCCAGGT(SEQ ID NO: 4021; Length: 12) |
| | PBS: GTACATGTTCCT(SEQ ID NO: 4011; Length: 12) |
| PEgRNA | TCACCAGGAACATGTACCTGGTTTTAGAGCTAGAAATAGCAAGTTA |
| | AAATAAGGCTAGTCCGTTATCAACTTGAAAAAGTGGCACCGAGTCG |
| | GTGCTTTCCCCCAGGTGTACATGTTCCTGTTTT |
| | (SEQ ID NO: 5659; Length: 125) |
| | spacer: TCACCAGGAACATGTACCTG(SEQ ID NO: 3999; Length: 20) |
| | RTT: TTTCCCCCAGGT(SEQ ID NO: 4021; Length: 12) |
| | PBS: GTACATGTTCCTG(SEQ ID NO: 4012; Length: 13) |
| PERNA | TCACCAGGAACATGTACCTGGTTTTAGAGCTAGAAATAGCAAGTTA |
| | AAATAAGGCTAGTCCGTTATCAACTTGAAAAAGTGGCACCGAGTCG |
| | GTGCTTTCCCCCAGGTGTACATGTTCCTG |
| | (SEQ ID NO: 5569; Length: 121) |
| | spacer: TCACCAGGAACATGTACCTG(SEQ ID NO: 3999; Length: 20) |
| | RTT: TTTCCCCCAGGT(SEQ ID NO: 4021; Length: 12) |
| | PBS: GTACATGTTCCTG(SEQ ID NO: 4012; Length: 13) |
| PEgRNA | TCACCAGGAACATGTACCTGGTTTTAGAGCTAGAAATAGCAAGTTA |
| | AAATAAGGCTAGTCCGTTATCAACTTGAAAAAGTGGCACCGAGTCG |
| | GTGCTTTCCCCCAGGTGTACATGTTCCTGGTTTT |
| | (SEQ ID NO: 5683; Length: 126) |
| | spacer: TCACCAGGAACATGTACCTG(SEQ ID NO: 3999; Length: 20) |
| | RTT: TTTCCCCCAGGT(SEQ ID NO: 4021; Length: 12) |
| | PBS: GTACATGTTCCTGG(SEQ ID NO: 4013; Length: 14) |
| PEgRNA | TCACCAGGAACATGTACCTGGTTTTAGAGCTAGAAATAGCAAGTTA |
| | AAATAAGGCTAGTCCGTTATCAACTTGAAAAAGTGGCACCGAGTCG |
| | GTGCTTTCCCCCAGGTGTACATGTTCCTGG |
| | (SEQ ID NO: 5605; Length: 122) |
| | spacer: TCACCAGGAACATGTACCTG(SEQ ID NO: 3999; Length: 20) |
| | RTT: TTTCCCCCAGGT(SEQ ID NO: 4021; Length: 12) |
| | PBS: GTACATGTTCCTGG(SEQ ID NO: 4013; Length: 14) |
| PE3ngRNA | GGTCCTCACCCCAATCCTCTGTTTTAGAGCTAGAAATAGCAAGTTAA |
| | AATAAGGCTAGTCCGTTATCAACTTGAAAAAGTGGCACCGAGTCGG |
| | TGCTTTT(SEQ ID NO: 600; Length: 100) |
| | spacer: GGTCCTCACCCCAATCCTCT(SEQ ID NO: 422; Length: 20) |
| PE3ngRNA | GGTCCTCACCCCAATCCTCTGTTTTAGAGCTAGAAATAGCAAGTTAA |
| | AATAAGGCTAGTCCGTTATCAACTTGAAAAAGTGGCACCGAGTCGG |
| | TGC(SEQ ID NO: 590; Length: 96) |
| | spacer: GGTCCTCACCCCAATCCTCT(SEQ ID NO: 422; Length: 20) |
| PE3ngRNA | TCCTCTGGGCTTCCTCCAGTGTTTTAGAGCTAGAAATAGCAAGTTAA |
| | AATAAGGCTAGTCCGTTATCAACTTGAAAAAGTGGCACCGAGTCGG |
| | TGCTTTT(SEQ ID NO: 598; Length: 100) |
| | spacer: TCCTCTGGGCTTCCTCCAGT(SEQ ID NO: 424; Length: 20) |
| PE3ngRNA | TCCTCTGGGCTTCCTCCAGTGTTTTAGAGCTAGAAATAGCAAGTTAA |
| | AATAAGGCTAGTCCGTTATCAACTTGAAAAAGTGGCACCGAGTCGG |
| | TGC(SEQ ID NO: 591; Length: 96) |
| | spacer: TCCTCTGGGCTTCCTCCAGT(SEQ ID NO: 424; Length: 20) |
| PE3ngRNA | TGCACACAGCAAAGCCTCTTGTTTTAGAGCTAGAAATAGCAAGTTA |
| | AAATAAGGCTAGTCCGTTATCAACTTGAAAAAGTGGCACCGAGTCG |
| | GTGCTTTT(SEQ ID NO: 599; Length: 100) |
| | spacer: TGCACACAGCAAAGCCTCTT(SEQ ID NO: 436; Length: 20) |
| PE3ngRNA | TGCACACAGCAAAGCCTCTTGTTTTAGAGCTAGAAATAGCAAGTTA |
| | AAATAAGGCTAGTCCGTTATCAACTTGAAAAAGTGGCACCGAGTCG |
| | GTGC(SEQ ID NO: 585; Length: 96) |
| | spacer: TGCACACAGCAAAGCCTCTT(SEQ ID NO: 436; Length: 20) |
| PE3ngRNA | ACACAGCAAAGCCTCTTTGGGTTTTAGAGCTAGAAATAGCAAGTTA |
| | AAATAAGGCTAGTCCGTTATCAACTTGAAAAAGTGGCACCGAGTCG |
| | GTGCTTTT(SEQ ID NO: 606; Length: 100) |
| | spacer: ACACAGCAAAGCCTCTTTGG(SEQ ID NO: 429; Length: 20) |
| PE3ngRNA | ACACAGCAAAGCCTCTTTGGGTTTTAGAGCTAGAAATAGCAAGTTA |
| | AAATAAGGCTAGTCCGTTATCAACTTGAAAAAGTGGCACCGAGTCG |
| | GTGC(SEQ ID NO: 584; Length: 96) |
| | spacer: ACACAGCAAAGCCTCTTTGG(SEQ ID NO: 429; Length: 20) |

### Table 38

Table 38 provides Prime Editing guide RNAs (PEgRNAs) that can be used with any Prime Editor containing a Cas9 protein capable of recognizing an AG or AGG PAM sequence. The PEgRNAs of Table 38 can also be used in Prime Editing systems further comprising a nick guide RNA (ngRNA). Such PEgRNAs and Prime Editing systems can be used, for example, to correct or edit an c.73_74 ΔGT mutation in NCF1, or to edit the corresponding site in NCF1B or NCF1C.

The PEgRNAs exemplified in Table 38 comprise: (a) a spacer comprising at its 3′ end a sequence corresponding to sequence number 19077; (b) a gRNA core capable of complexing with a Cas9 protein, and (c) an extension arm comprising: (i) an editing template at least 10 nucleotides in length and comprising at its 3′ end a sequence corresponding to sequence number 19101, 19103, 19100, or 19102, and (ii) a prime binding site (PBS) comprising at its 5′ end a sequence corresponding to sequence number 16085.

The PEgRNA spacer can be, for example, 16-22 nucleotides in length and can comprise the sequence corresponding to any one of sequence numbers 19077-19083. In some embodiments, the PEgRNA spacer comprises sequence number 19081. The PEgRNA spacers in Table 38 are annotated with their PAM sequence(s), enabling the selection of an appropriate Cas9 protein.

The editing template can be referred to as a reverse transcription template (RTT). The editing template comprises one or more intended nucleotide edits for incorporation into the target NCF1 (or NCF1B/NCF1C) gene by Prime Editing. In some embodiments, the editing template comprises at least 4 nucleotides that is 5′ to the 5′ most intended edit, wherein the at least 4 nucleotides are complementary to the edit strand of the target gene.

In some embodiments, the editing template encodes a wildtype NCF1 gene sequence. For example, the editing template can comprise at its 3′ end the sequence corresponding to sequence number 19103, 19106, 19108, 19112, 19116, 19123, 19126, 19129, 19133, 19136, 19142, 19144, 19150, 19154, 19159, 19160, 19165, 19169, 19174, 19176, 19180, 19185, 19190, 19194, 19197, 19203, 19206, 19211, 19214, 19216, 19221, 19226, 19229, 19232, 19238, 19241, 19247, 19250, 19254, 19257, 19263, 19265, 19268, 19275, 19279, 19283, 19284, 19289, 19293, 19298, 19303, 19305, 19308, 19314, 19318, 19321, 19327, 19328, 19333, 19338, 19343, 19345, 19349, 19352, 19357, 19361, 19364, 19368, 19373, 19379, 19383, 19385, 19390, 19392, 19399, 19403, 19405, 19408, 19415, 19417, 19420, 19426, 19429, 19433, 19438, 19441, 19446, 19451, 19453, 19456, 19461, 19466, 19471, 19473. In some embodiments, the editing template can comprise at its 3′ end the sequence corresponding to sequence number 19103. In some embodiments, the editing template can encode one or more synonymous edits relative to the wildtype NCF1 gene. In some cases, a synonymous edit is a Valine recode edit. For example, the editing template can comprise at its 3′ end the sequence corresponding to sequence number 19101, 19100, 19102, 19105, 19104, 19107, 19110, 19109, 19111, 19113, 19115, 19114, 19118, 19119, 19117, 19122, 19121, 19120, 19127, 19124, 19125, 19131, 19130, 19128, 19135, 19134, 19132, 19139, 19138, 19137, 19141, 19140, 19143, 19145, 19147, 19146, 19149, 19148, 19151, 19155, 19152, 19153, 19158, 19157, 19156, 19161, 19163, 19162, 19167, 19166, 19164, 19168, 19171, 19170, 19173, 19175, 19172, 19179, 19177, 19178, 19181, 19182, 19183, 19187, 19184, 19186, 19189, 19191, 19188, 19193, 19195, 19192, 19199, 19196, 19198, 19201, 19202, 19200, 19207, 19204, 19205, 19209, 19210, 19208, 19212, 19213, 19215, 19218, 19217, 19219, 19222, 19220, 19223, 19227, 19224, 19225, 19228, 19230, 19231, 19234, 19235, 19233, 19236, 19239, 19237, 19240, 19243, 19242, 19244, 19245, 19246, 19248, 19249, 19251, 19253, 19255, 19252, 19256, 19258, 19259, 19260, 19262, 19261, 19266, 19267, 19264, 19269, 19270, 19271, 19272, 19273, 19274, 19277, 19276, 19278, 19281, 19280, 19282, 19287, 19286, 19285, 19290, 19291, 19288, 19295, 19292, 19294, 19296, 19297, 19299, 19302, 19301, 19300, 19304, 19307, 19306, 19311, 19310, 19309, 19313, 19312, 19315, 19316, 19317, 19319, 19320, 19323, 19322, 19326, 19325, 19324, 19330, 19331, 19329, 19332, 19334, 19335, 19339, 19337, 19336, 19340, 19341, 19342, 19347, 19344, 19346, 19350, 19351, 19348, 19355, 19354, 19353, 19358, 19356, 19359, 19362, 19363, 19360, 19367, 19366, 19365, 19370, 19369, 19371, 19372, 19375, 19374, 19377, 19376, 19378, 19381, 19380, 19382, 19384, 19387, 19386, 19388, 19389, 19391, 19395, 19394, 19393, 19396, 19398, 19397, 19400, 19401, 19402, 19406, 19407, 19404, 19410, 19411, 19409, 19412, 19414, 19413, 19419, 19418, 19416, 19423, 19422, 19421, 19424, 19425, 19427, 19428, 19431, 19430, 19432, 19434, 19435, 19439, 19436, 19437, 19440, 19442, 19443, 19445, 19444, 19447, 19450, 19449, 19448, 19454, 19452, 19455, 19458, 19459, 19457, 19460, 19463, 19462, 19464, 19467, 19465, 19468, 19469, 19470, 19474, 19475, 19472. In some embodiments, the editing template encodes a Valine recode for Valine codon GTA, and comprises at its 3′ end a sequence corresponding to sequence number 19101, 19107, 19111, 19113, 19117, 19121, 19125, 19131, 19135, 19139, 19141, 19146, 19151, 19153, 19157, 19163, 19166, 19170, 19172, 19177, 19182, 19184, 19188, 19192, 19196, 19201, 19204, 19208, 19215, 19218, 19223, 19224, 19231, 19235, 19236, 19242, 19245, 19248, 19253, 19256, 19262, 19266, 19269, 19272, 19278, 19281, 19287, 19288, 19295, 19297, 19302, 19304, 19311, 19312, 19316, 19320, 19326, 19331, 19335, 19336, 19341, 19347, 19348, 19353, 19359, 19363, 19367, 19370, 19375, 19376, 19382, 19387, 19388, 19395, 19398, 19401, 19407, 19409, 19414, 19418, 19423, 19425, 19431, 19435, 19436, 19443, 19444, 19449, 19452, 19458, 19462, 19464, 19469, or 19475. In some embodiments, the editing template encodes a Valine recode for Valine codon GTC, and comprises at its 3′ end a sequence corresponding to sequence number 19102, 19105, 19109, 19114, 19119, 19122, 19124, 19128, 19132, 19137, 19140, 19145, 19149, 19152, 19158, 19162, 19164, 19171, 19173, 19179, 19183, 19187, 19189, 19193, 19198, 19200, 19207, 19210, 19213, 19217, 19220, 19225, 19228, 19233, 19239, 19243, 19246, 19251, 19252, 19258, 19261, 19267, 19270, 19274, 19277, 19282, 19286, 19290, 19292, 19296, 19300, 19307, 19310, 19313, 19317, 19323, 19324, 19329, 19334, 19339, 19340, 19344, 19351, 19354, 19356, 19362, 19365, 19371, 19372, 19378, 19381, 19386, 19389, 19393, 19396, 19402, 19404, 19410, 19413, 19416, 19421, 19424, 19430, 19434, 19437, 19442, 19445, 19448, 19455, 19457, 19463, 19467, 19470, or 19474. In some embodiments, the editing template encodes a Valine recode for Valine codon GTT, and comprises at its 3′ end a sequence corresponding to sequence number 19100, 19104, 19110, 19115, 19118, 19120, 19127, 19130, 19134, 19138, 19143, 19147, 19148, 19155, 19156, 19161, 19167, 19168, 19175, 19178, 19181, 19186, 19191, 19195, 19199, 19202, 19205, 19209, 19212, 19219, 19222, 19227, 19230, 19234, 19237, 19240, 19244, 19249, 19255, 19259, 19260, 19264, 19271, 19273, 19276, 19280, 19285, 19291, 19294, 19299, 19301, 19306, 19309, 19315, 19319, 19322, 19325, 19330, 19332, 19337, 19342, 19346, 19350, 19355, 19358, 19360, 19366, 19369, 19374, 19377, 19380, 19384, 19391, 19394, 19397, 19400, 19406, 19411, 19412, 19419, 19422, 19427, 19428, 19432, 19439, 19440, 19447, 19450, 19454, 19459, 19460, 19465, 19468, or 19472.

In some embodiments, the editing template is at least 13 nucleotides in length and comprises at its 3′ end the sequence corresponding to sequence number 19112, 19113, 19114, or 19115. In some embodiments, the editing template encoding a wildtype NCF1 sequence is at least 13 nucleotides in length and comprises at its 3′ end the sequence corresponding to sequence number 19112. In some embodiments, the editing template encoding a Valine recode is at least 13 nucleotides in length and comprises at its 3′ end the sequence corresponding to sequence number 19113, 19114, or 19115.

In some embodiments, the editing template is at least 15 nucleotides in length and comprises at its 3′ end the sequence corresponding to sequence number 19120, 19121, 19122, or 19123. In some embodiments, the editing template encoding a wildtype NCF1 sequence is at least 15 nucleotides in length and comprises at its 3′ end the sequence corresponding to sequence number 19123. In some embodiments, the editing template encoding a Valine recode is at least 15 nucleotides in length and comprises at its 3′ end the sequence corresponding to sequence number 19120, 19121, or 19122.

In some embodiments, the editing template is at least 16 nucleotides in length and comprises at its 3′ end the sequence corresponding to sequence number 19127, 19126, 19124, or 19125. In some embodiments, the editing template encoding a wildtype NCF1 sequence is at least 16 nucleotides in length and comprises at its 3′ end the sequence corresponding to sequence number 19126. In some embodiments, the editing template encoding a Valine recode is at least 16 nucleotides in length and comprises at its 3′ end the sequence corresponding to sequence number 19127, 19124, or 19125.

In some embodiments, the editing template encoding a wildtype NCF1 sequence is at least 17 nucleotides in length and comprises at its 3′ end the sequence corresponding to sequence number 19128, 19129, 19230, or 19131. In some embodiments, the editing template is at least 17 nucleotides in length and comprises at its 3′ end the sequence corresponding to sequence number 19129. In some embodiments, the editing template encoding a Valine recode is at least 17 nucleotides in length and comprises at its 3′ end the sequence corresponding to sequence number 19128, 19230, or 19131.

In some embodiments, the editing template is 13 to 17 nucleotides in length and comprises the sequence corresponding to sequence number 19112, 19116, 19123, 19126, 19129, 19115, 19114, 19118, 19119, 19117, 19122, 19121, 19120, 19127, 19124, 19125, 19131, 19130, or 19128. In some embodiments, the editing template encoding a wildtype NCF1 sequence is 13 to 17 nucleotides in length, and comprises the sequence corresponding to sequence number 19112, 19116, 19123, 19126, or 19129. In some embodiments, the editing template encoding a Valine recode is 13-17 nucleotides in length and comprises the sequence corresponding to sequence number 19115, 19114, 19118, 19119, 19117, 19122, 19121, 19120, 19127, 19124, 19125, 19131, 19130, or 19128.

In some embodiments, the editing template is 13 nucleotides in length and comprises the sequence corresponding to sequence number 19112, 19113, 19114, or 19115. In some embodiments, the editing template encoding a wildtype NCF1 sequence is 13 nucleotides in length and comprises the sequence corresponding to sequence number 19112. In some embodiments, the editing template encoding a Valine recode is 13 nucleotides in length and comprises the sequence corresponding to sequence number 19113, 19114, or 19115.

In some embodiments, the editing template is 15 nucleotides in length and comprises the sequence corresponding to sequence number 19120, 19121, 19122, or 19123. In some embodiments, the editing template encoding a wildtype NCF1 sequence is 15 nucleotides in length and comprises the sequence corresponding to sequence number 19123. In some embodiments, the editing template encoding a Valine recode is 15 nucleotides in length and comprises the sequence corresponding to sequence number 19120, 19121, or 19122.

In some embodiments, the editing template is 16 nucleotides in length and comprises the sequence corresponding to sequence number 19127, 19126, 19124, or 19125. In some embodiments, the editing template encoding a wildtype NCF1 sequence is 16 nucleotides in length and comprises the sequence corresponding to sequence number 19126. In some embodiments, the editing template encoding a Valine recode is 16 nucleotides in length and comprises the sequence corresponding to sequence number 19127, 19124, or 19125.

In some embodiments, the editing template encoding a wildtype NCF1 sequence is 17 nucleotides in length and comprises the sequence corresponding to sequence number 19128, 19129, 19230, or 19131. In some embodiments, the editing template is 17 nucleotides in length and comprises the sequence corresponding to sequence number 19129. In some embodiments, the editing template encoding a Valine recode is 17 nucleotides in length and comprises the sequence corresponding to sequence number 19128, 19230, or 19131.

The PBS can be, for example, 3 to 19 nucleotides in length and can comprise the sequence corresponding to any one of sequence numbers 16085, 19084, 19085, 19086, 19087, 19088, 19089, 19090, 19091, 19092, 19093, 19094, 19095, 19096, 19097, 19098, and 19099. In some cases, a PBS length of no more than 3 nucleotides less than the PEgRNA spacer length is chosen. In some embodiments, the PBS is at least 12 nucleotides in length and comprises at its 5′ end the sequence corresponding to any one of sequence numbers 19092-19099. In some embodiments, the PBS is 12 nucleotides in length and comprises the sequence corresponding to sequence number 19092. In some embodiments, the PBS is at least 13 nucleotides in length and comprises at its 5′ end the sequence corresponding to any one of sequence numbers 19093-19099. In some embodiments, the PBS is 13 nucleotides in length and comprises the sequence corresponding to sequence number 19093. In some embodiments, the PBS is at least 14 nucleotides in length and comprises at its 5′ end the sequence corresponding to any one of sequence numbers 19094-19099. In some embodiments, the PBS is 14 nucleotides in length and comprises the sequence corresponding to sequence number 19094. In some embodiments, the PBS is 12 to 14 nucleotides in length, and comprises the sequence corresponding to anyone of sequence numbers 19092-19094.

The PEgRNA can comprise, from 5′ to 3′, the spacer, the gRNA core, the editing template, and the PBS. The 3′ end of the editing template can be contiguous with the 5′ end of the PBS. The PEgRNA can comprise multiple RNA molecules or can be a single RNA molecule. Exemplary PEgRNAs provided in Table 38 can comprise a sequence corresponding to sequence number 19481, 19482, 19483, 19484, 19486, 19485, 19488, 19490, 19489, 19487, 19493, 19491, 19492, 19495, 19499, 19498, 19502, 19500, 19496, 19501, 19494, 19497, 19503, 19509, 19506, 19507, 19505, 19510, 19504, 19508, 19514, 19519, 19517, 19518, 19511, 19515, 19513, 19516, 19512, 19523, 19527, 19522, 19526, 19525, 19520, 19521, 19524, 19534, 19532, 19530, 19536, 19531, 19529, 19528, 19537, 19533, 19535, 19538, 19541, 19543, 19544, 19542, 19540, 19545, 19539, 19549, 19552, 19551, 19550, 19547, 19548, 19546, 19553, 19554, 19556, 19557, 19555, 19558, 19560, 19559, 19562, 19561, 19563. In some embodiments, the PEgRNA comprises a sequence corresponding to sequence number 19534, 19559, 19550, 19563, 19497, 19531, 19508, 19542, 19516, 19547, 19546, 19512, 19526, 19557, 19524, 19554, 19537, 19560, 19545, 19561, 19535, 19558, 19543, or 19562. In some embodiments, the PEgRNA comprises a sequence corresponding to sequence number 19560 or 19537. In some embodiments, the PEgRNA comprises a sequence corresponding to sequence number 19562 or 19543.

Any PEgRNA exemplified in Table 38 may comprise, or further comprise, a 3′ motif at the 3′ end of the extension arm, for example, a hairpin-forming motif or a series of 1, 2, 3, 4, 5, 6, 7 or more U nucleotides. In some embodiments, the PEgRNA comprises 4 U nucleotides at its 3′ end. Without being bound by theory, such 3′ motifs are believed to increase PEgRNA stability. The PEgRNA may alternatively or additionally comprise one or more chemical modifications, such as phosphorothioate (PS) bond(s), 2′-O-methylated (2′-Ome) nucleotides, or a combination thereof. In some embodiments, the PEgRNA comprise 3′ mN*mN*mN*N and 5′mN*mN*mN* modifications, where m indicates that the nucleotide contains a 2′-O-Me modification and a * indicates the presence of a phosphorothioate bond. PEgRNA sequences exemplified in Table 38 may alternatively be adapted for expression from a DNA template, for example, by including a 5′ terminal G if the spacer of the PEgRNA begins with a nucleotide, other than G by including 6 or 7 U nucleotides at the 3′ end of the extension arm, or both.

Any of the PEgRNAs of Table 38 can be used in a Prime Editing system further comprising a nick guide RNA (ngRNA). Such ngRNA can comprise (a) a spacer comprising at its 3′ end a sequence corresponding to nucleotides 5-20 of any ngRNA spacer corresponding to sequence number 840, 830, 809, 829, 431, 460, 838, 839, 2133, 848, 806, 461, 794, 803, 19478, 2131, 2130, 796, 842, 2139, 856, 849, 833, 828, 462, 467, 810, 464, 843, 832, 801, 2134, 804, 807, 802, 19479, 2138, 800, 857, 792, 2132, 808, 2137, 2135, 19480, 835, 841, 455, 19477, or 2136, and (b) a gRNA core capable of complexing with a Cas9 protein. For example, the sequence in the spacer of the ngRNA can comprise nucleotides 5-20, 4-20, 3-20, 2-20, or 1-20 of sequence number 840, 830, 809, 829, 431, 460, 838, 839, 2133, 848, 806, 461, 794, 803, 19478, 2131, 2130, 796, 842, 2139, 856, 849, 833, 828, 462, 467, 810, 464, 843, 832, 801, 2134, 804, 807, 802, 19479, 2138, 800, 857, 792, 2132, 808, 2137, 2135, 19480, 835, 841, 455, 19477, or 2136. In some embodiments, the ngRNA spacer comprises a sequence corresponding to sequence number 766, 767, 768, 769, 770, 404, 2129, 409, 1820, 772, 774, 407, 406, 405, 777, 790, 408, 840, 830, 809, 829, 431, 460, 838, 839, 2133, 848, 806, 461, 794, 803, 19478, 2131, 2130, 796, 842, 2139, 856, 849, 833, 828, 462, 467, 810, 464, 843, 832, 801, 2134, 804, 807, 802, 19479, 2138, 800, 857, 792, 2132, 808, 2137, 2135, 19480, 835, 841, 455, 19477, 2136, 473, 472, or 479. In some embodiments, the ngRNA spacer is 16 nucleotides in length and comprises the sequence corresponding the sequence number 766. In some embodiments, the ngRNA spacer is 17 nucleotides in length and comprises the sequence corresponding to sequence number 767, 768, 769, or 770. In some embodiments, the ngRNA spacer is 17 nucleotides in length and comprises the sequence corresponding to sequence number 770. In some embodiments, the spacer of the ngRNA is a ngRNA spacer listed in Table 38. The ngRNA spacers in Table 38 are annotated with their PAM sequences, enabling selection of an appropriate Cas9 protein. It can be advantageous to select a ngRNA spacer that has a PAM sequence compatible with the Cas9 protein used in the Prime Editor, thus avoiding the need to use two different Cas9 proteins. The ngRNA is capable of directing a complexed Cas9 protein to bind the edit strand of the NCF1 gene (or NCF1B/NCF1C pseudogene); thus, a complexed Cas9 nickase containing a nuclease inactivating mutation in the HNH domain will nick the non-edit strand.

A PE3 ngRNA spacer has perfect complementarity to the edit strand both pre- and post-edit; a PE3b ngRNA spacer has perfect complementarity to the edit strand post-edit. A PE3b*N ngRNA spacer, where N is the integer following the * as indicated in the table, has perfect complementarity to the edit strand post-edit containing the specific edit encoded by the editing template annotated with the same *N. For example, a PE3b*1 ngRNA spacer has 100% complementary with the portion of the edit strand containing the edit encoded by a RTT annotated with * 1.

In some embodiments, the PE3b ngRNA spacer has perfect complementarity to a wild type NCF1 sequence encoded by the RTT, and comprises at its 3′ end a sequence corresponding to sequence number 766, 767, 768, 769, 770, 829, 839, 803, 849, 833. In some embodiments, the PE3b ngRNA spacer has perfect complementarity to a Valine recode for Valine codon GTA encoded by the RTT, and comprises at its 3′ end a sequence corresponding to sequence number 832, 801, 807, 800, 792. In some embodiments, the PE3b ngRNA spacer has perfect complementarity to a Valine recode for Valine codon GTC encoded by the RTT, and comprises at its 3′ end a sequence corresponding to sequence 809, 828, 802, 857, 808. In some embodiments, the PE3b ngRNA spacer has perfect complementarity to a Valine recode for Valine codon GTT encoded by the RTT, and comprises at its 3′ end a sequence corresponding to sequence number 840, 830, 810, 804, or 835.

In some embodiments, the nick-to-nick distance between the ngRNA generated nick and the PEgRNA generated nick is 4-88 nucleotides. In some embodiments, the nick-to-nick distance between the ngRNA generated nick and the PEgRNA generated nick is 61-72 nucleotides. In some embodiments, the nick-to-nick distance between the ngRNA generated nick and the PEgRNA generated nick is 72-88 nucleotides. In some embodiments, the nick-to-nick distance between the ngRNA generated nick and the PEgRNA generated nick is 4 to 7 nucleotides and comprises at its 3′ end the sequence corresponding the sequence number 770, 767, 768, 769, 766, 849, 803, 839, 833, or 842. In some embodiments, the sequence in the spacer of the ngRNA can comprise at its 3′ end sequence number 838, 2139, 2130, 2133, 19478, 849, 803, 839, 833, 843, 841, 848, or 842. In some embodiments, the sequence in the spacer of the ngRNA can comprise at its 3′ end the sequence of sequence number 838, 2139, 2130, 2133, 19478, 849, 803, 839, 833, 843, 841, 848, 770, 767, 768, 769, 766, or 842. In some embodiments, the ngRNA spacer is a PE3b spacer comprising at its 3′ end nucleotides 5-20 of sequence number 840, 830, 809, 829, 839, 803, 849, 833, 828, 810, 832, 801, 804, 807, 802, 800, 857, 792, 808, 835, nucleotides 2-17 of sequence number 767,768,769, or 770, or sequence number 766. In some embodiments, the ngRNA spacer is a PE3b spacer comprising at its 3′ end sequence number 840, 830, 809, 829, 839, 803, 849, 833, 828, 810, 832, 801, 804, 807, 802, 800, 857, 792, 808, 835, 767, 768, 769, 770, or 766. In some embodiments, the ngRNA spacer is a PE3b spacer having perfect complementarity to a RT encoding a wild type NCF1 sequence and comprises at its 3′ end sequence number 766, nucleotides 2-17 of sequence number 767, 768, 769, or 770, or nucleotides 5-20 of sequence number 829, 839, 803, 849, or 833. In some embodiments, the ngRNA spacer is a PE3b spacer having perfect complementarity to a RT encoding a wild type NCF1 sequence and comprises at its 3′ end sequence number 766, 767, 768, 769, 770, 829, 839, 803, 849, or 833. In some embodiments, the ngRNA spacer is a PE3b spacer having perfect complementarity to a RT encoding a Valine recode edit and comprises at its 3′ end nucleotides 5-20 of sequence number 840, 830, 809, 828, 810, 832, 801, 804, 807, 802, 800, 857, 792, 808, or 835. In some embodiments, the ngRNA spacer is a PE3b spacer having perfect complementarity to a RT encoding a Valine recode edit and comprises at its 3′ end the sequence corresponding to sequence number 840, 830, 809, 828, 810, 832, 801, 804, 807, 802, 800, 857, 792, 808, or 835.

In some embodiments, the sequence in the spacer of the ngRNA can comprise at its 3′ end nucleotides 5-20 of sequence number 833. In some embodiments, the sequence in the spacer of the ngRNA can comprise at its 3′ end nucleotides 5-20 of sequence number 849. In some embodiments, the sequence in the spacer of the ngRNA can comprise at its 3′ end nucleotides 2-17 of sequence number 770. In some embodiments, the sequence in the spacer of the ngRNA is 20 nucleotide in length and comprises sequence number 833. In some embodiments, the sequence in the spacer of the ngRNA is 20 nucleotide in length and comprises sequence number 849. In some embodiments, the sequence in the spacer of the ngRNA is 17 nucleotide in length and comprises sequence number 770.

Exemplary ngRNA provided in Table 38 can comprise a sequence corresponding to sequence number 2140, 19564, 877, 878, 881, 879, 880, 19565, 2141, 892, 891, 884, 883, 882, 888, 887, 889, 885, 886, 2142, 19566, 890, 2143, 895, 893, 896, 894, 899, 906, 900, 904, 2144, 903, 905, 2145, 19567, 902, 897, 901, 898. In some embodiments, the ngRNA comprises a sequence corresponding to sequence number 896, 904, 890, 895, 903, 894, 906, 893, 901, 2145, 2144, 19567, 2141, 19565, 769, 803, 766, 768, 839, 767, 833, 770, 849, 2130, 2133, 19478. In some embodiments, the ngRNA is a PE3b ngRNA and comprises a sequence corresponding to sequence number 893, 894, 895, 896, 890, 901, 878, 880, 881, 879, 877, or 888. In some embodiments. In some embodiments, the ngRNA is a PE3b ngRNA and comprises a sequence corresponding to sequence number 893 or 878. In some embodiments, the ngRNA is a PE3b ngRNA and comprises a sequence corresponding to sequence number 901 or 888. In some embodiments, the ngRNA is a PE3b ngRNA and comprises a sequence corresponding to sequence number 906 or 883.

Any ngRNA sequence provided in Table 38 may comprise, or further comprise, a 3′ motif at their 3′ end, for example, a series of 1, 2, 3, 4, 5, 6, 7 or more U nucleotides. In some embodiments, the ngRNA comprises 4 U nucleotides at its 3′ end. Without being bound by theory, such 3′ motifs are believed to increase ngRNA stability. The ngRNA may alternatively or additionally comprise one or more chemical modifications, such as phosphorothioate (PS) bond(s), 2′-O-methylated (2′-Ome) nucleotides, or a combination thereof. In some embodiments, the ngRNA comprise 3′ mN*mN*mN*N and 5′mN*mN*mN* modifications, where m indicates that the nucleotide contains a 2′-O-Me modification and a * indicates the presence of a phosphorothioate bond. NgRNA sequences may alternatively be adapted for expression from a DNA template, for example, by including a 5′ terminal G if the spacer of the ngRNA begins with a nucleotide other than G, by including 6 or 7 U nucleotides at the 3′ end of the ngRNA, or both.

Exemplary PEgRNA and ngRNA from Table 38 are further excerpted in Table 78 below. All these sequences contained in Table 78 are RNA sequences; however, the Us are presented as Ts to be consistent with ST.26 convention.

TABLE 78
Exemplary PEgRNA and ngRNA from Table 38

| Description | Sequence |
|---|---|
| PEgRNA | CCCGACTCTGGCTTTCCCCCGTTTTAGAGCTAGAAATAGCAAGTTAA |
| | AATAAGGCTAGTCCGTTATCAACTTGAAAAAGTGGCACCGAGTCGG |
| | TGCATGTACACCTGGGGGAAAGCCAGAGTTTT(SEQ ID NO. 19531); |
| | Length: 125 |
| | spacer: CCCGACTCTGGCTTTCCCCC(SEQ ID NO. 19081); |
| | Length: 20 |
| | RTT: ATGTACACCTGGG(SEQ ID NO. 19112); Length: 13 |
| | PBS: GGAAAGCCAGAG(SEQ ID NO. 19092); Length: 12 |
| PEgRNA | CCCGACTCTGGCTTTCCCCCGTTTTAGAGCTAGAAATAGCAAGTTAA |
| | AATAAGGCTAGTCCGTTATCAACTTGAAAAAGTGGCACCGAGTCGG |
| | TGCATGTACACCTGGGGGAAAGCCAGAG(SEQ ID NO. 19497); |
| | Length: 121 |
| | spacer: CCCGACTCTGGCTTTCCCCC(SEQ ID NO. 19081); |
| | Length: 20 |
| | RTT: ATGTACACCTGGG(SEQ ID NO. 19112); Length: 13 |
| | PBS: GGAAAGCCAGAG(SEQ ID NO. 19092); Length: 12 |
| PEgRNA | CCCGACTCTGGCTTTCCCCCGTTTTAGAGCTAGAAATAGCAAGTTAA |
| | AATAAGGCTAGTCCGTTATCAACTTGAAAAAGTGGCACCGAGTCGG |
| | TGCACATGTACACCTGGGGGAAAGCCAGAGTTTT(SEQ ID NO. 19548); |
| | Length: 127 |
| | spacer: CCCGACTCTGGCTTTCCCCC(SEQ ID NO. 19081); |
| | Length: 20 |
| | RTT: ACATGTACACCTGGG(SEQ ID NO. 19123); Length: 15 |
| | PBS: GGAAAGCCAGAG(SEQ ID NO. 19092); Length: 12 |
| PEgRNA | CCCGACTCTGGCTTTCCCCCGTTTTAGAGCTAGAAATAGCAAGTTAA |
| | AATAAGGCTAGTCCGTTATCAACTTGAAAAAGTGGCACCGAGTCGG |
| | TGCACATGTACACCTGGGGGAAAGCCAGAG(SEQ ID NO. 19515); |
| | Length: 123 |
| | spacer: CCCGACTCTGGCTTTCCCCC(SEQ ID NO. 19081); |
| | Length: 20 |
| | RTT: ACATGTACACCTGGG(SEQ ID NO. 19123); Length: 15 |
| | PBS: GGAAAGCCAGAG(SEQ ID NO. 19092); Length: 12 |
| PEgRNA | CCCGACTCTGGCTTTCCCCCGTTTTAGAGCTAGAAATAGCAAGTTAA |
| | AATAAGGCTAGTCCGTTATCAACTTGAAAAAGTGGCACCGAGTCGG |
| | TGCAACATGTACACCTGGGGGAAAGCCAGAGTTTT(SEQ ID |
| | NO. 19554); Length: 128 |
| | spacer: CCCGACTCTGGCTTTCCCCC(SEQ ID NO. 19081); |
| | Length: 20 |
| | RTT: AACATGTACACCTGGG(SEQ ID NO. 19126); Length: 16 |
| | PBS: GGAAAGCCAGAG(SEQ ID NO. 19092); Length: 12 |
| PEgRNA | CCCGACTCTGGCTTTCCCCCGTTTTAGAGCTAGAAATAGCAAGTTAA |
| | AATAAGGCTAGTCCGTTATCAACTTGAAAAAGTGGCACCGAGTCGG |
| | TGCAACATGTACACCTGGGGGAAAGCCAGAG(SEQ ID NO. 19524); |
| | Length: 124 |
| | spacer: CCCGACTCTGGCTTTCCCCC(SEQ ID NO. 19081); |
| | Length: 20 |
| | RTT: AACATGTACACCTGGG(SEQ ID NO. 19126); Length: 16 |
| | PBS: GGAAAGCCAGAG(SEQ ID NO. 19092); Length: 12 |
| PEgRNA | CCCGACTCTGGCTTTCCCCCGTTTTAGAGCTAGAAATAGCAAGTTAA |
| | AATAAGGCTAGTCCGTTATCAACTTGAAAAAGTGGCACCGAGTCGG |
| | TGCGAACATGTACACCTGGGGGAAAGCCAGAGTTTT(SEQ ID |
| | NO. 19558); Length: 129 |
| | spacer: CCCGACTCTGGCTTTCCCCC(SEQ ID NO. 19081); |
| | Length: 20 |
| | RTT: GAACATGTACACCTGGG(SEQ ID NO. 19129); Length: 17 |
| | PBS: GGAAAGCCAGAG(SEQ ID NO. 19092); Length: 12 |
| PEgRNA | CCCGACTCTGGCTTTCCCCCGTTTTAGAGCTAGAAATAGCAAGTTAA |
| | AATAAGGCTAGTCCGTTATCAACTTGAAAAAGTGGCACCGAGTCGG |
| | TGCGAACATGTACACCTGGGGGAAAGCCAGAG(SEQ ID NO. 19535); |
| | Length: 125 |
| | spacer: CCCGACTCTGGCTTTCCCCC(SEQ ID NO. 19081); |
| | Length: 20 |
| | RTT: GAACATGTACACCTGGG(SEQ ID NO. 19129); Length: 17 |
| | PBS: GGAAAGCCAGAG(SEQ ID NO. 19092); Length: 12 |
| PEgRNA | CCCGACTCTGGCTTTCCCCCGTTTTAGAGCTAGAAATAGCAAGTTAA |
| | AATAAGGCTAGTCCGTTATCAACTTGAAAAAGTGGCACCGAGTCGG |
| | TGCATGTACACCTGGGGGAAAGCCAGAGTTTTT(SEQ ID NO. 19542); |
| | Length: 126 |
| | spacer: CCCGACTCTGGCTTTCCCCC(SEQ ID NO. 19081); |
| | Length: 20 |
| | RTT: ATGTACACCTGGG(SEQ ID NO. 19112); Length: 13 |
| | PBS: GGAAAGCCAGAGT(SEQ ID NO. 19093); Length: 13 |
| PEgRNA | CCCGACTCTGGCTTTCCCCCGTTTTAGAGCTAGAAATAGCAAGTTAA |
| | AATAAGGCTAGTCCGTTATCAACTTGAAAAAGTGGCACCGAGTCGG |
| | TGCATGTACACCTGGGGGAAAGCCAGAGT(SEQ ID NO. 19508); |
| | Length: 122 |
| | spacer: CCCGACTCTGGCTTTCCCCC(SEQ ID NO. 19081); |
| | Length: 20 |
| | RTT: ATGTACACCTGGG(SEQ ID NO. 19112); Length: 13 |
| | PBS: GGAAAGCCAGAGT(SEQ ID NO. 19093); Length: 13 |
| PEgRNA | CCCGACTCTGGCTTTCCCCCGTTTTAGAGCTAGAAATAGCAAGTTAA |
| | AATAAGGCTAGTCCGTTATCAACTTGAAAAAGTGGCACCGAGTCGG |
| | TGCACATGTACACCTGGGGGAAAGCCAGAGTTTTT(SEQ ID |
| | NO. 19557); Length: 128 |
| | spacer: CCCGACTCTGGCTTTCCCCC(SEQ ID NO. 19081); |
| | Length: 20 |
| | RTT: ACATGTACACCTGGG(SEQ ID NO. 19123); Length: 15 |
| | PBS: GGAAAGCCAGAGT(SEQ ID NO. 19093); Length: 13 |
| PEgRNA | CCCGACTCTGGCTTTCCCCCGTTTTAGAGCTAGAAATAGCAAGTTAA |
| | AATAAGGCTAGTCCGTTATCAACTTGAAAAAGTGGCACCGAGTCGG |
| | TGCACATGTACACCTGGGGGAAAGCCAGAGT(SEQ ID NO. 19526); |
| | Length: 124 |
| | spacer: CCCGACTCTGGCTTTCCCCC(SEQ ID NO. 19081); |
| | Length: 20 |
| | RTT: ACATGTACACCTGGG(SEQ ID NO. 19123); Length: 15 |
| | PBS: GGAAAGCCAGAGT(SEQ ID NO. 19093); Length: 13 |
| PEgRNA | CCCGACTCTGGCTTTCCCCCGTTTTAGAGCTAGAAATAGCAAGTTAA |
| | AATAAGGCTAGTCCGTTATCAACTTGAAAAAGTGGCACCGAGTCGG |
| | TGCAACATGTACACCTGGGGGAAAGCCAGAGTTTTT(SEQ ID |
| | NO. 19560); Length: 129 |
| | spacer: CCCGACTCTGGCTTTCCCCC(SEQ ID NO. 19081); |
| | Length: 20 |
| | RTT: AACATGTACACCTGGG(SEQ ID NO. 19126); Length: 16 |
| | PBS: GGAAAGCCAGAGT(SEQ ID NO. 19093); Length: 13 |
| PEgRNA | CCCGACTCTGGCTTTCCCCCGTTTTAGAGCTAGAAATAGCAAGTTAA |
| | AATAAGGCTAGTCCGTTATCAACTTGAAAAAGTGGCACCGAGTCGG |
| | TGCAACATGTACACCTGGGGGAAAGCCAGAGT(SEQ ID NO. 19537); |
| | Length: 125 |
| | spacer: CCCGACTCTGGCTTTCCCCC(SEQ ID NO. 19081); |
| | Length: 20 |
| | RTT: AACATGTACACCTGGG(SEQ ID NO. 19126); Length: 16 |
| | PBS: GGAAAGCCAGAGT(SEQ ID NO. 19093); Length: 13 |
| PEgRNA | CCCGACTCTGGCTTTCCCCCGTTTTAGAGCTAGAAATAGCAAGTTAA |
| | AATAAGGCTAGTCCGTTATCAACTTGAAAAAGTGGCACCGAGTCGG |
| | TGCGAACATGTACACCTGGGGGAAAGCCAGAGTTTTT(SEQ ID |
| | NO. 19562); Length: 130 |
| | spacer: CCCGACTCTGGCTTTCCCCC(SEQ ID NO. 19081); |
| | Length: 20 |
| | RTT: GAACATGTACACCTGGG(SEQ ID NO. 19129); Length: 17 |
| | PBS: GGAAAGCCAGAGT(SEQ ID NO. 19093); Length: 13 |
| PEgRNA | CCCGACTCTGGCTTTCCCCCGTTTTAGAGCTAGAAATAGCAAGTTAA |
| | AATAAGGCTAGTCCGTTATCAACTTGAAAAAGTGGCACCGAGTCGG |
| | TGCGAACATGTACACCTGGGGGAAAGCCAGAGT(SEQ ID NO. 19543); |
| | Length: 126 |
| | spacer: CCCGACTCTGGCTTTCCCCC(SEQ ID NO. 19081); |
| | Length: 20 |
| | RTT: GAACATGTACACCTGGG(SEQ ID NO. 19129); Length: 17 |
| | PBS: GGAAAGCCAGAGT(SEQ ID NO. 19093); Length: 13 |
| PEgRNA | CCCGACTCTGGCTTTCCCCCGTTTTAGAGCTAGAAATAGCAAGTTAA |
| | AATAAGGCTAGTCCGTTATCAACTTGAAAAAGTGGCACCGAGTCGG |
| | TGCATGTACACCTGGGGGAAAGCCAGAGTCTTTT(SEQ ID NO. 19547); |
| | Length: 127 |
| | spacer: CCCGACTCTGGCTTTCCCCC(SEQ ID NO. 19081); |
| | Length: 20 |
| | RTT: ATGTACACCTGGG(SEQ ID NO. 19112); Length: 13 |
| | PBS: GGAAAGCCAGAGTC(SEQ ID NO. 19094); Length: 14 |
| PEgRNA | CCCGACTCTGGCTTTCCCCCGTTTTAGAGCTAGAAATAGCAAGTTAA |
| | AATAAGGCTAGTCCGTTATCAACTTGAAAAAGTGGCACCGAGTCGG |
| | TGCATGTACACCTGGGGGAAAGCCAGAGTC(SEQ ID NO. 19516); |
| | Length: 123 |
| | spacer: CCCGACTCTGGCTTTCCCCC(SEQ ID NO. 19081); |
| | Length: 20 |
| | RTT: ATGTACACCTGGG(SEQ ID NO. 19112); Length: 13 |
| | PBS: GGAAAGCCAGAGTC(SEQ ID NO. 19094); Length: 14 |
| PEgRNA | CCCGACTCTGGCTTTCCCCCGTTTTAGAGCTAGAAATAGCAAGTTAA |
| | AATAAGGCTAGTCCGTTATCAACTTGAAAAAGTGGCACCGAGTCGG |
| | TGCACATGTACACCTGGGGGAAAGCCAGAGTCTTTT(SEQ ID |
| | NO. 19559); Length: 129 |
| | spacer: CCCGACTCTGGCTTTCCCCC(SEQ ID NO. 19081); |
| | Length: 20 |
| | RTT: ACATGTACACCTGGG(SEQ ID NO. 19123); Length: 15 |
| | PBS: GGAAAGCCAGAGTC(SEQ ID NO. 19094); Length: 14 |
| PEgRNA | CCCGACTCTGGCTTTCCCCCGTTTTAGAGCTAGAAATAGCAAGTTAA |
| | AATAAGGCTAGTCCGTTATCAACTTGAAAAAGTGGCACCGAGTCGG |
| | TGCACATGTACACCTGGGGGAAAGCCAGAGTC(SEQ ID NO. 19534); |
| | Length: 125 |
| | spacer: CCCGACTCTGGCTTTCCCCC(SEQ ID NO. 19081); |
| | Length: 20 |
| | RTT: ACATGTACACCTGGG(SEQ ID NO. 19123); Length: 15 |
| | PBS: GGAAAGCCAGAGTC(SEQ ID NO. 19094); Length: 14 |
| PEgRNA | CCCGACTCTGGCTTTCCCCCGTTTTAGAGCTAGAAATAGCAAGTTAA |
| | AATAAGGCTAGTCCGTTATCAACTTGAAAAAGTGGCACCGAGTCGG |
| | TGCAACATGTACACCTGGGGGAAAGCCAGAGTCTTTT(SEQ ID |
| | NO. 19561); Length: 130 |
| | spacer: CCCGACTCTGGCTTTCCCCC(SEQ ID NO. 19081); |
| | Length: 20 |
| | RTT: AACATGTACACCTGGG(SEQ ID NO. 19126); Length: 16 |
| | PBS: GGAAAGCCAGAGTC(SEQ ID NO. 19094); Length: 14 |
| PEgRNA | CCCGACTCTGGCTTTCCCCCGTTTTAGAGCTAGAAATAGCAAGTTAA |
| | AATAAGGCTAGTCCGTTATCAACTTGAAAAAGTGGCACCGAGTCGG |
| | TGCAACATGTACACCTGGGGGAAAGCCAGAGTC(SEQ ID NO. 19545); |
| | Length: 126 |
| | spacer: CCCGACTCTGGCTTTCCCCC(SEQ ID NO. 19081); |
| | Length: 20 |
| | RTT: AACATGTACACCTGGG(SEQ ID NO. 19126); Length: 16 |
| | PBS: GGAAAGCCAGAGTC(SEQ ID NO. 19094); Length: 14 |
| PEgRNA | CCCGACTCTGGCTTTCCCCCGTTTTAGAGCTAGAAATAGCAAGTTAA |
| | AATAAGGCTAGTCCGTTATCAACTTGAAAAAGTGGCACCGAGTCGG |
| | TGCGAACATGTACACCTGGGGGAAAGCCAGAGTCTTTT(SEQ ID |
| | NO. 19563); Length: 131 |
| | spacer: CCCGACTCTGGCTTTCCCCC(SEQ ID NO. 19081); |
| | Length: 20 |
| | RTT: GAACATGTACACCTGGG(SEQ ID NO. 19129); Length: 17 |
| | PBS: GGAAAGCCAGAGTC(SEQ ID NO. 19094); Length: 14 |
| PEgRNA | CCCGACTCTGGCTTTCCCCCGTTTTAGAGCTAGAAATAGCAAGTTAA |
| | AATAAGGCTAGTCCGTTATCAACTTGAAAAAGTGGCACCGAGTCGG |
| | TGCGAACATGTACACCTGGGGGAAAGCCAGAGTC(SEQ ID |
| | NO. 19550); Length: 127 |
| | spacer: CCCGACTCTGGCTTTCCCCC(SEQ ID NO. 19081); |
| | Length: 20 |
| | RTT: GAACATGTACACCTGGG(SEQ ID NO. 19129); Length: 17 |
| | PBS: GGAAAGCCAGAGTC(SEQ ID NO. 19094); Length: 14 |
| PE3bngRNA | CCAGGAACATGTACACCGTTTTAGAGCTAGAAATAGCAAGTTAAAA |
| | TAAGGCTAGTCCGTTATCAACTTGAAAAAGTGGCACCGAGTCGGTG |
| | CTTTT(SEQ ID NO: 893; length: 97) |
| | spacer: CCAGGAACATGTACACC(SEQ ID NO: 770; length: 17) |
| PE3bngRNA | CCAGGAACATGTACACCGTTTTAGAGCTAGAAATAGCAAGTTAAAA |
| | TAAGGCTAGTCCGTTATCAACTTGAAAAAGTGGCACCGAGTCGGTG |
| | C(SEQ ID NO: 878; length: 93) |
| | spacer: CCAGGAACATGTACACC(SEQ ID NO: 770; length: 17) |
| PE3bngRNA | CAGGAACATGTACACCTGTTTTAGAGCTAGAAATAGCAAGTTAAAA |
| | TAAGGCTAGTCCGTTATCAACTTGAAAAAGTGGCACCGAGTCGGTG |
| | CTTTT(SEQ ID NO: 894; length: 97) |
| | spacer: CAGGAACATGTACACCT(SEQ ID NO: 767; length: 17) |
| PE3bngRNA | CAGGAACATGTACACCTGTTTTAGAGCTAGAAATAGCAAGTTAAAA |
| | TAAGGCTAGTCCGTTATCAACTTGAAAAAGTGGCACCGAGTCGGTG |
| | C(SEQ ID NO: 880; length: 93) |
| | spacer: CAGGAACATGTACACCT(SEQ ID NO: 767; length: 17) |
| PE3bngRNA | AGGAACATGTACACCTGGTTTTAGAGCTAGAAATAGCAAGTTAAAA |
| | TAAGGCTAGTCCGTTATCAACTTGAAAAAGTGGCACCGAGTCGGTG |
| | CTTTT(SEQ ID NO: 895; length: 97) |
| | spacer: AGGAACATGTACACCTG(SEQ ID NO: 768; length: 17) |
| PE3bngRNA | AGGAACATGTACACCTGGTTTTAGAGCTAGAAATAGCAAGTTAAAA |
| | TAAGGCTAGTCCGTTATCAACTTGAAAAAGTGGCACCGAGTCGGTG |
| | C(SEQ ID NO: 881; length: 93) |
| | spacer: AGGAACATGTACACCTG(SEQ ID NO: 768; length: 17) |
| PE3bngRNA | GGAACATGTACACCTGGGTTTTAGAGCTAGAAATAGCAAGTTAAAA |
| | TAAGGCTAGTCCGTTATCAACTTGAAAAAGTGGCACCGAGTCGGTG |
| | CTTTT(SEQ ID NO: 896; length: 97) |
| | spacer: GGAACATGTACACCTGG(SEQ ID NO: 769; length: 17) |
| PE3bngRNA | GGAACATGTACACCTGGGTTTTAGAGCTAGAAATAGCAAGTTAAAA |
| | TAAGGCTAGTCCGTTATCAACTTGAAAAAGTGGCACCGAGTCGGTG |
| | C(SEQ ID NO: 879; length: 93) |
| | spacer: GGAACATGTACACCTGG(SEQ ID NO: 769; length: 17) |
| PE3bngRNA | GGAACATGTACACCTGGTTTTAGAGCTAGAAATAGCAAGTTAAAAT |
| | AAGGCTAGTCCGTTATCAACTTGAAAAAGTGGCACCGAGTCGGTGC |
| | TTTT(SEQ ID NO: 890; length: 96) |
| | spacer: GGAACATGTACACCTG(SEQ ID NO: 766; length: 16) |
| PE3bngRNA | GGAACATGTACACCTGGTTTTAGAGCTAGAAATAGCAAGTTAAAAT |
| | AAGGCTAGTCCGTTATCAACTTGAAAAAGTGGCACCGAGTCGGTGC |
| | (SEQ ID NO: 877; length: 92) |
| | spacer: GGAACATGTACACCTG(SEQ ID NO: 766; length: 16) |
| PE3bngRNA | TCACCAGGAACATGTACACCGTTTTAGAGCTAGAAATAGCAAGTTA |
| | AAATAAGGCTAGTCCGTTATCAACTTGAAAAAGTGGCACCGAGTCG |
| | GTGCTTTT(SEQ ID NO: 901; length: 100) |
| | spacer: TCACCAGGAACATGTACACC(SEQ ID NO: 849; |
| | length: 20) |
| PE3bngRNA | TCACCAGGAACATGTACACCGTTTTAGAGCTAGAAATAGCAAGTTA |
| | AAATAAGGCTAGTCCGTTATCAACTTGAAAAAGTGGCACCGAGTCG |
| | GTGC(SEQ ID NO: 888; length: 96) |
| | spacer: TCACCAGGAACATGTACACC(SEQ ID NO: 849; |
| | length: 20) |
| PE3bngRNA | CCAGGAACATGTACACCTGGGTTTTAGAGCTAGAAATAGCAAGTTA |
| | AAATAAGGCTAGTCCGTTATCAACTTGAAAAAGTGGCACCGAGTCG |
| | GTGCTTTT(SEQ ID NO: 904; length: 100) |
| | spacer: CCAGGAACATGTACACCTGG(SEQ ID NO: 803; |
| | length: 20) |
| PE3bngRNA | CCAGGAACATGTACACCTGGGTTTTAGAGCTAGAAATAGCAAGTTA |
| | AAATAAGGCTAGTCCGTTATCAACTTGAAAAAGTGGCACCGAGTCG |
| | GTGC(SEQ ID NO: 887; length: 96) |
| | spacer: CCAGGAACATGTACACCTGG(SEQ ID NO: 803; |
| | length: 20) |
| PE3bngRNA | ACCAGGAACATGTACACCTGGTTTTAGAGCTAGAAATAGCAAGTTA |
| | AAATAAGGCTAGTCCGTTATCAACTTGAAAAAGTGGCACCGAGTCG |
| | GTGCTTTT(SEQ ID NO: 903; length: 100) |
| | spacer: ACCAGGAACATGTACACCTG(SEQ ID NO: 839; |
| | length: 20) |
| PE3bngRNA | ACCAGGAACATGTACACCTGGTTTTAGAGCTAGAAATAGCAAGTTA |
| | AAATAAGGCTAGTCCGTTATCAACTTGAAAAAGTGGCACCGAGTCG |
| | GTGC(SEQ ID NO: 892; length: 96) |
| | spacer: ACCAGGAACATGTACACCTG(SEQ ID NO: 839; |
| | length: 20) |
| PE3bngRNA | CACCAGGAACATGTACACCTGTTTTAGAGCTAGAAATAGCAAGTTA |
| | AAATAAGGCTAGTCCGTTATCAACTTGAAAAAGTGGCACCGAGTCG |
| | GTGCTTTT(SEQ ID NO: 906; length: 100) |
| | spacer: CACCAGGAACATGTACACCT(SEQ ID NO: 833; |
| | length: 20) |
| PE3bngRNA | CACCAGGAACATGTACACCTGTTTTAGAGCTAGAAATAGCAAGTTA |
| | AAATAAGGCTAGTCCGTTATCAACTTGAAAAAGTGGCACCGAGTCG |
| | GTGC(SEQ ID NO: 883; length: 96) |
| | spacer: CACCAGGAACATGTACACCT(SEQ ID NO: 833; |
| | length: 20) |

In some embodiments, an ng spacer sequence is complementary to, and may hybridize with the second search target sequence only after an intended nucleotide edit has been incorporated on the edit strand, by the editing template of a PEgRNA. In some embodiments, such a prime editing system maybe referred to as a “PE3b” prime editing system or composition. In some embodiments, the ngRNA comprises a spacer sequence that matches only the edit strand after incorporation of the nucleotide edits, but not the endogenous target gene sequence on the edit strand. Accordingly, in some embodiments, an intended nucleotide edit is incorporated within the ng search target sequence. A ngRNA protospacer may be in close proximity to the PEgRNA spacer, or may be upstream or downstream of the PEgRNA spacer. In some embodiments, the distance generated by the PEgRNA nick site and the ngRNA nick site (referred to as the nick-to-nick distance) is about 3 to about 100 nucleotides. In some embodiments, the distance generated by the PEgRNA nick site and the ngRNA nick site (referred to as the nick-to-nick distance) is about 4-90, 4-80, 4-70, 4-60, 4-50, 4-40, 4-30, 4-20, or 4-10 nucleotides. In some embodiments, the distance generated by the PEgRNA nick site and the ngRNA nick site (referred to as the nick-to-nick distance) is about 10-20, 20-30, 30-40, 40-50, 50-60, 60-70, 70-80, 80-90, or 90-100 nucleotides. In some embodiments, the nick-to-nick distance is about 4-88 nucleotides. In some embodiments, the nick-to-nick distance is about 4-72 nucleotides. In some embodiments, the nick-to-nick distance is about 4-61 nucleotides. In some embodiments, the nick-to-nick distance is about 61-72 nucleotides. In some embodiments, the nick-to-nick distance is about 61-88 nucleotides. In some embodiments, the nick-to-nick distance is about 72-88 nucleotides. In some embodiments, the nick-to-nick distance is about 4-7 nucleotides. In some embodiments, the nick-to-nick distance is 4, 5, 6, or 7 nucleotides. In some embodiments, the nick-to-nick distance is about 41-96 nucleotides. In some embodiments, the nick-to-nick distance is about 41-82 nucleotides. In some embodiments, the nick-to-nick distance is about 41-44 nucleotides. In some embodiments, the nick-to-nick distance is about 44-82 nucleotides. In some embodiments, the nick-to-nick distance is about 44-96 nucleotides. In some embodiments, the nick-to-nick distance is about 82-96 nucleotides. In some embodiments, the nick-to-nick distance is 41, 44, 82, or % nucleotides. In some embodiments, the intended nucleotide edit is incorporated within about 1-10 nucleotides of the position corresponding to the PAM of the ng search target sequence.

The gRNA core of a PEgRNA or ngRNA can be any gRNA scaffold sequence that is capable of interacting with a Cas protein that recognizes the corresponding PAM of the PEgRNA or ngRNA. In some embodiments, gRNA core of a PEgRNA or a ngRNA comprises a sequence selected from SEQ ID Nos:34424-34428.

A PEgRNA and/or an ngRNA of this disclosure, in some embodiments, may include modified nucleotides, e.g., chemically modified DNA or RNA nucleobases, and may include one or more nucleobase analogs (e.g., modifications which might add functionality, such as temperature resilience). In some embodiments, PEgRNAs and/or ngRNAs as described herein may be chemically modified. The phrase “chemical modifications,” as used herein, can include modifications which introduce chemistries which differ from those seen in naturally occurring DNA or RNAs, for example, covalent modifications such as the introduction of modified nucleotides, (e.g., nucleotide analogs, or the inclusion of pendant groups which are not naturally found in DNA or RNA molecules).

In some embodiments, the PEgRNAs provided in the disclosure may further comprise nucleotides added to the 5′ of the PEgRNAs. In some embodiments, the PEgRNA further comprises 1, 2, or 3 additional nucleotides added to the 5′ end. The additional nucleotides can be guanine, cytosine, adenine, or uracil. In some embodiments, the additional nucleotide at the 5′ end of the PEgRNA is a guanine or cytosine. In some embodiments, the additional nucleotides can be chemically or biologically modified.

In some embodiments, the PEgRNAs provided in the disclosure may further comprise nucleotides to the 3′ of the PEgRNAs. In some embodiments, the PEgRNA further comprises 1, 2, or 3 additional nucleotides to the 3′ end. The additional nucleotides can be guanine, cytosine, adenine, or uracil. In some embodiments, the additional nucleotides at the 3′ end of the PEgRNA is a polynucleotide comprising at least 1 uracil. In some embodiments, the additional nucleotides can be chemically or biologically modified.

In some embodiments, a PEgRNA or ngRNA is produced by transcription from a template nucleotide, for example, a template plasmid. In some embodiments, a polynucleotide encoding the PEgRNA or ngRNA is appended with one or more additional nucleotides that improves PEgRNA or ngRNA function or expression, e.g., expression from a plasmid that encodes the PEgRNA or ngRNA. In some embodiments, a polynucleotide encoding a PEgRNA or ngRNA is appended with one or more additional nucleotides at the 5′ end or at the 3′ end. In some embodiments, the polynucleotide encoding the PEgRNA or ngRNA is appended with a guanine at the 5′ end, for example, if the first nucleotide at the 5′ end of the spacer is not a guanine. In some embodiments, a polynucleotide encoding the PEgRNA or ngRNA is appended with nucleotide sequence CACC at the 5′ end. In some embodiments, the polynucleotide encoding the PEgRNA or ngRNA is appended with an additional nucleotide adenine at the 3′ end, for example, if the last nucleotide at the 3′ end of the PBS is a Thymine. In some embodiments, the polynucleotide encoding the PEgRNA or ngRNA is appended with additional nucleotide sequence TTTTT, TTTTTTT, TTTTT, or TTTT at the 3′ end. In some embodiments, the PEgRNA or ngRNA comprises the appended nucleotides from the transcription template. In some embodiments, the PEgRNA or ngRNA further comprises one or more nucleotides at the 5′ end or the 3′ end in addition to spacer, PBS, and RTT sequences. in some embodiments, the PEgRNA or ngRNA further comprises a guanine at the 5′ end, for example, when the first nucleotide at the 5′ end of the spacer is not a guanine. In some embodiments, the PEgRNA or ngRNA further comprises nucleotide sequence CACC at the 5′ end. In some embodiments, the PEgRNA or ngRNA further comprises an adenine at the 3′ end, for example, if the last nucleotide at the 3′ end of the PBS is a thymine. In some embodiments, the PEgRNA or ngRNA further comprises nucleotide sequence UUUUUUU, UUUUUU, UUUUU, or UUUU at the 3′ end.

In some embodiments, the PEgRNAs and/or ngRNAs provided in this disclosure may have undergone a chemical or biological modifications. Modifications may be made at any position within a PEgRNA or ngRNA, and may include modification to a nucleobase or to a phosphate backbone of the PEgRNA or ngRNA. In some embodiments, chemical modifications can be a structure guided modifications. In some embodiments, a chemical modification is at the 5′ end and/or the 3′ end of a PEgRNA. In some embodiments, a chemical modification is at the 5′ end and/or the 3′ end of a ngRNA. In some embodiments, a chemical modification may be within the spacer sequence, the extension arm, the editing template sequence, or the primer binding site of a PEgRNA. In some embodiments, a chemical modification may be within the spacer sequence or the gRNA core of a PEgRNA or a ngRNA. In some embodiments, a chemical modification may be within the 3′ most nucleotides of a PEgRNA or ngRNA. In some embodiments, a chemical modification may be within the 3′ most end of a PEgRNA or ngRNA comprises 1, 2, 3, 4, 5, 6, 7, 8, 9, 10 or more chemically modified nucleotides at the 3′ end. In some embodiments, a PEgRNA or ngRNA comprises 3 contiguous chemically modified nucleotides at the 3′ end. In some embodiments, a chemical modification may be within the 5′ most end of a PEgRNA or ngRNA. In some embodiments, a PEgRNA or ngRNA comprises 1, 2, 3, 4, 5, 6, 7, 8, 9, 10 or more chemically modified nucleotides at the 3′ end. In some embodiments, a PEgRNA or ngRNA comprises 1, 2, 3, 4, 5, 6, 7, 8, 9, 10 or more chemically modified nucleotides at the 5′ end. In some embodiments, a PEgRNA or ngRNA comprises 1, 2, 3, 4, or 5 or more chemically modified nucleotides at the 3′ end. In some embodiments, a PEgRNA or ngRNA comprises 1, 2, 3, 4, or 5 more chemically modified nucleotides at the 5′ end. In some embodiments, a PEgRNA or ngRNA comprises 1, 2, or 3 or more chemically modified nucleotides at the 3′ end. In some embodiments, a PEgRNA or ngRNA comprises 1, 2, or 3 more chemically modified nucleotides at the 5′ end. In some embodiments, a PEgRNA or ngRNA comprises 1, 2, 3, 4, 5, 6, 7, 8, 9, 10 or more contiguous chemically modified nucleotides at the 3′ end. In some embodiments, a PEgRNA or ngRNA comprises 1, 2, 3, 4, 5, 6, 7, 8, 9, 10 or more contiguous chemically modified nucleotides at the 5′ end. In some embodiments, a PEgRNA or ngRNA comprises 1, 2, 3, 4, or 5 contiguous chemically modified nucleotides at the 3′ end. In some embodiments, a PEgRNA or ngRNA comprises 1, 2, 3, 4, or 5 contiguous chemically modified nucleotides at the 5′ end. In some embodiments, a PEgRNA or ngRNA comprises 1, 2, or 3 contiguous chemically modified nucleotides at the 3′ end. In some embodiments, a PEgRNA or ngRNA comprises 1, 2, or 3 contiguous chemically modified nucleotides at the 5′ end. In some embodiments, a PEgRNA or ngRNA comprises 3 contiguous chemically modified nucleotides at the 3′ end. In some embodiments, a PEgRNA or ngRNA comprises 1, 2, 3, 4, 5, or more chemically modified nucleotides near the 3′ end. In some embodiments, a PEgRNA or ngRNA comprises 3 contiguous chemically modified nucleotides at the 3′ end. In some embodiments, a PEgRNA or ngRNA comprises 3 contiguous chemically modified nucleotides at the 5′ end. In some embodiments, a PEgRNA or ngRNA comprises 1, 2, 3, 4, 5, or more chemically modified nucleotides near the 3′ end. In some embodiments, a PEgRNA or ngRNA comprises 1, 2, 3, 4, 5, or more contiguous chemically modified nucleotides near the 3′ end. In some embodiments, a PEgRNA or ngRNA comprises 1, 2, 3, 4, 5, or more chemically modified nucleotides near the 3′ end, where the 3′ most nucleotide is not modified, and the 1, 2, 3, 4, 5, or more chemically modified nucleotides precede the 3′ most nucleotide in a 5′-to-3′ order. In some embodiments, a PEgRNA or ngRNA comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more chemically modified nucleotides near the 3′ end, where the 3′ most nucleotide is not modified, and the 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more chemically modified nucleotides precede the 3′ most nucleotide in a 5′-to-3′ order.

In some embodiments, a PEgRNA or ngRNA comprises one or more chemical modified nucleotides in the gRNA core. As exemplified in FIG. 3, the gRNA core of a PEgRNA may comprise one or more regions of a base paired lower stem, a base paired upper stem, where the lower stem and upper stem may be connected by a bulge comprising unpaired RNAs. The gRNA core may further comprise a nexus distal from the spacer sequence. In some embodiments, the gRNA core comprises one or more chemically modified nucleotides in the lower stem, upper stem, and/or the hairpin regions. In some embodiments, all of the nucleotides in the lower stem, upper stem, and/or the hairpin regions are chemically modified.

A chemical modification to a PEgRNA or ngRNA can comprise a 2′-O-thionocarbamate-protected nucleoside phosphoramidite, a 2′-O-methyl (M), a 2′-O-methyl 3′phosphorothioate (MS), or a 2′-O-methyl 3′thioPACE (MSP), or any combination thereof. In some embodiments, a chemically modified PEgRNA and/or ngRNA can comprise a ′-O-methyl (M) RNA, a 2′-O-methyl 3′phosphorothioate (MS) RNA, a 2′-O-methyl 3′thioPACE (MSP) RNA, a 2′-F RNA, a phosphorothioate bond modification, any other chemical modifications known in the art, or any combination thereof. A chemical modification may also include, for example, the incorporation of non-nucleotide linkages or modified nucleotides into the PEgRNA and/or ngRNA (e.g., modifications to one or both of the 3′ and 5′ ends of a guide RNA molecule). Such modifications can include the addition of bases to an RNA sequence, complexing the RNA with an agent (e.g., a protein or a complementary nucleic acid molecule), and inclusion of elements which change the structure of an RNA molecule (e.g., which form secondary structures).

### Prime Editing Compositions

Disclosed herein, in some embodiments, are compositions, systems, and methods using a prime editing composition. The term “prime editing composition” or “prime editing system” refers to compositions involved in the method of prime editing as described herein. A prime editing composition may include a prime editor, e.g., a prime editor fusion protein, and a PEgRNA. A prime editing composition may further comprise additional elements, such as second strand nicking ngRNAs. Components of a prime editing composition may be combined to form a complex for prime editing, or may be kept separately, e.g., for administration purposes. In some embodiments, a prime editing composition comprises a prime editor fusion protein complexed with a PEgRNA and optionally complexed with a ngRNA. In some embodiments, the prime editing composition comprises a prime editor comprising a DNA binding domain and a DNA polymerase domain associated with each other through a PEgRNA. For example, the prime editing composition may comprise a prime editor comprising a DNA binding domain and a DNA polymerase domain linked to each other by an RNA-protein recruitment aptamer RNA sequence, which is linked to a PEgRNA. In some embodiments, a prime editing composition comprises a PEgRNA and a polynucleotide, a polynucleotide construct, or a vector that encodes a prime editor fusion protein. In some embodiments, a prime editing composition comprises a PEgRNA, a ngRNA, and a polynucleotide, a polynucleotide construct, or a vector that encodes a prime editor fusion protein. In some embodiments, a prime editing composition comprises multiple polynucleotides, polynucleotide constructs, or vectors, each of which encodes one or more prime editing composition components. In some embodiments, the PEgRNA of a prime editing composition is associated with the DNA binding domain, e.g., a Cas9 nickase, of the prime editor. In some embodiments, the PEgRNA of a prime editing composition complexes with the DNA binding domain of a prime editor and directs the prime editor to the target DNA.

In some embodiments, a prime editing composition comprises one or more polynucleotides that encode prime editor components and/or PEgRNA or ngRNAs. In some embodiments, a prime editing composition comprises a polynucleotide encoding a fusion protein comprising a DNA binding domain and a DNA polymerase domain. In some embodiments, a prime editing composition comprises (i) a polynucleotide encoding a fusion protein comprising a DNA binding domain and a DNA polymerase domain, and (ii) a PEgRNA or a polynucleotide encoding the PEgRNA. In some embodiments, a prime editing composition comprises (i) a polynucleotide encoding a fusion protein comprising a DNA binding domain and a DNA polymerase domain, (ii) a PEgRNA or a polynucleotide encoding the PEgRNA, and (iii) an ngRNA or a polynucleotide encoding the ngRNA. In some embodiments, a prime editing composition comprises (i) a polynucleotide encoding a DNA binding domain of a prime editor, e.g., a Cas9 nickase, (ii) a polynucleotide encoding a DNA polymerase domain of a prime editor, e.g., a reverse transcriptase, and (iii) a PEgRNA or a polynucleotide encoding the PEgRNA. In some embodiments, a prime editing composition comprises (i) a polynucleotide encoding a DNA binding domain of a prime editor, e.g., a Cas9 nickase, (ii) a polynucleotide encoding a DNA polymerase domain of a prime editor, e.g., a reverse transcriptase, (iii) a PEgRNA or a polynucleotide encoding the PEgRNA, and (iv) an ngRNA or a polynucleotide encoding the ngRNA. In some embodiments, the polynucleotide encoding the DNA biding domain or the polynucleotide encoding the DNA polymerase domain further encodes an additional polypeptide domain, e.g., an RNA-protein recruitment domain, such as a MS2 coat protein domain. In some embodiments, a prime editing composition comprises (i) a polynucleotide encoding a N-terminal half of a prime editor fusion protein and an intein-N and (ii) a polynucleotide encoding a C-terminal half of a prime editor fusion protein and an intein-C. In some embodiments, a prime editing composition comprises (i) a polynucleotide encoding a N-terminal half of a prime editor fusion protein and an intein-N(ii) a polynucleotide encoding a C-terminal half of a prime editor fusion protein and an intein-C, (iii) a PEgRNA or a polynucleotide encoding the PEgRNA, and/or (iv) an ngRNA or a polynucleotide encoding the ngRNA. In some embodiments, a prime editing composition comprises (i) a polynucleotide encoding a N-terminal portion of a DNA binding domain and an intein-N, (ii) a polynucleotide encoding a C-terminal portion of the DNA binding domain, an intein-C, and a DNA polymerase domain. In some embodiments, the DNA binding domain is a Cas protein domain, e.g., a Cas9 nickase. In some embodiments, the prime editing composition comprises (i) a polynucleotide encoding a N-terminal portion of a DNA binding domain and an intein-N, (ii) a polynucleotide encoding a C-terminal portion of the DNA binding domain, an intein-C, and a DNA polymerase domain, (iii) a PEgRNA or a polynucleotide encoding the PEgRNA, and/or (iv) a ngRNA or a polynucleotide encoding the ngRNA.

In some embodiments, a prime editing system comprises one or more polynucleotides encoding one or more prime editor polypeptides, wherein activity of the prime editing system can be temporally regulated by controlling the timing in which the vectors are delivered. For example, in some embodiments, a polynucleotide encoding the prime editor and a polynucleotide encoding a PEgRNA can be delivered simultaneously. For example, in some embodiments, a polynucleotide encoding the prime editor and a polynucleotide encoding a PEgRNA can be delivered sequentially.

In some embodiments, a polynucleotide encoding a component of a prime editing system can further comprise an element that is capable of modifying the intracellular half-life of the polynucleotide and/or modulating translational control. In some embodiments, the polynucleotide is a RNA, for example, an mRNA. In some embodiments, the half-life of the polynucleotide, e.g., the RNA may be increased. In some embodiments, the half-life of the polynucleotide, e.g., the RNA may be decreased. In some embodiments, the element may be capable of increasing the stability of the polynucleotide, e.g., the RNA. In some embodiments, the element may be capable of decreasing the stability of the polynucleotide, e.g., the RNA. In some embodiments, the element may be within the 3′ UTR of the RNA. In some embodiments, the element may include a polyadenylation signal (PA). In some embodiments, the element may include a cap, e.g., an upstream mRNA or PEgRNA end. In some embodiments, the RNA may comprise no PA such that it is subject to quicker degradation in the cell after transcription.

In some embodiments, the element may include at least one AU-rich element (ARE). The AREs may be bound by ARE binding proteins (ARE-BPs) in a manner that is dependent upon tissue type, cell type, timing, cellular localization, and environment. In some embodiments the destabilizing element may promote RNA decay, affect RNA stability, or activate translation. In some embodiments, the ARE may comprise 50 to 150 nucleotides in length. In some embodiments, the ARE may comprise at least one copy of the sequence AUUUA. In some embodiments, at least one ARE may be added to the 3′ UTR of the RNA. In some embodiments, the element may be a Woodchuck Hepatitis Virus Posttranscriptional Regulatory Element (WPRE). In further embodiments, the element is a modified and/or truncated WPRE sequence that is capable of enhancing expression from the transcript. In some embodiments, the WPRE or equivalent may be added to the 3′ UTR of the RNA. In some embodiments, the element may be selected from other RNA sequence motifs that are enriched in either fast- or slow-decaying transcripts. In some embodiments, the polynucleotide, e.g., a vector, encoding the PE or the PEgRNA may be self-destroyed via cleavage of a target sequence present on the polynucleotide, e.g., a vector. The cleavage may prevent continued transcription of a PE or a PEgRNA.

Polynucleotides encoding prime editing composition components can be DNA, RNA, or any combination thereof. In some embodiments, a polynucleotide encoding a prime editing composition component is an expression construct. In some embodiments, a polynucleotide encoding a prime editing composition component is a vector. In some embodiments, the vector is a DNA vector. In some embodiments, the vector is a plasmid. In some embodiments, the vector is a virus vector, e.g., a retroviral vector, adenoviral vector, lentiviral vector, herpesvirus vector, or an adeno-associated virus vector (AAV).

In some embodiments, polynucleotides encoding polypeptide components of a prime editing composition are codon optimized by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence. In some embodiments, a polynucleotide encoding a polypeptide component of a prime editing composition are operably linked to one or more expression regulatory elements, for example, a promoter, a 3′ UTR, a 5′ UTR, or any combination thereof. In some embodiments, a polynucleotide encoding a prime editing composition component is a messenger RNA (mRNA). In some embodiments, the mRNA comprises a Cap at the 5′ end and/or a poly A tail at the 3′ end.

### Pharmaceutical Compostions

Disclosed herein are pharmaceutical compositions comprising any of the prime editing composition components, for example, prime editors, fusion proteins, polynucleotides encoding prime editor polypeptides, PEgRNAs, ngRNAs, and/or prime editing complexes described herein.

The term “pharmaceutical composition”, as used herein, refers to a composition formulated for pharmaceutical use. In some embodiments, the pharmaceutical composition further comprises a pharmaceutically acceptable carrier. In some embodiments, the pharmaceutical composition comprises additional agents, e.g., for specific delivery, increasing half-life, or other therapeutic compounds.

In some embodiments, a pharmaceutically-acceptable carrier comprises any vehicle, such as a liquid or solid filler, diluent, excipient, manufacturing aid (e.g., lubricant, talc magnesium, calcium or zinc stearate, or steric acid), or solvent encapsulating material, involved in carrying or transporting the compound from one site (e.g., the delivery site) of the body, to another site (e.g., organ, tissue or portion of the body). A pharmaceutically acceptable carrier is “acceptable” in the sense of being compatible with the other ingredients of the formulation and not injurious to the tissue of the subject (e.g., physiologically compatible, sterile, physiologic pH, etc.)

Formulations of the pharmaceutical compositions described herein can be prepared by any method known or hereafter developed in the art of pharmacology. In general, such preparatory methods include the step of bringing the active ingredient(s) into association with an excipient and/or one or more other accessory ingredients, and then, if necessary and/or desirable, shaping and/or packaging the product into a desired single- or multi-dose unit. Pharmaceutical formulations can additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired.

### Methods of Editing

The methods and compositions disclosed herein can be used to edit a target gene of interest by prime editing.

In some embodiments, the prime editing method comprises contacting a target gene, e.g., e.g., an NCF1 gene, an NCF1B pseudogene, or an NCF1C pseudogene. with a PEgRNA and a prime editor (PE) polypeptide described herein. In some embodiments, the target gene is double stranded, and comprises two strands of DNA complementary to each other. In some embodiments, the contacting with a PEgRNA and the contacting with a prime editor are performed sequentially. In some embodiments, the contacting with a prime editor is performed after the contacting with a PEgRNA. In some embodiments, the contacting with a PEgRNA is performed after the contacting with a prime editor. In some embodiments, the contacting with a PEgRNA, and the contacting with a prime editor are performed simultaneously. In some embodiments, the PEgRNA and the prime editor are associated in a complex prior to contacting a target gene.

In some embodiments, contacting the target gene with the prime editing composition results in binding of the PEgRNA to a target strand of the target gene, e.g., an NCF1 gene, an NCF1B pseudogene, or an NCF1C pseudogene. In some embodiments, contacting the target gene with the prime editing composition results in binding of the PEgRNA to a search target sequence on the target strand of the target gene upon contacting with the PEgRNA. In some embodiments, contacting the target gene with the prime editing composition results in binding of a spacer sequence of the PEgRNA to a search target sequence with the search target sequence on the target strand of the target gene upon said contacting of the PEgRNA.

In some embodiments, contacting the target gene with the prime editing composition results in binding of the prime editor to the target gene, e.g., an NCF1 gene, an NCF1B pseudogene, or an NCF1C pseudogene, upon the contacting of the PE composition with the target gene. In some embodiments, the DNA binding domain of the PE associates with the PEgRNA. In some embodiments, the PE binds the target gene, e.g., an NCF1 gene, an NCF1B pseudogene, or an NCF1C pseudogene, directed by the PEgRNA. Accordingly, in some embodiments, the contacting of the target gene results in binding of a DNA binding domain of a prime editor of the target gene, e.g., an NCF1 gene, an NCF1B pseudogene, or an NCF1C pseudogene, directed by the PEgRNA.

In some embodiments, contacting the target gene with the prime editing composition results in a nick in an edit strand of the target gene, e.g., an NCF1 gene, an NCF1B pseudogene, or an NCF1C pseudogene by the prime editor upon contacting with the target gene, thereby generating a nicked on the edit strand of the target gene. In some embodiments, contacting the target gene with the prime editing composition results in a single-stranded DNA comprising a free 3′ end at the nick site of the edit strand of the target gene. In some embodiments, contacting the target gene with the prime editing composition results in a nick in the edit strand of the target gene by a DNA binding domain of the prime editor, thereby generating a single-stranded DNA comprising a free 3′ end at the nick site. In some embodiments, the DNA binding domain of the prime editor is a Cas domain. In some embodiments, the DNA binding domain of the prime editor is a Cas9. In some embodiments, the DNA binding domain of the prime editor is a Cas9 nickase.

In some embodiments, contacting the target gene with the prime editing composition results in hybridization of the PEgRNA with the 3′ end of the nicked single-stranded DNA, thereby priming DNA polymerization by a DNA polymerase domain of the prime editor. In some embodiments, the free 3′ end of the single-stranded DNA generated at the nick site hybridizes to a primer binding site sequence (PBS) of the contacted PEgRNA, thereby priming DNA polymerization. In some embodiments, the DNA polymerization is reverse transcription catalyzed by a reverse transcriptase domain of the prime editor. In some embodiments, the method comprises contacting the target gene with a DNA polymerase, e.g., a reverse transcriptase, as a part of a prime editor fusion protein or prime editing complex (in cis), or as a separate protein (in trans).

In some embodiments, contacting the target gene with the prime editing composition generates an edited single stranded DNA that is coded by the editing template of the PEgRNA by DNA polymerase mediated polymerization from the 3′ free end of the single-stranded DNA at the nick site. In some embodiments, the editing template of the PEgRNA comprises one or more intended nucleotide edits compared to endogenous sequence of the target gene, e.g., an NCF1 gene, an NCF1B pseudogene, or an NCF1C pseudogene. In some embodiments, the intended nucleotide edits are incorporated in the target gene, by excision of the 5′ single stranded DNA of the edit strand of the target gene generated at the nick site and DNA repair. In some embodiments, the intended nucleotide edits are incorporated in the target gene by excision of the editing target sequence and DNA repair. In some embodiments, excision of the 5′ single stranded DNA of the edit strand generated at the nick site is by a flap endonuclease. In some embodiments, the flap nuclease is FEN1. In some embodiments, the method further comprises contacting the target gene with a flap endonuclease. In some embodiments, the flap endonuclease is provided as a part of a prime editor fusion protein. In some embodiments, the flap endonuclease is provided in trans.

In some embodiments, contacting the target gene with the prime editing composition generates a mismatched heteroduplex comprising the edit strand of the target gene that comprises the edited single stranded DNA, and the unedited target strand of the target gene. Without being bound by theory, the endogenous DNA repair and replication may resolve the mismatched edited DNA to incorporate the nucleotide change(s) to form the desired edited target gene.

In some embodiments, the method further comprises contacting the target gene, e.g., an NCF1 gene, an NCF1B pseudogene, or an NCF1C pseudogene, with a nick guide (ngRNA) disclosed herein. In some embodiments, the ngRNA comprises a spacer that binds a second search target sequence on the edit strand of the target gene. In some embodiments, the contacted ngRNA directs the PE to introduce a nick in the target strand of the target gene. In some embodiments, the nick on the target strand (non-edit strand) results in endogenous DNA repair machinery to use the edit strand to repair the non-edit strand, thereby incorporating the intended nucleotide edit in both strand of the target gene and modifying the target gene. In some embodiments, the ngRNA comprises a spacer sequence that is complementary to, and may hybridize with, the second search target sequence on the edit strand only after the intended nucleotide edit(s) are incorporated in the edit strand of the target gene.

In some embodiments, the target gene is contacted by the ngRNA, the PEgRNA, and the PE simultaneously. In some embodiments, the ngRNA, the PEgRNA, and the PE form a complex when they contact the target gene. In some embodiments, the target gene is contacted with the ngRNA, the PEgRNA, and the prime editor sequentially. In some embodiments, the target gene is contacted with the ngRNA and/or the PEgRNA after contacting the target gene with the PE. In some embodiments, the target gene is contacted with the ngRNA and/or the PEgRNA before contacting the target gene with the prime editor.

In some embodiments, the target gene, e.g., an NCF1 gene, an NCF1B pseudogene, or an NCF1C pseudogene, is in a cell. Accordingly, also provided herein are methods of modifying a cell.

In some embodiments, the prime editing method comprises introducing a PEgRNA, a prime editor, and/or a ngRNA into the cell that has the target gene. In some embodiments, the prime editing method comprises introducing into the cell that has the target gene with a prime editing composition comprising a PEgRNA, a prime editor polypeptide, and/or a ngRNA. In some embodiments, the PEgRNA, the prime editor polypeptide, and/or the ngRNA form a complex prior to the introduction into the cell. In some embodiments, the PEgRNA, the prime editor polypeptide, and/or the ngRNA form a complex after the introduction into the cell. The prime editors, PEgRNA and/or ngRNAs, and prime editing complexes may be introduced into the cell by any delivery approaches described herein or any delivery approach known in the art, including ribonucleoprotein (RNPs), lipid nanoparticles (LNPs), viral vectors, non-viral vectors, mRNA delivery, and physical techniques such as cell membrane disruption by a microfluidics device. The prime editors, PEgRNA and/or ngRNAs, and prime editing complexes may be introduced into the cell simultaneously or sequentially.

In some embodiments, the prime editing method comprises introducing into the cell a PEgRNA or a polynucleotide encoding the PEgRNA, a prime editor polynucleotide encoding a prime editor polypeptide, and optionally an ngRNA or a polynucleotide encoding the ngRNA. In some embodiments, the method comprises introducing the PEgRNA or the polynucleotide encoding the PEgRNA, the polynucleotide encoding the prime editor polypeptide, and/or the ngRNA or the polynucleotide encoding the ngRNA into the cell simultaneously. In some embodiments, the method comprises introducing the PEgRNA or the polynucleotide encoding the PEgRNA, the polynucleotide encoding the prime editor polypeptide, and/or the ngRNA or the polynucleotide encoding the ngRNA into the cell sequentially. In some embodiments, the method comprises introducing the polynucleotide encoding the prime editor polypeptide into the cell before introduction of the PEgRNA or the polynucleotide encoding the PEgRNA and/or the ngRNA or the polynucleotide encoding the ngRNA. In some embodiments, the polynucleotide encoding the prime editor polypeptide is introduced into and expressed in the cell before introduction of the PEgRNA or the polynucleotide encoding the PEgRNA and/or the ngRNA or the polynucleotide encoding the ngRNA into the cell. In some embodiments, the polynucleotide encoding the prime editor polypeptide is introduced into the cell after the PEgRNA or the polynucleotide encoding the PEgRNA and/or the ngRNA or the polynucleotide encoding the ngRNA are introduced into the cell. The polynucleotide encoding the prime editor polypeptide, the PEgRNA or the polynucleotide encoding the PEgRNA, and/or the ngRNA or the polynucleotide encoding the ngRNA, may be introduced into the cell by any delivery approaches described herein or any delivery approach known in the art, for example, by RNPs, LNPs, viral vectors, non-viral vectors, mRNA delivery, and physical. In some embodiments, the polynucleotide is a DNA polynucleotide. In some embodiments, the polynucleotide is a RNA polynucleotide, e.g., mRNA polynucleotide.

In some embodiments, the polynucleotide encoding the prime editor polypeptide, the polynucleotide encoding the PEgRNA, and/or the polynucleotide encoding the ngRNA integrate into the genome of the cell after being introduced into the cell. In some embodiments, the polynucleotide encoding the prime editor polypeptide, the polynucleotide encoding the PEgRNA, and/or the polynucleotide encoding the ngRNA are introduced into the cell for transient expression. Accordingly, also provided herein are cells modified by prime editing.

In some embodiments, the cell is a eukaryotic cell. In some embodiments, the cell is a mammalian cell. In some embodiments, the cell is a non-human primate cell, bovine cell, porcine cell, rodent or mouse cell. In some embodiments, the cell is a human cell. In some embodiments, the cell is a primary cell. In some embodiments, the cell is a human primary cell. In some embodiments, the cell is a progenitor cell. In some embodiments, the cell is a human progenitor cell. In some embodiments, the cell is a stem cell. In some embodiments, the cell is a human stem cell. In some embodiments, the cell is an iPSC. In some embodiments, the cell is a human iPSC. In some embodiments, the cell is a hematopoietic stem cell (HSC). In some embodiments, the cell is a human HSC. In some embodiments, the HSC is from bone marrow or mobilized peripheral blood. In some embodiments, the cell is a hematopoietic stem and progenitor cell. In some embodiments the human stem cell is an induced pluripotent stem cell (iPSC). In some embodiments, the cell is a human HSC. In some embodiments, the cell is a human CD34⁺ cell. In some embodiments, the cell is a hematopoietic stem and progenitor cell (HSPC). A human HSPC cell may also be generally referred to as a CD34+ cell. In some embodiments, the cell is a hematopoietic progenitor cell, multipotent progenitor cell, a lymphoid progenitor cell, a myeloid progenitor cell, a megakaryocyte-erythroid progenitor cell, a granulocyte-megakaryocyte progenitor cell, a granulocyte, a promyelocyte, a neutrophil, an eosinophil, a basophil, an erythrocyte, a reticulocyte, a thrombocyte, a megakaryoblast, a platelet-producing megakaryocyte, a monocyte, a macrophage, a dendritic cell, a microglia, an osteoclast, a lymphocyte, a NK cell, a B-cell, or a T-cell. In some embodiments, the cell edited by prime editing can be differentiated into, or give rise to recovery of a population of cells, e.g., common lymphoid progenitor cells, common myeloid progenitor cells, megakaryocyte-erythroid progenitor cells, granulocyte-megakaryocyte progenitor cells, granulocytes, promyelocytes, neutrophils, eosinophils, basophils, erythrocytes, reticulocytes, thrombocytes, megakaryoblasts, platelet-producing megakaryocytes, platelets, monocytes, macrophages, dendritic cells, microglia, osteoclasts, lymphocytes, such as NK cells, B-cells or T-cells. In some embodiments, the cell edited by prime editing can be differentiated into, or give rise to recovery of a population of cells, e.g., neutrophils, platelets, red blood cells, monocytes, macrophages, antigen-presenting cells, microglia, osteoclasts, dendritic cells, and/or lymphocytes. In some embodiments, the cell is an ex vivo cell. In some embodiments, the cell is an ex vivo cell obtained from a human subject. For example, in some embodiments, the cell is a stem cell, a bone marrow cell, a LCL cell, or a CD34+ cell obtained from a subject having CGD disease prior to editing. After correction of the mutation by prime editing, the cell may be administered to the subject, e.g., by infusion. In some embodiments, the cell is in a subject, e.g., a human subject.

In some embodiments, the target gene edited by prime editing is in a chromosome of the cell. In some embodiments, the intended nucleotide edits incorporate in the chromosome of the cell and are inheritable by progeny cells. In some embodiments, the intended nucleotide edits introduced to the cell by the prime editing compositions and methods are such that the cell and progeny of the cell also include the intended nucleotide edits. In some embodiments, the cell is autologous, allogeneic, or xenogeneic to a subject. In some embodiments, the cell is from or derived from a subject. In some embodiments, the cell is from or derived from a human subject. In some embodiments, the cell is introduced back into the subject, e.g., a human subject, after incorporation of the intended nucleotide edits by prime editing.

In some embodiments, the method provided herein comprises introducing the prime editor polypeptide or the polynucleotide encoding the prime editor polypeptide, the PEgRNA or the polynucleotide encoding the PEgRNA, and/or the ngRNA or the polynucleotide encoding the ngRNA into a plurality or a population of cells that comprise the target gene. In some embodiments, the population of cells is of the same cell type. In some embodiments, the population of cells is of the same tissue or organ. In some embodiments, the population of cells is heterogeneous. In some embodiments, the population of cells is homogeneous. In some embodiments, the population of cells is from a single tissue or organ, and the cells are heterogeneous. In some embodiments, the introduction into the population of cells is ex vivo. In some embodiments, the introduction into the population of cells is in vivo, e.g., into a human subject.

In some embodiments, the target gene is in a genome of each cell of the population. In some embodiments, introduction of the prime editor polypeptide or the polynucleotide encoding the prime editor polypeptide, the PEgRNA or the polynucleotide encoding the PEgRNA, and/or the ngRNA or the polynucleotide encoding the ngRNA results in incorporation of one or more intended nucleotide edits in the target gene in at least one of the cells in the population of cells. In some embodiments, introduction of the prime editor polypeptide or the polynucleotide encoding the prime editor polypeptide, the PEgRNA or the polynucleotide encoding the PEgRNA, and/or the ngRNA or the polynucleotide encoding the ngRNA results in incorporation of the one or more intended nucleotide edits in the target gene in a plurality of the population of cells. In some embodiments, introduction of the prime editor polypeptide or the polynucleotide encoding the prime editor polypeptide, the PEgRNA or the polynucleotide encoding the PEgRNA, and/or the ngRNA or the polynucleotide encoding the ngRNA results in incorporation of the one or more intended nucleotide edits in the target gene in each cell of the population of cells. In some embodiments, introduction of the prime editor polypeptide or the polynucleotide encoding the prime editor polypeptide, the PEgRNA or the polynucleotide encoding the PEgRNA, and/or the ngRNA or the polynucleotide encoding the ngRNA results in incorporation of the one or more intended nucleotide edits in the target gene in sufficient number of cells such that the disease or disorder is treated, prevented or ameliorated.

In some embodiments, editing efficiency of the prime editing compositions and method described herein can be measured by calculating the percentage of edited target genes in a population of cells introduced with the prime editing composition. In some embodiments, the editing efficiency is determined after 1 hour, 2 hours, 6 hours, 12 hours, 24 hours, 36 hours, 48 hours, 3 days, 4 days, 5 days, 7 days, 10 days, or 14 days of exposing a target gene (e.g., an NCF1 gene, an NCF1B pseudogene, or an NCF1C pseudogene gene within the genome of a cell) to a prime editing composition. In some embodiments, editing efficiency of the prime editing compositions and method described herein can be measured by calculating the percentage of edited target genes in a population of cells introduced with the prime editing composition. In some embodiments, the editing efficiency is determined after 5, 6, 7, 8, 9, 10, 11, 12, 13, 14, 15, or 16 weeks of exposing a target gene (e.g., an NCF1 gene, an NCF1B pseudogene, or an NCF1C pseudogene gene within the genome of a cell) to a prime editing composition. In some embodiments, the population of cells introduced with the prime editing composition is ex vivo. In some embodiments, the population of cells introduced with the prime editing composition is in vitro. In some embodiments, the population of cells introduced with the prime editing composition is in vivo. In some embodiments, the prime editing methods disclosed herein have an editing efficiency of at least about 1%, at least about 5%, at least about 10%, at least about 20%, at least about 30%, at least about 40%, at least about 50%, at least about 60%, at least about 70%, at least about 80%, at least about 90%, at least about 95%, or at least about 99% relative to a suitable control. In some embodiments, the prime editing methods disclosed herein have an editing efficiency of at least 25% relative to a suitable control. In some embodiments, the prime editing methods disclosed herein have an editing efficiency of at least 35% relative to a suitable control. prime editing method disclosed herein has an editing efficiency of at least 30% relative to a suitable control. In some embodiments, the prime editing methods disclosed herein have an editing efficiency of at least 45% relative to a suitable control. In some embodiments, the prime editing methods disclosed herein have an editing efficiency of at least 50% relative to a suitable control. In some embodiments, editing efficiency of prime the prime editing compositions and method described herein can be measured by calculating the percentage of edited target genes in a population of cells after in vivo engraftment of the edited cells. In some embodiments, the editing efficiency is determined after 5, 6, 7, 8, 9, 10, 11, 12, 13, 14, 15, or 16 weeks of engraftment. In some embodiments, the editing efficiency is determined after 8 or 16 weeks of engraftment. In some embodiments, prime editing is able to maintain in edited cells at least about 10%, about 15%, about 20%, about 25%, about 30%, about 35%, about 40%, about 45%, about 50%, about 55%, about 60%, about 65%, about 70%, about 75%, about 80%, about 85%, about 90%, about 95% or more than 95% of editing efficiency after 8 or 16 weeks post engraftment.

In some embodiments, the methods disclosed herein have an editing efficiency of at least about 1%, at least about 5%, at least about 7.5%, at least about 10%, at least about 15%, at least about 20%, at least about 30%, at least about 40%, at least about 50%, at least about 60%, at least about 70%, at least about 80%, at least about 90%, or at least about 95% of editing a primary cell (as measured in a population of primary cells) relative to a suitable control.

In some embodiments, the methods disclosed herein have an editing efficiency of at least about 5%, at least about 7.5%, at least about 10%, at least about 15%, at least about 20%, at least about 30%, at least about 40%, at least about 50%, at least about 60%, at least about 70%, at least about 80%, at least about 90%, or at least about 95% of editing HSPC (as measured in a population of HSPC cells) relative to a corresponding control HSPC. In some embodiments, the HSC is a human HSPC.

In some embodiments, the prime editing compositions provided herein are capable of incorporating one or more intended nucleotide edits without generating a significant proportion of indels. The term “indel(s)”, as used herein, refers to the insertion or deletion of a nucleotide base within a polynucleotide, for example, a target gene. Such insertions or deletions can lead to frame shift mutations within a coding region of a gene. Indel frequency of editing can be calculated by methods known in the art. In some embodiments, indel frequency can be calculated based on sequence alignment such as the CRISPResso 2 algorithm as described in Clement et al., Nat. Biotechnol. 37(3): 224-226 (2019), which is incorporated herein in its entirety. In some embodiments, the prime editing methods disclosed herein can have an indel frequency of less than 30%, less than 20%, less than 10%, less than 9%, less than 8%, less than 7%, less than 6%, less than 5%, less than 4%, less than 3%, less than 2%, less than 1.5%, or less than 1%. In some embodiments, any number of indels is determined after at least 1 hour, at least 2 hours, at least 6 hours, at least 12 hours, at least 24 hours, at least 36 hours, at least 48 hours, at least 3 days, at least 4 days, at least 5 days, at least 7 days, at least 10 days, or at least 14 days of exposing a target gene (e.g., an NCF1 gene, an NCF1B pseudogene, or an NCF1C pseudogene within the genome of a cell) to a prime editing composition.

In some embodiments, the prime editing compositions provided herein are capable of incorporating one or more intended nucleotide edits efficiently without generating a significant proportion of indels. In some embodiments, the prime editing methods disclosed herein have an editing efficiency of at least about 1% and an indel frequency of less than 10% in a population of target cells, e.g., a population of human HSPCs. In some embodiments, the prime editing methods disclosed herein have an editing efficiency of at least about 1% and an indel frequency of less than 7.5% in a population of target cells, e.g., a population of human HSPCs. In some embodiments, the prime editing methods disclosed herein have an editing efficiency of at least about 1% and an indel frequency of less than 5% in a population of target cells, e.g., a population of human HSPCs. In some embodiments, the prime editing methods disclosed herein have an editing efficiency of at least about 1% and an indel frequency of less than 2.5% in a population of target cells, e.g., a population of human HSPCs. In some embodiments, the prime editing methods disclosed herein have an editing efficiency of at least about 1% and an indel frequency of less than 1% in a population of target cells, e.g., a population of human HSPCs. In some embodiments, the prime editing methods disclosed herein have an editing efficiency of at least about 1% and an indel frequency of less than 0.5% in a population of target cells, e.g., a population of human HSPCs. In some embodiments, the prime editing methods disclosed herein have an editing efficiency of at least about 1% and an indel frequency of less than 0.1% in a population of target cells, e.g., a population of human HSPCs. In some embodiments, the prime editing methods disclosed herein have an editing efficiency of at least about 5% and an indel frequency of less than 1% in a population of target cells, e.g., a population of human HSPCs. In some embodiments, the prime editing methods disclosed herein have an editing efficiency of at least about 5% and an indel frequency of less than 0.5% in a population of target cells, e.g., a population of human HSPCs. In some embodiments, the prime editing methods disclosed herein have an editing efficiency of at least about 5% and an indel frequency of less than 0.1% in a population of target cells, e.g., a population of human HSPCs.

In some embodiments, the prime editing methods disclosed herein have an editing efficiency of at least about 7.5% and an indel frequency of less than 10% in a population of target cells, e.g., a population of human HSCs. In some embodiments, the prime editing methods disclosed herein have an editing efficiency of at least about 7.5% and an indel frequency of less than 7.5% in a population of target cells, e.g., a population of human HSCs. In some embodiments, the prime editing methods disclosed herein have an editing efficiency of at least about 7.5% and an indel frequency of less than 5% in a population of target cells, e.g., a population of human HSCs. In some embodiments, the prime editing methods disclosed herein have an editing efficiency of at least about 7.5% and an indel frequency of less than 2.5% in a population of target cells, e.g., a population of human HSCs. In some embodiments, the prime editing methods disclosed herein have an editing efficiency of at least about 7.5% and an indel frequency of less than 1% in a population of target cells, e.g., a population of human HSCs. In some embodiments, the prime editing methods disclosed herein have an editing efficiency of at least about 7.5% and an indel frequency of less than 0.5% in a population of target cells, e.g., a population of human HSCs. In some embodiments, the prime editing methods disclosed herein have an editing efficiency of at least about 7.5% and an indel frequency of less than 0.1% in a population of target cells, e.g., a population of human HSCs.

In some embodiments, the prime editing methods disclosed herein have an editing efficiency of at least about 10% and an indel frequency of less than 10% in a population of target cells, e.g., a population of human HSCs. In some embodiments, the prime editing methods disclosed herein have an editing efficiency of at least about 10% and an indel frequency of less than 7.5% in a population of target cells, e.g., a population of human HSCs. In some embodiments, the prime editing methods disclosed herein have an editing efficiency of at least about 10% and an indel frequency of less than 5% in a population of target cells, e.g., a population of human HSCs. In some embodiments, the prime editing methods disclosed herein have an editing efficiency of at least about 10% and an indel frequency of less than 2.5% in a population of target cells, e.g., a population of human HSCs. In some embodiments, the prime editing methods disclosed herein have an editing efficiency of at least about 10% and an indel frequency of less than 1% in a population of target cells, e.g., a population of human HSCs. In some embodiments, the prime editing methods disclosed herein have an editing efficiency of at least about 10% and an indel frequency of less than 0.5% in a population of target cells, e.g., a population of human HSCs. In some embodiments, the prime editing methods disclosed herein have an editing efficiency of at least about 10% and an indel frequency of less than 0.1% in a population of target cells, e.g., a population of human HSCs.

In some embodiments, the prime editing methods disclosed herein have an editing efficiency of at least about 15% and an indel frequency of less than 10% in a population of target cells, e.g., a population of human HSCs. In some embodiments, the prime editing methods disclosed herein have an editing efficiency of at least about 15% and an indel frequency of less than 7.5% in a population of target cells, e.g., a population of human HSCs. In some embodiments, the prime editing methods disclosed herein have an editing efficiency of at least about 15% and an indel frequency of less than 5% in a population of target cells, e.g., a population of human HSCs. In some embodiments, the prime editing methods disclosed herein have an editing efficiency of at least about 15% and an indel frequency of less than 2.5% in a population of target cells, e.g., a population of human HSCs. In some embodiments, the prime editing methods disclosed herein have an editing efficiency of at least about 15% and an indel frequency of less than 1% in a population of target cells, e.g., a population of human HSCs. In some embodiments, the prime editing methods disclosed herein have an editing efficiency of at least about 15% and an indel frequency of less than 0.5% in a population of target cells, e.g., a population of human HSCs. In some embodiments, the prime editing methods disclosed herein have an editing efficiency of at least about 15% and an indel frequency of less than 0.1% in a population of target cells, e.g., a population of human HSCs.

In some embodiments, the prime editing methods disclosed herein have an editing efficiency of at least about 15% and an indel frequency of less than 10% in a population of target cells, e.g., a population of human HSCs. In some embodiments, the prime editing methods disclosed herein have an editing efficiency of at least about 15% and an indel frequency of less than 7.5% in a population of target cells, e.g., a population of human HSCs. In some embodiments, the prime editing methods disclosed herein have an editing efficiency of at least about 15% and an indel frequency of less than 5% in a population of target cells, e.g., a population of human HSCs. In some embodiments, the prime editing methods disclosed herein have an editing efficiency of at least about 15% and an indel frequency of less than 2.5% in a population of target cells, e.g., a population of human HSCs. In some embodiments, the prime editing methods disclosed herein have an editing efficiency of at least about 20% and an indel frequency of less than 1% in a population of target cells, e.g., a population of human HSCs. In some embodiments, the prime editing methods disclosed herein have an editing efficiency of at least about 20% and an indel frequency of less than 0.5% in a population of target cells, e.g., a population of human HSCs. In some embodiments, the prime editing methods disclosed herein have an editing efficiency of at least about 20% and an indel frequency of less than 0.1% in a population of target cells, e.g., a population of human HSCs.

In some embodiments, the prime editing methods disclosed herein have an editing efficiency of at least about 30% and an indel frequency of less than 10% in a population of target cells, e.g., a population of human HSCs. In some embodiments, the prime editing methods disclosed herein have an editing efficiency of at least about 30% and an indel frequency of less than 7.5% in a population of target cells, e.g., a population of human HSCs. In some embodiments, the prime editing methods disclosed herein have an editing efficiency of at least about 30% and an indel frequency of less than 5% in a population of target cells, e.g., a population of human HSCs. In some embodiments, the prime editing methods disclosed herein have an editing efficiency of at least about 30% and an indel frequency of less than 2.5% in a population of target cells, e.g., a population of human HSCs. In some embodiments, the prime editing methods disclosed herein have an editing efficiency of at least about 30% and an indel frequency of less than 1% in a population of target cells, e.g., a population of human HSCs. In some embodiments, the prime editing methods disclosed herein have an editing efficiency of at least about 30% and an indel frequency of less than 0.5% in a population of target cells, e.g., a population of human HSCs. In some embodiments, the prime editing methods disclosed herein have an editing efficiency of at least about 30% and an indel frequency of less than 0.1% in a population of target cells, e.g., a population of human HSCs.

In some embodiments, the prime editing methods disclosed herein have an editing efficiency of at least about 40% and an indel frequency of less than 10% in a population of target cells, e.g., a population of human HSCs. In some embodiments, the prime editing methods disclosed herein have an editing efficiency of at least about 40% and an indel frequency of less than 7.5% in a population of target cells, e.g., a population of human HSCs. In some embodiments, the prime editing methods disclosed herein have an editing efficiency of at least about 40% and an indel frequency of less than 5% in a population of target cells, e.g., a population of human HSCs. In some embodiments, the prime editing methods disclosed herein have an editing efficiency of at least about 40% and an indel frequency of less than 2.5% in a population of target cells, e.g., a population of human HSCs. In some embodiments, the prime editing methods disclosed herein have an editing efficiency of at least about 40% and an indel frequency of less than 1% in a population of target cells, e.g., a population of human HSCs. In some embodiments, the prime editing methods disclosed herein have an editing efficiency of at least about 40% and an indel frequency of less than 0.5% in a population of target cells, e.g., a population of human HSCs. In some embodiments, the prime editing methods disclosed herein have an editing efficiency of at least about 40% and an indel frequency of less than 0.1% in a population of target cells, e.g., a population of human HSCs.

In some embodiments, the prime editing methods disclosed herein have an editing efficiency of at least about 50% and an indel frequency of less than 10% in a population of target cells, e.g., a population of human HSCs. In some embodiments, the prime editing methods disclosed herein have an editing efficiency of at least about 50% and an indel frequency of less than 7.5% in a population of target cells, e.g., a population of human HSCs. In some embodiments, the prime editing methods disclosed herein have an editing efficiency of at least about 50% and an indel frequency of less than 5% in a population of target cells, e.g., a population of human HSCs. In some embodiments, the prime editing methods disclosed herein have an editing efficiency of at least about 50% and an indel frequency of less than 2.5% in a population of target cells, e.g., a population of human HSCs. In some embodiments, the prime editing methods disclosed herein have an editing efficiency of at least about 50% and an indel frequency of less than 1% in a population of target cells, e.g., a population of human HSCs. In some embodiments, the prime editing methods disclosed herein have an editing efficiency of at least about 50% and an indel frequency of less than 0.5% in a population of target cells, e.g., a population of human HSCs. In some embodiments, the prime editing methods disclosed herein have an editing efficiency of at least about 50% and an indel frequency of less than 0.1% in a population of target cells, e.g., a population of human HSCs.

In some embodiments, the prime editing methods disclosed herein have an editing efficiency of at least about 60% and an indel frequency of less than 10% in a population of target cells, e.g., a population of human HSCs. In some embodiments, the prime editing methods disclosed herein have an editing efficiency of at least about 60% and an indel frequency of less than 7.5% in a population of target cells, e.g., a population of human HSCs. In some embodiments, the prime editing methods disclosed herein have an editing efficiency of at least about 60% and an indel frequency of less than 5% in a population of target cells, e.g., a population of human HSCs. In some embodiments, the prime editing methods disclosed herein have an editing efficiency of at least about 60% and an indel frequency of less than 2.5% in a population of target cells, e.g., a population of human HSCs. In some embodiments, the prime editing methods disclosed herein have an editing efficiency of at least about 60% and an indel frequency of less than 1% in a population of target cells, e.g., a population of human HSCs. In some embodiments, the prime editing methods disclosed herein have an editing efficiency of at least about 60% and an indel frequency of less than 0.5% in a population of target cells, e.g., a population of human HSCs. In some embodiments, the prime editing methods disclosed herein have an editing efficiency of at least about 60% and an indel frequency of less than 0.1% in a population of target cells, e.g., a population of human HSCs.

In some embodiments, the prime editing methods disclosed herein have an editing efficiency of at least about 70% and an indel frequency of less than 10% in a population of target cells, e.g., a population of human HSCs. In some embodiments, the prime editing methods disclosed herein have an editing efficiency of at least about 70% and an indel frequency of less than 7.5% in a population of target cells, e.g., a population of human HSCs. In some embodiments, the prime editing methods disclosed herein have an editing efficiency of at least about 70% and an indel frequency of less than 5% in a population of target cells, e.g., a population of human HSCs. In some embodiments, the prime editing methods disclosed herein have an editing efficiency of at least about 70% and an indel frequency of less than 2.5% in a population of target cells, e.g., a population of human HSCs. In some embodiments, the prime editing methods disclosed herein have an editing efficiency of at least about 70% and an indel frequency of less than 1% in a population of target cells, e.g., a population of human HSCs. In some embodiments, the prime editing methods disclosed herein have an editing efficiency of at least about 70% and an indel frequency of less than 0.5% in a population of target cells, e.g., a population of human HSCs. In some embodiments, the prime editing methods disclosed herein have an editing efficiency of at least about 70% and an indel frequency of less than 0.1% in a population of target cells, e.g., a population of human HSCs.

In some embodiments, the prime editing methods disclosed herein have an editing efficiency of at least about 80% and an indel frequency of less than 10% in a population of target cells, e.g., a population of human HSCs. In some embodiments, the prime editing methods disclosed herein have an editing efficiency of at least about 80% and an indel frequency of less than 7.5% in a population of target cells, e.g., a population of human HSCs. In some embodiments, the prime editing methods disclosed herein have an editing efficiency of at least about 80% and an indel frequency of less than 5% in a population of target cells, e.g., a population of human HSCs. In some embodiments, the prime editing methods disclosed herein have an editing efficiency of at least about 80% and an indel frequency of less than 2.5% in a population of target cells, e.g., a population of human HSCs. In some embodiments, the prime editing methods disclosed herein have an editing efficiency of at least about 80% and an indel frequency of less than 1% in a population of target cells, e.g., a population of human HSCs. In some embodiments, the prime editing methods disclosed herein have an editing efficiency of at least about 80% and an indel frequency of less than 0.5% in a population of target cells, e.g., a population of human HSCs. In some embodiments, the prime editing methods disclosed herein have an editing efficiency of at least about 80% and an indel frequency of less than 0.1% in a population of target cells, e.g., a population of human HSCs.

In some embodiments, the prime editing methods disclosed herein have an editing efficiency of at least about 90% and an indel frequency of less than 10% in a population of target cells, e.g., a population of human HSCs. In some embodiments, the prime editing methods disclosed herein have an editing efficiency of at least about 90% and an indel frequency of less than 7.5% in a population of target cells, e.g., a population of human HSCs. In some embodiments, the prime editing methods disclosed herein have an editing efficiency of at least about 90% and an indel frequency of less than 5% in a population of target cells, e.g., a population of human HSCs. In some embodiments, the prime editing methods disclosed herein have an editing efficiency of at least about 90% and an indel frequency of less than 2.5% in a population of target cells, e.g., a population of human HSCs. In some embodiments, the prime editing methods disclosed herein have an editing efficiency of at least about 90% and an indel frequency of less than 1% in a population of target cells, e.g., a population of human HSCs. In some embodiments, the prime editing methods disclosed herein have an editing efficiency of at least about 90% and an indel frequency of less than 0.5% in a population of target cells, e.g., a population of human HSCs. In some embodiments, the prime editing methods disclosed herein have an editing efficiency of at least about 90% and an indel frequency of less than 0.1% in a population of target cells, e.g., a population of human HSCs.

In some embodiments, the prime editing methods disclosed herein have an editing efficiency of at least about 95% and an indel frequency of less than 10% in a population of target cells, e.g., a population of human HSCs. In some embodiments, the prime editing methods disclosed herein have an editing efficiency of at least about 95% and an indel frequency of less than 7.5% in a population of target cells, e.g., a population of human HSCs. In some embodiments, the prime editing methods disclosed herein have an editing efficiency of at least about 95% and an indel frequency of less than 5% in a population of target cells, e.g., a population of human HSCs. In some embodiments, the prime editing methods disclosed herein have an editing efficiency of at least about 95% and an indel frequency of less than 2.5% in a population of target cells, e.g., a population of human HSCs. In some embodiments, the prime editing methods disclosed herein have an editing efficiency of at least about 95% and an indel frequency of less than 1% as measured in a population of target cells, e.g., a population of human HSCs. In some embodiments, the prime editing methods disclosed herein have an editing efficiency of at least about 95% and an indel frequency of less than 0.5% as measured in a population of target cells, e.g., a population of human HSCs. In some embodiments, the prime editing methods disclosed herein have an editing efficiency of at least about 95% and an indel frequency of less than 0.1% as measured in a population of target cells, e.g., population of human HSCs. In some embodiments, any number of indels is determined after at least 1 hour, at least 2 hours, at least 6 hours, at least 12 hours, at least 24 hours, at least 36 hours, at least 48 hours, at least 3 days, at least 4 days, at least 5 days, at least 7 days, at least 10 days, or at least 14 days of exposing a target gene (e.g., an NCF1 gene, an NCF1B pseudogene, or an NCF1C pseudogene, within the genome of a cell) to a prime editing composition. In some embodiments, the editing efficiency is determined after 1 hour, 2 hours, 6 hours, 12 hours, 24 hours, 36 hours, 48 hours, 3 days, 4 days, 5 days, 7 days, 10 days, or 14 days of exposing a target gene (e.g., e.g., an NCF1 gene, an NCF1B pseudogene, or an NCF1C pseudogene within the genome of a cell) to a prime editing composition.

In some embodiments, the prime editing composition described herein result in less than 50%, less than 40%, less than 30%, less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, less than 1%, less than 0.9%, less than 0.8%, less than 0.7%, less than 0.6%, less than 0.5%, less than 0.4%, less than 0.3%, less than 0.2%, less than 0.1%, less than 0.09%, less than 0.08%, less than 0.07%, less than 0.06%, less than 0.05%, less than 0.04%, less than 0.03%, less than 0.02%, or less than 0.01% off-target editing in a chromosome that includes the target gene. In some embodiments, off-target editing is determined after at least 1 hour, at least 2 hours, at least 6 hours, at least 12 hours, at least 24 hours, at least 36 hours, at least 48 hours, at least 3 days, at least 4 days, at least 5 days, at least 7 days, at least 10 days, or at least 14 days of exposing a target gene (e.g., a nucleic acid within the genome of a cell) to a prime editing composition.

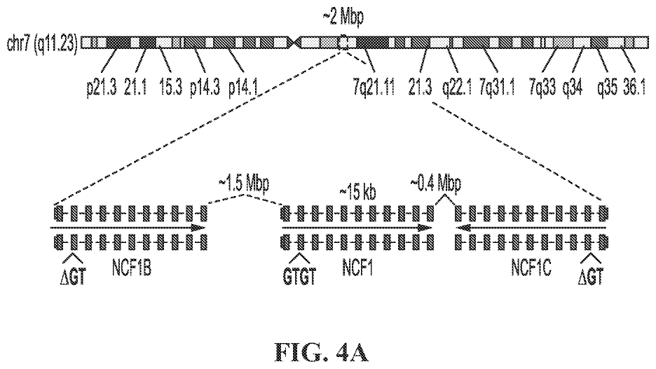

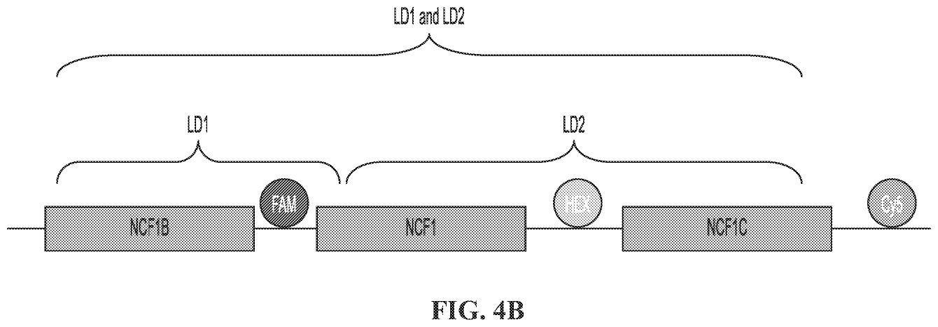

Because the NCF1 gene is flanked by two pseudogenes that have highly similar sequences as NCF1 (>99% sequence identity), genome editing of NCF1, NCF1B, and/or NCF1C may result in large deletion events of the regions between NCF1 and NCF1B, between NCF1 and NCF1C, or the entire region between NCF1B and NCF1C. As shown in FIG. 4B, deletion event between NCF1B and NCF1, between NCF1 and NCF1C, and between NCF1B and NCF1C would result in about 1.5 Mb, about 0.4 Mb, and about 2 Mb of deletion, respectively. In some embodiments, the prime editing method described herein can correct a NCF1 gene having a c-73_74delGT mutation (or a NCF1B/NCF1C pseudogene) with low level of large deletion events. Such deletion events may be measured by designing probes that bind to the sequences between NCF1B and NCF1 and/or between NCF1 and NCF1C. In some embodiments, the prime editing methods described herein result in less than 10%, less than 9%, less than 8%, less than 7%, less than 6%, less than 5%, less than 4%, less than 3%, less than 2%, less than 1%, or less than 0.5% large deletion in edited cells. In some embodiments, the prime editing methods described herein result in less than 4/large deletion in edited cells. In some embodiments, the prime editing methods described herein result in less than 3/large deletion in edited cells. In some embodiments, the prime editing methods described herein result in less than 2% large deletion in edited cells. In some embodiments, the prime editing methods described herein result in less than 1/large deletion in edited cells. In some embodiments, the prime editing methods described herein does not result in detectable level of large deletion in edited cells.

In some embodiments, the prime editing methods disclosed herein have an editing efficiency of at least about 90% and an indel frequency of less than 10% in a population of target cells, e.g., a population of human HSCs. In some embodiments, the prime editing methods disclosed herein have an editing efficiency of at least about 90% and an indel frequency of less than 7.5% in a population of target cells, e.g., a population of human HSCs. In some embodiments, the prime editing methods disclosed herein have an editing efficiency of at least about 90% and an indel frequency of less than 5% in a population of target cells, e.g., a population of human HSCs. In some embodiments, the prime editing methods disclosed herein have an editing efficiency of at least about 90% and an indel frequency of less than 2.5% in a population of target cells, e.g., a population of human HSCs. In some embodiments, the prime editing methods disclosed herein have an editing efficiency of at least about 90% and an indel frequency of less than 1% in a population of target cells, e.g., a population of human HSCs. In some embodiments, the prime editing methods disclosed herein have an editing efficiency of at least about 90%.

In some embodiments, the prime editing compositions (e.g., PEgRNAs and prime editors as described herein) and prime editing methods disclosed herein can be used to edit a target NCF1 gene, a target NCF1B pseudogene, or a target NCF1C pseudogene. In some embodiments, the target NCF1 gene comprises a mutation compared to a wild type NCF1 gene. In some embodiments, the mutation is associated with CGD. In some embodiments, the target NCF1 gene comprises an editing target sequence that contains the mutation associated with CGD. In some embodiments, the mutation is in a coding region of the target NCF1 gene. In some embodiments, the mutation is in an exon of the target NCF1 gene. In some embodiments, the mutation is in exon 1, exon 2, exon 3, exon 4, exon 5, exon 6, exon 7, exon 8, exon 9, exon 10, exon 11 of the NCF1 gene as compared to a wild type NCF1 gene. In some embodiments, the mutation is exon 2 of the NCF1 gene as compared to a wild type NCF1 gene. In some embodiments, the mutation is located between positions 74777167-74777368 of human chromosome 7 as set forth in human genome assembly consortium Human build 38 (GRCh38), GenBank accession GCF_000001405.38.

In some embodiments, the target NCF1 gene is in a chromosome in a cell. In some embodiments, the cell comprises a second copy of NCF1 gene. In some embodiments, the second copy of NCF1 gene comprises a mutation compared to a wild type NCF1 gene. In some embodiments, the second copy of NCF1 gene in the cell comprises a mutation that is different from the mutation in the target NCF1 gene. In some embodiments, editing of the target gene with the prime editing compositions (compositions (e.g., PEgRNAs and prime editors as described herein) and prime editing methods disclosed herein) restores expression and function of a NCF1 p47^{phox} protein subunit in the cell. In some embodiments, editing of the target gene with the prime editing compositions (compositions (e.g., PEgRNAs and prime editors as described herein) and prime editing methods disclosed herein) restores expression and function of a p47^{phox} in the cell.

In some embodiments, the prime editing compositions (e.g., PEgRNAs and prime editors as described herein) and prime editing methods disclosed herein can be used to edit a target pseudogene NCF1B. In some embodiments, the target NCF1 pseudogene comprises an editing target sequence in an exon of NCF1B. In some embodiments, the target NCF1 pseudogene comprises an editing target sequence in exon 2 of NCF1B. In some embodiments, the editing target sequence is located between positions 73223778-73223979 of human chromosome 7 as set forth in human genome assembly consortium Human build 38 (GRCh38), GenBank accession GCF_000001405.39. In some embodiments, the editing target sequence comprises nucleotides corresponding to positions 73223878-73223879 of human chromosome 7 as set forth in human genome assembly consortium Human build 38 (GRCh38), GenBank accession GCF_000001405.39.

In some embodiments, the prime editing compositions (e.g., PEgRNAs and prime editors as described herein) and prime editing methods disclosed herein can be used to edit a target NCF1 pseudogene, NCF1C. In some embodiments, the target NCF1 pseudogene comprises an editing target sequence in an exon of NCF1C. In some embodiments, the editing target sequence comprises a sequence in exon 2 of NCF1C. In some embodiments, the editing target sequence is located between positions 75168610-75168811 of human chromosome 7 as set forth in human genome assembly consortium Human build 38 (GRCh38), GenBank accession GCF_000001405.39. In some embodiments, the editing target sequence comprises nucleotides corresponding to positions 75168709-75168710 of human chromosome 7 as set forth in human genome assembly consortium Human build 38 (GRCh38), GenBank accession GCF_000001405.39.

In some embodiments, the prime editing method comprises contacting a target NCF1 gene with a prime editing composition comprising a prime editor, a PEgRNA, and/or a ngRNA. In some embodiments, contacting the target NCF1 gene with the prime editing composition results in incorporation of one or more intended nucleotide edits in the target NCF1 gene. In some embodiments, the incorporation is in a region of the target NCF1 gene that corresponds to an editing target sequence in the NCF1 gene. In some embodiments, the one or more intended nucleotide edits comprises a single nucleotide substitution, an insertion, a deletion, or any combination thereof, compared to the endogenous sequence of the target NCF1 gene. In some embodiments, incorporation of the one or more intended nucleotide edits results in replacement of one or more mutations with the corresponding sequence that encodes a wild type NCF1 polypeptide set forth in SEQ ID NO: 34422. In some embodiments, incorporation of the one or more intended nucleotide edits results in replacement of the one or more mutations with the corresponding sequence in a wild type NCF1 gene. In some embodiments, incorporation of the one more intended nucleotide edits results in correction of a mutation in the target NCF1 gene. In some embodiments, the target NCF1 gene comprises an editing template sequence that contains the mutation. In some embodiments, contacting the target NCF1 gene with the prime editing composition results in incorporation of one or more intended nucleotide edits in the target NCF1 gene, which corrects the mutation in the editing target sequence (or a double stranded region comprising the editing target sequence and the complementary sequence to the editing target sequence on a target strand) in the target NCF1 gene.

In some embodiments, incorporation of the one more intended nucleotide edits results in correction of a mutation in exon 2 of the target NCF1 gene as compared to a wild type NCF1 gene. In some embodiments, incorporation of the one more intended nucleotide edits results in correction of a mutation located between positions 74777167-74777368 of human chromosome 7 in the target NCF1 gene as set forth in human genome assembly consortium Human build 38 (GRCh38), GenBank accession GCF_000001405.38. In some embodiments, incorporation of the one more intended nucleotide edits results in a nucleotide insertion at position 74777167-74777368 in human chromosome 7 in the target NCF1 gene as compared to the endogenous sequence of the target NCF1 gene, thereby correcting c. 73_74GT (ΔGT) mutation at position 74777167-74777368 in human chromosome 7 in the target NCF1 gene as set forth in human genome assembly consortium Human build 38 (GRCh38), GenBank accession GCF_000001405.38. In some embodiments, incorporation of the one more intended nucleotide edits results in correction of an NCF1 gene sequence that comprises a c. 73_74GT (ΔGT) mutation, and restores wild type expression and function of the NCF1 protein. In some embodiments, the nucleotide insertion at positions 74777269 comprises a 2 nucleotide insertion. In some embodiments, the 2 nucleotide insertion is a GT, TT, CT, or AT.

In some embodiments, incorporation of the one more intended nucleotide edits result in insertion of a nucleotide sequence into exon 2 of a target NCF1B pseudogene. In some embodiments, incorporation of the one more intended nucleotide edits results in an insertion of a nucleotide sequence located between positions 73223778-73223979 and of human chromosome 7 in the target NCF1B pseudogene gene as set forth in human genome assembly consortium Human build 38 (GRCh38), GenBank accession GCF_000001405.39. In some embodiments, incorporation of the one more intended nucleotide edits results in a nucleotide insertion located between positions 73223778-73223979 in human chromosome 7 in the target NCF1B pseudogene as compared to the endogenous sequence of the target NCF1B pseudogene. In some embodiments, incorporation of the one more intended nucleotide edits results in a nucleotide insertion of a sequence into NCF1B pseudogene sequence, and restores the reading frame of NCF1B, thereby facilitating expression of NCF1B. In some embodiments, the intended nucleotide edit comprises a two nucleotide insertion at a position corresponding to position 73223878 in human chromosome 7 as compared to the region corresponding to the editing target sequence in the NCF1B pseudogene. In some embodiments, the two nucleotide insertion is a GT. In some embodiments, the intended nucleotide edit comprises a two nucleotide insertion at a position corresponding to position 73223880 in human chromosome 7 as compared to the region corresponding to the editing target sequence in the NCF1B pseudogene. In some embodiments, the two nucleotide insertion is a GT, TT, CT, or AT.

In some embodiments, incorporation of the one more intended nucleotide edits result in insertion of a nucleotide sequence into exon 2 of a target NCF1C pseudogene. In some embodiments, incorporation of the one more intended nucleotide edits results in an insertion of a nucleotide sequence located between positions 75168610-75168811 and of human chromosome 7 in the target NCF1C pseudogene gene as set forth in human genome assembly consortium Human build 38 (GRCh38), GenBank accession GCF_000001405.39. In some embodiments, incorporation of the one more intended nucleotide edits results in a nucleotide insertion located between positions 75168610-75168811 in human chromosome 7 in the target NCF1C pseudogene as compared to the endogenous sequence of the target NCF1C pseudogene. In some embodiments, incorporation of the one more intended nucleotide edits results in a nucleotide insertion of a sequence into NCF1C pseudogene sequence, and restores the reading frame of NCF1C, thereby facilitating expression of NCF1C. In some embodiments, the intended nucleotide edit comprises a two nucleotide insertion at a position corresponding to position 75168710 in human chromosome 7 as compared to the region corresponding to the editing target sequence in the NCF1C pseudogene. In some embodiments, the two nucleotide insertion is a GT. In some embodiments, the intended nucleotide edit comprises a two nucleotide insertion at a position corresponding to position 75168708 in human chromosome 7 as compared to the region corresponding to the editing target sequence in the NCF1C pseudogene. In some embodiments, the two nucleotide insertion is a GT, TT, CT, or AT.

In some embodiments, the target NCF1 gene, the target NCF1B pseudogene, or the target NCF1C pseudogene is in a target cell. Accordingly, in one aspect provided herein is a method of editing a target cell comprising a target NCF1 gene that encodes a p47^{phox} polypeptide that comprises one or more mutations relative to a wild type NCF1 gene. In some embodiments, provided herein is a method of editing a target cell comprising a target NCF1B pseudogene or a target NCF1C pseudogene to result in expression of a p47^{phox} polypeptide encoded by the target NCF1B pseudogene or the target NCF1C pseudogene. In some embodiments, the methods of the present disclosure comprise introducing a prime editing composition comprising a PEgRNA, a prime editor polypeptide, and/or a ngRNA into the target cell that has the target NCF1 gene, the target NCF1B pseudogene, or the target NCF1C pseudogene to edit the target NCF1 gene, the target NCF1B pseudogene, or the target NCF1C pseudogene, thereby generating an edited cell. In some embodiments, the target cell is a mammalian cell. In some embodiments, the target cell is a human cell. In some embodiments, the target cell is a primary cell. In some embodiments, the target cell is a human primary cell. In some embodiments, the target cell is a progenitor cell. In some embodiments, the target cell is a human progenitor cell. In some embodiments, the target cell is a stem cell. In some embodiments, the target cell is a human stem cell. In some embodiments, the target cell is a stem cell. In some embodiments, the target cell is a human stem cell. In some embodiments, the target cell is an iPSC. In some embodiments, the target cell is a human iPSC. In some embodiments, the target cell is a hematopoietic stem cell (HSC). In some embodiments, the target cell is a human HSC. In some embodiments, the HSC is from bone marrow or mobilized peripheral blood. In some embodiments, the cell is a hematopoietic stem and progenitor cell. In some embodiments the human stem cell is an induced pluripotent stem cell (iPSC). In some embodiments, the cell is a human HSC. In some embodiments, the cell is a human CD34⁺ cell. In some embodiments, the cell is a hematopoietic stem and progenitor cell (HSPC). In some embodiments, the target cell is a hematopoietic progenitor cell, multipotent progenitor cell, a lymphoid progenitor cell, a myeloid progenitor cell, a megakaryocyte-erythroid progenitor cell, a granulocyte-megakaryocyte progenitor cell, a granulocyte, a promyelocyte, a neutrophil, an eosinophil, a basophil, an erythrocyte, a reticulocyte, a thrombocyte, a megakaryoblast, a platelet-producing megakaryocyte, a monocyte, a macrophage, a dendritic cell, a microglia, an osteoclast, a lymphocyte, a NK cell, a B-cell, or a T-cell. In some embodiments, the cell edited by prime editing can be differentiated into, or give rise to recovery of a population of cells, e.g., common lymphoid progenitor cells, common myeloid progenitor cells, megakaryocyte-erythroid progenitor cells, granulocyte-megakaryocyte progenitor cells, granulocytes, promyelocytes, neutrophils, eosinophils, basophils, erythrocytes, reticulocytes, thrombocytes, megakaryoblasts, platelet-producing megakaryocytes, platelets, monocytes, macrophages, dendritic cells, microglia, osteoclasts, lymphocytes, such as NK cells, B-cells or T-cells. In some embodiments, the cell edited by prime editing can be differentiated into, or give rise to recovery of a population of cells, e.g., neutrophils, platelets, red blood cells, monocytes, macrophages, antigen-presenting cells, microglia, osteoclasts, dendritic cells, and/or lymphocytes. In some embodiments, the target cell is an ex vivo cell. In some embodiments, the target cell is an ex vivo cell obtained from a human subject. In some embodiments, the target cell is in a subject, e.g., a human subject.

In some embodiments, components of a prime editing composition described herein are provided to a target cell in vitro. In some embodiments, components of a prime editing composition described herein are provided to a target cell ex vivo. In some embodiments, components of a prime editing composition described herein are provided to a target cell in vivo.

In some embodiments, incorporation of the one or more intended nucleotide edits in the target NCF1 gene that comprises one or more mutations restores wild type expression and function of the NCF1 protein encoded by the NCF1 gene. In some embodiments, the target NCF1 gene comprises a c. 73_74GT (ΔGT) mutation as compared to the wild type NCF1 gene prior to incorporation of the one or more intended nucleotide edits. In some embodiments, expression and/or function of the NCF1 protein may be measured when expressed in a target cell. In some embodiments, incorporation of the one or more intended nucleotide edits in the target NCF1 gene comprising one or more mutations lead to a fold change in a level of NCF1 gene expression, NCF1 protein expression, or a combination thereof. In some embodiments, a change in the level of NCF1 expression level can comprise a fold change of, e.g., 2-fold, 3-fold, 4-fold, 5-fold, 6-fold, 7-fold, 8-fold, 9-fold, 10-fold, 15-fold, 20-fold, 25-fold, 30-fold, 40-fold, 50-fold, 60-fold, 70-fold, 80-fold, 90-fold, 100-fold or greater as compared to expression in a suitable control cell not introduced with a prime editing composition described herein. In some embodiments, incorporation of the one or more intended nucleotide edits in the target NCF1 gene that comprises one or more mutations restores wild type expression of the NCF1 protein by at least 10%, 20%, 30%, 40%, 50%, 60%, 70%, 80%, 90%, 95%, or 99% or more as compared to wild type expression of the NCF1 protein in a suitable control cell that comprises a wild type NCF1 gene.

In some embodiments, incorporation of the one or more intended nucleotide edits in the target NCF1B or NCF1C pseudogene that comprises one or more mutations or nucleotide alterations results in expression of a p47^{phox} polypeptide encoded by the target NCF1B or NCF1C pseudogene. In some embodiments, incorporation of the one or more intended nucleotide edits in the target NCF1B or NCF1C pseudogene that comprises one or more mutations or nucleotide alterations results in an edited NCF1B or NCF1C pseudogene that has a wild type NCF1 gene sequence. In some embodiments, incorporation of the one or more intended nucleotide edits in the target NCF1B or NCF1C pseudogene that comprises one or more mutations or nucleotide alterations results in expression of a functional p47^{phox} polypeptide. In some embodiments, incorporation of the one or more intended nucleotide edits in the target NCF1B or NCF1C pseudogene that comprises one or more mutations or nucleotide alterations results in expression of a functional p47^{phox} polypeptide that has the same sequence as a wild type p47^{phox} polypeptide. In some embodiments, expression and/or function of the p47^{phox} polypeptide (NCF1 polypeptide) may be measured when expressed in a target cell. In some embodiments, incorporation of the one or more intended nucleotide edits in the target NCF1B or NCF1C pseudogene leads to a fold change in a level of NCF1B or NCF1C transcript expression, p47^{phox} protein expression, or a combination thereof. In some embodiments, a change in the level of NCF1B or NCF1C transcript expression level can comprise a fold change of, e.g., 2-fold, 3-fold, 4-fold, 5-fold, 6-fold, 7-fold, 8-fold, 9-fold, 10-fold, 15-fold, 20-fold, 25-fold, 30-fold, 40-fold, 50-fold, 60-fold, 70-fold, 80-fold, 90-fold, 100-fold or greater as compared to expression in a suitable control cell not introduced with a prime editing composition described herein. In some embodiments, incorporation of the one or more intended nucleotide edits in the target NCF1B or NCF1C pseudogene restores wild type expression of the p47^{phox} protein by at least 10%, 20%, 30%, 40%, 50%, 60%, 70%, 80%, 90%, 95%, or 99% or more as compared to wild type expression of the NCF1 protein in a suitable control cell that comprises a wild type NCF1 gene. In some embodiments, a p47^{phox} protein expression increase can be measured by a functional assay. In some embodiments, the functional assay can comprise measurement of ferricytochrome c reduction, luminol-enhanced chemiluminescence, or ROS-mediated dihydrorhodamine (DHR) oxidation, which may be detected using flow cytometry, or any other functional assay known in the art or a combination thereof. In some embodiments, protein expression can be measured using a protein assay. In some embodiments, protein expression can be measured using antibody testing. In some embodiments, an antibody can comprise anti-p47^{phox}. In some embodiments, protein expression can be measured using ELISA, mass spectrometry, Western blot, sodium dodecyl sulfate polyacrylamide gel electrophoresis (SDS-PAGE), high performance liquid chromatography (HPLC), electrophoresis, or any combination thereof. In some embodiments, a protein assay can comprise SDS-PAGE and densitometric analysis of a Coomassie Blue-stained gel.

### Methods of Treating CGD

In some embodiments, provided herein are methods for treatment of a subject diagnosed with a disease associated with or caused by one or more pathogenic mutations. In some embodiments, provided herein are methods for treatment of a subject diagnosed with a disease associated with or caused by one or more pathogenic mutations that can be corrected by prime editing. In some embodiments, methods of treatment provided herein comprises editing one or more genes other than the gene that harbors the one or more pathogenic mutations. In some embodiments, provided herein are methods for treating CGD that comprise administering to a subject a therapeutically effective amount of a prime editing composition, or a pharmaceutical composition comprising a prime editing composition as described herein. In some embodiments, administration of the prime editing composition results in incorporation of one or more intended nucleotide edits in a target NCF1 gene in the subject. In some embodiments, administration of the prime editing composition results in correction of one or more pathogenic mutations, e.g., point mutations, insertions, or deletions, associated with CGD in the subject. In some embodiments, administration of the prime editing composition results in correction of one or more pathogenic mutations, e.g., point mutations, insertions, or deletions in the target NCF1 gene associated with CGD in the subject. In some embodiments, the target gene comprise an editing target sequence in the NCF1 gene that contains the pathogenic mutation. In some embodiments, administration of the prime editing composition results in incorporation of one or more intended nucleotide edits in the target NCF1 gene that corrects the pathogenic mutation in the editing target sequence (or a double stranded region comprising the editing target sequence and the complementary sequence to the editing target sequence on a target strand) of the target gene in the subject. In some embodiments, the subject has been diagnosed with CGD by sequencing of a NCF1 gene in the subject. In some embodiments, the subject comprises at least a copy of NCF1 gene that comprises one or more mutations compared to a wild type NCF1 gene. In some embodiments, the subject comprises at least a copy of NCF1 gene that comprises a mutation in a coding region of the NCF1 gene. In some embodiments, the subject comprises at least a copy of NCF1 gene that comprises a mutation in exon 1, exon 2, exon 3, exon 4, exon 5, exon 6, exon 7, exon 8, exon 9, exon 10, exon 11, as compared to a wild type NCF1 gene. In some embodiments, the subject comprises at least a copy of NCF1 gene that comprises a mutation in exon 2 as compared to a wild type NCF1 gene. In some embodiments, the subject comprises at least a copy of NCF1 gene comprising a c. 73_74GT (ΔGT) mutation, resulting in the deletion of a 2 nucleotide sequence located at positions 74777267-74777268 as compared to a wild type NCF1 gene. In some embodiments, the subject comprises at least a copy of NCF1 gene, wherein the copy of NCF1 gene comprises one or more of a c. 73_74GT (ΔGT) mutation, a c.125 G→A mutation, a c.124 C→T mutation, a c.157 A→G mutation, a c.229_1delG mutation, a c.247 G→A mutation, a c.268 C→T mutation, a c.269 G→A mutation, a c.271 C→T mutation, a c.292 T→G mutation, a c.295 A→G mutation, a c.328 C→T mutation, a c.333 T→A mutation, a c.331_339delTGTCCCCAC mutation, a c.347delA mutation, a c.353_354delTCinsAA mutation, a c.403 A→G mutation, a c.446 C→A mutation, a c.574 G→A mutation, a c.541 delG mutation, a c.502 delG mutation, a c.579 G→A mutation, a c.581 G→A mutation, a c.604 C→T mutation, a c.612 G→A mutation, a c.678 T→G mutation, a c.730 G→A mutation, a c.784 G→A mutation, a c.784 G→C mutation, a c.734_748delTGTCCCTGCTCGAGG mutation, a c.81ldelG mutation, a c.838delC mutation, a c.923 C→T mutation, a c.72+1 G→A mutation, a c.72+3 G→T mutation, a c.153+1 G→A mutation, a c.153+5 G→C mutation, a c.574 G→A mutation, a c.574+1 G→A mutation, a c.682+1 G→A mutation, and a c.682+1 G→C mutation compared to a wild type NCF1 gene, which may be corrected by the prime editing composition provided herein. In some embodiments, the subject comprises two copies of NCF1 gene, wherein each of the both copies of the NCF1 gene comprises a c. 73_74GT (ΔGT) mutation. In some embodiments, the subject comprises two copies of NCF1 gene, wherein both copies of the NCF1 gene comprises one or more of a c. 73_74GT (ΔGT) mutation, a c.125 G→A mutation, a c.124 C→T mutation, a c.157 A→G mutation, a c.229_ldelG mutation, a c.247 G→A mutation, a c.268 C→T mutation, a c.269 G→A mutation, a c.271 C→T mutation, a c.292 T→G mutation, a c.295 A→G mutation, a c.328 C→T mutation, a c.333 T→A mutation, a c.331_339delTGTCCCCAC mutation, a c.347delA mutation, a c.353_354delTCinsAA mutation, a c.403 A→G mutation, a c.446 C→A mutation, a c.574 GA mutation, a c.541 delG mutation, a c.502 delG mutation, a c.579 GA mutation, a c.581 GA mutation, a c.604 C→T mutation, a c.612 GA mutation, a c.678 T→G mutation, a c.730 GA mutation, a c.784 GA mutation, a c.784 G→C mutation, a c.734_748delTGTCCCTGCTCGAGG mutation, a c.81ldelG mutation, a c.838delC mutation, a c.923 C→T mutation, a c.72+1 G→A mutation, a c.72+3 G→T mutation, a c.153+1 G→A mutation, a c.153+5 G→C mutation, a c.574 G→A mutation, a c.574+1 G→A mutation, a c.682+1 G→A mutation, and a c.682+1 G→C mutation compared to a wild type NCF1 gene, which may be corrected by the prime editing composition provided herein. The mutations on both copies of the NCF1 gene in the subject may or may not be the same. In some embodiments, incorporation of the one or more intended nucleotide edits results in a wild type sequence of the target NCF1 gene, thereby treating CGD. In some embodiments, incorporation of the one or more intended nucleotide edits results in a GT two-nucleotide insertion at positions corresponding to positions 74777267-74777268, and/or a GT, AT, CT, or TT two-nucleotide insertion at corresponding to positions 74777269-74777270 of human chromosome 7 in the target NCF1 gene. In some embodiments, incorporation of the one or more intended nucleotide edits results in correction of the one or more mutations in the NCF1 gene. In some embodiments, incorporation of the one or more intended nucleotide edits restores expression of a wild type p47^{phox} polypeptide encoded by the target gene, thereby treating CGD.

In some embodiments, administration of the prime editing composition results in incorporation of one or more intended nucleotide edits in a target gene that is not the gene that harbors the pathogenic mutation. In some embodiments, administration of the prime editing composition results in incorporation of one or more intended nucleotide edits in a pseudogene that is not the gene that harbors the pathogenic mutation, wherein incorporation of the one or more intended nucleotide edits results in expression of a functional protein encoded by the pseudogene. In some embodiments, administration of the prime editing composition results in incorporation of one or more intended nucleotide edits in a NCF1B pseudogene. In some embodiments, the subject comprises at least a copy of NCF1 gene that comprises a mutation in exon 1, exon 2, exon 3, exon 4, exon 5, exon 6, exon 7, exon 8, exon 9, exon 10, exon 11, as compared to a wild type NCF1 gene. In some embodiments, the subject comprises at least a copy of NCF1 gene that comprises a mutation in exon 2 as compared to a wild type NCF1 gene. In some embodiments, the subject comprises at least a copy of NCF1 gene comprising a c. 73_74GT (ΔGT) mutation, resulting in the deletion of a 2 nucleotide sequence located at positions 74777267-74777268 as compared to a wild type NCF1 gene. In some embodiments, the subject comprises at least a copy of NCF1 gene, wherein the copy of NCF1 gene comprises one or more of a c. 73_74GT (ΔGT) mutation, a c.125 G→A mutation, a c.124 C→T mutation, a c.157 A→G mutation, a c.229_ldelG mutation, a c.247 G→A mutation, a c.268 C→T mutation, a c.269 G→A mutation, a c.271 C→T mutation, a c.292 T→G mutation, a c.295 A→G mutation, a c.328 C→T mutation, a c.333 T→A mutation, a c.331_339delTGTCCCCAC mutation, a c.347delA mutation, a c.353_354delTCinsAA mutation, a c.403 A→G mutation, a c.446 C→A mutation, a c.574 G→A mutation, a c.541 delG mutation, a c.502 delG mutation, a c.579 G→A mutation, a c.581 G→A mutation, a c.604 C→T mutation, a c.612 G→A mutation, a c.678 T→G mutation, a c.730 G→A mutation, a c.784 G→A mutation, a c.784 G→C mutation, a c.734_748delTGTCCCTGCTCGAGG mutation, a c.81ldelG mutation, a c.838delC mutation, a c.923 C→T mutation, a c.72+1 G→A mutation, a c.72+3 G→T mutation, a c.153+1 G→A mutation, a c.153+5 G→C mutation, a c.574 G→A mutation, a c.574+1 G→A mutation, a c.682+1 G→A mutation, and a c.682+1 G→C mutation compared to a wild type NCF1 gene, which may be corrected by the prime editing composition provided herein. In some embodiments, the subject comprises at least one copy of a NCF1 gene that comprises a mutation other than a c. 73_74GT (ΔGT) mutation compared to a wild type NCF1 gene. In some embodiments, the subject comprises two copies of NCF1 gene, wherein each of the both copies of the NCF1 gene comprises a c. 73_74GT (ΔGT) mutation. In some embodiments, the subject comprises two copies of a NCF1 gene, wherein each of the both copies of the NCF1 gene comprises a mutation other than a c. 73_74GT (ΔGT) mutation compared to a wild type NCF1 gene. In some embodiments, the subject comprises two copies of NCF1 gene, wherein both copies of the NCF1 gene comprises one or more of a c. 73_74GT (ΔGT) mutation, a c.125 G→A mutation, a c.124 C→T mutation, a c.157 A→G mutation, a c.229_ldelG mutation, a c.247 G→A mutation, a c.268 C→T mutation, a c.269 G→A mutation, a c.271 C→T mutation, a c.292 T→G mutation, a c.295 A→G mutation, a c.328 C→T mutation, a c.333 T→A mutation, a c.331_339delTGTCCCCAC mutation, a c.347delA mutation, a c.353_354delTCinsAA mutation, a c.403 A→G mutation, a c.446 C→A mutation, a c.574 G→A mutation, a c.541 delG mutation, a c.502 delG mutation, a c.579 G→A mutation, a c.581 G→A mutation, a c.604 C→T mutation, a c.612 G→A mutation, a c.678 T→G mutation, a c.730 G→A mutation, a c.784 G→A mutation, a c.784 G→C mutation, a c.734_748delTGTCCCTGCTCGAGG mutation, a c.81ldelG mutation, a c.838delC mutation, a c.923 C→T mutation, a c.72+1 G→A mutation, a c.72+3 G→T mutation, a c.153+1 G→A mutation, a c.153+5 G→C mutation, a c.574 G→A mutation, a c.574+1 G→A mutation, a c.682+1 G→A mutation, and a c.682+1 G→C mutation compared to a wild type NCF1 gene. The mutations on both copies of the NCF1 gene in the subject may or may not be the same. In some embodiments, administration of the prime editing composition results in incorporation of an insertion of GT nucleotides in a NCF1B pseudogene. In some embodiments, administration of the prime editing composition results in incorporation of an insertion of GT, AT, CT, or TT nucleotides in the exon 2 of a NCF1B pseudogene. In some embodiments, administration of the prime editing composition results in incorporation of an insertion of GT nucleotides in the exon 2 of a NCF1B pseudogene. In some embodiments, incorporation of the one or more intended nucleotide edits results in NCF1B pseudogene that has the same sequence as a wild type NCF1 gene. In some embodiments, incorporation of the one or more intended nucleotide edits results in expression of a functional p47^{phox} polypeptide encoded by the NCF1B pseudogene that compensates for the lack of functional p47^{phox} polypeptide caused by the one or more mutations in the NCF1 gene, thereby treating CGD. In some embodiments, incorporation of the one or more intended nucleotide edits results in expression of a wild type p47^{phox} polypeptide encoded by the NCF1B pseudogene, thereby treating CGD. In some embodiments, administration of the prime editing composition results in incorporation of one or more intended nucleotide edits in a NCF1C pseudogene. In some embodiments, administration of the prime editing composition results in incorporation of an insertion of GT nucleotides in a NCF1C pseudogene. In some embodiments, administration of the prime editing composition results in incorporation of an insertion of GT nucleotides in the exon 2 of a NCF1C pseudogene. In some embodiments, administration of the prime editing composition results in incorporation of an insertion of GT, AT, CT, or T nucleotides in the exon 2 of a NCF1C pseudogene. In some embodiments, incorporation of the one or more intended nucleotide edits results in NCF1C pseudogene that has the same sequence as a wild type NCF1 gene. In some embodiments, incorporation of the one or more intended nucleotide edits results in expression of a functional p47^{phox} polypeptide encoded by the NCF1C pseudogene that compensates for the lack of functional p47^{phox} polypeptide caused by the one or more mutations in the NCF1 gene thereby treating CGD. In some embodiments, incorporation of the one or more intended nucleotide edits results in expression of a wild type p47^{phox} polypeptide encoded by the NCF1C pseudogene, thereby treating CGD. In some embodiments, administration of the prime editing composition results in incorporation of one or more intended nucleotide edits in a NCF1B pseudogene and a NCF1C pseudogene. In some embodiments, administration of the prime editing composition results in incorporation of an insertion of GT nucleotides in a NCF1B pseudogene and a NCF1C pseudogene. In some embodiments, administration of the prime editing composition results in incorporation of an insertion of GT nucleotides in the exon 2 of a NCF1B pseudogene and a NCF1C pseudogene. In some embodiments, administration of the prime editing composition results in incorporation of an insertion of GT, AT, CT, or TT nucleotides in the exon 2 of a NCF1B pseudogene and a NCF1C pseudogene. In some embodiments, incorporation of the one or more intended nucleotide edits results in a NCF1B pseudogene and a NCF1C pseudogene that both have the same sequence as a wild type NCF1 gene. In some embodiments, incorporation of the one or more intended nucleotide edits results in expression of a functional p47^{phox} polypeptide encoded by the NCF1B pseudogene and the NCF1C pseudogene that compensates for the lack of functional p47^{phox} polypeptide caused by the one or more mutations in the NCF1 gene thereby treating CGD.

In some embodiments, the method provided herein comprises administering to a subject an effective amount of a prime editing composition, for example, a PEgRNA, a prime editor, and/or a ngRNA. In some embodiments, the method comprises administering to the subject an effective amount of a prime editing composition described herein, for example, polynucleotides, vectors, or constructs that encode prime editing composition components, or RNPs, LNPs, and/or polypeptides comprising prime editing composition components. Prime editing compositions can be administered to target the NCF1 gene in a subject, e.g., a human subject, suffering from, having, susceptible to, or at risk for CGD. Identifying a subject in need of such treatment can be in the judgment of a subject or a health care professional and can be subjective (e.g., opinion) or objective (e.g., measurable by a test or diagnostic method). In some embodiments, the subject has CGD.

In some embodiments, a population of patients each having one or more mutations in the target gene (e.g., an NCF1 gene) may be treated with a prime editing composition (e.g., a PEgRNA, a prime editor, and optionally an ngRNA as described herein) disclosed herein. In some embodiments, a population of patients with different mutations in the target gene can be treated with the same prime editing composition comprising a single PEgRNA, a prime editor, and optionally an ngRNA. In some embodiments, a single prime editing composition comprising a single PEgRNA and a prime editor can be used to correct one or more, or two or more, mutations in the target gene in a populations of patients, wherein one or more patients in the population have different mutations from one another. In some embodiments, the prime editing composition comprising a single pair of PEgRNA, a prime editor, and optionally an ngRNA can be used to correct 2, 3, 4, 5, 6, 7, 8, 9, 10, 15, 20, 25 or more mutations in the target gene in a population of patients, wherein one or more patients in the population have different mutations from one another. In some embodiments, the PEgRNA comprises an editing template comprising a wild type sequence of the gene.

In some embodiments, a patient with multiple mutations in the target gene (e.g., an NCF1 gene) can be treated with a prime editing composition (e.g., a PEgRNAs, a prime editor, and optionally an ngRNA as described herein). For example, in some embodiments, a subject may comprise two copies of the gene, each comprising one or more different mutations. In some embodiments, a patient with one or more different mutations in the target gene can be treated with a single prime editing composition comprising a PEgRNAs, a prime editor, and optionally an ngRNA.

In some embodiments, the method comprises directly administering prime editing compositions provided herein to a subject. The prime editing compositions described herein can be delivered with in any form as described herein, e.g., as LNPs, RNPs, polynucleotide vectors such as viral vectors, or mRNAs. The prime editing compositions can be formulated with any pharmaceutically acceptable carrier described herein or known in the art for administering directly to a subject. Components of a prime editing composition or a pharmaceutical composition thereof may be administered to the subject simultaneously or sequentially. For example, in some embodiments, the method comprises administering a prime editing composition, or pharmaceutical composition thereof, comprising a complex that comprises a prime editor fusion protein and a PEgRNA and/or a ngRNA, to a subject. In some embodiments, the method comprises administering a polynucleotide or vector encoding a prime editor to a subject simultaneously with a PEgRNA and/or a ngRNA. In some embodiments, the method comprises administering a polynucleotide or vector encoding a prime editor to a subject before administration with a PEgRNA and/or a ngRNA.

Suitable routes of administrating the prime editing compositions to a subject include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration. In some embodiments, the compositions described are administered intraperitoneally, intravenously, or by direct injection or direct infusion. In some embodiments, the compositions described are administered by direct injection or infusion into a subject. In some embodiments, the compositions described herein are administered to a subject by injection, by means of a catheter, by means of a suppository, or by means of an implant.

In some embodiments, the method comprises administering cells edited with a prime editing composition described herein to a subject. In some embodiments, the cells are allogeneic. In some embodiments, allogeneic cells are or have been contacted ex vivo with a prime editing composition or pharmaceutical composition thereof and are introduced into a human subject in need thereof. In some embodiments, the cells are autologous to the subject. In some embodiments, cells are removed from a subject and contacted ex vivo with a prime editing composition or pharmaceutical composition thereof and are re-introduced into the subject.

In some embodiments, cells are contacted er vivo with one or more components of a prime editing composition. In some embodiments, the ex vivo-contacted cells are introduced into the subject, and the subject is administered in vivo with one or more components of a prime editing composition. For example, in some embodiments, cells are contacted ex vivo with a prime editor and introduced into a subject. In some embodiments, the subject is then administered with a PEgRNA and/or a ngRNA, or a polynucleotide encoding the PEgRNA and/or the ngRNA.

In some embodiments, cells contacted with the prime editing composition are determined for incorporation of the one or more intended nucleotide edits in the genome before re-introduction into the subject. In some embodiments, the cells are enriched for incorporation of the one or more intended nucleotide edits in the genome before re-introduction into the subject. In some embodiments, the edited cells are primary cells. In some embodiments, the edited cells are progenitor cells. In some embodiments, the edited cells are stem cells. In some embodiments, the edited cells are hematopoietic stem cells (HSCs). In some embodiments, the edited cells are primary human cells. In some embodiments, the edited cells are human progenitor cells. In some embodiments, the edited cells are human stem cells. In some embodiments, the edited cells are hematopoietic stem cells (HSCs). In some embodiments, the edited cells are human HSCs. In some embodiments, the edited cells are hematopoietic stem and progenitor cells (HSPCs). In some embodiments, the edited cells are human hematopoietic stem and progenitor cells (HSPCs). In some embodiments, the edited cells are human CD34⁺ cells. In some embodiments, the edited cells are iPSCs. In some embodiments, the edited cells are HSC is from bone marrow or mobilized peripheral blood. In some embodiments, the cell is a hematopoietic stem and progenitor cell. In some embodiments, the cell is a human HSC. In some embodiments, the cell is a HSPC. In some embodiments, the cell is a human HSPC. In some embodiments, the cell is a human CD34⁺ cell. In some embodiments, the edited cells are hematopoietic progenitor cells, multipotent progenitor cells, lymphoid progenitor cells, myeloid progenitor cells, megakaryocyte-erythroid progenitor cells, granulocyte-megakaryocyte progenitor cells, granulocytes, promyelocytes, neutrophils, eosinophils, basophil, erythrocytes, reticulocytes, thrombocytes, megakaryoblasts, platelet-producing megakaryocytes, monocytes, macrophages, dendritic cells, microglia, osteoclasts, lymphocytes, NK cells, B-cells, or T-cells. In some embodiments, the cell edited by prime editing can be differentiated into, or give rise to recovery of a population of cells, e.g., common lymphoid progenitor cells, common myeloid progenitor cells, megakaryocyte-erythroid progenitor cells, granulocyte-megakaryocyte progenitor cells, granulocytes, promyelocytes, neutrophils, eosinophils, basophils, erythrocytes, reticulocytes, thrombocytes, megakaryoblasts, platelet-producing megakaryocytes, platelets, monocytes, macrophages, dendritic cells, microglia, osteoclasts, lymphocytes, such as NK cells, B-cells or T-cells. In some embodiments, the cell edited by prime editing can be differentiated into, or give rise to recovery of a population of cells, e.g., neutrophils, platelets, red blood cells, monocytes, macrophages, antigen-presenting cells, microglia, osteoclasts, dendritic cells, and/or lymphocytes. In some embodiments, the edited cells are an ex vivo cells. In some embodiments, the edited cells are an ex vivo cells obtained from a human subject. In some embodiments, the edited cells are in a subject, e.g., a human subject. The prime editing composition or components thereof may be introduced into a cell by any delivery approaches as described herein, including LNP administration, RNP administration, electroporation, nucleofection, transfection, viral transduction, microinjection, cell membrane disruption and diffusion, or any other approach known in the art.

The cells edited with prime editing can be introduced into the subject by any route known in the art. In some embodiments, the edited cells are administered to a subject by direct infusion. In some embodiments, the edited cells are administered to a subject by intravenous infusion. In some embodiments, the edited cells are administered to a subject as implants.

The pharmaceutical compositions, prime editing compositions, and cells, as described herein, can be administered in effective amounts. In some embodiments, the effective amount depends upon the mode of administration. In some embodiments, the effective amount depends upon the stage of the condition, the age and physical condition of the subject, the nature of concurrent therapy, if any, and like factors well-known to the medical practitioner.

The specific dose administered can be a uniform dose for each subject. Alternatively, a subject's dose can be tailored to the approximate body weight of the subject. Other factors in determining the appropriate dosage can include the disease or condition to be treated or prevented, the severity of the disease, the route of administration, and the age, sex and medical condition of the patient.

In embodiments wherein components of a prime editing composition are administered sequentially, the time between sequential administration can be at least 1 hour, at least 2 hours, at least 6 hours, at least 12 hours, at least 24 hours, at least 36 hours, at least 48 hours, at least 3 days, at least 4 days, at least 5 days, at least 7 days, at least 10 days, or at least 14 days.

In some embodiments, a method of monitoring treatment progress is provided. In some embodiments, the method includes the step of determining a level of diagnostic marker, for example, correction of a mutation in NCF1 gene, expression level of a functional p47^{phox} polypeptide, or diagnostic measurement associated with CGD, (e.g., ROS-mediated dihydrorhodamine (DHR) oxidation assay) in a subject suffering from CGD symptoms and has been administered an effective amount of a prime editing composition described herein. The level of the diagnostic marker determined in the method can be compared to known levels of the marker in either healthy normal controls or in other afflicted subjects to establish the subject's disease status.

### Delivery

Prime editing compositions described herein can be delivered to a cellular environment with any approach known in the art. Components of a prime editing composition can be delivered to a cell by the same mode or different modes. For example, in some embodiments, a prime editor can be delivered as a polypeptide or a polynucleotide (DNA or RNA) encoding the polypeptide. In some embodiments, a PEgRNA can be delivered directly as an RNA or as a DNA encoding the PEgRNA.

In some embodiments, a prime editing composition component is encoded by a polynucleotide, a vector, or a construct. In some embodiments, a prime editor polypeptide, a PEgRNA and/or a ngRNA is encoded by a polynucleotide. In some embodiments, the polynucleotide encodes a prime editor fusion protein comprising a DNA binding domain and a DNA polymerase domain. In some embodiments, the polynucleotide encodes a DNA polymerase domain of a prime editor. In some embodiments, the polynucleotide encodes a DNA polymerase domain of a prime editor. In some embodiments, the polynucleotide encodes a portion of a prime editor protein, for example, a N-terminal portion of a prime editor fusion protein connected to an intein-N. In some embodiments, the polynucleotide encodes a portion of a prime editor protein, for example, a C-terminal portion of a prime editor fusion protein connected to an intein-C. In some embodiments, the polynucleotide encodes a PEgRNA and/or a ngRNA. In some embodiments, the polypeptide encodes two or more components of a prime editing composition, for example, a prime editor fusion protein and a PEgRNA.

In some embodiments, the polynucleotide encoding one or more prime editing composition components is delivered to a target cell is integrated into the genome of the cell for long-term expression, for example, by a retroviral vector. In some embodiments, the polynucleotide delivered to a target cell is expressed transiently. For example, the polynucleotide may be delivered in the form of a mRNA, or a non-integrating vector (non-integrating virus, plasmids, minicircle DNAs) for episomal expression.

In some embodiments, a polynucleotide encoding one or more prime editing system components can be operably linked to a regulatory element, e.g., a transcriptional control element, such as a promoter. In some embodiments, the polynucleotide is operably linked to multiple control elements. Depending on the expression system utilized, any of a number of suitable transcription and translation control elements, including constitutive and inducible promoters, transcription enhancer elements, transcription terminators, etc. may be used in the expression vector (e.g., U6 promoter, H1 promoter).

In some embodiments, the polynucleotide encoding one or more prime editing composition components is a part of, or is encoded by, a vector. In some embodiments, the vector is a viral vector. In some embodiments, the vector is a non-viral vector.

Non-viral vector delivery systems can include DNA plasmids, RNA (e.g., a transcript of a vector described herein), naked nucleic acid, and nucleic acid complexed with a delivery vehicle, such as a liposome. In some embodiments, the polynucleotide is provided as an RNA, e.g., a mRNA or a transcript. Any RNA of the prime editing systems, for example a guide RNA or a base editor-encoding mRNA, can be delivered in the form of RNA. In some embodiments, one or more components of the prime editing system that are RNAs is produced by direct chemical synthesis or may be transcribed in vitro from a DNA. In some embodiments, a mRNA that encodes a prime editor polypeptide is generated using in vitro transcription. Guide polynucleotides (e.g., PEgRNA or ngRNA) can also be transcribed using in vitro transcription from a cassette containing a T7 promoter, followed by the sequence “GG”, and guide polynucleotide sequence. In some embodiments, the prime editor encoding mRNA, PEgRNA, and/or ngRNA are synthesized in vitro using an RNA polymerase enzyme (e.g., T7 polymerase, T3 polymerase, SP6 polymerase, etc.). Once synthesized, the RNA can directly contact a target NCF1 gene or can be introduced into a cell using any suitable technique for introducing nucleic acids into cells (e.g., microinjection, electroporation, transfection). In some embodiments, the prime editor-coding sequences, the PEgRNAs, and/or the ngRNAs are modified to include one or more modified nucleoside e.g. using pseudo-U or 5-Methyl-C.

Methods of non-viral delivery of nucleic acids can include lipofection, electroporation, nucleofection, microinjection, biolistics, virosomes, liposomes, immunoliposomes, polycation or lipid:nucleic acid conjugates, nanoparticles, cell penetrating peptides and associated conjugated molecules and chemistry, naked DNA, artificial virions, cell membrane disruption by a microfluidics device, and agent-enhanced uptake of DNA. Cationic and neutral lipids that are suitable for efficient receptor-recognition lipofection of polynucleotides can be used. Delivery can be to cells (e.g., in vitro or ex vivo administration) or target tissues (e.g., in vivo administration). The preparation of lipid:nucleic acid complexes, including targeted liposomes such as immunolipid complexes, can be used.

Viral vector delivery systems can include DNA and RNA viruses, which can have either episomal or integrated genomes after delivery to the cell. RNA or DNA viral based systems can be used to target specific cells and trafficking the viral payload to an organelle of the cell. Viral vectors can be administered directly (in vivo) or they can be used to treat cells in vitro, and the modified cells can optionally be administered after delivery (ex vivo).

In some embodiments, the viral vector is a retroviral, lentiviral, adenoviral, adeno-associated viral or herpes simplex viral vector. Retroviral vectors can include those based upon murine leukemia virus (MuLV), gibbon ape leukemia virus (GaLV), Simian Immuno deficiency virus (SIV), human immuno deficiency virus (HIV), and combinations thereof. In some embodiments, the retroviral vector is a lentiviral vector. In some embodiments, the retroviral vector is a gamma retroviral vector. In some embodiments, the viral vector is an adenoviral vector. In some embodiments, the viral vector is an adeno-associated virus (“AAV”) vector.

In some embodiments, polynucleotides encoding one or more prime editing composition components are packaged in a virus particle. Packaging cells can be used to form virus particles that can infect a target cell. Such cells can include 293 cells, (e.g., for packaging adenovirus), and .psi.2 cells or PA317 cells (e.g., for packaging retrovirus). Viral vectors can be generated by producing a cell line that packages a nucleic acid vector into a viral particle. The vectors can contain the minimal viral sequences required for packaging and subsequent integration into a host. The vectors can contain other viral sequences being replaced by an expression cassette for the polynucleotide(s) to be expressed. The missing viral functions can be supplied in trans by the packaging cell line. For example, AAV vectors can comprise ITR sequences from the AAV genome which are required for packaging and integration into the host genome. In some embodiment, the polynucleotides are a DNA polynucleotide. In some embodiment, the polynucleotides are an RNA polynucleotide: e.g., an mRNA polynucleotide.

In some embodiments, the AAV vector is selected for tropism to a particular cell, tissue, organism. In some embodiments, the AAV vector is pseudotyped, e.g., AAV5/8. In some embodiments, polynucleotides encoding one or more prime editing composition components are packaged in a first AAV and a second AAV. In some embodiments, the polynucleotides encoding one or more prime editing composition components are packaged in a first rAAV and a second rAAV.

In some embodiments, dual AAV vectors are generated by splitting a large transgene expression cassette in two separate halves (5′ and 3′ ends that encode N-terminal portion and C-terminal portion of, e.g., a prime editor polypeptide), where each half of the cassette is no more than 5 kb in length, optionally no more than 4.7 kb in length, and is packaged in a single AAV vector. In some embodiments, the full-length transgene expression cassette is reassembled upon co-infection of the same cell by both dual AAV vectors. In some embodiments, a portion or fragment of a prime editor polypeptide, e.g., a Cas9 nickase, is fused to an intein. The portion or fragment of the polypeptide can be fused to the N-terminus or the C-terminus of the intein. In some embodiments, a N-terminal portion of the polypeptide is fused to an intein-N, and a C-terminal portion of the polypeptide is separately fused to an intein-C. In some embodiments, a portion or fragment of a prime editor fusion protein is fused to an intein and fused to an AAV capsid protein. In some embodiments, intein-N may be fused to the N-terminal portion of a first domain described herein, and and intein-C may be fused to the C-terminal portion of a second domain described herein for the joining of the N-terminal portion to the C-terminal portion, thereby joining the first and second domains. In some embodiments, the first and second domains are each independently chosen from a DNA binding domain or a DNA polymerase domain. The intein, nuclease and capsid protein can be fused together in any arrangement (e.g., nuclease-intein-capsid, intein-nuclease-capsid, capsid-intein-nuclease, etc.). In some embodiments, a polynucleotide encoding a prime editor fusion protein is split in two separate halves, each encoding a portion of the prime editor fusion protein and separately fused to an intein. In some embodiments, each of the two halves of the polynucleotide is packaged in an individual AAV vector of a dual AAV vector system. In some embodiments, each of the two halves of the polynucleotide is no more than 5 kb in length, optionally no more than 4.7 kb in length. In some embodiments, the fill-length prime editor fusion protein is reassembled upon co-infection of the same cell by both dual AAV vectors, expression of both halves of the prime editor fusion protein, and self-excision of the inteins. In some embodiments, the in vivo use of dual AAV vectors results in the expression of full-length full-length prime editor fusion proteins. In some embodiments, the use of the dual AAV vector platform allows viable delivery of transgenes of greater than about 4.5, 4.6, 4.7, 4.8, 4.9, or 5.0 kb in size.

In some embodiments, an intein is inserted at a splice site within a Cas protein. In some embodiments, insertion of an intein disrupts a Cas activity. As used herein, “intein” refers to a self-splicing protein intron (e.g., peptide), e.g., which ligates flanking N-terminal and C-terminal exteins (e.g., fragments to be joined). In some embodiments, an intein may comprise a polypeptide that is able to excise itself and join exteins with a peptide bond (e.g., protein splicing). In some embodiments, an intein of a precursor gene comes from two genes (e.g., split intein). In some embodiments, an intein may be a synthetic intein. Non-limiting examples of intein pairs that may be used in accordance with the present disclosure include: dnaE-n and dnaE-c. a 4-hydroxytamoxifen (4-HT)-responsive intein, an iCas molecule, a Ssp DnaX intein, Ter DnaE3 intein, Ter ThyX intein, Rma DnaB intein, Cfa DnaE intein, Ssp GyrB intein, and Rma DnaB intein. In some embodiments, intein fragments may be fused to the N terminal and C-terminal portion of a split Cas protein respectively for joining the fragments of split Cas9.

In some embodiments, the split Cas9 system may be used in general to bypass the packing limit of the viral delivery vehicles. In some embodiments, a split Cas9 may be a Type II CRISPR system Cas9. In some embodiments, a first nucleic acid encodes a first portion of the Cas9 protein having a first split-intein and wherein the second nucleic acid encodes a second portion of the Cas9 protein having a second split-intein complementary to the first split-intein and wherein the first portion of the Cas9 protein and the second portion of the Cas9 protein are joined together to form the Cas9 protein. In some embodiments, the first portion of the Cas9 protein is the N-terminal fragment of the Cas9 protein and the second portion of the Cas9 protein is the C-terminal fragment of the Cas9 protein. In some embodiments, a split site may be selected which are surface exposed due to the sterical need for protein splicing.

In some embodiments, a Cas protein may be split into two fragments at any C, T, A, or S. In some embodiments, a Cas9 may be intein split at residues 203-204, 280-292, 292-364, 311-325, 417-438, 445-483, 468-469, 481-502, 513-520, 522-530, 565-637, 696-707, 713-714, 795-804, 803-810, 878-887, and 1153-1154. In some embodiments, protein is divided into two fragments at SpCas9 T310, T313, A456, S469, or C574. In some embodiments, split Cas9 fragments across different split pairs yield combinations that provided the complete polypeptide sequence activate gene expression even when fragments are partially redundant. In some embodiments, a functional Cas9 protein may be reconstituted from two inactive split-Cas9 peptides in the presence of gRNA by using a split-intein protein splicing strategy. In some embodiment, the split Cas9 fragments are fused to either a N-terminal intein fragment or a C-terminal intein fragment, which can associate with each other and catalytically splice the two split Cas9 fragments into a functional reconstituted Cas9 protein. In some embodiments, a split-Cas9 can be packaged into self-complementary AAV. In some embodiments, a split-Cas9 comprises a 2.5 kb and a 2.2 kb fragment of S. pyogenes Cas9 coding sequences.

In some embodiments, a split-Cas9 architecture reduces the length and/or size of the coding sequences of a viral vector, e.g., AAV.

A target cell can be transiently or non-transiently transfected with one or more vectors described herein. A cell can be transfected as it naturally occurs in a subject. A cell can be taken or derived from a subject and transfected. A cell can be derived from cells taken from a subject, such as a cell line. In some embodiments, a cell transfected with one or more vectors described herein can be used to establish a new cell line comprising one or more vector-derived sequences. In some embodiments, a cell transiently transfected with the compositions of the disclosure (such as by transient transfection of one or more vectors, or transfection with RNA), and modified through the activity of a prime editor, can be used to establish a new cell line comprising cells containing the modification but lacking any other exogenous sequence. Any suitable vector compatible with the host cell can be used with the methods of the disclosure. Non-limiting examples of vectors include pXT1, pSG5, pSVK3, pBPV, pMSG, and pSVLSV40.

In some embodiments, a prime editor protein can be provided to cells as a polypeptide. In some embodiments, the prime editor protein is fused to a polypeptide domain that increases solubility of the protein. In some embodiments, the prime editor protein is formulated to improve solubility of the protein.

In some embodiment, a prime editor polypeptide is fused to a polypeptide permeant domain to promote uptake by the cell. In some embodiments, the permeant domain is a including peptide, a peptidomimetic, or a non-peptide carrier. For example, a permeant peptide may be derived from the third alpha helix of Drosophila melanogaster transcription factor Antennapaedia, referred to as penetratin. As another example, the permeant peptide can comprise the HIV-1 tat basic region amino acid sequence, which may include, for example, amino acids 49-57 of naturally-occurring tat protein. Other permeant domains can include poly-arginine motifs, for example, the region of amino acids 34-56 of HIV-1 rev protein, nona-arginine, and octa-arginine. The nona-arginine (R9) sequence can be used. The site at which the fusion can be made may be selected in order to optimize the biological activity, secretion or binding characteristics of the polypeptide.

In some embodiments, a prime editor polypeptide is produced in vitro or by host cells, and it may be further processed by unfolding, e.g., heat denaturation, DTT reduction, etc. and may be further refolded. In some embodiments, a prime editor polypeptide is prepared by in vitro synthesis. Various commercial synthetic apparatuses can be used. By using synthesizers, naturally occurring amino acids can be substituted with unnatural amino acids. In some embodiments, a prime editor polypeptide is isolated and purified in accordance with recombinant synthesis methods, for example, by expression in a host cell and the lysate purified using HPLC, exclusion chromatography, gel electrophoresis, affinity chromatography, or other purification technique.

In some embodiments, a prime editing composition, for example, prime editor polypeptide components and PEgRNA/ngRNA are introduced to a target cell by nanoparticles. In some embodiments, the prime editor polypeptide components and the PEgRNA and/or ngRNA form a complex in the nanoparticle. Any suitable nanoparticle design can be used to deliver genome editing system components or nucleic acids encoding such components. In some embodiments, the nanoparticle is inorganic. In some embodiments, the nanoparticle is organic. In some embodiments, a prime editing composition is delivered to a target cell, e.g., a HSC, in an organic nanoparticle, e.g., a lipid nanoparticle (LNP) or polymer nanoparticle.

In some embodiments, LNPs are formulated from cationic, anionic, neutral lipids, or combinations thereof. In some embodiments, neutral lipids, such as the fusogenic phospholipid DOPE or the membrane component cholesterol, are included to enhance transfection activity and nanoparticle stability. In some embodiments, LNPs are formulated with hydrophobic lipids, hydrophilic lipids, or combinations thereof. Lipids may be formulated in a wide range of molar ratios to produce an LNP. Any lipid or combination of lipids that are known in the art can be used to produce an LNP. Exemplary lipids used to produce LNPs are provided in Table 3 below.

In some embodiments, components of a prime editing composition form a complex prior to delivery to a target cell. For example, a prime editor fusion protein, a PEgRNA, and/or a ngRNA can form a complex prior to delivery to the target cell. In some embodiments, a prime editing polypeptide (e.g., a prime editor fusion protein) and a guide polynucleotide (e.g. a PEgRNA or ngRNA) form a ribonucleoprotein (RNP) for delivery to a target cell. In some embodiments, the RNP comprises a prime editor fusion protein in complex with a PEgRNA. RNPs may be delivered to cells using known methods, such as electroporation, nucleofection, or cationic lipid-mediated methods, or any other approaches known in the art. In some embodiments, delivery of a prime editing composition or complex to the target cell does not require the delivery of foreign DNA into the cell. In some embodiments, the RNP comprising the prime editing complex is degraded overtime in the target cell. Exemplary lipids for use in nanoparticle formulations and/or gene transfer are shown in Table 85 below.

TABLE 85
Exemplary lipids for nanoparticle formulation or gene transfer

| Lipid | Abbreviation | Feature |
|---|---|---|
| 1,2-Dioleoyl-sn-glycero-3-phosphatidylcholine | DOPC | Helper |
| 1,2-Dioleoyl-sn-glycero-3-phosphatidylethanolamine | DOPE | Helper |
| Cholesterol | | Helper |
| N41-(2,3-Dioleyloxy)prophyliN,N,N-trimethylammonium | DOTMA | Cationic |
| Chloride | | |
| 1,2-Dioleoyloxy-3-trimethylammonium-propane | DOGS | Cationic |
| Dioctadecylamidoglycylspermine | | |
| N-(3-Aminopropy1)-N,N-dimethy1-2,3-bis(dodecyloxy)-1- | GAP- | Cationic |
| propanaminium bromide | DLRIE | |
| Cetyltrimethylammonium bromide | CTAB | Cationic |
| 6-Lauroxyhexyl omithinate | LHON | Cationic |
| 1-(2,3-Dioleoyloxypropy1)-2,4,6-trimethylpyridinium | 2Oc | Cationic |
| 2,3-Dioleyloxy-N-P(spenninecarboxamido-ethy1J-N,Ndimethyl- | DOSPA | Cationic |
| 1-propanatninium trifluoroacetate | | |
| 1,2-Dioley1-3-trimethylamtnonium-propane | DOPA | Cationic |
| N-(2-Hydroxyethyl)-N,N-dimethy1-2,3-bis(tetradecyloxy)-1- | MDRIE | Cationic |
| propanaminium bromide | | |
| Dimyristooxypropyl dimethyl hydroxyethyl ammonium bromide | DMRI | Cationic |
| 3β-[N-(N′,N′-Dimethylaminoethane)-carbamoyl]cholesterol | DC-Chol | Cationic |
| Bis-guanidium-tren-cholesterol | BGTC | Cationic |
| 1,3-Diodeoxy-2-(6-carboxy-spermy1)-propylamide | DOSPER | Cationic |
| Dimethyloctadecylammonium bromide | DDAB | Cationic |
| Dioctadecylamidoglicylspermidin | DSL | Cationic |
| rac-[(2,3-Dioctadecyloxypropyl)(2-hydroxyethyl)]- | CLIP-1 | Cationic |
| dimethylammonium chloride | | |
| rac-[2(2,3-Dihexadecyloxypropyloxymethyloxy) | CLIP-6 | Cationic |
| ethyl]trimethylammoniun bromide | | |
| Ethyldimyristoylphosphatidylcholine | EDMPC | Cationic |
| 1,2-Distearyloxy-N,N-dimethyl-3-aminopropane | DSDMA | Cationic |
| 1,2-Dimyristoyl-trimethylammonium propane | DMTAP | Cationic |
| O,O′-Dimyristyl-N-lysyl aspartate | DMKE | Cationic |
| 1,2-Distearoyl-sn-glycero-3-ethylpho sphocholine | DSEPC | Cationic |
| N-Palmitoy1D-erythro-sphingosyl carbamoyl-spenmine | CCS | Cationic |
| N-t-Butyl-N0-tetradecyl-3-tetradecylaminopropionamidine | diC14- | Cationic |
| | amidine | |
| Octadecenolyoxy[ethyl-2-heptadecenyl-3 hydroxyethyl] | DOTIM | Cationic |
| imidazolinium chloride | | |
| N1-Cholesteryloxycarbonyl-3,7-diazanonane-1,9-diamine | CDAN | Cationic |
| 2-(3-Bis(3-amino-propy1)-amino]propylamino)- | RPR209120 | Cationic |
| Nditetradecylcarbamoylme-ethyl-acetamide | | |
| 1,2-dilinoleyloxy-3-dimethylaminopropane | DLinDMA | Cationic |
| 2,2-dilinoley1-4-dimethylaminoethyl-[1,3]-dioxolane | DLin-KC2- | Cationic |
| | DMA | |
| dilinoleyl-methyl-4-dimethylaminobutyrate | DLin-MC3- | Cationic |
| | DMA | |

Exemplary polymers for use in nanoparticle formulations and/or gene transfer are shown in Table 86 below.

TABLE 86
Exemplary lipids for nanoparticle formulation or gene transfer

| Polymer | Abbreviation |
|---|---|
| Poly(ethylene)glycol | PEG |
| Polyethylenimine | PEI |
| Dithiobis (succinimidylpropionate) | DSP |
| Dimethyl-3,3′-dithiobispropionimidate | DTBP |
| Poly(ethylene imine)biscarbamate | PEIC |
| Poly(L-lysine) | PLL |
| Histidine modified PLL | |
| Poly(N-vinylpyrrolidone) | PVP |
| Poly(propylenimine) | PPI |
| Poly(amidoamine) | PAMAM |
| Poly(amidoethylenimine) | SS_PAEI |
| Triethylenetetramine | TETA |
| Poly(β-aminoester) | |
| Poly(4-hydroxy-L-proline ester) | PHP |
| Poly(allylamine) | |
| Poly(α-[4-aminobutyl]-L-glycolic acid) | PAGA |
| Poly(D,L-lactic-co-glycolic acid) | PLGA |
| Poly(N-ethyl-4-vinylpyridinium bromide) | |
| Poly(phosphazene)s | PPZ |
| Poly(phosphoester)s | PPE |
| Poly(phosphoramidate)s | PPA |
| Poly(N-2-hydroxypropylmethacrylamide) | pHPMA |
| Poly (2-(dimethylamino)ethyl methacrylate) | pDMAEMA |
| Poly(2-aminoethyl propylene phosphate) | PPE-EA |
| Chitosan | |
| Galactosylated chitosan | |
| N-dodacylated chitosam | |
| Histone | |
| Collagen | |
| Dextran-spermine | D-SPM |

Exemplary delivery methods for polynucleotides encoding prime editing composition components are shown in Table 87 below.

TABLE 87
Exemplary polynucleotide delivery methods

| | | Delivery into | | | Type of |
|---|---|---|---|---|---|
| | | Non-Dividing | Duration of | Genome | Molecule |
| Delivery | Vector/Mode | Cells | Expression | Integration | Delivered |
| Physical | (e.g., | YES | Transient | NO | Nucleic Acids |
| | electroporation, | | | | and Proteins |
| | particle gun, | | | | |
| | Calcium phosphate | | | | |
| | transfection) | | | | |
| Viral | Retrovirus | NO | Stable | YES | RNA |
| | Lentivirus | YES | Stable | YES/NO with | RNA |
| | | | | modification | |
| | Adenovirus | YES | Transient | NO | DNA |
| | Adeno-Associated | YES | Stable | NO | DNA |
| | Virus (AAV) | | | | |
| | Vaccinia Virus | YES | Very | NO | DNA |
| | | | Transient | | |
| | Herpes Simplex | YES | Stable | NO | DNA |
| | Virus | | | | |
| Non-Viral | Cationic | YES | Transient | Depends on | Nucleic acids |
| | | | | what is | and Proteins |
| | | | | delivered | |
| | Polymeric | YES | Transient | NO | Nucleic Acids |
| | Nanoparticles | | | | |
| Biological | Attenuated Bacteria | YES | Transient | NO | Nucleic Acids |
| Non-Viral | Engineered | YES | Transient | NO | Nucleic Acids |
| Delivery | Bacteriophages | | | | |
| Vehicles | Mammalian Virus- | YES | Transient | NO | Nucleic Acids |
| | like Particles | | | | |
| | Biological | YES | Transient | NO | Nucleic Acids |
| | liposomes: | | | | |
| | Erythrocyte Ghosts | | | | |
| | and Exosomes | | | | |

The prime editing compositions of the disclosure, whether introduced as polynucleotides or polypeptides, can be provided to the cells for about 30 minutes to about 24 hours, e.g., 1 hour, 1.5 hours, 2 hours, 2.5 hours, 3 hours, 3.5 hours 4 hours, 5 hours, 6 hours, 7 hours, 8 hours, 12 hours, 16 hours, 18 hours, 20 hours, or any other period from about 30 minutes to about 24 hours, which can be repeated with a frequency of about every day to about every 4 days, e.g., every 1.5 days, every 2 days, every 3 days, or any other frequency from about every day to about every four days. The compositions may be provided to the subject cells one or more times, e.g., one time, twice, three times, or more than three times, and the cells allowed to incubate with the agent(s) for some amount of time following each contacting event e.g., 16-24 hours. In cases in which two or more different prime editing system components, e.g. two different polynucleotide constructs are provided to the cell (e.g., different components of the same prim editing system, or two different guide nucleic acids that are complementary to different sequences within the same or different target genes), the compositions may be delivered simultaneously (e.g., as two polypeptides and/or nucleic acids). Alternatively, they may be provided sequentially, e.g., one composition being provided first, followed by a second composition.

The prime editing compositions and pharmaceutical compositions of the disclosure, whether introduced as polynucleotides or polypeptides, can be administered to subjects in need thereof for about 30 minutes to about 24 hours, e.g., 1 hour, 1.5 hours, 2 hours, 2.5 hours, 3 hours, 3.5 hours 4 hours, 5 hours, 6 hours, 7 hours, 8 hours, 12 hours, 16 hours, 18 hours, 20 hours, or any other period from about 30 minutes to about 24 hours, which can be repeated with a frequency of about every day to about every 4 days, e.g., every 1.5 days, every 2 days, every 3 days, or any other frequency from about every day to about every four days. The compositions may be provided to the subject one or more times, e.g., one time, twice, three times, or more than three times. In cases in which two or more different prime editing system components, e.g. two different polynucleotide constructs are administered to the subject (e.g., different components of the same prim editing system, or two different guide nucleic acids that are complementary to different sequences within the same or different target genes), the compositions may be administered simultaneously (e.g., as two polypeptides and/or nucleic acids). Alternatively, they may be provided sequentially, e.g., one composition being provided first, followed by a second composition.

The corresponding sequences to the SEQ ID NOs. disclosed herein is provided in the sequence listing filed herewith.

### EXAMPLES

The following examples are provided for illustrative purposes only and are not intended to limit the scope of the claims provided herein.

### Example 1: Installation of c.73_74 ΔGT Mutation in Wildtype HEK293T Cells by Prime Editing

To examine accessibility of the NCF1 gene for Prime Editing, prime editing guide RNAs (PEgRNAs) were designed to install a c.73_74 ΔGT mutation in wildtype HEK293T cells.

PEgRNA assembly: PEgRNAs were assembled by one of three methods: in the first method, pooled synthesized DNA oligos encoding the PEgRNA and flanking U6 expression plasmid homology regions were cloned into U6 expression plasmids via Gibson cloning and sequencing of bacterial colonies via Sanger or Next-generation sequencing. In the second method, double-stranded linear DNA fragments encoding PEgRNA and homology sequences as above were individually Gibson-cloned into U6 expression plasmids. In the third method, for each PEgRNA, separate oligos encoding a protospacer, a gRNA scaffold, and PEgRNA extension (PBS and RT) were ligated, and then cloned into a U6 expression plasmid as described in Anzalone et al., Nature. 2019 December; 576(7785):149-157. Bacterial colonies carrying sequence-verified plasmids were propagated in LB or TB. Plasmid DNA was purified by minipreps for mammalian transfection.

HEK cell culture and transfection: HEK293T cells were propagated in DMEM with 10% FBS. Prior to transfection, cells were seeded in 96-well plates and then transfected with Lipofectamine 2000 according to the manufacturer's directions with DNA encoding PE2 and PEgRNA. Three days after transfection, gDNA was harvested in lysis buffer for high throughput sequencing and sequenced using Miseq. Generally, indel frequency was quantified using standard quantification techniques via CRISPResso2 algorithm as described in Clement, K. et al., Nat. Biotechnol. 37, 224-226 (2019), with the quantification window defined as at least 5 bases 5′ and 3′ of pegRNA (and ngRNA) nick site

PEgRNAs spacers were designed to be 20 nt in length and for the protospacer to be adjacent to an “5′-NGG-3′” PAM, where N is any one of nucleotides A, G, C, or T. DNA sequences encoding each PEgRNA was cloned into an expression plasmid driven by a U6 promoter. The PEgRNA expression plasmid and a plasmid encoding a prime editor fusion protein were co-transfected into wild type HEK293T cells. Seventy-two hours after transfection, genomic DNA was extracted from the HEK293T cells and the region of NCF1 targeted by Prime Editing was PCR amplified. Editing of NCF1 was evaluated by Miseq and scored for the percentage of sequence reads with the intended edit. PEgRNA sequences used to install the c.73_74 ΔGT mutation and Prime Editing efficiency are provided in Table 88 below. The results indicate that Prime Editing is able to target the NCF1 gene near the c.73_74 ΔGT location.

TABLE 88
Sequence and activity of PEgRNAs for installation
of NCF1 c.73_74delGT in HEK293T cells.

| | Spacer | PBS | PBS | % Editing |
|---|---|---|---|---|
| PEgRNA¹ | Sequence | sequence | length | Efficiency ² |
| 34314 | 849 | 4007 | 8 | + |
| 34315 | 849 | 4007 | 8 | +++ |
| 34316 | 849 | 4007 | 8 | ++ |
| 34317 | 849 | 4007 | 8 | +++ |
| 34318 | 849 | 4009 | 10 | ++ |
| 34319 | 849 | 4009 | 10 | +++ |
| 34320 | 849 | 4009 | 10 | ++ |
| 34321 | 849 | 4009 | 10 | ++ |
| 34322 | 849 | 4011 | 12 | +++ |
| 34323 | 849 | 4011 | 12 | +++ |
| 34324 | 849 | 4011 | 12 | ++ |
| 34325 | 849 | 4011 | 12 | +++ |
| 34326 | 849 | 4013 | 14 | ++ |
| 34327 | 849 | 4013 | 14 | +++ |
| 34328 | 849 | 4013 | 14 | +++ |
| 34329 | 849 | 4013 | 14 | +++ |
| 34330 | 19081 | 19088 | 8 | − |
| 34331 | 19081 | 19088 | 8 | − |
| 34332 | 19081 | 19088 | 8 | + |
| 34333 | 19081 | 19088 | 8 | ++ |
| 34334 | 19081 | 19090 | 10 | + |
| 34335 | 19081 | 19090 | 10 | + |
| 34336 | 19081 | 19090 | 10 | + |
| 34337 | 19081 | 19090 | 10 | ++ |
| 34338 | 19081 | 19092 | 12 | − |
| 34339 | 19081 | 19092 | 12 | − |
| 34340 | 19081 | 19092 | 12 | + |
| 34341 | 19081 | 19092 | 12 | ++ |
| 34342 | 19081 | 19094 | 14 | − |
| 34343 | 19081 | 19094 | 14 | − |
| 34344 | 19081 | 19094 | 14 | ++ |
| 34345 | 19572 | 19580 | 10 | + |
| 34346 | 19572 | 19582 | 12 | + |
| 34347 | 19572 | 19582 | 12 | + |
| 34348 | 19572 | 19582 | 12 | + |
| 34349 | 19572 | 19584 | 14 | + |
| 34350 | 19572 | 19584 | 14 | + |
| 34351 | 19572 | 19584 | 14 | + |
| 34352 | 19572 | 19584 | 14 | − |
| 34353 | 22506 | 22514 | 8 | − |
| 34354 | 22506 | 22514 | 8 | + |
| 34355 | 22506 | 22514 | 8 | − |
| 34356 | 22506 | 22516 | 10 | + |
| 34357 | 22506 | 22516 | 10 | − |
| 34358 | 22506 | 22516 | 10 | + |
| 34359 | 22506 | 22518 | 12 | + |
| 34360 | 22506 | 22518 | 12 | + |

¹Indicated PEgRNA sequence contains addition of a 5′Guanine for adaptations for transcription from the expression plasmid used.
² “−”: 0%; “+”: 1%-5%; “+”: 5%-10%; “+++”: greater than 10%. Each range includes the upper limit and not the lower limit. For example, 1%-2% means any value larger than 1% and no less than 2%.

### Example 2: PEgRNA Screen for Prime Editing of Pseudogenes NCF1B and/or NCF1C in HEK293T Cells

PEgRNAs were designed to install a dinucleotide “GT” (or “AC” on the opposite strand) insertion at the c.73_74 ΔGT target site in human NCF1 gene. Two pseudogenes, NCF1B and NCF1C, share more than 99% sequence identity with the NCF1 gene: the pseudogenes each carries a deletion of “GT” compared to wild type NCF1, at the location corresponding to c.73_74. Therefore, PEgRNAs and ngRNAs designed to target the c.73_74 site in NCF1 would also be able to target corresponding target sequences in pseudogene NCF1C and NCF1B. Editing of NCF1C/NCF1B in wild-type HEK293T cells were thus used to access Prime Editing efficiency.

With methods as described in Example 1, PEgRNAs were assembled, HEK293T cells were transfected with plasmids encoding the PEgRNA and prime editor, and genomic DNA were extracted for sequencing analysis. Editing efficiency was assessed by first obtaining sequencing reads that have the sequence “GTGT” in the + strand at chromosome positions 74777267-74777270 according to GRCh38, or corresponding positions in NCF1B or NCF1C. These reads are referred to herein as the “GTGT reads”. The total GTGT reads and the total reads of NCF1, NCF1B, and NCF1C sequences flanking the c.73-74 site (or corresponding sites in NCF1B and NCF1C) in NCF1 were obtained by Miseq analysis, and the ratio of GTGT reads over total reads (GTGT reads %) were calculated. Because the HEK293T cells carry a wild-type NCF1 gene, the cells have two copies of endogenous “GTGT” NCF1, and 4 copies of ΔGT from NCF1C/NCF1B. Editing efficiency was thus calculated by the following formula:

Editing efficiency=(GTGT reads %−0.33)/0.66

The screen included 3 PEgRNA spacers, each designed with a different PBS and/or RTT. Each spacer is 20 nt in length and the corresponding protospacer is adjacent to an “5′-NGG-3′” PAM. A total of 46 PEgRNAs were examined. The PEgRNAs were examined for Prime Editing efficiency with PE2 editing strategy. The results are shown in Table 89 below. The distance from the PEgRNA's nick site to the intended GT insert in number of nucleotides (the nick-to-edit distance; for GT insertion, the nick-to-edit distance looks at the edit between the first GT and the second GT) is also provided. For two spacers having the sequences of SEQ ID No. 3999 and SEQ ID No. 19081, a majority of the PEgRNAs designed were able to achieve successful Prime Editing.

TABLE 89
PEgRNA screen for Prime Editing of pseudogenes
NCF1B and/or NCF1C in HEK293T cells cells.

| PEgRNA | | Nick-to- | | | | | |
|---|---|---|---|---|---|---|---|
| SEQ ID | Spacer | edit | PBS | PBS | RTT | RTT | % |
| No.¹ | SEQ ID | distance | SEQ ID | length | SEQ ID | length | editing² |
| 5528 | 3999 | 2 | 4007 | 8 | 4019 | 10 | + |
| 5531 | 3999 | 2 | 4007 | 8 | 4021 | 12 | ++ |
| 5539 | 3999 | 2 | 4007 | 8 | 4054 | 14 | + |
| 5547 | 3999 | 2 | 4007 | 8 | 4098 | 16 | + |
| 5532 | 3999 | 2 | 4009 | 10 | 4019 | 10 | ++ |
| 5536 | 3999 | 2 | 4009 | 10 | 4021 | 12 | ++ |
| 5548 | 3999 | 2 | 4009 | 10 | 4054 | 14 | ++ |
| 5597 | 3999 | 2 | 4009 | 10 | 4098 | 16 | ++ |
| 5535 | 3999 | 2 | 4011 | 12 | 4019 | 10 | ++ |
| 5563 | 3999 | 2 | 4011 | 12 | 4021 | 12 | ++ |
| 5606 | 3999 | 2 | 4011 | 12 | 4054 | 14 | ++ |
| 5647 | 3999 | 2 | 4011 | 12 | 4098 | 16 | ++ |
| 5556 | 3999 | 2 | 4013 | 14 | 4019 | 10 | ++ |
| 5635 | 3999 | 2 | 4013 | 14 | 4021 | 12 | ++ |
| 5644 | 3999 | 2 | 4013 | 14 | 4054 | 14 | − |
| 5672 | 3999 | 2 | 4013 | 14 | 4098 | 14 | ++ |
| 482 | 5 | 4 | 13 | 8 | 35 | 12 | − |
| 488 | 5 | 4 | 13 | 8 | 43 | 14 | − |
| 515 | 5 | 4 | 13 | 8 | 60 | 18 | − |
| 487 | 5 | 4 | 15 | 10 | 35 | 12 | − |
| 502 | 5 | 4 | 15 | 10 | 43 | 14 | + |
| 519 | 5 | 4 | 15 | 10 | 51 | 16 | − |
| 541 | 5 | 4 | 15 | 10 | 60 | 18 | − |
| 500 | 5 | 4 | 17 | 12 | 35 | 12 | − |
| 517 | 5 | 4 | 17 | 12 | 43 | 14 | − |
| 540 | 5 | 4 | 17 | 12 | 51 | 16 | − |
| 553 | 5 | 4 | 17 | 12 | 60 | 18 | − |
| 518 | 5 | 4 | 19 | 14 | 35 | 12 | − |
| 539 | 5 | 4 | 19 | 14 | 43 | 14 | − |
| 552 | 5 | 4 | 19 | 14 | 51 | 16 | − |
| 566 | 5 | 4 | 19 | 14 | 60 | 18 | − |
| 19481 | 19081 | 7 | 19088 | 8 | 19106 | 11 | − |
| 19484 | 19081 | 7 | 19088 | 8 | 19112 | 13 | + |
| 19487 | 19081 | 7 | 19088 | 8 | 19123 | 15 | ++ |
| 19496 | 19081 | 7 | 19088 | 8 | 19129 | 17 | + |
| 19483 | 19081 | 7 | 19090 | 10 | 19106 | 11 | − |
| 19488 | 19081 | 7 | 19090 | 10 | 19112 | 13 | + |
| 19498 | 19081 | 7 | 19090 | 10 | 19123 | 15 | + |
| 19514 | 19081 | 7 | 19090 | 10 | 19129 | 17 | + |
| 19489 | 19081 | 7 | 19092 | 12 | 19106 | 11 | − |
| 19497 | 19081 | 7 | 19092 | 12 | 19112 | 13 | + |
| 19515 | 19081 | 7 | 19092 | 12 | 19123 | 15 | + |
| 19535 | 19081 | 7 | 19092 | 12 | 19129 | 17 | + |
| 19495 | 19081 | 7 | 19094 | 14 | 19106 | 11 | − |
| 19516 | 19081 | 7 | 19094 | 14 | 19112 | 13 | + |
| 19534 | 19081 | 7 | 19094 | 14 | 19123 | 15 | ++ |

¹Indicated PEgRNA sequence does not contain the adaptations for transcription from a DNA template used experimentally (i.e., addition of a 5′G if the spacer did not already start with a G).
²“−”: 0%; “+”: 1%-5%; “++”: 5%-10%; “+++”: greater than 10%. Each range includes the upper limit and not the lower limit. For example, 1%-2% means any value larger than 1% and no less than 2%.

Two additional spacers were screened. 16 PEgRNAs, each having a different combination of PBS and RTT sequences, were designed for each spacer. The spacers are 20 nt in length and the corresponding protospacers are adjacent to an “5′-NGA-3′” PAM. PEgRNAs were synthesized, wild-type HEK293T cells transfected, and genomnic DNA extracted and analyzed as described in Example 1. The PEgRNAs were examined for Prime Editing efficiency with PE2 editing strategy, with two replicates for each PEgRNA. Editing efficiency was assess by the formula (GTGT reads−0.33)/0.66 as described above. The results are shown in Table 90 below. The PEgRNAs tested, having the spacer sequence of SEQ ID No. 33242 or SEQ ID No. 31334, showed low editing efficiency.

TABLE 90
PE2 Prime Editing of NCF1B/NCF1C pseudogenes in HEK293T cells.

| ¹PEgRNA | Spacer | PBS | | RTT | | | | | |
|---|---|---|---|---|---|---|---|---|---|
| SEQ | SEQ | SEQ | PBS | SEQ | RTT | % Editing | % Editing | % Indels | % Indels |
| ID NO. | ID No. | ID No. | length | ID No. | length | Rep1* | Rep2* | rep** | rep 2** |
| 33974 | 33242 | 33249 | 8 | 33329 | 24 | − | + | + | + |
| 33978 | 33242 | 33249 | 8 | 33347 | 26 | − | − | + | + |
| 33983 | 33242 | 33249 | 8 | 33363 | 28 | − | + | + | + |
| 33995 | 33242 | 33249 | 8 | 33376 | 30 | − | − | + | + |
| 33977 | 33242 | 33251 | 10 | 33329 | 24 | + | − | + | − |
| 33984 | 33242 | 33251 | 10 | 33347 | 26 | − | − | + | + |
| 33994 | 33242 | 33251 | 10 | 33363 | 28 | + | − | + | + |
| 34002 | 33242 | 33251 | 10 | 33376 | 30 | ++ | − | + | + |
| 33985 | 33242 | 33253 | 12 | 33329 | 24 | + | ++ | + | + |
| 33993 | 33242 | 33253 | 12 | 33347 | 26 | ++ | − | + | + |
| 34004 | 33242 | 33253 | 12 | 33363 | 28 | + | − | − | + |
| 34012 | 33242 | 33253 | 12 | 33376 | 30 | ++ | − | + | + |
| 33992 | 33242 | 33255 | 14 | 33329 | 24 | + | + | + | + |
| 34003 | 33242 | 33255 | 14 | 33347 | 26 | − | + | + | + |
| 34011 | 33242 | 33255 | 14 | 33363 | 28 | + | + | + | + |
| 34017 | 33242 | 33255 | 14 | 33376 | 30 | + | − | + | + |
| 31667 | 31334 | 31342 | 8 | 31370 | 29 | − | − | + | + |
| 31669 | 31334 | 31342 | 8 | 31375 | 31 | − | − | + | + |
| 31673 | 31334 | 31342 | 8 | 31388 | 34 | + | ++ | + | + |
| 31678 | 31334 | 31342 | 8 | 31396 | 36 | − | − | + | − |
| 31668 | 31334 | 31344 | 10 | 31370 | 29 | − | − | + | + |
| 31672 | 31334 | 31344 | 10 | 31375 | 31 | − | + | + | − |
| 31679 | 31334 | 31344 | 10 | 31388 | 34 | − | − | + | + |
| 31685 | 31334 | 31344 | 10 | 31396 | 36 | − | − | + | + |
| 31671 | 31334 | 31346 | 12 | 31370 | 29 | ++ | − | + | + |
| 31675 | 31334 | 31346 | 12 | 31375 | 31 | ++ | − | + | + |
| 31684 | 31334 | 31346 | 12 | 31388 | 34 | − | − | + | + |
| 31690 | 31334 | 31346 | 12 | 31396 | 36 | − | + | + | + |
| 31677 | 31334 | 31348 | 14 | 31370 | 29 | − | + | − | + |
| 31681 | 31334 | 31348 | 14 | 31375 | 31 | + | − | + | − |
| 31691 | 31334 | 31348 | 14 | 31388 | 34 | + | ++ | + | + |

¹Indicated PEgRNA sequence contains addition of a 5′Guanine for adaptations for transcription from the expression plasmid used.
*“−”: 0% or less (negative values resulted from formula used to calculate editing efficiency. If GTGT reads are lower than 33%, the result is negative); “+”: 0%-1%; “++”: 1%-3%; “+++”: greater than 3%. Each range includes the upper limit and not the lower limit. For example, 1%-2% means any value larger than 1% and no less than 2%.
**“−”: 0%; “+”: 0%-0.5%; “++”: 0.5%-1%; “+++”: greater than 11%. Each range includes the upper limit and not the lower limit. For example, 1%-2% means any value larger than 1% and no less than 2%.

A subset of PEgRNAs, with a spacer having the sequence of either SEQ ID No. 3999 or SEQ ID No. 19081 from the above screens, were further examined for Prime Editing efficiency with PE3 strategy. The results are shown in Tables 91 and 92 below. Successful Prime Editing was observed in most of PEgRNA/ngRNA combinations (with three exceptions).

TABLE 91
PE3 Prime Editing of NCF1B/NCF1C
pseudogenes in HEK293T cells

| | % editing | % editing | % editing |
|---|---|---|---|
| PEgRNA | w/ngRNA | w/ngRNA | w/ngRNA |
| SEQ ID | SEQ ID | SEQ ID | SEQ ID |
| No.¹ | No. 590 | No. 589 | No. 584 |
| 5528 | ++ | ++ | +++ |
| 5531 | ++ | ++ | +++ |
| 5539 | ++ | ++ | +++ |
| 5547 | ++ | ++ | +++ |
| 5532 | ++ | +++ | +++ |
| 5536 | +++ | +++ | +++ |
| 5548 | ++ | +++ | +++ |
| 5597 | ++ | +++ | +++ |
| 5535 | ++ | +++ | +++ |
| 5563 | +++ | +++ | +++ |
| 5606 | +++ | +++ | +++ |

¹Indicated PEgRNA sequence does not contain the adaptations for transcription from a DNA template used experimentally (i.e., addition of a 5′G if the spacer did not already start with a G).
For all editing efficiency in Table 91, “−”: 0%; “+”: 1%-5%; “++”: 5%-10%; “+++”: greater than 10%.

TABLE 92
PE3 Prime Editing of NCF1B/NCF1C
pseudogenes in HEK293T cells.

| PEgRNA | % editing w/ | % editing w/ |
|---|---|---|
| SEQ ID | ngRNA SEQ ID | ngRNA SEQ ID |
| No.¹ | No. 19566 | No. 2143 |
| 19496 | ++ | +++ |
| 19483 | − | − |
| 19488 | ++ | +++ |
| 19498 | ++ | +++ |
| 19514 | + | +++ |
| 19489 | − | − |
| 19497 | + | ++ |
| 19515 | + | ++ |
| 19535 | ++ | +++ |
| 19495 | − | + |
| 19516 | ++ | +++ |
| 19534 | ++ | +++ |

¹Indicated PEgRNA sequence does not contain the adaptations for transcription from a DNA template used experimentally (i.e., addition of a 5′G if the spacer did not already start with a G).
For all editing efficiency in Table 92, “−”: 0%; “+”: 1%-5%; “++”: 5%-10%; “+++”: greater than 10%. Each range includes the upper limit and not the lower limit. For example, 1%-2% means any value larger than 1% and no less than 2%.

Although PEgRNAs having the spacer sequence of SEQ ID No. 5 showed lower editing efficiency in experiment shown in Table B1, the spacer was further tested with different combinations of PBSs and RTTs. PEgRNAs were chemically synthesized. Without wishing to be bound by theory, synthesized PEgRNAs may have improve quality compared to PEgRNA transcribed from plasmid. Wildtype HEK293T cells were transfected with the synthesized PEgRNAs and plasmid encoding a Prime Editor fusion protein. Genomic DNA was extracted and sequencing result was analyzed as described in Example 1. Two replicates were performed for each PEgRNA. The results are shown in Table 93 below.

TABLE 93
PE2 Prime Editing of NCF1B/NCF1C
pseudogenes in HEK293T cells.

| PEgRNA | | | Editing | Editing | Indels | Indels |
|---|---|---|---|---|---|---|
| SEQ ID | PBS SEQ | RTT SEQ | Rep 1 | Rep 2 | rep 1 | rep 2 |
| NO¹ | ID NO: | ID NO: | (%)² | (%)² | (%)³ | (%)³ |
| 482 | 13 | 35 | + | + | + | + |
| 488 | 13 | 43 | + | + | + | + |
| 498 | 13 | 51 | + | + | + | + |
| 515 | 13 | 60 | + | + | + | + |
| 536 | 13 | 66 | + | + | + | + |
| 487 | 15 | 35 | ++ | ++ | +++ | + |
| 502 | 15 | 43 | ++ | ++ | +++ | ++ |
| 519 | 15 | 51 | ++ | ++ | +++ | ++ |
| 541 | 15 | 60 | ++ | ++ | ++ | + |
| 556 | 15 | 66 | ++ | ++ | ++ | ++ |
| 500 | 17 | 35 | ++++ | ++++ | ++++ | +++ |
| 517 | 17 | 43 | ++++ | ++++ | ++++ | ++++ |
| 540 | 17 | 51 | ++++ | ++++ | ++++ | +++ |
| 553 | 17 | 60 | ++++ | +++ | ++++ | ++++ |
| 564 | 17 | 66 | +++ | +++ | +++ | ++++ |
| 518 | 19 | 35 | ++++ | +++ | ++++ | +++ |
| 539 | 19 | 43 | ++++ | ++++ | ++++ | ++++ |
| 552 | 19 | 51 | +++ | ++ | +++ | ++ |
| 566 | 19 | 60 | +++ | +++ | ++ | +++ |
| 571 | 19 | 66 | +++ | +++ | +++ | +++ |

¹Indicated sequence does not contain the 3′ mU*mU*mU*U additional nucleotides and does not contain a 5′mN*mN*mN* modifications used experimentally, where m indicates that the indicated nucleotide contains a 2′-O-Me modification and a * indicates a phosphorothioate bond.
²“−”: 0%; “+”: 0%-4%; “++”: 4%-8%; “+++”: 8% to 11%; “++++”: greater than 11%. Each range includes the upper limit and not the lower limit. For example, 1%-2% means any value larger than 1% and no less than 2%.
³“−”: 0%; “+”: 0%-0.4%; “++”: 0.4%-0.5%; “+++”: 0.5% to 0.9%; “++++”: greater than 9%.

### Example 3: Prime Editing of NCF1, NCF1B, and/or NCF1C in HEK293T Cells Bearing a c.73_74 ΔGT Mutation

A clonal cell line homozygous for NCF1 c.73_74ΔGT mutation was generated as follows: HEK293T cells were co-transfected with plasmids expressing a prime editor fusion protein, PEgRNA SEQ ID No. 34323 and ngRNA SEQ ID No. 584 as described in Example 1. Seventy-two hours after transfection, cells were detached from the growth surface and plated at a limiting dilution to generate clonal colonies. After seven days of growth, single colonies were mechanically detached from the dish and transferred into separate wells. Once grown to near-confluence, cells were split into two fractions: fraction #1 was propagated while gDNA was harvested from fraction #2. Editing efficiency was determined by Miseq of NCF1 c.73_74GT region as described in Example 1. Clones exhibiting 100% editing for installation of the ΔGT mutation were used to examine prime editing correction of NCF1 c.73_74 ΔGT.

A subset of five highly active PEgRNAs from Example 2 were examined for Prime Editing efficiency, either with PE2 strategy or with PE3 strategy combined with the ngRNA having the sequence of SEQ ID No. 584. Each of the five PEgRNAs has a spacer of SEQ ID No. 3999. The clonal HEK293T cells described above were co-transfected with plasmids encoding a Prime Editor fusion protein, a PEgRNA, and the ngRNA respectively, as described in Example 1. Seventy-two hours after transfection, gDNA was extracted and the PE-targeted region of NCF1 was PCR amplified. The results are shown in Table 94. Efficient editing was observed for all five PEgRNAs. In addition, improved editing was observed with PE3 strategy.

TABLE 94
Prime Editing of NCF1, NCF1B, and/or NCF1C in HEK293T
cells bearing a c.73_74 ΔGT mutation

| PEgRNA SEQ ID No.¹ | % Editing (PE2)² | % Editing (PE3)² |
|---|---|---|
| 5535 | ++ | +++ |
| 5563 | ++ | +++ |
| 5606 | ++ | +++ |
| 5556 | ++ | +++ |
| 5644 | ++ | +++ |

¹Indicated PEgRNA sequence does not contain the adaptations for transcription from a DNA template used experimentally (i.e., addition of a 5′G if the spacer did not already start with a G).
²“−”: 0%; “+”: 1%-15%; “++”: 15%-30%; “+++”: 30%-50%.; “++++”: greater than 50%. Each range includes the upper limit and not the lower limit. For example, 1%-2% means any value larger than 1% and no less than 2%.

A summary of ngRNAs used in Examples 2 and 3 are provided in Table 95 below.

TABLE 95

| | ngRNA Spacer | Spacer |
|---|---|---|
| ngRNA SEQ ID NO: | SEQ ID No. | SEQ ID of PERNAs |
| 2143 | 2130 | SEQ ID No. 19081 |
| 19566 | 19478 | SEQ ID No. 19081 |
| 590 | 422 | SEQ ID No. 3999 |
| 589 | 458 | SEQ ID No. 3999 |
| 584 | 429 | SEQ ID No. 3999 |

### Example 4: Prime Editing with PEgRNAs Harboring Additional PAM Silencing Edits

An additional set of 30 PEgRNAs having the spacer of SEQ ID No.3999 were tested. The PEgRNAs were designed by including additional edits, using PEgRNAs having the sequence of SEQ ID No. 5563 and 5606 as a starting point. Besides the GT insertion, the RTT of each of the 30 PEgRNAs further include 1 or 2 nt substitutions at a position corresponding to intron 1 of the NCF1, NCF1B, and/or NCF1C gene. All 30 PEgRNAs have the same PBS sequence (SEQ ID No. 4011). 15 PEgRNAs have a 12 nucleotide RTT, designed by including a nucleotide substitution at position 5 and/or 6 of SEQ ID NO. 402. The other 15 PEgRNAs have a 14 nucleotide RTT, designed by including a nucleotide substitution at position 7 and/or 8 of SEQ ID No. 4054. Incorporation of these substitutions alters the PAM sequence GGG on the edit strand to a non-NGG sequence. Without wishing to be bound by any particular theory, PAM silencing edits may prevent the Cas9 nickase from re-nicking the edit strand, and thus improve editing efficiency.

The additional PAM silencing edits allow PE3b ngRNAs to be designed for each of the PEgRNAs, where the ngRNA spacer contain perfect complementarity to the portion of the edit strand that contains the intended edit (i.e., perfectly complementarity to the edit strand only after the intended edits are made). The PEgRNAs and ngRNAs were assembled, HEK293T cells transfected, and genomic DNA extracted and analyzed as described in Example 1. Pseudogene editing efficiency was assessed using the formula as described in Example 2.

Prime Editing efficiency and indel formation were assessed for both PE2 and PE3 editing strategies. The results are provided in Tables 96-99 below: Table 96 summarizes the results from PE2 editing, and Tables 97-99 provide the results of PE3b editing with the 12 nt RTT PEgRNAs and 14 nt RTT PEgRNAs, respectively. Table 99 provides the average editing efficiency of the PAM silencing PEgRNAs using PE2 and PE3b editing strategy. The PAM silencing edits besides the GT insertion are well tolerated by Prime Editing, as shown by successful editing observed. In addition, the PE3b strategy generally improved Prime Editing efficiency.

For Tables 96-99, editing efficiency are grouped as follows: “+”=9% to 20%, “++”=20% to 26%, “+++”=26% to 35%, “++++”=35% and more. Each range includes the upper limit and not the lower limit. For example, “1% to 2%” means any value larger than 1% and no less than 2%.

TABLE 96
PE2 Prime Editing of NCF1BINCF1C pseudogenes
with PEgRNAs having PAM silencing edits.

| PEgRNA SEQ ID¹² | RTT SEQ ID No.² | RTT Length | % Editing Rep 1 | % Editing Rep 2 |
|---|---|---|---|---|
| 5563 | 4021 | 12 | ++ | ++ |
| 5564* | 4030* | 12 | ++ | ++ |
| 5555* | 4032* | 12 | + | ++ |
| 5554* | 4034* | 12 | + | + |
| 5559* | 4029* | 12 | + | + |
| 5566* | 4033* | 12 | + | + |
| 5562* | 4024* | 12 | ++ | ++ |
| 5551* | 4023* | 12 | + | + |
| 5546* | 4022* | 12 | ++ | ++ |
| 5565* | 4028* | 12 | + | + |
| 5552* | 4025* | 12 | + | + |
| 5560* | 4031* | 12 | + | + |
| 5549* | 4035* | 12 | + | ++ |
| 5558* | 4036* | 12 | + | ++ |
| 5567* | 4027* | 12 | + | ++ |
| 5553* | 4026* | 12 | + | + |
| 5606 | 4054 | 14 | ++ | ++ |
| 5610* | 4062* | 14 | ++ | +++ |
| 5592* | 4066* | 14 | +++ | +++ |
| 5591* | 4058* | 14 | + | ++ |
| 5607* | 4065* | 14 | ++ | +++ |
| 5604* | 4064* | 14 | ++ | ++ |
| 5593* | 4061* | 14 | +++ | +++ |
| 5595* | 4063* | 14 | +++ | +++ |
| 5602* | 4068* | 14 | +++ | +++ |
| 5594* | 4057* | 14 | + | + |
| 5596* | 4056* | 14 | ++ | ++ |
| 5608* | 4060* | 14 | ++ | ++ |
| 5601* | 4059* | 14 | ++ | ++ |
| 5598* | 4053* | 14 | + | + |
| 5603* | 4055* | 14 | + | + |
| 5611* | 4067* | 14 | ++ | ++ |

¹Indicated PEgRNA sequence does not contain the adaptations for transcription from a DNA template used experimentally (i.e., addition of a 5′G if the spacer did not already start with a G).
²A star (*) indicates the RTT, and corresponding PEgRNA, contains an additional PAM silencing mutation.

TABLE 97
PE3b Prime Editing of NCF1BINCF1C pseudogenes with PEgRNAs
having PAM silencing edits and corresponding PE3b ngRNAs.

| | ngRNA | Paired | | |
|---|---|---|---|---|
| ngRNA SEQ | spacer SEQ | PEgRNA | % Editing | % Editing |
| ID No.¹ | ID No.¹ | SEQ ID¹ | Rep 1 | Rep 2 |
| 5702(*9) | 5504(*9) | 5564(*9) | + | +++ |
| 5693(*10) | 5498(*10) | 5555(*10) | ++++ | ++++ |
| 5699(*14) | 5503(*14) | 5554(*14) | +++ | +++ |
| 5707(*3) | 5510(*3) | 5559(*3) | ++++ | +++ |
| 5703(*13) | 5505(*13) | 5566(*13) | ++++ | ++++ |
| 5704(*8) | 5506(*8) | 5562(*8) | ++++ | ++++ |
| 5705(*12) | 5508(*12) | 5551(*13) | ++++ | ++++ |
| 5697(*1) | 5495(*1) | 5546(*1) | ++++ | ++++ |
| 5701(*4) | 5502(*4) | 5565(*4) | +++ | +++ |
| 5698(*2) | 5501(*2) | 5552(*2) | ++++ | ++++ |
| 5700(*7) | 5497(*7) | 5560(*7) | ++++ | ++++ |
| 5695(*6) | 5496(*6) | 5549(*6) | +++ | +++ |
| 5694(*15) | 5500(*15) | 5558(*15) | +++ | ++++ |
| 5696(*11) | 5499(*11) | 5567(*11) | +++ | ++ |
| 5706(*5) | 5509(*5) | 5553(*5) | ++++ | +++ |

¹A star (*n), where n is an integer, is used to indicate the paring relationship between PE3b ngRNAs and PEgRNA RTTs. An RTT annotated with (*n) indicates that the RTT has a specific PAM silencing edit, and a PE3b ngRNA annotated with the same number *n indicates that the ngRNA spacer has perfect complementarity to the portion of the edit strand including the PAM silencing edit after the edit is incorporated (i.e. the ngRNA spacer has a region of identity to the RTT that includes the PAM silencing edit). The type of PAM silencing edits and sequences are provided in Table 12, which are annotated under the same system.

TABLE 98
PE3b Prime Editing of NCF1BINCF1C pseudogenes with PEgRNAs
having PAM silencing edits and corresponding PE3b ngRNAs.

| nGRNA | spacer | Paired PEgRNA | % Editing | % Editing |
|---|---|---|---|---|
| SEQ ID No.¹ | SEQ ID No.¹ | SEQ ID¹ | Rep 1 | Rep 2 |
| 5702(*9) | 5504(*9) | 5610(*9) | +++ | +++ |
| 5693(*10) | 5498(*10) | 5592(*10) | ++++ | ++++ |
| 5699(*14) | 5503(*14) | 5591(*14) | +++ | +++ |
| 5707(*3) | 5510(*3) | 5607(*3) | ++++ | ++++ |
| 5703(*13) | 5505(*13) | 5604(*13) | ++++ | ++++ |
| 5704(*8) | 5506(*8) | 5593(*8) | ++++ | ++++ |
| 5705(*12) | 5508(*12) | 5595(*12) | ++++ | ++++ |
| 5697(*1) | 5495(*1) | 5602(*1) | ++++ | ++++ |
| 5701(*4) | 5502(*4) | 5594(*4) | ++ | ++ |
| 5698(*2) | 5501(*2) | 5596(*2) | +++ | ++++ |
| 5700(*7) | 5497(*7) | 5608(*7) | +++ | +++ |
| 5695(*6) | 5496(*6) | 5601(*6) | +++ | +++ |
| 5694(*15) | 5500(*15) | 5598(*15) | +++ | +++ |
| 5696(*11) | 5499(*11) | 5603(*11) | +++ | ++ |
| 5706(*5) | 5509(*5) | 5611(*5) | ++++ | ++++ |

¹A star (*n), where n is an integer, is used to indicate the paring relationship between PE3b ngRNAs and PEgRNA RTTs. An RTT annotated with (*n) indicates that the RTT has a specific PAM silencing edit, and a PE3b ngRNA annotated with the same number *n indicates that the ngRNA spacer has perfect complementarity to the portion of the edit strand including the PAM silencing edit after the edit is incorporated (i.e. the ngRNA spacer has a region of identity to the RTT that includes the PAM silencing edit). The type of PAM silencing edits and sequences are provided in Table 12, which are annotated under the same system.

TABLE 99
Average editing efficiency of PAM silencing PEgRNAs
using PE2 and PE3 strategyand corresponding ngRNAs

| Editing strategy | Prime Editing reagents | Average editing efficiency |
|---|---|---|
| PE2 | PEgRNAs with 12 nt RTT | + |
| PE2 | PEgRNAs with 14 nt RTT | ++ |
| PE3 | PEgRNAs with 12 nt RTT | +++ |
| PE3 | PERNAs with 14 nt RTT | ++++ |

### Example 5: Prime Editing with PEgRNAs that Edit c.73_74 Deletion with Alternative Codons

In wild-type NCF1 gene, the first four nucleotides in exon 2 are GTGT, the first three of which encodes a Valine. Deletion of one of the two “GT” s correspond to the c.73_74 ΔGT mutation. In this experiment, PEgRNAs whose RTTs contain a “NT” insert in place of “GT”, or a “AN” insert on the opposite strand in place of “AC”, where N is any one of A, G, or C, were tested (inserts correspond to the second “Gr” in “GTGT”, underlined above). These PEgRNAs can correct the frameshift resulted from the c.73_74 ΔGT mutation, and can generate a synonymous edit without changing the amino acid sequence encoded by NCF1.

30 PEgRNAs were designed. 16 of the PEgRNAs have the spacer sequence of SEQ ID No.5, and 14 have the spacer sequence SEQ ID NO. 19081. The PEgRNAs were chemically synthesized. HEK293T cells were transfected, and genomic DNA extracted and analyzed as described in Example 1. Editing efficiency was calculated it the formula as described in Example 2. Results of Prime Editing are summarized in Table 100.

Successful Prime Editing with RTTs containing alternative codons for Valine were observed.

TABLE 100
Prime Editing of NCF1B/NCF1C pseudogenes with Valine recode PEgRNAs.
For editing efficiency, “+”: 1.5%-4%, “++”: 4%-6%, “+++”: 6%-11%, “++++”: greater than 11%.
For indels: “+”: 0-0.1%; “++”: 0.1% to 0.2”; “+++”: 0.2% to 0.4%; “++++”: greater than
0.4%. Each range includes the upper limit and not the lower limit. For example,
1%-2% means any value larger than 1% and no less than 2%.

| PEgRNA | Spacer | PBS | | RTT | | % | % | % | % |
|---|---|---|---|---|---|---|---|---|---|
| SEQ ID | SEQ ID | SEQ ID | PBS | SEQ ID | RTT | Editing | Editing | Indels | Indels |
| No.¹ | NO: | NO: | length | NO: | length | Rep1 | Rep2 | Rep1 | Rep2 |
| 485 | 5 | 14 | 9 | 34 | 12 | ++ | + | + | + |
| 496 | 5 | 14 | 9 | 41 | 14 | + | + | − | − |
| 497 | 5 | 16 | 11 | 34 | 12 | ++ | + | + | + |
| 513 | 5 | 16 | 11 | 41 | 14 | + | + | − | − |
| 510 | 5 | 18 | 13 | 34 | 12 | +++ | +++ | ++ | + |
| 521 | 5 | 18 | 13 | 41 | 14 | ++ | +++ | + | ++ |
| 19501 | 19081 | 19090 | 10 | 19121 | 15 | +++ | +++ | ++++ | ++ |
| 19507 | 19081 | 19090 | 10 | 19125 | 16 | +++ | +++ | ++ | ++ |
| 19512 | 19081 | 19092 | 12 | 19121 | 15 | +++ | +++ | +++ | +++ |
| 19527 | 19081 | 19092 | 12 | 19125 | 16 | ++++ | +++ | +++ | ++ |
| 483 | 5 | 14 | 9 | 36 | 12 | ++ | +++ | − | − |
| 489 | 5 | 14 | 9 | 42 | 14 | + | + | − | + |
| 492 | 5 | 16 | 11 | 36 | 12 | + | ++ | − | + |
| 512 | 5 | 16 | 11 | 42 | 14 | + | + | − | − |
| 503 | 5 | 18 | 13 | 36 | 12 | ++ | +++ | − | + |
| 522 | 5 | 18 | 13 | 42 | 14 | ++ | +++ | − | + |
| 19499 | 19081 | 19090 | 10 | 19120 | 15 | ++++ | +++ | +++ | +++ |
| 19503 | 19081 | 19090 | 10 | 19127 | 16 | ++++ | ++++ | +++ | +++ |
| 19511 | 19081 | 19092 | 12 | 19120 | 15 | ++++ | ++++ | +++ | +++ |
| 19521 | 19081 | 19092 | 12 | 19127 | 16 | ++++ | ++++ | +++ | +++ |
| 484 | 5 | 14 | 9 | 33 | 12 | ++ | ++ | + | + |
| 494 | 5 | 14 | 9 | 44 | 14 | ++ | + | + | − |
| 495 | 5 | 16 | 11 | 33 | 12 | ++ | + | − | + |
| 506 | 5 | 16 | 11 | 44 | 14 | + | + | − | − |
| 504 | 5 | 18 | 13 | 33 | 12 | +++ | ++ | + | + |
| 520 | 5 | 18 | 13 | 44 | 14 | +++ | +++ | ++ | − |
| 19502 | 19081 | 19090 | 10 | 19122 | 15 | ++++ | ++++ | ++ | +++ |
| 19509 | 19081 | 19090 | 10 | 19124 | 16 | ++++ | ++++ | ++ | +++ |
| 19513 | 19081 | 19092 | 12 | 19122 | 15 | ++++ | ++++ | +++ | +++ |
| 19522 | 19081 | 19092 | 12 | 19124 | 16 | ++++ | ++++ | ++ | +++ |

¹Indicated sequence does not contain the 3′ mU*mU*mU*U additional nucleotides and does not contain a 5′mN*mN*mN* modifications used experimentally, where m indicates that the indicated nucleotide contains a 2′-O-Me modification and a * indicates a phosphorothioate bond. In case of ngRNA, the indicate. Italics: PEgRNAs for installing an “AT” in place of the second GT in GTGT as described above. underlined: PEgRNAs for installing a “TT” in place of the second GT in GTGT as described above. Italics: PEgRNAs for installing a “CT” in place of the second GT in GTGT as described above.

### Example 6: Ex Vivo Editing of Human CD34+ Cells at NCF1B, NCF1C, or NCF1 Sites

Prime Editing activity was tested for various PEgRNAs having the spacer of SEQ ID NO: 3999 or SEQ ID NO: 19081 were examined in human hematopoietic pluripotent stem cells (HSPCs, or CD34+ cells).

Cryopreserved human CD34+ cells were obtained from healthy donors and CGD patients having a c.73_74 GT deletion in NCF1 gene (referred to as ΔGT CGD patients). Prior to electroporation, cells were thawed and cultured for 2 days in complete media (X-VIVO 10, Lonza) supplemented with 100 ng/mL of each of Stem cell factor, Thrombopoietin, and Fms-related tyrosine kinase 3 ligand at 37° C./5% CO2 prior to electroporation. At the time of electroporation, cells were counted and resuspended to between 3.5E+7 to 7E+7 cells/mL in Maxcyte electroporation buffer.

mRNA encoding Prime Editor fusion proteins was used to electroporate the cells. Each Prime Editor fusion protein contains a Streptococcus pyogenes Cas9 (SpCas9) H840A nickase fused to the N-terminus of a full length MMLV-RT or a truncated MMLV-RT, where the truncation is at the C-terminus to reduce RNaseH activity. A Prime Editor fusion protein indicated as “Fx”, e.g., F1, has a full length MMLV-RT, and a Prime Editor fusion protein indicated as “Tx”, e.g., T1, has a C-terminal truncated MMLV-RT.

Prime editing guide RNAs (pegRNAs) and nick guide RNAs (ngRNAs) were synthesized and HPLC purified. Resuspended cells were mixed with PEgRNA, ngRNA, and mRNA encoding a Prime Editor fusion protein and transferred to a Maxcyte Cartridge for electroporation. Cells were then electroporated on Macyte program HSC-6 and transferred to a tissue culture plate and returned to the 37° C. incubator to recover for 20 minutes before adding fresh complete media, to achieve a density of approximately 1xe\hat{ }6 cells/mL. Cells were returned to culture for the desired time for downstream analysis.

At desired timepoints post electroporation (24 hours-120 hours), genomic DNA was extracted using Quick extract buffer (Lucigen). Samples were then PCR amplified at the NCF1 (or NCF1B/NCF1C) target site and processed for amplicon sequencing (Illumina). Sequence reads were assessed for insertion of the GT dinucleotide at the target site(s) as well as indels at the target sites.

CD34+ cells from healthy human donors as well as from ΔGT CGD patients were used to access editing efficiency and indel frequency by prime editing. Editing efficiency is represented as a percentage of all reads at a given timepoint.

For Editing in Cells from Healthy Donors:

Unless otherwise indicated, a “healthy donor” in Examples 6 to 11 refers to a donor having a wild type NCF1 gene bearing two copies of “GTGT”, as well as a NCF1B and a NCF1C pseudogene each having two copies of “GT” at the site corresponding to c.73_74 of NCF1.

Healthy donor CD34+ cells contain a wild-type NCF1 gene as well as both NCF1B and NCF1C pseudogenes. Compared to the wild type NCF1 gene, NCF1B and NCF1C both contain a dinucleotide deletion corresponding to the c.73-74GT deletion in the CGD-associated NCF1 gene, and are otherwise identical to the NCF1 gene at the target site corresponding to the c.73-74 location. Therefore, editing of NCF1B/NCF1C in healthy CD34+ cells is used an estimate for prime editing efficiency of the disease associated NCF1 gene, as well as for prime editing's potential to restore p47 function in CD34+ cells, as editing of NCF1B or NCF1C to resemble the wild type NCF1 sequence may be able to restore expression of a functional p47 protein.

Similar to calculation of editing efficiency as described in Example 2 above, the percentage of “GTGT” reads over the total reads of NCF1, NCF1B, and NCF1C sequences flanking the c.73-74 site (or corresponding sequences in NCF1B and NCF1C) sequence in exon 2 of NCF1 was used to calculate editing efficiency.

For editing in healthy donor cells, GTGT read percentage represents the percentage of all reads containing GTGT at a NCF1, NCF1B, or NCF1C site, and thus includes reads from wild-type NCF1 (˜33.3% of reads in an unedited healthy donor cells). The pseudogene correction efficiency calculates the level of correction within the pseudogenes, by deducting 33.3% from the GTGT reads percentage and dividing the remainder by 66.6% (percentage of NCF1B or NCF1C pseudogene reads), as shown in the formula below:

$$Editing efficiency = ( GTGT reads \% - 0.33 ) / 0.66$$

For editing in cells from donor in, e.g., samples No.s 54-59, 83, and 111-118: the donor cells used in these samples are derived from a healthy donor bearing three copies of “GTGT” and three copies of “GT” at the NCF1, NCF1B, and NCF1C loci. Accordingly, for such donor derived cells, GTGT reads include reads from endogenous loci (˜50% in an unedited cell). The pseudogene correction efficiency is thus calculated as below:

$$Editing efficiency = ( GTGT reads \% - 0.5 ) / 0.5$$

For editing in ΔGT CGD patients cells: Because these cells, e.g., those used in Sample No.s 133 and 134, lack a wild-type copy of the NCF1 gene, the percentage of “GTGT” reads at the c.73-74 site over the total reads of NCF1, NCF1B, and NCF1C sequences flanking the c.73-74 site (or corresponding sequences in NCF1B and NCF1C) sequence in exon 2 of NCF1 directly reflects editing efficiency.

In each table, nick-to-edit distance refers to the distance from the PEgRNA's nick site to the intended GT insert in number of nucleotides. Nick-to-nick distance refers to the distance between the PEgRNA's nick site and the ngRNA's nick site in number of nucleotides. A subset of the ngRNAs were designed for a PE3b strategy, and contains spacers complementary to the portion of the edit strand containing the intended edit.

Editing efficiency and indel frequency of each specific pegRNA and ngRNA combination are summarized below. The results demonstrate successful editing of endogenous NCF1, NCF1B and/or NCF1C in CD34+ cells using a PE3 or PE3b Prime Editing strategy.

For each of Tables 101-130, Indicated PEgRNA sequences and ngRNA sequences do not contain the 3′ mU*mU*mU*U additional nucleotides and does not contain the 5′mN*mN*mN* modifications used experimentally, where m indicates that the indicated nucleotide contains a 2′-O-Me modification and a * indicates a phosphorothioate bond.

Indel frequency (%), GTGT reads (%), and editing efficiency (%) in Tables 101-130 are as follows:

Indel frequency: “−”=0; “+”=0-1%; “++”=1%-2%; “+++”=2%-6%; “++++”=greater than 6%. For boarder values, each range includes the upper limit and not the lower limit. For example, 1%-2% means any value larger than 1% and no less than 2%.

Editing efficiency: “−” refers to less than 3%; “+”=3%-10%; “++”=10%-18%; “+++”=18%-25%; “++++”=greater than 25%. For boarder values, each range includes the upper limit and not the lower limit. For example, 3%-10% means any value larger than 3% and no less than 10%.

GTGT reads percentage for cells from healthy donors: “−” refers to less than 35%; “+”=35%-40%; “++”=40%, 45%; “+++”=45%-50%; “++++”=greater than 50%. For boarder values, each range includes the upper limit and not the lower limit. For example, 35%-40% means any value larger than 35% and no less than 40%.

GTGT reads percentage for cells from donors that have three copies of “GTGT”:“−” refers to less than 49%; “+”=49%-53%; “++”=53%-54%; “+++”=54%-68%; “++++”=greater than 68%. For boarder values, each range includes the upper limit and not the lower limit. For example, 49%-53% means any value larger than 49% and no less than 53%.

TABLE 101
CD34+ cells from a healthy donor were electroporated
with mRNA encoding Prime Editor, PEgRNA, and ngRNA in a
25uL electroporation cartridge. Editing efficiency and
indel frequency were measured 72 hours post electroporation.

| Sample | Prime | pegRNA | ngRNA | Indels | GTGT | editing |
|---|---|---|---|---|---|---|
| No. | Editor | SEQ ID | SEQ ID | (%) | (%) | (%) |
| Mock | NA | NA | NA | + | − | − |
| 1 | T1 | 5563 | 584 | ++++ | ++++ | ++++ |
| 2 | F1 | 5563 | 584 | ++++ | +++ | +++ |
| 3 | T1 | 5606 | 584 | +++ | ++ | ++ |
| 4 | T1 | 5647 | 584 | +++ | +++ | +++ |
| 5 | T1 | 5605 | 584 | ++++ | ++++ | ++++ |
| 6 | T1 | 5644 | 584 | ++++ | ++ | ++ |
| 7 | T1 | 5672 | 584 | ++++ | +++ | +++ |

TABLE 102
CD34+ cells from a healthy donor were electroporated with mRNA encoding
the prime editor, PEgRNA, and ngRNA in 25 uL cartridge. Editing efficiency
and indel frequency were measured at 72, 96, and 120 hours post electroporation.

| Sample | Prime | PEgRNA | ngRNA | Indels (%) | | | GTGT (%) | | | Editing (%) | | |
|---|---|---|---|---|---|---|---|---|---|---|---|---|
| No. | Editor | SEQ ID | SEQ ID | 72 h | 96 h | 120 h | 72 h | 96 h | 120 h | 72 h | 96 h | 120 h |
| Mock | NA | NA | NA | + | + | + | − | − | − | − | − | − |
| 8 | T1 | 5529 | 591 | ++ | ++ | ++ | − | − | − | − | − | − |
| 9 | T1 | 5537 | 591 | +++ | +++ | ++++ | ++ | ++ | ++ | ++ | ++ | ++ |
| 10 | T1 | 5542 | 591 | +++ | +++ | +++ | +++ | +++ | +++ | +++ | +++ | +++ |
| 11 | T1 | 5571 | 591 | +++ | +++ | +++ | ++ | ++ | ++ | ++ | ++ | ++ |
| 12 | T1 | 5543 | 591 | +++ | +++ | ++++ | ++ | ++ | ++ | ++ | ++ | ++ |
| 13 | T1 | 5563 | 591 | +++ | ++++ | ++++ | +++ | ++++ | ++++ | +++ | ++++ | ++++ |
| 14 | T1 | 5561 | 591 | +++ | +++ | +++ | + | + | + | + | + | + |
| 15 | T1 | 5569 | 591 | +++ | +++ | +++ | ++++ | ++++ | ++++ | ++++ | ++++ | ++++ |
| 16 | T1 | 5634 | 591 | +++ | +++ | ++++ | ++ | +++ | +++ | ++ | +++ | +++ |
| 17 | T1 | 5570 | 591 | +++ | ++++ | +++ | ++ | + | ++ | ++ | + | ++ |
| 18 | T1 | 5672 | 591 | − | +++ | ++++ | ++ | ++ | ++ | ++ | ++ | ++ |

TABLE 103
The PEgRNA having the sequence of SEQ ID No. 5563 combined with ngRNA having
the sequence of SEQ ID No. 584 was also re-examined as below. CD34+
cells from a healthy donor were electroporated with mRNA encoding the prime
editor, PEgRNA, and ngRNA in 100 uL cartridge. Editing efficiency and indel
frequency were measured at 72, 96, and 120 hours post electroporation.

| Sample | Prime | PEg | ngRNA | Indels (%) | | | GTGT (%) | | | Editing (%) | | |
|---|---|---|---|---|---|---|---|---|---|---|---|---|
| No. | Editor | SEQ ID | SEQ ID | 72 h | 96 h | 120 h | 72 h | 96 h | 120 h | 72 h | 96 h | 120 h |
| Mock | NA | NA | NA | + | + | + | − | − | − | − | − | − |
| 19 | Prime | 5563 | 584 | ++++ | ++++ | ++++ | ++++ | ++++ | ++++ | ++++ | ++++ | ++++ |
| | Editor | | | | | | | | | | | |
| | T1 | | | | | | | | | | | |

The PEgRNA having the sequence of SEQ ID No.5563 combined with ngRNA having the sequence of SEQ ID No. 584 was also tested for editing of NCF1B/NCF1C pseudogenes in healthy CD34+ cells with mRNAs encoding 26 different prime editor fusion proteins each having a SpCas9H840A nickase and a MMLV-RT. Successful Prime Editing was observed in each case (data not shown).

TABLE 104
CD34+ cells from healthy donor were electroporated with mRNA encoding
the prime editor, pegRNA, and ngRNA in 25 uL cartridge. Editing efficiency
and indel frequency were measured at 72, 96, and 120 hours post electroporation.

| Sample | Prime | PEgRNA | ngRNA | Indels (%) | | | GTGT (%) | | | Editing (%) | | |
|---|---|---|---|---|---|---|---|---|---|---|---|---|
| No. | Editor | SEQ ID | SEQ ID | 72 h | 96 h | 120 h | 72 h | 96 h | 120 h | 72 h | 96 h | 120 h |
| Mock | NA | NA | NA | + | + | + | − | − | − | − | − | − |
| 20 | T1 | 5563 | 585 | +++ | +++ | +++ | +++ | +++ | ++++ | +++ | +++ | ++++ |
| 21 | T1 | 5563 | 2143 | + | + | + | + | + | + | + | + | + |
| 22 | T1 | 5563 | 595 | +++ | +++ | +++ | ++ | ++ | +++ | ++ | ++ | +++ |
| 23 | T1 | 5563 | 594 | ++ | ++ | ++ | + | ++ | ++ | + | ++ | ++ |
| 24 | T1 | 5563 | 583 | ++ | ++ | +++ | ++ | ++ | +++ | ++ | ++ | +++ |
| 25 | T1 | 5563 | 592 | ++ | ++ | ++ | + | + | ++ | + | + | ++ |
| 26 | T1 | 5563 | 591 | +++ | +++ | +++ | ++++ | ++++ | ++++ | ++++ | ++++ | ++++ |
| 27 | T1 | 5563 | 590 | ++ | ++ | ++ | ++ | ++ | ++ | ++ | ++ | ++ |

TABLE 105
CD34+ cells from healthy donor were electroporated with mRNA encoding
the prime editor, pegRNA, and ngRNA in 25 uL cartridge. Editing efficiency
and indel frequency were measured at 72, 96, and 120 hours post electroporation.

| Sample | Prime | PEgRNA | ngRNA | Indels (%)² | | | GTGT (%)² | | | Editing (%)² | | |
|---|---|---|---|---|---|---|---|---|---|---|---|---|
| No. | Editor | SEQ ID | SEQ ID¹ | 72 h | 96 h | 120 h | 72 h | 96 h | 120 h | 72 h | 96 h | 120 h |
| Mock | NA | NA | NA | + | + | + | − | − | − | − | − | − |
| 28 | T1 | 5563 | 584 | ++++ | ++++ | +++ | ++++ | ++++ | ++++ | ++++ | ++++ | ++++ |
| 29 | T1 | 5563 | 585 | ++++ | ++++ | ++++ | +++ | ++++ | ++++ | +++ | ++++ | ++++ |
| 30 | T1 | 5563 | 591 | +++ | +++ | +++ | ++ | +++ | +++ | ++ | +++ | +++ |
| 31 | T1 | 5563 | 579^{C} | + | + | + | + | + | − | + | + | − |
| 32 | T1 | 5563 | 578^{C} | + | + | + | − | − | − | − | − | − |
| 33 | T1 | 5606 | 584 | +++ | +++ | +++ | + | + | ++ | + | + | ++ |
| 34 | T1 | 5606 | 585 | +++ | ++++ | NA | ++ | + | NA | ++ | + | NA |
| 35 | T1 | 5606 | 591 | +++ | +++ | ++++ | ++ | ++ | ++ | ++ | ++ | ++ |
| 36 | T1 | 5606 | 579^{C} | + | + | + | − | − | − | − | − | − |

¹A superscript “C” indicates that the ngRNA is a control ngRNA that does not efficiently nick the target DNA strand.
²“NA” indicates sample not taken or sequencing issue at given timepoint.

TABLE 106
CD34+ cells from a healthy donor were electroporated with mRNA encoding
the prime editor, PEgRNA, and ngRNA in 100 uL cartridge. Editing efficiency
and indel frequency were measured at 72, 96, and 120 hours post electroporation.

| Sample | Prime | PEgRNA | ngRNA | Indels (%) | | | GTGT (%) | | | Editing (%) | | |
|---|---|---|---|---|---|---|---|---|---|---|---|---|
| No. | Editor | SEQ ID | SEQ ID | 72 h | 96 h | 120 h | 72 h | 96 h | 120 h | 72 h | 96 h | 120 h |
| Mock | NA | NA | NA | + | + | + | − | − | + | − | − | − |
| 37 | T1 | 5647 | 584 | ++++ | ++++ | ++++ | ++++ | ++++ | ++++ | ++++ | ++++ | ++++ |
| 38 | T1 | 5647 | 591 | +++ | +++ | +++ | ++++ | ++++ | ++++ | ++++ | ++++ | ++++ |
| 39 | T1 | 5647 | 590 | +++ | +++ | +++ | +++ | +++ | ++++ | +++ | +++ | ++++ |

PEgRNA having the sequence of SEQ ID NO 5647 in combination with ngRNA having the sequence of SEQ ID NO 584 and ngRNA having the sequence of SEQ ID NO 590 were also re-examined with miRNA encoding Prime Editor fusion protein T1 or F1. Successful Prime Editing was observed in all cases (data not shown).

TABLE 107
CD34+ cells from a healthy donor were electroporated with mRNA encoding the prime
editor, PEgRNA, and ngRNA in 400 uL cartridge. Editing efficiency and indel frequency
were measured and indel frequency measured at 48, 72, and 96 hours post electroporation.

| Sample | Prime | PEgRNA | ngRNA | Indels (%) | | | GTGT (%) | | | Editing (%) | | |
|---|---|---|---|---|---|---|---|---|---|---|---|---|
| No. | Editor | SEQ ID | SEQ ID | 72 h | 96 h | 120 h | 72 h | 96 h | 120 h | 72 h | 96 h | 120 h |
| Mock | NA | NA | NA | +++ | +++ | + | − | − | − | − | − | − |
| 40 | T1 | 5569 | 585 | ++++ | ++++ | ++++ | ++++ | ++++ | ++++ | ++++ | ++++ | ++++ |
| 41 | T1 | 5569 | 585 | ++++ | ++++ | ++++ | ++++ | ++++ | ++++ | ++++ | ++++ | ++++ |
| 42 | F1 | 5569 | 585 | ++++ | ++++ | ++++ | ++++ | ++++ | ++++ | ++++ | ++++ | ++++ |

TABLE 108
CD34+ cells from a healthy donor were electroporated with mRNA encoding the prime
editor, PEgRNA, and ngRNA in 100 uL cartridge. Editing efficiency and indel frequency
were measured and indel frequency measured at 72, 96, and 120 hours post electroporation.
In cell sample 43, Prime Editing was performed using PE2 strategy, and no ngRNA was
used. The PEgRNA and ngRNA component information is provided in Table 101.

| Sample | Prime | PEgRNA | ngRNA | Indels (%) | | | GTGT (%) | | | Editing (%) | | |
|---|---|---|---|---|---|---|---|---|---|---|---|---|
| No. | Editor | SEQ ID | SEQ ID¹ | 72 h | 96 h | 120 h | 72 h | 96 h | 120 h | 72 h | 96 h | 120 h |
| Mock | NA | NA | NA | + | + | + | − | − | − | − | − | − |
| 43 | T1 | 5605 | PE2 | + | + | + | + | + | + | + | + | + |
| 44 | T1 | 5605 | 584 | ++++ | ++++ | ++++ | ++++ | ++++ | ++++ | ++++ | ++++ | ++++ |

TABLE 109
CD34+ cells from a healthy donor were electroporated with mRNA encoding the prime editor,
PEgRNA, and ngRNA in 25 uL cartridge. Editing efficiency and indel frequency were measured.
Different concentrations of ngRNA were tested. Editing efficiency and indel frequency measured
at 72, 96, and 120 hours post electroporation. For amples 45-47, 48-50, and 51-53, high-concentration,
mid-concentration, and low concentration of ngRNA was used, respectively.

| Sample | Prime | PEgRNA | ngRNA | Indels (%) | | | GTGT (%) | | | Editing (%) | | |
|---|---|---|---|---|---|---|---|---|---|---|---|---|
| No. | Editor | SEQ ID | SEQ ID | 72 h | 96 h | 120 h | 72 h | 96 h | 120 h | 72 h | 96 h | 120 h |
| Mock | NA | NA | NA | + | + | + | − | − | − | − | − | − |
| 45 | F1 | 5605 | 590 | ++++ | ++++ | ++++ | +++ | +++ | +++ | +++ | +++ | +++ |
| 46 | F1 | 5605 | 590 | +++ | +++ | +++ | ++ | +++ | +++ | ++ | +++ | +++ |
| 47 | F1 | 5605 | 590 | +++ | +++ | +++ | ++ | ++ | ++ | ++ | ++ | ++ |
| 48 | F1 | 5605 | 591 | ++++ | ++++ | ++++ | +++ | ++++ | +++ | +++ | ++++ | +++ |
| 49 | F1 | 5605 | 591 | +++ | +++ | +++ | ++ | ++ | ++ | ++ | ++ | ++ |
| 50 | F1 | 5605 | 591 | +++ | +++ | ++++ | ++ | +++ | +++ | ++ | +++ | ++ |
| 51 | F1 | 5605 | 584 | ++++ | ++++ | ++++ | +++ | ++++ | +++ | +++ | ++++ | +++ |
| 52 | F1 | 5605 | 584 | ++++ | ++++ | ++++ | +++ | ++++ | +++ | +++ | ++++ | +++ |
| 53 | F1 | 5605 | 584 | ++++ | ++++ | ++++ | +++ | +++ | +++ | +++ | +++ | +++ |

The PEgRNA having the sequence of SEQ ID No. 5605, combined with the ngRNA having the sequence of SEQ ID No.584, were also tested with mRNA that encode 9 different prime editor fusion proteins. Successful Prime Editing was observed (data not shown).

TABLE 110
CD34+ cells from a donor having 3 copies of “GTGT”
at the c.73_74 or corresponding sites in NCF1, NCF1B and/or
NCF1C were electroporated with mRNA encoding the prime editor,
pegRNA, and ngRNA in 400 uL cartridge. Editing efficiency
and indel frequency were measured at 24 hours post electroporation.

| Sample | Prime | PEgRNA | ngRNA | Indels | GTGT | editing |
|---|---|---|---|---|---|---|
| No. | Editor | SEQ ID | SEQ ID | (%) | (%) | (%) |
| Mock | NA | NA | NA | + | − | − |
| 58 | T1 | 5605 | 584 | ++++ | ++++ | ++++ |
| 59 | T1 | 5605 | 584 | ++++ | ++++ | ++++ |

TABLE 111
CD34+ cells from a donor having 3 copies of “GTGT”
at the c.73_74 or corresponding sites in NCF1, NCF1B and/or
NCF1C were also tested for editing at 72 hours post electroporation.
The cells were electroporated with mRNA encoding the prime
editor, pegRNA, and ngRNA in 400 uL cartridge.

| Sample | Prime | PEgRNA | ngRNA | Indels | GTGT | editing |
|---|---|---|---|---|---|---|
| No. | Editor | SEQ ID | SEQ ID1 | (%) | (%) | (%) |
| Mock | NA | NA | NA | + | − | − |
| 54 | T1 | 5605 | 584 | +++ | ++++ | ++++ |
| 55 | T1 | 5605 | 584 | +++ | +++ | ++++ |
| 56 | T1 | 5605 | 584 | +++ | ++++ | ++++ |
| 57 | T1 | 5605 | 584 | +++ | +++ | ++++ |

TABLE 112
CD34+ cells from a healthy donor were electroporated with mRNA encoding
the prime editor, PEgRNA, and ngRNA in 25 uL cartridge. Editing efficiency
and indel frequency were measured and indel frequency measured at 48 and
120 hours post electroporation. Mock sample was not taken at 120 h.

| Sample | Prime | PEgRNA | ngRNA | Indels(%) | | GTGT (%) | | Editing (%) | |
|---|---|---|---|---|---|---|---|---|---|
| No. | Editor | SEQ ID | SEQ ID | 48 h | 120 h | 48 h | 120 h | 48 h | 120 h |
| Mock | NA | NA | NA | + | NA | − | NA | − | NA |
| 60 | T1 | 5647 | 590 | ++ | ++ | + | + | + | + |
| 61 | T1 | 5570 | 590 | +++ | +++ | + | + | + | + |

TABLE 113
Summary of PEgRNAs used in experiments in Tables 101-112 (spacer
SEQ ID NO. 3999). Nick-to-edit distance for all PEgRNAs is 2 nt.
The RTTs introduce an edit in the edit strand that is in the protospacer.
Incorporation of the edit alters protospacer sequence and may therefore
prevent Cas9 from re-nicking the edit strand.

| pegRNA | | | | |
|---|---|---|---|---|
| SEQ ID s | PBS SEQ ID | PBS length | RTT SEQ ID | RTT length |
| 5529 | 4006 | 7 | 4020 | 11 |
| 5537 | 4010 | 11 | 4020 | 11 |
| 5543 | 4011 | 12 | 4020 | 11 |
| 5542 | 4010 | 11 | 4021 | 12 |
| 5563 | 4011 | 12 | 4021 | 12 |
| 5561 | 4012 | 13 | 4020 | 11 |
| 5569 | 4012 | 13 | 4021 | 12 |
| 5571 | 4010 | 11 | 4054 | 14 |
| 5570 | 4013 | 14 | 4020 | 11 |
| 5606 | 4011 | 12 | 4054 | 14 |
| 5605 | 4013 | 14 | 4021 | 12 |
| 5634 | 4012 | 13 | 4054 | 14 |
| 5647 | 4011 | 12 | 4098 | 16 |
| 5644 | 4013 | 14 | 4054 | 14 |

TABLE 114
Summary of ngRNAs used in experiments in Tables 101-112. The
nick-to-nick distance is based on the sequence pre-editing.

| ngRNA SEQ ID | ngRNA spacer SEQ ID | Nick-to-nick distance |
|---|---|---|
| 592 | 451 | 76 |
| 594 | 421 | 68 |
| 591 | 424 | 82 |
| 585 | 436 | 44 |
| 590 | 422 | 96 |
| 583 | 445 | 75 |
| 595 | 442 | 67 |
| 584 | 429 | 41 |
| 2143 | 2130 | 56 |

TABLE 115
CD34+ cells from a healthy donor were electroporated with mRNA encoding
the prime editor, PEgRNA, and ngRNA in 25 uL cartridge. Editing efficiency
and indel frequency measured at 72, 96, and 120 hours post electroporation.

| Sample | Prime | PEgRNA | ngRNA | Indels (%) | | | GTGT (%) | | | Editing (%) | | |
|---|---|---|---|---|---|---|---|---|---|---|---|---|
| No. | Editor | SEQ ID | SEQ ID | 72 h | 96 h | 120 h | 72 h | 96 h | 120 h | 72 h | 96 h | 120 h |
| Mock | NA | NA | NA | + | + | + | − | − | − | − | − | − |
| 61 | F1 | 19534 | 2142 | +++ | +++ | ++++ | ++ | ++ | +++ | ++ | ++ | +++ |
| 62 | F1 | 19550 | 2142 | +++ | +++ | +++ | +++ | +++ | +++ | +++ | +++ | +++ |
| 63 | F1 | 19497 | 2142 | ++++ | ++++ | ++++ | +++ | +++ | +++ | +++ | +++ | +++ |
| 64 | F1 | 19508 | 2142 | ++++ | ++++ | ++++ | +++ | +++ | +++ | +++ | +++ | +++ |
| 65 | F1 | 19516 | 2142 | ++++ | ++++ | ++++ | ++ | ++ | ++ | ++ | ++ | ++ |
| 66 | F1 | 19526 | 2142 | ++++ | ++++ | ++++ | ++ | ++ | +++ | ++ | ++ | +++ |
| 67 | F1 | 19524 | 2142 | ++++ | ++++ | ++++ | +++ | +++ | +++ | +++ | NA | +++ |
| 68 | F1 | 19537 | 2142 | ++++ | ++++ | ++++ | +++ | +++ | ++++ | +++ | +++ | ++++ |
| 69 | F1 | 19545 | 2142 | ++++ | ++++ | ++++ | +++ | +++ | +++ | +++ | +++ | +++ |
| 70 | F1 | 19535 | 2142 | +++ | +++ | +++ | ++ | ++++ | +++ | ++ | NA | +++ |
| 71 | F1 | 19543 | 2142 | ++++ | +++ | ++++ | ++++ | +++ | ++++ | ++++ | NA | ++++ |

TABLE 116
CD34+ cells from a healthy donor were electroporated
with mRNA encoding the prime editor, PEgRNA, and ngRNA
in 25 uL cartridge. Editing efficiency and indel frequency
were measured 72 hours post electroporation.

| Sample | Prime | pegRNA | ngRNA | Indels | GTGT | Editing |
|---|---|---|---|---|---|---|
| No. | Editor | SEQ ID s | SEQ ID | (%) | (%) | (%) |
| Mock | NA | NA | NA | + | − | − |
| 72 | T1 | 19534 | 2142 | +++ | + | + |
| 73 | T1 | 19534 | 19566 | ++ | + | + |
| 74 | T1 | 19550 | 2142 | +++ | ++ | ++ |
| 75 | T1 | 19550 | 2142 | +++ | ++ | ++ |
| 76 | T1 | 19550 | 19566 | ++ | + | + |
| 77 | T1 | 19550 | 19566 | ++ | + | + |

TABLE 117
CD34+ cells from a healthy donor were electroporated with mRNA encoding
the prime editor, PEgRNA, and ngRNA in 25 uL cartridge. Editing efficiency
and indel frequency measured at 72, 96, and 120 hours post electroporation.

| Sample | Prime | PERNA | ngRNA | Indels (%) | | | GTGT (%) | | | Editing (%) | | |
|---|---|---|---|---|---|---|---|---|---|---|---|---|
| No. | Editor | SEQ ID | SEQ ID¹ | 72 h | 96 h | 120 h | 72 h | 96 h | 120 h | 72 h | 96 h | 120 h |
| Mock | NA | NA | NA | + | + | + | − | − | − | − | − | − |
| 78 | T1 | 19550 | 2142 | +++ | +++ | +++ | + | + | + | + | + | + |
| 79 | T1 | 19550 | 19566 | +++ | +++ | ++ | + | + | + | + | + | + |
| 80 | T1 | 19550 | 2140^{C} | + | + | + | − | − | − | − | − | − |
| 81 | T1 | 19550 | 19564^{C} | + | + | + | − | − | − | − | − | − |

¹A superscript “C” indicates that the ngRNA is a control ngRNA that does not efficiently nick the target DNA strand.

TABLE 118
CD34+ cells from a healthy donor were electroporated with mRNA encoding
the prime editor, PERNA, and ngRNA in 25 uL cartridge. Editing efficiency
and indel frequency measured at 72, 96, and 120 hours post electroporation.

| Sample | Prime | PEgRNA | ngRNA | Indels (%) | | | GTGT (%) | | | Editing (%) | | |
|---|---|---|---|---|---|---|---|---|---|---|---|---|
| No. | Editor | SEQ ID | SEQ ID | 72 h | 96 h | 120 h | 72 h | 96 h | 120 h | 72 h | 96 h | 120 h |
| Mock | NA | NA | NA | + | + | + | − | − | + | − | − | − |
| 82 | T1 | 19550 | 2142 | +++ | +++ | +++ | +++ | +++ | +++ | +++ | +++ | +++ |

TABLE 119
CD34+ cells from a healthy donor were electroporated
with mRNA encoding the prime editor, PEgRNA, and ngRNA
in 400 uL cartridge. Editing efficiency and indel frequency
measured at 24 hours post electroporation

| Sample | Prime | PERNA | ngRNA | Indels | GTGT | Editing |
|---|---|---|---|---|---|---|
| No. | Editor | SEQ ID | SEQ ID | (%) | (%) | (%) |
| Mock | NA | NA | NA | + | − | − |
| 83 | T1 | 19550 | 888 | + | + | + |

TABLE 120
CD34+ cells from a healthy donor were electroporated with mRNA encoding
the prime editor, PERNA, and ngRNA in 25 uL cartridge. Editing efficiency
and indel frequency measured at 72 and 96 hours post electroporation

| Sample | Prime | PEgRNA | ngRNA | Indels(%) | | GTGT percentage(%) | | Editing (%) | |
|---|---|---|---|---|---|---|---|---|---|
| No. | Editor | SEQ ID | SEQ ID | 72 h | 96 h | 72 h | 96 h | 72 h | 96 h |
| Mock | NA | NA | NA | + | + | − | − | − | − |
| 84 | F1 | 19543 | 2143 | ++++ | ++++ | ++++ | ++++ | ++++ | ++++ |
| 85 | F1 | 19543 | 2142 | +++ | +++ | +++ | +++ | +++ | +++ |
| 86 | F1 | 19543 | 888 | + | + | ++ | ++ | ++ | ++ |

TABLE 121
CD34+ cells from a healthy donor were electroporated with mRNA encoding
the prime editor, PEgRNA, and ngRNA in 100 uL cartridge. Editing efficiency
and indel frequency measured at 24, 48, and 120 hours post electroporation.

| Sample | Prime | PEgRNA | ngRNA | Indels (%) | | | GTGT (%) | | | Editing (%) | | |
|---|---|---|---|---|---|---|---|---|---|---|---|---|
| No. | Editor | SEQ ID | SEQ ID | 24 h | 48 h | 120 h | 24 h | 48 h | 120 h | 24 h | 48 h | 120 h |
| Mock | NA | NA | NA | + | + | + | − | − | − | − | − | − |
| 87 | T1 | 19543 | 888 | + | + | + | + | + | ++ | + | + | ++ |
| 88 | T1 | 19543 | 2143 | +++ | +++ | ++++ | ++ | +++ | ++++ | ++ | +++ | ++++ |
| 89 | T1 | 19543 | 2142 | +++ | +++ | ++++ | + | + | +++ | + | + | +++ |

TABLE 122
CD34+ cells from a healthy donor were electroporated with mRNA encoding
the prime editor, PEgRNA, and ngRNA in 100 uL cartridge. Editing efficiency
and indel frequency measured at 24, 48, and 120 hours post electroporation

| | | | | Indels (%) | | | GTGT (%) | | | Editing (%) | | |
|---|---|---|---|---|---|---|---|---|---|---|---|---|
| Sample | Prime | PEgRNA | ngRNA | 24 | 48 | 120 | 24 | 48 | 120 | 24 | 48 | 120 |
| No. | Editor | SEQ ID | SEQ ID | h | h | h | h | h | h | h | h | h |
| Mock | NA | NA | NA | + | + | + | − | − | − | − | − | − |
| 90 | T1 | 19543 | 878 | + | + | + | ++ | +++ | ++++ | ++ | +++ | ++++ |
| 91 | T1 | 19543 | 880 | + | + | + | ++ | +++ | ++++ | ++ | +++ | ++++ |
| 92 | T1 | 19543 | 888 | + | + | + | ++ | +++ | ++++ | ++ | +++ | ++++ |
| 93 | T1 | 19543 | 883 | + | + | ++ | +++ | ++++ | ++++ | +++ | ++++ | ++++ |

TABLE 123
CD34+ cells from a healthy donor were electroporated
with mRNA encoding the prime editor, PEgRNA, and ngRNA
in 100 uL cartridge. Editing efficiency and indel frequency
measured at 72 hours post electroporation

| Sample | Prime | PEgRNA | ngRNA | Indels | GTGT | Editing |
|---|---|---|---|---|---|---|
| No. | Editor | SEQ ID | SEQ ID | (%) | (%) | (%) |
| Mock | NA | NA | NA | + | − | − |
| 94 | T1 | 19543 | 883 | + | +++ | +++ |
| 95 | T1 | 19543 | 880 | + | ++ | ++ |
| 96 | T1 | 19543 | 883 | ++ | ++++ | ++++ |

TABLE 124
CD34+ cells from a healthy donor were electroporated with mRNA encoding the
prime editor, PEgRNA, and ngRNA in 25 uL cartridge. Editing efficiency and indel
frequency measured at 24, 48, and 120 hours post electroporation. The Mock, sample
numbers 97, 98, and 99, each had 100, 400, 400, and 100 uL cartridge volume.

| | | | | Indels (%) | | | GTGT (%) | | | Editing (%) | | |
|---|---|---|---|---|---|---|---|---|---|---|---|---|
| Sample | Prime | PEgRNA | ngRNA | 24 | 48 | 120 | 24 | 48 | 120 | 24 | 48 | 120 |
| No. | Editor | SEQ ID | SEQ ID | h | h | h | h | h | h | h | h | h |
| Mock | NA | NA | NA | + | + | + | − | − | − | − | − | − |
| 97 | F1 | 19543 | 883 | + | +++ | ++ | ++++ | ++++ | ++++ | ++++ | ++++ | ++++ |
| 98 | T1 | 19543 | 883 | ++ | ++ | +++ | ++++ | ++++ | ++++ | ++++ | ++++ | ++++ |
| 99 | F2 | 19543 | 883 | + | + | ++ | ++ | +++ | ++++ | ++ | +++ | ++++ |

TABLE 125
CD34+ cells from a healthy donor were electroporated
with mRNA encoding the prime editor, PEgRNA, and ngRNA
in 25 uL cartridge. Editing efficiency and indel frequency
measured at 120 hours post electroporation. Samples 176-
178, 179-182, and 183-186 were each contacted with high,
mid, and low concentration of pegRNA and low, mid, and
high concentration ngRNA, respectively.

| Sample | Prime | PEgRNA | ngRNA | Indels | GTGT | Editing |
|---|---|---|---|---|---|---|
| No. | Editor | SEQ ID | SEQ ID | (%) | (%) | (%) |
| Mock | NA | NA | NA | NA | NA | NA |
| 100 | T1 | 19537 | 881 | + | + | + |
| 101 | T1 | 19537 | 879 | + | + | + |
| 102 | T1 | 19537 | 888 | + | ++ | ++ |
| 103 | T1 | 19537 | 880 | + | ++ | ++ |
| 104 | T1 | 19537 | 881 | + | + | + |
| 105 | T1 | 19537 | 879 | + | ++ | ++ |
| 106 | T1 | 19537 | 888 | + | ++ | ++ |
| 107 | T1 | 19537 | 880 | ++ | +++ | ++ |
| 108 | T1 | 19537 | 881 | + | ++ | ++ |
| 109 | T1 | 19537 | 879 | + | + | + |
| 110 | T1 | 19537 | 888 | + | ++ | ++ |

TABLE 126
CD34+ cells (4.00E+07 cells/mL at electroporation)
from a donor having 3 copies of “GTGT” at the
c.73_74 or corresponding sites in NCF1, NCF1B and/or
NCF1C were electroporated with mRNA encoding prime editor,
pegRNA, and ngRNA. Editing efficiency and indel frequency
measured at 72 hours post electroporation.

| Sample | Prime | PEgRNA | ngRNA | Indels | GTGT | Editing |
|---|---|---|---|---|---|---|
| No. | Editor | SEQ ID | SEQ ID | (%) | (%) | (%) |
| Mock | NA | NA | NA | + | − | − |
| 111 | T1 | 19537 | 878 | + | + | + |
| 112 | T1 | 19537 | 880 | + | +++ | + |
| 113 | T1 | 19537 | 881 | + | + | + |
| 114 | T1 | 19537 | 879 | + | + | + |
| 115 | T1 | 19537 | 888 | + | + | + |
| 116 | T1 | 19545 | 888 | + | + | + |
| 117 | T1 | 19535 | 888 | + | + | + |
| 118 | T1 | 19543 | 888 | + | +++ | ++ |

TABLE 127
CD34+ cells from a healthy donor were electroporated with mRNA encoding
the prime editor, PEgRNA, and ngRNA in 25 uL cartridge. Editing efficiency
and indel frequency measured at 72 and 120 hours post electroporation.

| | | | | | | GTGT | | | |
|---|---|---|---|---|---|---|---|---|---|
| | | | | Indels(%)¹ | | percentage(%)¹ | | Editing (%)¹ | |
| Sample | Prime | PEgRNA | ngRNA | 72 | 120 | 72 | 120 | 72 | 120 |
| No. | Editor | SEQ ID | SEQ ID | h | h | h | h | h | h |
| Mock | NA | NA | NA | + | NA | − | NA | − | NA |
| 119 | T1 | 19524 | 878 | + | + | + | ++ | + | ++ |
| 120 | T1 | 19524 | 880 | + | + | + | ++ | + | ++ |
| 121 | T1 | 19524 | 881 | + | + | + | + | + | + |
| 122 | T1 | 19524 | 879 | + | + | + | + | + | + |
| 123 | T1 | 19524 | 877 | + | + | + | + | + | + |
| 124 | T1 | 19524 | 888 | + | + | + | + | + | + |
| 125 | T1 | 19524 | 887 | + | + | − | − | − | − |
| 126 | T1 | 19524 | 892 | + | + | + | + | + | + |
| 127 | T1 | 19524 | 883 | + | + | ++ | ++ | ++ | ++ |
| 128 | F2 | 19524 | 880 | + | + | + | + | + | + |
| 129 | F2 | 19524 | 888 | + | + | + | + | + | + |
| 130 | F2 | 19524 | 887 | NA | +++ | NA | + | NA | + |
| 131 | F2 | 19524 | 892 | NA | + | NA | + | NA | − |
| 132 | F2 | 19524 | 883 | NA | + | NA | + | NA | + |

¹“NA” indicates sample not taken or sequencing issue at given timepoint.

TABLE 128
Summary of PEgRNAs used in experiments in Tables
115-127 (spacer SEQ ID NO. 19081). Nick-to-
edit distance for all PEgRNAs is 7 nt.

| PEgRNA | | PBS | | RTT |
|---|---|---|---|---|
| SEQ ID | PBS SEQ ID | length | RTT SEQ ID | length |
| 19497 | 19092 | 12 | 19112 | 13 |
| 19524 | 19092 | 12 | 19126 | 16 |
| 19535 | 19092 | 12 | 19129 | 17 |
| 19508 | 19093 | 13 | 19112 | 13 |
| 19526 | 19093 | 13 | 19123 | 15 |
| 19537 | 19093 | 13 | 19126 | 16 |
| 19543 | 19093 | 13 | 19129 | 17 |
| 19516 | 19094 | 14 | 19112 | 13 |
| 19534 | 19094 | 14 | 19123 | 15 |
| 19545 | 19094 | 14 | 19126 | 16 |
| 19550 | 19094 | 14 | 19129 | 17 |

TABLE 129
Summary of ngRNAs used in experiments in Tables 115-
127. The nick-to-nick distance is based on the sequence
pre-editing. A star (*) indicates a PE3b ngRNA.

| ngRNA SEQ ID | ngRNA spacer SEQ ID | Nick-to-nick distance |
|---|---|---|
| 879* | 769 | 4 |
| 887* | 803 | 4 |
| 877* | 766 | 5 |
| 881* | 768 | 5 |
| 892* | 839 | 5 |
| 880* | 767 | 6 |
| 883* | 833 | 6 |
| 878* | 770 | 7 |
| 888* | 849 | 7 |
| 2143 | 2130 | 61 |
| 2142 | 2133 | 72 |
| 19566* | 19478 | 88 |

TABLE 130
Two pairs of PEgRNA and ngRNA were tested in CD34+ cells derived from a ΔGT CGD patient
and in CD34+ cells from a healthy donor. The cells were electroporated with mRNA encoding
prime editor, and different concentrations of PEgRNA and ngRNA in different cartridge volumes.
Pseudogene and/or NCF1 editing efficiency and indel frequency were measured at 24, 48, and
120 hours post electroporation. Samples 133 and 135 had mid concentration and high concentration
of ngRNA, and samples 134 and 136 high concentrations of PEgRNA and low concentration of ngRNA

| | | | | | | | | GTGT | | | | | |
|---|---|---|---|---|---|---|---|---|---|---|---|---|---|
| | | | | | Indels (%) | | | percentage (%) | | | Editing (%) | | |
| Sample No. | Cell | Prime | PEgRNA | ngRNA | 24 | 48 | 120 | 24 | 48 | 120 | 24 | 48 | 120 |
| for filing | source | Editor | SEQ ID | SEQ ID | h | h | h | h | h | h | h | h | h |
| Mock1 | ΔGT | NA | | | + | + | + | NA | NA | NA | − | − | − |
| | CGD | | | | | | | | | | | | |
| | patient | | | | | | | | | | | | |
| 133 | ΔGT | T1 | 19537 | 880 | + | ++ | ++ | NA | NA | NA | + | +++ | ++++ |
| | CGD | | | | | | | | | | | | |
| | patient | | | | | | | | | | | | |
| 134 | ΔGT | T1 | 5605 | 584 | ++++ | ++++ | ++++ | NA | NA | NA | ++++ | ++++ | ++++ |
| | CGD | | | | | | | | | | | | |
| | patient | | | | | | | | | | | | |
| Mock2 | Healthy | Mock | | | + | ++ | + | − | − | − | − | − | − |
| 135 | Healthy | T1 | 19537 | 880 | + | ++ | ++ | + | +++ | ++++ | + | +++ | ++++ |
| 136 | Healthy | T1 | 5605 | 584 | +++ | +++ | ++++ | ++++ | ++++ | ++++ | ++++ | ++++ | ++++ |

### Example 7: Analysis of Cones Derived from Ex Vivo Edited CD34+ Cells

To confirm that the editing efficiency observed in Example 6 throughout the population of CD34+ cells contacted with the prime editing components, a subset of cells edited in Example 6 were further examined with clonal analysis. Edited cells from sample numbers 19, 37-39, 40-42, 44, 58, 59, 82, 87-89, 97-99, 133, and 134 were each plated separately in Methocult (Stem Cell Technologies, H4435) at 300 to 500 cells per well and incubated for 14 days for the clones to grow. Following incubation, clones were picked individually into 15 μL of Quick extract buffer for genomic DNA extraction. The extracted genomic DNA was PCR amplified for amplicon sequencing

For analysis No.6, cells with at least 66% GTGT reads at the NCF1, NCF1B, and NCF1C target sites corresponding to c.73_74 in NCF1 were deemed to carry a desired GT insertion at the at the target site of at least 1 pseudogene. For the other analysis groups, cells with at least 50% GTGT reads at the NCF1, NCF1B, and NCF1C target sites corresponding to c.73_74 in NCF1 were deemed to carry a desired GT insertion at the at the target site of at least 1 pseudogene. When calculated, percentage of GTGT reads and indels were bucketed with +/−3%.

Results of the clonal analysis are summarized in Table 131 below. The sample numbers of edited cells as well as the pegRNA and ngRNA used in editing are also shown. The percentage of clones carrying desired edit indicate that the prime editing efficiency reflects efficient editing throughout the population of cells.

TABLE 131
Ex vivo clonal analysis of CD34+ cells edited with Prime Editing.

| | | | | | | Clone | |
|---|---|---|---|---|---|---|---|
| | Sample No. | | | | | with indel | |
| Analysis | (as in | Prime | PEgRNA | ngRNA | Clones | at ≥1 | Clones |
| No. | Example 6) | Editor | SEQ ID | SEQ ID | corrected¹ | allele² | assessed |
| 1 | Mock | NA | NA | NA | − | − | 2 |
| | 44 | T1 | 5605 | 584 | +++ | ++++ | 34 |
| 2 | Mock | NA | NA | NA | − | − | 11 |
| | 19 | T1 | 5563 | 584 | +++ | ++++ | 84 |
| 3 | Mock | NA | NA | NA | − | − | 12 |
| | 37 | T1 | 5647 | 584 | ++++ | +++ | 70 |
| | 38 | T1 | 5647 | 591 | ++ | ++ | 60 |
| | 39 | T1 | 5647 | 590 | + | ++ | 69 |
| | 82 | T1 | 19550 | 2142 | + | ++ | 64 |
| 4 | Mock | NA | NA | NA | − | − | 20 |
| | 40 | T1 | 5569 | 585 | ++ | ++ | 71 |
| | 41 | T1 | 5569 | 585 | ++++ | +++ | 69 |
| | 42 | F1 | 5569 | 585 | +++ | ++++ | 67 |
| 5 | Mock | NA | NA | NA | − | − | 12 |
| | 87 | T1 | 19543 | 888 | + | + | 91 |
| | 88 | T1 | 19543 | 2143 | ++ | +++ | 58 |
| | 89 | T1 | 19543 | 2142 | + | ++++ | 58 |
| 6 | Mock | NA | NA | NA | − | − | 8 |
| | 58 | T1 | 5605 | 584 | ++++ | ++ | 83 |
| | 59 | T1 | 5605 | 584 | +++ | ++ | 84 |
| 7 | Mock | NA | NA | NA | − | − | 79 |
| | 133 | T1 | 19537 | 880 | ++ | + | 232 |
| | 134 | T1 | 5605 | 584 | ++++ | ++++ | 337 |
| 8 | Mock | NA | NA | NA | − | − | 24 |
| | 97 | F1 | 19543 | 883 | ++++ | + | 36 |
| | 98 | T1 | 19543 | 883 | ++++ | ++ | 35 |
| | 99 | F2 | 19543 | 883 | + | + | 34 |

¹“−” refers to 0%. “+” = 58%-79%; “++” = 79%-88%; “+++” = 88%-94%; “++++” = greater than 94%. For boarder values, each range includes the upper limit and not the lower limit. For example, 58%-79% means any value larger than 58% and no less than 79%.
²“−” refers to 0%; “+” = 0-13%; “++” = 13%-25%; “+++” = 25%-34%; “++++” = greater than 34%. For boarder values, each range includes the upper limit and not the lower limit. For example, 13%-25% means any value larger than 13% and no less than 25%.

### Example 8: In Vivo Engraftment of Prime Edited CD34+ Cells and Chimerism Analysis Post Engraftment

A subset of edited cells in Example 6 were cryopreserved 24 hours post electroporation and used for in vivo engraftment studies. On the day of engraftment, cells were thawed, counted, and resuspended to 5E+6 cells per mL in PBS+0.1% BSA. Cells were then injected intravenously into the tail veins of 6-week-old female NBSGW mice (Jackson Laboratory, Stock Number 026622) at 200 μL/mouse. At desired timepoints (7, 8, or 16 weeks) after engraftment, mice were euthanized, and bone marrow and spleen collected for analysis.

Firstly, samples from each mouse were assessed by flow cytometry for human chimerism to ensure that the Prime edited cells were able to successfully engraft. Chimerism was calculated as percentage of human CD45+ cells divided by the percentage of (human CD45⁺ cells+mouse CD45⁺ cells):

$$Chimerism = \frac{huCD {45}^{+}}{huCD {45}^{+} + mCD {45}^{+}}$$

Samples were also assessed for human monocytes (CD14), B cells (CD19), myeloid cells (CD33) hematopoietic stem and progenitor cells (HSPC, CD34) and erythroid (GlyA) fractions. Each engraftment group contained 4 to 5 mice. For each group, Dunnett's multiple comparison test was used to analyze chimerism in each fraction compared to mouse engrafted with the control CD34+ cell (“Mock”) in each group.

Mice engrafted with CD34+ cells edited in Sample No.s 40-42, 58, 59, 87-89, and 97-99 as described in Example 6 were examined. For each Sample No. and Mock control, 4 to 5 mice were engrafted. Bone marrow samples from each of the engrafted mice were sorted into their respective human cell fractions (CD19, CD33, CD34, and GlyA) (BD Aria fusion). The fractions were analyzed for chimerism and compared to Mock treatment. Dunnett's multiple comparison test was performed for the statistical analysis. 128 mice, including 30 Mocks, were analyzed. Except for 13 mice (all from one engraftment experiment), no significant was detected in engrafted mice compared to Mock controls.

The percentage of Prime Edited cells having the intended GT insertion and indels in each of the fractions are summarized in Tables 132-138 below.

The detection of the desired Prime edit in each of the isolated fractions demonstrates that the edited cells were able to engraft, and that Prime Editing can be achieved without hindering normal HSPC function.

TABLE 132
Percentage of edited cells and indels 7 weeks after engraftment. The first
column indicates the sample number of the CD34+ cells edited as described
in Example 6. Sample No.s 40-42 were edited with PEgRNA having the sequence
of SEQ ID No. 5569 and ngRNA having the sequence of SEQ ID No. 585.

| | | % Indels¹ | | | | | % Editing² | | | | |
|---|---|---|---|---|---|---|---|---|---|---|---|
| Sample | | Bulk | | | | | Bulk | | | | |
| No. | Mouse # | BM | CD34 | CD33 | CD19 | Erythroid | BM | CD34 | CD33 | CD19 | Erythroid |
| Mock | 3 | NA | NA | NA | NA | NA | NA | NA | NA | NA | NA |
| | 4 | NA | NA | NA | NA | NA | NA | NA | NA | NA | NA |
| | 7 | + | + | + | + | + | − | − | − | − | − |
| | 41 | + | + | + | + | + | − | − | − | − | − |
| | 42 | + | + | + | + | + | − | − | − | − | − |
| 40 | 11 | + | + | ++ | ++ | +++ | + | + | + | ++ | + |
| | 12 | ++ | ++ | ++ | + | ++ | ++ | ++ | ++ | +++ | ++ |
| | 13 | +++ | ++++ | ++ | ++++ | ++ | + | + | ++ | + | + |
| | 14 | ++ | +++ | ++ | ++ | ++ | + | + | + | + | + |
| | 15 | ++ | ++ | ++ | + | ++ | + | + | + | ++ | + |
| 41 | 21 | ++ | + | +++ | + | +++ | ++++ | ++++ | +++ | ++++ | +++ |
| | 22 | +++ | + | +++ | +++ | ++++ | ++++ | ++++ | ++++ | +++ | ++++ |
| | 23 | ++ | +++ | +++ | ++ | ++++ | ++++ | ++++ | ++++ | ++++ | ++ |
| | 24 | +++ | ++++ | ++ | ++ | ++++ | ++++ | +++ | ++++ | ++++ | +++ |
| | 25 | ++++ | +++ | +++ | ++++ | +++ | +++ | ++++ | ++++ | +++ | +++ |
| 42 | 31 | +++ | +++ | +++ | +++ | ++++ | +++ | +++ | ++++ | +++ | + |
| | 32 | ++++ | ++++ | ++++ | ++++ | ++++ | ++ | ++ | ++++ | +++ | + |
| | 33 | +++ | +++ | +++ | ++ | +++ | ++ | ++++ | +++ | +++ | ++ |
| | 34 | +++ | ++++ | +++ | +++ | ++++ | ++ | +++ | +++ | +++ | + |
| | 35 | +++ | ++++ | ++++ | ++++ | ++++ | +++ | +++ | ++ | + | + |

¹“−” refers to 0%. “+” = 0%-7%; “++” = 7%-9%; “+++” = 9%-12%; “++++” = larger than 12%. For boarder values, each range includes the upper limit and not the lower limit. For example, 7%-9% means any value larger than 7% and no less than 9%. “NA” indicates sample not taken.
²“−” refers to less than 2%; “+” = 2-34%; “++” = 34%-37%; “+++” = 37%-41%; “++++” = larger than 41%. For boarder values, each range includes the upper limit and not the lower limit. For example, 34%-37% means any value larger than 34% and no less than 37%. “NA” indicates sample not taken.

TABLE 133
Percentage of edited cells and indels 16 weeks after engraftment. The first column indicates the
sample number of the CD34+ cells edited as described in Example 6. Sample No.s 40-42 were edited
with PEgRNA having the sequence of SEQ ID No. 5569 and ngRNA having the sequence of SEQ ID No. 585.

| | | % Indels¹ | | | | | | % Editing² | | | | | |
|---|---|---|---|---|---|---|---|---|---|---|---|---|---|
| Sample | Mouse | Bulk | Bulk | | | | | Bulk | Bulk | | | | |
| No. | # | BM | Spleen | CD19 | CD33 | CD34 | Erythroid | BM | Spleen | CD19 | CD33 | CD34 | Erythroid |
| Mock | 5 | + | + | + | + | + | + | − | − | − | − | − | − |
| | 6 | + | + | NA | NA | NA | NA | − | − | NA | NA | NA | NA |
| | 8 | + | + | + | + | + | + | − | − | − | − | − | − |
| | 9 | + | + | NA | NA | NA | NA | − | − | NA | NA | NA | NA |
| | 10 | + | + | NA | NA | NA | NA | − | − | NA | NA | NA | NA |
| 40 | 16 | + | ++ | + | ++ | + | + | + | + | + | +++ | ++ | + |
| | 17 | ++ | +++ | ++ | ++ | ++ | ++ | + | + | + | ++ | ++ | + |
| | 18 | ++ | ++ | ++ | ++ | ++ | + | + | + | + | ++ | + | ++ |
| | 19 | +++ | + | ++ | +++ | +++ | ++ | + | + | ++ | + | + | + |
| | 20 | + | ++ | + | ++ | + | ++ | + | + | + | ++ | + | + |
| 41 | 26 | ++ | ++++ | ++ | ++ | +++ | ++++ | +++ | ++++ | ++++ | ++++ | ++++ | ++++ |
| | 27 | ++++ | ++++ | ++++ | ++++ | ++++ | ++++ | ++ | ++++ | +++ | ++++ | +++ | ++ |
| | 28 | ++++ | ++ | +++ | +++ | ++++ | ++++ | ++++ | ++++ | ++++ | ++++ | ++++ | ++++ |
| | 29 | ++++ | ++ | ++ | ++++ | +++ | ++ | +++ | ++++ | ++++ | ++++ | ++++ | ++++ |
| | 30 | ++++ | ++++ | ++++ | ++++ | ++++ | ++++ | ++++ | ++++ | ++++ | ++++ | ++++ | ++++ |
| 42 | 36 | ++++ | ++++ | ++++ | ++++ | +++ | ++++ | +++ | +++ | +++ | +++ | ++++ | +++ |
| | 37 | ++++ | ++++ | ++++ | ++++ | ++++ | ++++ | + | +++ | ++ | ++ | ++ | ++ |
| | 38 | ++ | ++++ | ++ | ++ | +++ | +++ | ++ | ++ | +++ | ++++ | ++ | +++ |
| | 39 | ++++ | ++++ | ++++ | +++ | ++++ | ++ | + | +++ | +++ | ++ | ++ | +++ |
| | 40 | ++ | ++ | ++ | ++++ | ++ | ++ | ++ | ++ | ++++ | +++ | +++ | ++ |

¹“−” refers to 0%. “+” = 0%-4%; “++” = 4%-6%; “+++” = 6%-7%; “++++” = larger than 7%. For boarder values, each range includes the upper limit and not the lower limit. For example, 6%-7% means any value larger than 6% and no less than 7%. “NA” indicates sample not taken.
²“−” refers to less than 9%; “+” = 9-26%; “++” = 26%-31%; “+++” = 31%-33%; “++++” = larger than 33%. For boarder values, each range includes the upper limit and not the lower limit. For example, 31%-33% means any value larger than 31% and no less than 33%. “NA” indicates sample not taken.

TABLE 134
Percentage of edited cells and indels 8 weeks after engraftment. The first column indicates the
sample number of the CD34+ cells edited as described in Example 6. Cells in sample No. 87
were edited with PEgRNA having the sequence of SEQ ID No. 19543 and ngRNA having the sequence
of SEQ ID No. 888. Cells in sample No. 88 were edited with PEgRNA having the sequence of SEQ ID
No. 19543 and ngRNA having the sequence of SEQ ID No. 2143. Cells in sample No. 89 were edited
with PEgRNA having the sequence of SEQ ID No. 19543 and ngRNA having the sequence of SEQ ID No. 2142.

| | | % Indels¹ | | | | | | % Editing² | | | | | |
|---|---|---|---|---|---|---|---|---|---|---|---|---|---|
| Sample | Mouse | Bulk | Bulk | | | | | Bulk | Bulk | | | | |
| No. | # | BM | Spleen | CD19 | CD33 | CD34 | Erythroid | BM | Spleen | CD19 | CD33 | CD34 | Erythroid |
| Mock | 48 | + | + | + | + | + | + | − | − | − | − | − | − |
| | 49 | + | + | NA | NA | NA | NA | − | − | NA | NA | NA | NA |
| | 50 | + | + | + | + | + | + | − | − | − | − | − | − |
| | 51 | + | + | NA | NA | NA | NA | − | − | NA | NA | NA | NA |
| | 52 | + | + | + | + | + | + | − | − | − | − | − | − |
| 87 | 60 | ++ | ++ | + | ++ | ++ | ++ | + | + | + | + | + | ++ |
| | 61 | ++ | ++ | ++ | ++ | ++ | ++ | + | + | + | + | + | + |
| | 62 | ++ | ++ | + | ++ | ++ | ++ | + | + | + | + | + | + |
| | 63 | ++ | + | ++ | +++ | ++ | ++ | + | + | + | + | + | + |
| 88 | 70 | ++++ | +++ | +++ | ++++ | ++++ | ++++ | +++ | +++ | ++++ | ++++ | ++++ | ++++ |
| | 71 | ++++ | ++++ | +++ | ++++ | ++++ | ++++ | ++++ | +++ | +++ | ++++ | ++++ | ++++ |
| | 72 | ++++ | ++++ | +++ | ++++ | ++++ | ++++ | +++ | ++++ | +++ | ++++ | ++++ | +++ |
| | 73 | +++ | ++++ | ++++ | ++++ | +++ | ++++ | +++ | +++ | +++ | ++++ | +++ | ++++ |
| | 74 | ++++ | ++++ | ++++ | ++++ | ++++ | ++++ | ++++ | ++++ | +++ | ++++ | ++++ | ++++ |
| 89 | 80 | +++ | +++ | +++ | +++ | +++ | +++ | ++ | ++ | ++ | ++ | ++ | +++ |
| | 81 | +++ | +++ | ++ | +++ | +++ | +++ | +++ | ++ | +++ | +++ | +++ | +++ |
| | 82 | +++ | +++ | +++ | +++ | +++ | +++ | +++ | +++ | ++ | ++ | +++ | ++ |
| | 83 | ++++ | +++ | +++ | +++ | +++ | +++ | ++ | ++ | ++ | ++ | ++ | ++ |

¹“−” refers to 0%. “+” = 0%-1%; “++” = 1%-2%; “+++” = 2%-4%; “++++” = larger than 4%. For boarder values, each range includes the upper limit and not the lower limit. For example, 1%-2% means any value larger than 1% and no less than 2%. “NA” indicates sample not taken.
²“−” refers to less than 1%; “+” = 1-15%; “++” = 15%-21%; “+++” = 21%-38%; “++++” = larger than 38%. For boarder values, each range includes the upper limit and not the lower limit. For example, 15%-21% means any value larger than 15% and no less than 21%. “NA” indicates sample not taken.

TABLE 135
Percentage of edited cells and indels 16 weeks after engraftment. The first column indicates the
sample number of the CD34+ cells edited as described in Example 6. Cells in sample No. 87
were edited with PEgRNA having the sequence of SEQ ID No. 19543 and ngRNA having the sequence
of SEQ ID No. 888. Cells in sample No. 88 were edited with PEgRNA having the sequence of SEQ ID
No. 19543 and ngRNA having the sequence of SEQ ID No. 2143. Cells in sample No. 89 were edited
with PEgRNA having the sequence of SEQ ID No. 19543 and ngRNA having the sequence of SEQ ID No. 2142.

| | | % Indels¹ | | | | | | % Editing² | | | | | |
|---|---|---|---|---|---|---|---|---|---|---|---|---|---|
| Sample | Mouse | Bulk | Bulk | | | | | Bulk | Bulk | | | | |
| No. | # | BM | Spleen | CD19 | CD33 | CD34 | Erythroid | BM | Spleen | CD19 | CD33 | CD34 | Erythroid |
| Mock | 43 | + | + | + | + | + | + | − | − | − | − | − | − |
| | 44 | + | + | + | + | + | + | − | − | − | − | − | − |
| | 45 | + | + | + | + | + | NA | − | − | − | − | − | NA |
| | 46 | + | + | NA | NA | NA | NA | − | − | NA | NA | NA | NA |
| | 47 | + | + | + | + | + | + | − | − | − | − | − | − |
| 87 | 55 | + | + | + | + | ++ | ++ | + | + | + | + | ++ | ++ |
| | 56 | + | + | + | + | + | + | + | ++ | + | + | + | + |
| | 57 | ++ | ++ | + | ++ | ++ | NA | + | + | + | ++ | ++ | NA |
| | 58 | + | ++ | + | ++ | + | + | + | + | ++ | ++ | + | ++ |
| | 59 | + | NA | ++ | ++ | + | + | + | NA | ++ | ++ | + | ++ |
| 88 | 65 | ++++ | ++++ | ++++ | ++++ | ++++ | ++++ | ++++ | ++++ | ++++ | ++++ | ++++ | ++++ |
| | 66 | ++++ | ++++ | +++ | ++++ | ++++ | NA | ++++ | ++++ | ++++ | ++++ | ++++ | NA |
| | 67 | ++++ | ++++ | ++++ | ++++ | ++++ | ++++ | +++ | ++++ | +++ | +++ | +++ | +++ |
| | 68 | ++++ | ++++ | ++++ | ++++ | ++++ | ++++ | ++++ | ++++ | ++++ | ++++ | ++++ | ++++ |
| | 69 | ++++ | ++++ | ++++ | ++++ | ++++ | +++ | +++ | ++++ | +++ | ++++ | ++++ | ++++ |
| 89 | 75 | +++ | +++ | +++ | +++ | +++ | ++ | ++ | +++ | ++ | +++ | ++ | +++ |
| | 76 | +++ | +++ | ++ | +++ | +++ | ++ | ++ | +++ | ++ | ++ | ++ | +++ |
| | 77 | +++ | ++ | +++ | +++ | +++ | +++ | +++ | +++ | +++ | +++ | ++ | +++ |
| | 78 | ++++ | +++ | +++ | +++ | +++ | +++ | + | ++ | +++ | +++ | +++ | +++ |
| | 79 | +++ | +++ | ++++ | +++ | +++ | ++ | +++ | +++ | ++ | +++ | +++ | +++ |

¹“−” refers to 0%. “+” = 0%-1%; “++” = 1%-2%; “+++” = 2%-4%; “++++” = larger than 4%. For boarder values, each range includes the upper limit and not the lower limit. For example, 1%-2% means any value larger than 1% and no less than 2%. “NA” indicates sample not taken or unsuccessful sequencing.
²“−” refers to less than 1%; “+” = 1-9%; “++” = 9%-14%; “+++” = 14%-32%; “++++” = larger than 32%. For boarder values, each range includes the upper limit and not the lower limit. For example, 14%-32% means any value larger than 14% and no less than 32%. “NA” indicates sample not taken or unsuccessful sequencing.

TABLE 136
Percentage of edited cells and indels 8 weeks after engraftment. The first column indicates the sample
number of the CD34+ cells edited as described in Example 6. Samples 58 and 59 were edited with
PEgRNA having the sequence of SEQ ID No. 5605 and ngRNA having the sequence of SEQ ID No. 584

| | | % Indels¹ | | | | | | % Editing² | | | | | |
|---|---|---|---|---|---|---|---|---|---|---|---|---|---|
| Sample | Mouse | Bulk | Bulk | | | | | Bulk | Bulk | | | | |
| No. | # | BM | Spleen | CD19 | CD33 | CD34 | Erythroid | BM | Spleen | CD19 | CD33 | CD34 | Erythroid |
| Mock | 85 | + | + | NA | NA | NA | NA | − | − | NA | NA | NA | NA |
| | 86 | + | + | NA | NA | NA | NA | − | − | NA | NA | NA | NA |
| | 87 | + | + | NA | NA | NA | NA | − | − | NA | NA | NA | NA |
| | 88 | + | + | + | + | + | + | − | − | − | − | − | − |
| | 89 | + | + | + | + | + | + | − | − | − | − | − | − |
| 58 | 95 | ++++ | +++ | ++++ | ++++ | ++++ | ++ | ++++ | ++++ | +++ | ++++ | +++ | ++++ |
| | 96 | ++ | ++ | ++ | +++ | +++ | ++++ | ++++ | ++++ | ++++ | ++++ | ++++ | ++++ |
| | 97 | +++ | +++ | +++ | ++ | +++ | ++++ | ++++ | ++++ | ++++ | ++++ | ++++ | +++ |
| | 98 | +++ | +++ | ++ | +++ | +++ | ++ | ++++ | +++ | ++++ | ++++ | ++++ | ++++ |
| | 99 | +++ | ++++ | ++ | +++ | +++ | +++ | ++++ | ++++ | ++++ | ++++ | ++++ | ++++ |
| 59 | 106 | NA | ++ | ++++ | + | ++++ | ++ | NA | +++ | ++ | ++ | ++ | ++ |
| | 107 | ++ | +++ | ++ | +++ | +++ | ++ | +++ | ++ | ++ | +++ | ++ | +++ |
| | 108 | +++ | ++++ | ++ | ++ | ++ | ++ | +++ | ++ | +++ | +++ | ++++ | ++++ |
| | 109 | ++ | ++ | ++ | +++ | ++++ | ++++ | +++ | +++ | ++++ | ++ | ++ | ++ |
| | 110 | ++++ | NA | ++ | +++ | +++ | ++++ | +++ | NA | +++ | ++ | ++ | ++ |

¹“−” refers to 0%. “+” = 0%-7%; “++” = 7%-9%; “+++” = 9%-11%; “++++” = larger than 11%. For boarder values, each range includes the upper limit and not the lower limit. For example, 7%-9% means any value larger than 7% and no less than 9%. “NA” indicates sample not taken or unsuccessful sequencing.
²“−” refers to less than 6%; “+” = 6-19%; “++” = 19%-44%; “+++” = 44%-51%; “++++” = larger than 51%. For boarder values, each range includes the upper limit and not the lower limit. For example, 19%-44% means any value larger than 19% and no less than 44%. “NA” indicates sample not taken or unsuccessful sequencing

TABLE 137
Percentage of edited cells and indels 16 weeks after engraftment. The first column indicates the
sample number of the CD34+ cells edited as described in Example 6. Samples 58 and 59 were edited
with PEgRNA having the sequence of SEQ ID No. 5605 and ngRNA having the sequence of SEQ ID No. 584.

| | | % Indels¹ | | | | | | % Editing² | | | | | |
|---|---|---|---|---|---|---|---|---|---|---|---|---|---|
| Sample | Mouse | Bulk | Bulk | | | | | Bulk | Bulk | | | | |
| No. | # | BM | Spleen | CD19 | CD33 | CD34 | Erythroid | BM | Spleen | CD19 | CD33 | CD34 | Erythroid |
| Mock | 90 | + | + | NA | NA | NA | NA | − | − | NA | NA | NA | NA |
| | 91 | + | + | NA | NA | NA | NA | − | − | NA | NA | NA | NA |
| | 92 | + | + | NA | NA | NA | NA | − | − | NA | NA | NA | NA |
| | 93 | + | + | NA | NA | NA | NA | − | − | NA | NA | NA | NA |
| | 94 | + | + | NA | NA | NA | NA | − | − | NA | NA | NA | NA |
| 58 | 101 | +++ | ++ | ++++ | ++++ | +++ | +++ | ++ | ++++ | + | ++ | + | ++++ |
| | 102 | +++ | +++ | ++++ | ++++ | ++++ | ++++ | ++++ | +++ | ++++ | +++ | ++++ | ++ |
| | 103 | ++++ | +++ | ++++ | ++++ | ++++ | ++++ | +++ | +++ | ++ | ++ | +++ | ++ |
| | 104 | ++ | +++ | +++ | +++ | + | +++ | ++++ | ++++ | ++++ | ++++ | ++++ | ++++ |
| | 105 | ++ | +++ | ++ | ++ | + | +++ | +++ | ++++ | ++++ | +++ | ++ | ++ |
| 59 | 100 | +++ | + | + | +++ | + | ++ | + | ++ | +++ | ++ | + | + |
| | 111 | ++++ | ++ | +++ | +++ | ++ | ++++ | + | ++ | ++ | + | + | + |
| | 112 | + | +++ | ++ | +++ | ++ | +++ | ++ | ++ | + | + | + | + |
| | 113 | ++++ | ++ | ++ | +++ | ++++ | +++ | +++ | +++ | ++++ | ++++ | ++++ | +++ |
| | 114 | +++ | ++ | +++ | ++ | + | ++ | + | +++ | + | ++ | + | + |

¹“−” refers to 0%. “+” = 0%-5%; “++” = 5%-6%; “+++” = 6%-8%; “++++” = larger than 8%. For boarder values, each range includes the upper limit and not the lower limit. For example, 5%-6% means any value larger than 5% and no less than 6%. “NA” indicates sample not taken.
²“−” refers to less than 1%; “+” = 1-39%; “++” = 39%-42%; “+++” = 42%-45%; “++++” = larger than 45%. For boarder values, each range includes the upper limit and not the lower limit. For example, 42%-45% means any value larger than 42% and no less than 45%. “NA” indicates sample not taken.

TABLE 138
Percentage of edited cells and indels 8 weeks after engraftment. The first
column indicates the sample number of the CD34+ cells edited as described
in Example 6. Samples 97-99 were edited with PEgRNA having the sequence of
SEQ ID No. 19543 and ngRNA having the sequence of SEQ ID No. 883.

| Sample | Mouse | % Indels¹ | | | | | | % Editing² | | | | | |
|---|---|---|---|---|---|---|---|---|---|---|---|---|---|
| No. | # | Spleen | BM | CD19 | CD33 | CD34 | GlyA | Spleen | BM | GlyA | CD33 | CD19 | CD34 |
| Mock | 145 | + | NA | NA | NA | NA | ++++ | − | − | NA | NA | NA | NA |
| | 146 | + | NA | NA | NA | NA | ++++ | − | − | NA | NA | NA | NA |
| | 147 | + | NA | NA | NA | NA | ++++ | − | − | NA | NA | NA | NA |
| | 148 | + | NA | NA | NA | NA | ++++ | − | − | NA | NA | NA | NA |
| | 149 | + | NA | NA | NA | NA | ++++ | − | − | NA | NA | NA | NA |
| 97 | 165 | ++ | ++ | + | ++ | ++ | + | +++ | +++ | +++ | ++ | ++ | +++ |
| | 166 | ++ | ++ | ++ | ++ | ++ | ++ | +++ | ++ | ++ | +++ | +++ | +++ |
| | 167 | ++ | ++ | ++ | +++ | +++ | ++ | +++ | +++ | +++ | +++ | +++ | +++ |
| | 168 | ++ | ++ | ++ | ++ | +++ | ++ | +++ | +++ | +++ | ++ | +++ | +++ |
| | 169 | ++ | ++ | ++ | ++ | + | ++ | +++ | +++ | +++ | ++ | +++ | ++ |
| 98 | 175 | ++ | +++ | +++ | ++ | +++ | ++ | ++++ | ++++ | ++++ | ++++ | ++++ | ++++ |
| | 176 | ++ | +++ | +++ | +++ | +++ | ++ | ++++ | ++++ | ++++ | ++++ | ++++ | ++++ |
| | 177 | ++ | ++ | ++ | ++ | + | ++ | ++++ | ++++ | ++++ | ++++ | ++++ | ++++ |
| | 178 | +++ | +++ | +++ | +++ | +++ | ++ | ++++ | ++++ | ++++ | ++++ | ++++ | ++++ |
| | 179 | ++ | ++ | ++ | ++ | ++ | +++ | ++++ | ++++ | ++++ | ++++ | ++++ | ++++ |
| 99 | 185 | ++ | + | + | ++ | + | + | + | + | + | + | + | + |
| | 186 | ++ | + | + | + | + | + | + | + | + | + | + | + |
| | 187 | + | ++ | ++ | ++ | ++ | + | + | + | + | + | + | + |
| | 188 | ++ | ++ | ++ | ++ | ++ | ++ | + | + | + | + | + | + |
| | 189 | + | + | + | ++ | + | + | + | + | + | + | + | + |

¹“−” refers to 0%. “+” = 0%-1%; “++” = 1%-2%; “+++” = 2%-6%; “++++” = larger than 6%. For boarder values, each range includes the upper limit and not the lower limit. For example, 1%-2% means any value larger than 1% and no less than 2%. “NA” indicates sample not taken.
²“−” refers to less than 1%; “+” = 1-35%; “++” = 35%-44%; “+++” = 44%-52%; “++++” = larger than 52%. For boarder values, each range includes the upper limit and not the lower limit. For example, 44%-52% means any value larger than 44% and no less than 52%. “NA” indicates sample not taken.

Clonal analysis was also performed with bone marrow collected from mice engrafted as shown in Tables 132-137 above. After Bone marrow was collected and the percentage of CD34+ cells was determined by flow cytometry. After plating on methocult (Stem Cell Technologies, H4435) and incubating 14 days, DNA was extracted for sequencing analysis. For bone marrow collected from mice engrafted with all samples, most showed about 50% or more clones having the correct edit, with indel frequency between 7%-30%. The percentage of clones carrying desired edit indicate that the prime editing efficiency reflects editing throughout the population of cells.

### Example 9: Clonal Assessment for Detection of Large Deletions Between NCF1 and its Pseudogenes

Because the NCF1 gene is flanked by two pseudogenes that have highly similar sequences as NCF1 (>99% sequence identity), genome editing of NCF1, NCF1B, and/or NCF1C may result in large deletion events of the regions between NCF1 and NCF1B, between NCF1 and NCF1C, or the entire region between NCF1B and NCF1C. As shown in FIG. 4A, deletion event between NCF1B and NCF1, between NCF1 and NCF1C, and between NCF1B and NCF1C would result in about 1.5 Mb, about 0.4 Mb, and about 2 Mb of deletion, respectively.

To assess any large scale deletion or alteration of sequences between of sequences between NCF1 and NCF1B/NCF1C pseudogenes that may result from contacting the cells with Prime Editing reagents, edited CD34+ cells of sample No.s 133-134, 40-42, and 87-89 as described in Example 6 were examined. Following electroporation, cells were plated in methocult at 500 cells per well and incubated for 14 days for clones to grow. Following incubation, clones were picked into 15 μL of Quick extract buffer to extract genomic DNA. Digital droplet PCR (ddPCR) was then used to analyze the genomic DNA and determine the presence of large deletions between the proposed Peg/Nick guide target sites. Two probes were designed to bind either between NCF1B and NCF1 (FAM) or NCF1 and NCF1C (HEX). A reference probe (Cy5) serves as a reference for unedited region as shown in FIG. 4B. The detected ratios of each probe were then used to determine the presence or loss of the regions and assess the deletion events post editing. In case of a deletion event, the binding sequence for FAM and/or HEX would be lost, leading to a change in ratios of FAM and/or HEX reads in comparison to Cy5 reads. The three possible outcomes are loss of region between NCF1B and NCF1 (referred to as % LD1-detected by loss of FAM), NCF1 and NCF1C (referred to as % LD2—detected by loss of HEX) and Loss of NCF1B-NCF1C (referred to as % LD1 and LD2 detected by loss of FAM and HEX).

The results are summarized in Table 139 below, where “% LD1” indicates deletion frequency between NCF1B and NCF1, “% LD2” indicates deletion frequency between NCF1 and NCF1C, and “% LD1 and LD2” indicates deletion frequency between the NCF1B-NCF1C region. The results demonstrate low to non-detectable frequency of large deletion between the genes examined. The first column refers to the Sample numbers of CD34+ cells used in the analysis, which were edited as described in Example 6.

TABLE 139
Analysis of large deletion events at Prime Editing site.
% LD1, % LD2, and % LD1 and 2 are annotated as follows:
“−” = 0; “+” = 0 to 5%;
“++” = 5% to 7.5%. Each range includes
the upper end and not the lower end. For example, 5%-7.5%
means larger than 5% and no less than 7.5%.

| Sample No. | total clones tested | % LD1 | % LD2 | % LD1 and 2 |
|---|---|---|---|---|
| 133 | 66 | − | − | − |
| 134 | 126 | + | + | − |
| Mock | 8 | − | − | − |
| 40 | 78 | + | + | − |
| 41 | 78 | ++ | + | − |
| 42 | 78 | + | − | − |
| Mock | 18 | − | − | − |
| 40 | 49 | − | − | − |
| 41 | 59 | ++ | − | − |
| 42 | 41 | + | − | − |
| Mock | 9 | − | − | − |
| 87 | 52 | − | − | − |
| 88 | 32 | ++ | − | − |
| 89 | 28 | + | − | − |
| 87 | 63 | + | − | − |
| 88 | 56 | − | − | − |
| 89 | 52 | − | − | − |

### Example 10: Prime Editing with PEgRNAs Harboring Additional PAM Silencing Edits

CD34+ cells obtained from ΔGT CGD patients were electroporated and allowed for recovery ias described in Example 6. Following recovery, cells from samples Mock1, 133, 134, and Mock2 (as described in Table 130, Example 6) were transferred to myeloid media and maintained in culture for 14 days, with feeds every 3-4 days, to drive differentiation from CD34+ to CD13+ neutrophils.

At day 14 of myeloid culture, cells were assessed by flow cytometry for CD13+ expression to determine successful differentiation. The cells were also assessed for p47 protein expression: the cells were permeabilized, and pre-conjugated anti-human p47 antibody were added to detect p47 protein. p47 protein expression level was then assessed by flow cytometry.

Restoration of NADPH complex functionality was also assessed via two independent methods:

Reduction of Nitroblue-Tetrazolium (NBT) to insoluble formazan: Following 14-days of culture in methocult, a solution containing NBT and Phorbol 12-myristate 13-acetate (PMA, to active ROS release from functioning cells) was added to cell culture. Reduction of NBT results in formazan, a dark blue compound. The reduction requires a properly functional NADPH complex. The NBT test was thus used to assess restored NADPH complex function in the myeloid cells. Percentage of cultured cells exhibiting NBT reduction as indicated by formazan was calculated to indicate restoration of NADPH function.

Dihydrorhodamine (DHR) Oxidation test: Oxidation of DHR results in a fluorescent compound, rhodamine 123. This oxidation also requires a functional NADPH complex containing the p47 protein. Oxidation rate of DHR in the myeloid cells were assessed by flow cytometry and used to determine restoration of NADPH function.

Mock treated (electroporation only, no correction) patient cells, Prime Edited patient cells and Healthy donor cells were all assessed. Results from the p47 protein expression and cell functionality analysis are summarized in Table 140. The results demonstrate that Prime Edited CD34+ cells are able to successfully differentiate, and that Prime Editing can restore expression of p47 protein as well as functionality of NADPH complex in patient derived cells.

TABLE 140

| | | | | | p47 |
|---|---|---|---|---|---|
| | | % NBT | | % DHR | expression |
| Sample | Cell | reduction | CD13+ | oxidation | (% positive |
| No. | Genotype | observed ¹ | cells² | observed³ | by flow)⁴ |
| Mock1 | ΔGT patient | − | ++++ | − | − |
| 133 | ΔGT patient | + | ++++ | ++++ | ++ |
| 134 | ΔGT patient | +++ | ++++ | NA | ++++ |
| Mock2 | Healthy | +++ | ++++ | ++++ | +++ |
| | donor | | | | |

¹ “−”: less than 5%; “+”: 5%-15%; “++”: 15%-25%; “+++”: 25%-35%; “++++”: larger than 35%.
²“+”: 5%-25%; “++”: 25%-50%; “+++”: 50%-75%; “++++”: larger than 75%.
³“−”: less than 2%; “+”: 2%-25%; “++”: 25%-50%; “+++”: 50%-75%; “++++”: larger than 75%.
⁴“−”: less than 1%; “+”: 1%-15%; “++”: 15%-25%; “+++”: 25%-35%; “++++”: larger than 35%.
All ranges in 1-4 above include the upper end and not the lower end. For example, 5%-7.5% means larger than 5% and no less than 7.5%.

### Example 11: Restoration of p47 Protein Expression in ΔGT CGD Patient and Non-ΔGT CGD Patient Derived Lymphoblastoid Cell Lines (LCLs)

LCLs derived from a healthy donor, a ΔGT CGD patient, and a CGD patient that has a disease associated mutation in NCF1 other than ΔGT (a non-ΔGT CGD patient) were contacted with a mRNA encoding a Prime Editor fusion protein, the PEgRNA having the sequence of SEQ ID NO. 5634 and the ngRNA having the sequence of SEQ ID NO.595. Particularly, the LCLs were electroporated in the same way for CD34+ cells as described in Example 6, with the exception that Protector RNAse inhibitor (Sigma) was added to each cartridge at 1:50 volumetric ratio prior to electroporation. 72 hours post electroporation, cells were analyzed by flow cytometry for p47 protein expression as described in Example 10. Editing efficiency of NCF1 gene and/or NCF1B/NCF1C pseudogenes in healthy (or non-ΔGT CGD patient) derived cells were calculated in the same way as for CD34+ cells derived from healthy donors, and editing ΔGT CGD patient derived cells were calculated in the same way as for CD34+ cells from ΔGT CGD patient as described in Example 6. The results are summarized in Table 141 below. Successful editing and restoration of p47 expression by Prime Editing was observed. In addition, in cells derived from the non-ΔGT patient, the NCF1 gene contains a disease associated mutation that is different from the c.73_74 delGT deletion, thus correction of the delGT mutation in NCF1 would not effectively restore expression of p47. The results observed in cells derived from the non-ΔGT patient therefore demonstrate successful editing of NCF1B and/or NCF1C pseudogene, and that prime editing of NCF1B/NCF1C can restore expression of p47.

TABLE 141
Restoration of p47 expression in LCL cells.

| | PEgRNA | | | p47 |
|---|---|---|---|---|
| | SEQ ID/ | | | expression |
| | ngRNA | % | % | (% positive |
| Cell Genotype | SEQ ID | indel ¹ | correction ² | by flow)³ |
| Healthy donor | Mock | + | − | +++ |
| Healthy donor | 5634/595 | + | +++ | ++++ |
| non-dGT | Mock | + | − | + |
| patient | | | | |
| non-dGT | 5634/595 | ++ | +++ | +++ |
| patient | | | | |
| dGT patient | Mock | + | − | − |
| dGT patient | 5634/595 | ++++ | +++ | ++ |

¹ “−”: 0; “+”: 0%-2%; “++”: 2%-4%; “+++”: 4%-6%; “++++”: larger than 6%.
² “−”: less than 1%; “+”: 1%-5%; “++”: 5%-10%; “+++”: 10%-20%; “++++”: larger than 20%.
³“−”: less than 1%; “+”: 1%-20%; “++”: 20%-40%; “+++”: 40%-60%; “++++”: larger than 60%.
All ranges in 1-4 above include the upper end and not the lower end. For example, 1%-2% means larger than 1% and no less than 2%.

LENGTHY TABLES
The patent application contains a lengthy table section. A copy of the table is available in electronic form from the USPTO web site (https://seqdata.uspto.gov/?pageRequest=docDetail&DocID=US20250243474A1). An electronic copy of the table will also be available from the USPTO upon request and payment of the fee set forth in 37 CFR 1.19(b)(3).

1.-139. (canceled)

140. A prime editing guide RNA (PEgRNA) comprising
a) a spacer that is complementary to a search target sequence on a first strand of a NCF1 gene, a NCF1b pseudogene, or a NCF1C pseudogene,
b) a guide RNA (gRNA) core capable of binding to a Cas9 protein to generate a nick site in a second strand of the NCF1 gene, the NCF1B pseudogene, or the NCF1C pseudogene, and
c) an extension arm comprising:
(i) an editing template that encodes a wildtype NCF1 gene sequence and comprises a region of complementarity to an editing target sequence downstream of the nick site in the second strand of the NCF1 gene, the NCF1B pseudogene, or the NCF1C pseudogene, and
(ii) a primer binding site (PBS) that is complementary to a region upstream of the nick site in the second strand of the NCF1 gene, the NCF1B pseudogene, or the NCF1C pseudogene.

141. The PEgRNA of claim 140, wherein the spacer comprises at its 3′ end a sequence selected from the group consisting of SEQ ID NOs: 1, 609, 907, 1064, 1117, 1238, 1463, 1827, 2146, 3298, 3595, 3995, 5723, 5816, 7499, 9612, 9861, 10268, 10389-10395, 10445, 10541, 10834, 11101, 11701, 12105, 13145, 13985, 14374, 14571, 14788, 14901, 14986, 15147, 15412, 16078, 17768, 17897, 19077, 19568, 21702, 21746, 21945, 22197, 22502, 22863, 22962, 23223, 24034, 24237, 24602, 25002, 25166, 25253, 25652, 26218, 27001, 27249, 27354, 27470, 27641, 27948, 27994, 28580, 28712, 28933, 29997, 30861, 31062, 31330, 31697, 32042, 32460, 32739, 33063, 33238, and 34021.

142. The PEgRNA of claim 141, wherein the spacer comprises a sequence selected from the group consisting of SEQ ID NOs: 19077-19083, and the PBS comprises at its 5′ end a sequence selected from the group consisting of GGAAAGCC, GGAAAGCCA, and SEQ ID NOs: 19090-19094.

143. The PEgRNA of claim 142, wherein the editing template comprises at its 3′ end a sequence selected from the group consisting of SEQ ID NOs: 19116, 19123, 19126, and 19129.

144. The PEgRNA of claim 142, wherein the PEgRNA comprises, from 5′ to 3′: i) a spacer consisting of SEQ ID NO: 19081, ii) a PBS consisting of SEQ ID NO: 19093, and iii) an editing template consisting of SEQ ID NO: 19129.

145. The PEgRNA of claim 144, wherein the PEgRNA comprises SEQ ID NO: 19543 or 19562.

146. A prime editing system comprising the PEgRNA of claim 140 or one or more polynucleotides encoding the PEgRNA and further comprising:
(A) a nick guide RNA (ngRNA) or one or more polynucleotides encoding the ngRNA, and
(B) a Prime Editor comprising a Cas9 nickase and a reverse transcriptase, or one or more polynucleotides encoding the Prime Editor,
wherein the ngRNA comprises: a ngRNA spacer comprising at its 3′ end nucleotides 4-20 of a sequence selected from the group consisting of SEQ ID NOs: 849, 833, and 770 and a ngRNA core capable of binding to a Cas9 protein.

147. The prime editing system of claim 146, wherein the ngRNA comprises SEQ ID NO: 883.

148. A method of generating modified human cells in a population of human cells comprising a NCF1 gene, a NCF1B pseudogene, and a NCF1C pseudogene, the method comprising: contacting the population of human cells with
a) a Prime Editing guide RNA (PEgRNA) or one or more polynucleotides encoding the PEgRNA, wherein the PEgRNA comprises:
(i) a spacer that is complementary to a search target sequence on a first strand of the NCF1 gene, the NCF1B pseudogene, or the NCF1C pseudogene,
(ii) a guide RNA (gRNA) core capable of binding to a Cas9 protein to generate a nick site in a second strand of the NCF1 gene, the NCF1B pseudogene, or the NCF1C pseudogene, and
(iii) an extension arm comprising:
(A) an editing template that encodes a wildtype NCF1 gene sequence and comprises a region of complementarity to an editing target sequence downstream of the nick site in the second strand of the NCF1 gene, the NCF1 pseudogene, or the NCF1C pseudogene,
and
(B) a primer binding site (PBS) that is complementary to a region upstream of the nick site in the second strand of the NCF1 gene, the NCF1B pseudogene, or the NCF1C pseudogene;
and
b) a Prime Editor comprising a Cas9 nickase and a reverse transcriptase, or one or more polynucleotides encoding the Prime Editor,
wherein, in the population of human cells, the modified human cells are edited in the NCF1 gene, the NCF1B pseudogene, and/or the NCF1C pseudogene.

149. The method of claim 148, wherein the spacer comprises a sequence selected from the group consisting of SEQ ID NOs: 1, 609, 907, 1064, 1117, 1238, 1463, 1827, 2146, 3298, 3595, 3995, 5723, 5816, 7499, 9612, 9861, 10268, 10389-10395, 10445, 10541, 10834, 11101, 11701, 12105, 13145, 13985, 14374, 14571, 14788, 14901, 14986, 15147, 15412, 16078, 17768, 17897, 19077, 19568, 21702, 21746, 21945, 22197, 22502, 22863, 22962, 23223, 24034, 24237, 24602, 25002, 25166, 25253, 25652, 26218, 27001, 27249, 27354, 27470, 27641, 27948, 27994, 28580, 28712, 28933, 29997, 30861, 31062, 31330, 31697, 32042, 32460, 32739, 33063, 33238, and 34021.

150. The method of claim 149, wherein the spacer comprises a sequence selected from the group consisting of SEQ ID NOs: 19077-19083, and wherein the PBS comprises at its 5′ end a sequence selected from the group consisting of GGAAAGCC, GGAAAGCCA, and SEQ ID NOs: 19090-19094.

151. The method of claim 150, wherein the editing template comprises at its 3′ end a sequence selected from the group consisting of SEQ ID NOs: 19116, 19123, 19126, and 19129.

152. The method of claim 151, wherein the PEgRNA comprises from 5′ to 3′ i) a spacer consisting of SEQ ID NO: 19081, ii) a PBS consisting of SEQ ID NO: 19093, iii) an editing template consisting of SEQ ID NO: 19129.

153. The method of claim 152, wherein the PEgRNA comprises SEQ ID NO: 19543 or 19562.

154. The method of claim 148, further comprising contacting the population of human cells with a nick guide RNA (ngRNA) or one or more polynucleotides encoding the ngRNA, wherein the ngRNA comprises: an ngRNA spacer that comprises a region of complementarity to the second strand of the NCF1 gene, the NCF1B pseudogene, or the NCF1C pseudogene, and a ngRNA core capable of binding to a Cas9 protein.

155. The method of claim 154, wherein the ngRNA spacer comprises at its 3′ end nucleotides 4-20 of any one of SEQ ID NOs. 849, 833, or 770.

156. The method of claim 154, wherein the ngRNA spacer comprises the sequence of SEQ ID NO: 833.

157. The method of claim 155, wherein the ngRNA comprises SEQ ID NO: 883.

158. The method of claim 156, wherein the PEgRNA and/or the ngRNA comprises 3′ mN*mN*mN*N and 5′mN*mN*mN* modifications, where m indicates that the nucleotide contains a 2′-O-Me modification and a * indicates the presence of a phosphorothioate bond.

159. A method of treating a patient with Chronic Granulomatous Disease, the method comprising
(a) modifying a human cell by editing a NCF1 gene, a NCF1B pseudogene, and/or a NCF1C pseudogene in the human cell, and
(b) administering the human cell to the patient,
wherein step (a) is performed by the method of claim 148.
